(19) United States
(12) Patent Application Publication
(10) Pub. No.: US 2024/0132894 A1
(43) Pub. Date: Apr. 25, 2024

(54) OLIGONUCLEOTIDE COMPOSITIONS AND METHODS OF USE THEREOF

(71) Applicant: WAVE LIFE SCIENCES LTD., Singapore (SG)

(72) Inventors: Chandra Vargeese, Schwenksville, PA (US); Naoki Iwamoto, Boston, MA (US); David Charles Donnell Butler, Medford, MA (US); Subramanian Marappan, Acton, MA (US); Genliang Lu, Winchester, MA (US); Jason Jingxin Zhang, Walpole, MA (US); Vinod Vathipadiekal, Stoneham, MA (US); Maria David Frank-Kamenetsky, Brookline, MA (US); Luciano Henrique Apponi, Lynn, MA (US); Young Jin Cho, Belmont, MA (US)

(21) Appl. No.: 17/956,741

(22) Filed: Sep. 29, 2022

Related U.S. Application Data
(62) Division of application No. 16/618,010, filed on Nov. 27, 2019, now Pat. No. 11,603,532.
(60) Provisional application No. 62/670,686, filed on May 11, 2018.
(60) Provisional application No. 62/670,709, filed on May 11, 2018.
(60) Provisional application No. 62/656,949, filed on Apr. 12, 2018.
(60) Provisional application No. 62/514,769, filed on Jun. 2, 2017.
(60) Provisional application No. 62/514,771, filed on Jun. 2, 2017.

(51) Int. Cl. C12N 15/113; A61K 31/712

(52) U.S. Cl. CPC C12N 15/1137; A61K 31/712; C12N 2310/14; C12N 2310/315; C12N 2310/343

(57) ABSTRACT

Among other things, the present disclosure provides designed oligonucleotides, compositions, and methods thereof. In some embodiments, provided oligonucleotide compositions provide improved single-stranded RNA interference and/or RNase H-mediated knockdown. Among other things, the present disclosure encompasses the recognition that structural elements of oligonucleotides, such as base sequence, chemical modifications (e.g., modifications of sugar, base, and/or internucleotidic linkages) or patterns thereof, conjugation with additional chemical moieties, and/or stereochemistry [e.g., stereochemistry of backbone chiral centers (chiral internucleotidic linkages)], and/or patterns thereof, can have significant impact on oligonucleotide properties and activities, e.g., RNA interference (RNAi) activity, stability, delivery, etc. In some embodiments, the present disclosure provides methods for treatment of diseases using provided oligonucleotide compositions, for example, in RNA interference and/or RNase H-mediated knockdown.

### CROSS-REFERENCE TO RELATED APPLICATIONS

This application is a divisional of U.S. application Ser. No. 17/956,741, filed Nov. 27, 2019, which issued as U.S. Pat. No. 11,603,532 on Mar. 14, 2023, which is a U.S. National Stage Application of International PCT Application No. PCT/US18/35687, which claims priority to United States Provisional Application Nos. 62/514,769, filed Jun. 2, 2017, 62/514,771, filed Jun. 2, 2017, 62/656,949, filed Apr. 12, 2018, 62/670,686, filed May 11, 2018, and 62/670,709, filed May 11, 2018, the entirety of each of which is incorporated herein by reference.

### SEQUENCE LISTING

The instant application contains a Sequence Listing which has been submitted electronically in XML format and is hereby incorporated by reference in its entirety. Said XML copy, created on Jun. 1, 2023, is named “2010581-1154.xml” and is 7,888,651 bytes in size.

### BACKGROUND

Oligonucleotides which target a gene are useful in various applications, e.g., therapeutic, diagnostic, research and nanomaterials applications. The use of naturally-occurring nucleic acids (e.g., unmodified DNA or RNA) can be limited, for example, by their susceptibility to endo- and exo-nucleases.

### SUMMARY

Among other things, the present disclosure encompasses the recognition that controlling structural elements of oligonucleotides, such as chemical modifications (e.g., modifications of a sugar, base and/or internucleotidic linkage) or patterns thereof, alterations in stereochemistry (e.g., stereochemistry of a backbone chiral internucleotidic linkage) or patterns thereof, and/or conjugation with an additional chemical moiety (e.g., a lipid moiety, a targeting moiety, carbohydrate moiety, a moiety that binds to a asialoglycoprotein receptor or ASGPR, e.g., a GalNAc moiety, etc.) can have a significant impact on oligonucleotide properties and/or activities. In some embodiments, the properties and/or activities include, but are not limited to, participation in, direction of a decrease in expression, activity or level of a gene or a gene product thereof, mediated, for example, by RNA interference (RNAi interference), single-stranded RNA interference (ssRNAi), RNase H-mediated knockdown, steric hindrance of translation, etc.

In some embodiments, the present disclosure demonstrates that compositions comprising oligonucleotides (and particularly single-stranded oligonucleotides) with controlled structural elements provide unexpected properties and/or activities.

In some embodiments, the present disclosure encompasses the recognition that stereochemistry, particularly stereochemistry of backbone chiral centers, can unexpectedly improve properties of oligonucleotides. In contrast to many prior observations that some structural elements that increase stability can also lower activity, for example, RNA interference, the present disclosure demonstrates that control of stereochemistry can, surprisingly, increase stability while not significantly decreasing activity.

In some embodiments, the present disclosure provides technologies (e.g., compounds, methods, etc.) for improving oligonucleotide stability while maintaining or increasing activity, including compositions of improved-stability oligonucleotides.

In some embodiments, the present disclosure provides oligonucleotides having certain 5′-end structures.

Among other things, the present disclosure demonstrates such oligonucleotides can have desired properties.

In some embodiments, the present disclosure provides 5′-end structures that, when used in accordance with the present disclosure, can provided oligonucleotides with high biological activities, e.g., RNAi activity.

Literature has reported that, in many cases, RNAi activity requires the presence of a 5′-phosphate (or modified phosphate) moiety; in some embodiments, the present disclosure demonstrates that, surprisingly, an oligonucleotide with an unmodified 5′-end (i.e., with a 5′-OH), can achieve comparable RNAi activity to an otherwise identical oligonucleotide comprising a 5′-phosphate (or modified phosphate) moiety. Thus, among other things, the present disclosure, in some embodiments, provides oligonucleotides whose sequence is directed to an RNAi target site, which oligonucleotides may include one or more other structural features, as described herein and/or otherwise known in the art, that are useful (or are not detrimental) for RNAi activity, wherein the oligonucleotides have a 5′-OH moiety.

In some embodiments, the present disclosure encompasses the recognition that various additional chemical moieties, such as lipid moieties and/or carbohydrate moieties, when incorporated into oligonucleotides, can improve one or more oligonucleotide properties, such as knock down of the target gene or a gene product thereof. In some embodiments, an additional chemical moiety is optional. In some embodiments, an oligonucleotide can comprise more than one additional chemical moiety. In some embodiments, an oligonucleotide can comprise two or more additional chemical moieties, wherein the additional chemical moieties are identical or non-identical, or of the same category (e.g., targeting moiety, carbohydrate moiety, a moiety that binds to ASPGR, lipid moiety, etc.) or not of the same category. In some embodiments, certain additional chemical moieties facilitate delivery of oligonucleotides to desired cells, tissues and/or organs. In some embodiments, certain additional chemical moieties facilitate internalization of oligonucleotides and/or increase oligonucleotide stability.

In some embodiments, the present disclosure provides technologies for incorporating various additional chemical moieties into oligonucleotides. In some embodiments, the present disclosure provides, for example, reagents and methods for introducing additional chemical moieties through nucleobases (e.g., by covalent linkage, optionally via a linker, to a site on a nucleobase).

In some embodiments, the present disclosure demonstrates that surprisingly high target specificity can be achieved with oligonucleotides whose structures include one or more features as described herein.

In some embodiments, the present disclosure provides technologies, e.g., oligonucleotide compositions and methods thereof, that achieve allele-specific suppression, wherein transcripts from one allele of a particular target gene is selectively knocked down relative to at least one other allele of the same gene.

Among other things, the present disclosure provides structural elements, technologies and/or features that can be incorporated into oligonucleotides and can impart or tune one or more properties thereof (e.g., relative to an otherwise identical oligonucleotide lacking the relevant technology or feature). In some embodiments, the present disclosure documents that one or more provided technologies and/or features can usefully be incorporated into oligonucleotide(s) of various sequences.

In some embodiments, the present disclosure demonstrates that certain provided structural elements, technologies and/or features are particularly useful for oligonucleotides that participate in and/or direct RNAi mechanisms (e.g., RNAi agents). Regardless, however, the teachings of the present disclosure are not limited to oligonucleotides that participate in or operate via any particular mechanism. In some embodiments, the present disclosure pertains to any oligonucleotide, useful for any purpose, which operates through any mechanism, and which comprises any sequence, structure or format (or portion thereof) described herein. In some embodiments, the present disclosure provides an oligonucleotide, useful for any purpose, which operates through any mechanism, and which comprises any sequence, structure or format (or portion thereof) described herein, including, but not limited to, any 5′-end structure; 5′-end region; a first region (including but not limited to, a seed region); a second region (including, but not limited to, a post-seed region); and a 3′-end region (which can be a 3′-terminal dinucleotide and/or a 3′-end cap); an optional additional chemical moiety (including but not limited to a targeting moiety, a carbohydrate moiety, a moiety that binds APGR, and a lipid moiety); stereochemistry or patterns of stereochemistry; modification or pattern of modification; internucleotidic linkage or pattern of internucleotidic linkages; modification of sugar(s) or pattern of modifications of sugars; modification of base(s) or patterns of modifications of bases. In some embodiments, provided oligonucleotides may participate in (e.g., direct) RNAi mechanisms. In some embodiments, provided oligonucleotides may participate in RNase H (ribonuclease H) mechanisms. In some embodiments, provided oligonucleotides may act as translational inhibitors (e.g., may provide steric blocks of translation).

In some embodiments, provided oligonucleotides may participate in exon skipping mechanisms. In some embodiments, provided oligonucleotides may be aptamers. In some embodiments, provided oligonucleotides may bind to and inhibit the function of a protein, small molecule, nucleic acid or cell. In some embodiments, provided oligonucleotides may participate in forming a triplex helix with a double-stranded nucleic acid in the cell. In some embodiments, provided oligonucleotides may bind to genomic (e.g., chromosomal) nucleic acid. In some embodiments, provided oligonucleotides may bind to genomic (e.g., chromosomal) nucleic acid, thus preventing or decreasing expression of the nucleic acid (e.g., by preventing or decreasing transcription, transcriptional enhancement, modification, etc.). In some embodiments, provided oligonucleotides may bind to DNA quadruplexes. In some embodiments, provided oligonucleotides may be immunomodulatory. In some embodiments, provided oligonucleotides may be immunostimulatory. In some embodiments, provided oligonucleotides may be immunostimulatory and may comprise a CpG sequence. In some embodiments, provided oligonucleotides may be immunostimulatory and may comprise a CpG sequence and may be useful as an adjuvant. In some embodiments, provided oligonucleotides may be immunostimulatory and may comprise a CpG sequence and may be useful as an adjuvant in treating a disease (e.g., an infectious disease or cancer). In some embodiments, provided oligonucleotides may be therapeutic. In some embodiments, provided oligonucleotides may be non-therapeutic. In some embodiments, provided oligonucleotides may be therapeutic or non-therapeutic. In some embodiments, provided oligonucleotides are useful in therapeutic, diagnostic, research and/or nanomaterials applications. In some embodiments, provided oligonucleotides may be useful for experimental purposes. In some embodiments, provided oligonucleotides may be useful for experimental purposes, e.g., as a probe, in a microarray, etc. In some embodiments, provided oligonucleotides may participate in more than one biological mechanism; in certain such embodiments, for example, provided oligonucleotides may participate in both RNAi and RNase H mechanisms.

In some embodiments, provided oligonucleotides are directed to a target (e.g., a target sequence, a target RNA, a target mRNA, a target pre-mRNA, a target gene, etc.). A target gene is a gene with respect to which expression and/or activity of one or more gene products (e.g., RNA and/or protein products) are intended to be altered. In many embodiments, a target gene is intended to be inhibited. Thus, when an oligonucleotide as described herein acts on a particular target gene, presence and/or activity of one or more gene products of that gene are altered when the oligonucleotide is present as compared with when it is absent.

In some embodiments, a target is a specific allele with respect to which expression and/or activity of one or more products (e.g., RNA and/or protein products) are intended to be altered. In many embodiments, a target allele is one whose presence and/or expression is associated (e.g., correlated) with presence, incidence, and/or severity, of one or more diseases and/or conditions. Alternatively or additionally, in some embodiments, a target allele is one for which alteration of level and/or activity of one or more gene products correlates with improvement (e.g., delay of onset, reduction of severity, responsiveness to other therapy, etc) in one or more aspects of a disease and/or condition.

In some embodiments, where presence and/or activity of a particular allele (a disease-associated allele) is associated (e.g., correlated) with presence, incidence and/or severity of one or more disorders, diseases and/or conditions, a different allele of the same gene exists and is not so associated, or is associated to a lesser extent (e.g., shows less significant, or statistically insignificant correlation). In some such embodiments, oligonucleotides and methods thereof as described herein may preferentially or specifically target the associated allele relative to the one or more less-associated/unassociated allele(s), thus mediating allele-specific suppression.

In some embodiments, a target sequence is a sequence to which an oligonucleotide as described herein binds. In many embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In some embodiments, a target-binding sequence is an exact complement of a target sequence of a transcript (e.g., pre-mRNA, mRNA, etc.). A target-binding sequence/target sequence can be of various lengths to provided oligonucleotides with desired activities and/or properties. In some embodiments, a target binding sequence/target sequence comprises 5-50 (e.g., 10-40, 15-30, 15-25, 16-25, 17-25, 18-25, 19-25, 20-25, 10, 11, 12, 13, 14, 15, 16, 17, 18, 19, 20, 21, 22, 23, 24, 25, or more) bases. In some embodiments, a small number of differences/mismatches is tolerated between (a relevant portion of) an oligonucleotide and its target sequence, including but not limited to the 5′ and/or 3′-end regions of the target and/or oligonucleotide sequence. In many embodiments, a target sequence is present within a target gene. In many embodiments, a target sequence is present within a transcript (e.g., an mRNA and/or a pre-mRNA) produced from a target gene.

In some embodiments, a target sequence includes one or more allelic sites (i.e., positions within a target gene at which allelic variation occurs). In some embodiments, an allelic site is a mutation. In some embodiments, an allelic site is a SNP. In some such embodiments, a provided oligonucleotide binds to one allele preferentially or specifically relative to one or more other alleles. In some embodiments, a provided oligonucleotide binds preferentially to a disease-associated allele. For example, in some embodiments, an oligonucleotide (or a target-binding sequence portion thereof) provided herein has a sequence that is, fully or at least in part, identical to, or an exact complement of a particular allelic version of a target sequence.

In some embodiments, an oligonucleotide (or a target-binding sequence portion thereof) provided herein has a sequence that is identical to, or an exact complement of a target sequence comprising an allelic site, or an allelic site, of a disease-associated allele. In some embodiments, an oligonucleotide provided herein has a target binding sequence that is an exact complement of a target sequence comprising an allelic site of a transcript of an allele (in many embodiments, a disease-associated allele), wherein the allelic site is a mutation. In some embodiments, an oligonucleotide provided herein has a target binding sequence that is an exact complement of a target sequence comprising an allelic site of a transcript of an allele (in many embodiments, a disease-associated allele), wherein the allelic site is a SNP. In some embodiments, a sequence is any sequence disclosed herein.

Unless otherwise noted, all sequences (including, but not limited to base sequences and patterns of chemistry, modification, and/or stereochemistry) are presented in 5′ to 3′ order.

In some embodiments, the present disclosure provides compositions and methods related to an oligonucleotide which is specific to a target and which has any format, structural element or base sequence of any oligonucleotide disclosed herein.

In some embodiments, the present disclosure provides compositions and methods related to an oligonucleotide which is specific to a target and which has or comprises the base sequence of any oligonucleotide disclosed herein, or a region of at least 15 contiguous nucleotides of the base sequence of any oligonucleotide disclosed herein, wherein the first nucleotide of the base sequence or the first nucleotide of the at least 15 contiguous nucleotides can be optionally replaced by T or DNA T. In some embodiments, the oligonucleotide is capable of directing ssRNAi.

In some embodiments, the present disclosure provides compositions and methods for RNA interference directed by a single-stranded RNAi agent. In some embodiments, oligonucleotides of such compositions can have a format, structural element or base sequence of an oligonucleotide disclosed herein.

In some embodiments, the present disclosure provides compositions and methods for RNase H-mediated knockdown of a target gene RNA directed by an oligonucleotide (e.g., an antisense oligonucleotide).

Provided oligonucleotides and oligonucleotide compositions can have any format, structural element or base sequence of any oligonucleotide disclosed herein. In some embodiments, a structural element is a 5′-end structure, 5′-end region, 5′-nucleotide, seed region, post-seed region, 3′-end region, 3′-terminal dinucleotide, 3′-end cap, or any portion of any of these structures, GC content, long GC stretch, and/or any modification, chemistry, stereochemistry, pattern of modification, chemistry or stereochemistry, or a chemical moiety (e.g., including but not limited to, a targeting moiety, a lipid moiety, a GalNAc moiety, a carbohydrate moiety, etc.), any component, or any combination of any of the above.

In some embodiments, the present disclosure provides compositions and methods of use of an oligonucleotide.

In some embodiments, the present disclosure provides compositions and methods of use of an oligonucleotide which can direct both RNA interference and RNase H-mediated knockdown of a target gene RNA. In some embodiments, oligonucleotides of such compositions can have a format, structural element or base sequence of an oligonucleotide disclosed herein.

In some embodiments, an oligonucleotide directing a particular event or activity participates in the particular event or activity, e.g., a decrease in the expression, level or activity of a target gene or a gene product thereof. In some embodiments, an oligonucleotide is deemed to “direct” a particular event or activity when presence of the oligonucleotide in a system in which the event or activity can occur correlates with increased detectable incidence, frequency, intensity and/or level of the event or activity.

In some embodiments, a provided oligonucleotide comprises any one or more structural elements of an oligonucleotide as described herein, e.g., a base sequence (or a portion thereof of at least 15 contiguous bases); a pattern of internucleotidic linkages (or a portion thereof of at least 5 contiguous internucleotidic linkage); a pattern of stereochemistry of internucleotidic linkages (or a portion thereof of at least 5 contiguous internucleotidic linkages); a 5′-end structure; a 5′-end region; a first region; a second region; and a 3′-end region (which can be a 3′-terminal dinucleotide and/or a 3′-end cap); and an optional additional chemical moiety; and, in some embodiments, at least one structural element comprises a chirally controlled chiral center. In some embodiments, a 3′-terminal dinucleotide can comprise two total nucleotides. In some embodiments, an oligonucleotide further comprises a chemical moiety selected from, as non-limiting examples, a targeting moiety, a carbohydrate moiety, a GalNAc moiety, a lipid moiety, and any other chemical moiety described herein or known in the art. In some embodiments, a moiety that binds APGR is a moiety of GalNAc, or a variant, derivative or modified version thereof, as described herein and/or known in the art. In some embodiments, an oligonucleotide is a single-stranded RNAi agent. In some embodiments, a first region is a seed region. In some embodiments, a second region is a post-seed region.

In some embodiments, a provided oligonucleotide comprises any one or more structural elements of a single-stranded RNAi agent as described herein, e.g., a 5′-end structure; a 5′-end region; a seed region; a post-seed region (the region between the seed region and the 3′-end region); and a 3′-end region (which can be a 3′-terminal dinucleotide and/or a 3′-end cap); and an optional additional chemical moiety; and, in some embodiments, at least one structural element comprises a chirally controlled chiral center. In some embodiments, a 3′-terminal dinucleotide can comprise two total nucleotides. In some embodiments, an oligonucleotide further comprises a chemical moiety selected from, as non-limiting examples, a targeting moiety, a carbohydrate moiety, a GalNAc moiety, and a lipid moiety. In some embodiments, a moiety that binds APGR is any GalNAc, or variant, derivative or modification thereof, as described herein or known in the art.

In some embodiments, a provided oligonucleotide comprises any one or more structural elements of an oligonucleotide as described herein, e.g., a 5′-end structure, a 5′-end region, a first region, a second region, a 3′-end region, and an optional additional chemical moiety, wherein at least one structural element comprises a chirally controlled chiral center. In some embodiments, the oligonucleotide comprises a span of at least 5 total nucleotides without 2′-modifications. In some embodiments, the oligonucleotide further comprises an additional chemical moiety selected from, as non-limiting examples, a targeting moiety, a carbohydrate moiety, a GalNAc moiety, and a lipid moiety. In some embodiments, a provided oligonucleotide is capable of directing RNA interference. In some embodiments, a provided oligonucleotide is capable of directing RNase H-mediated knockdown. In some embodiments, a provided oligonucleotide is capable of directing both RNA interference and RNase H-mediated knockdown. In some embodiments, a first region is a seed region. In some embodiments, a second region is a post-seed region.

In some embodiments, a provided oligonucleotide comprises any one or more structural elements of a single-stranded RNAi agent, e.g., a 5′-end structure, a 5′-end region, a seed region, a post-seed region, and a 3′-end region and an optional additional chemical moiety, wherein at least one structural element comprises a chirally controlled chiral center; and, in some embodiments, the oligonucleotide is also capable of directing RNase H-mediated knockdown of a target gene RNA. In some embodiments, the oligonucleotide comprises a span of at least 5 total 2′-deoxy nucleotides. In some embodiments, the oligonucleotide further comprises a chemical moiety selected from, as non-limiting examples, a targeting moiety, a carbohydrate moiety, a GalNAc moiety, and a lipid moiety, and any other additional chemical moiety described herein. In some embodiments, the present disclosure demonstrates that oligonucleotide properties can be modulated through chemical modifications. In some embodiments, the present disclosure provides an oligonucleotide composition comprising a first plurality of oligonucleotides which have a common base sequence and comprise one or more internucleotidic linkage, sugar, and/or base modifications. In some embodiments, the present disclosure provides an oligonucleotide composition capable of directing single-stranded RNA interference and comprising a first plurality of oligonucleotides which have a common base sequence and comprise one or more internucleotidic linkage, and/or one or more sugar, and/or one or more base modifications. In some embodiments, an oligonucleotide or oligonucleotide composition is also capable of directing RNase H-mediated knockdown of a target gene RNA. In some embodiments, the present disclosure demonstrates that oligonucleotide properties, e.g., activities, toxicities, etc., can be modulated through chemical modifications of sugars, nucleobases, and/or internucleotidic linkages. In some embodiments, the present disclosure provides an oligonucleotide composition comprising a plurality of oligonucleotides which have a common base sequence, and comprise one or more modified internucleotidic linkages (or “non-natural internucleotidic linkages”, linkages that can be utilized in place of a natural phosphate internucleotidic linkage (—OP(O)(OH)O—, which may exist as a salt form (—OP(O)(O⁻)O—) at a physiological pH) found in natural DNA and RNA), one or more modified sugar moieties, and/or one or more natural phosphate linkages. In some embodiments, provided oligonucleotides may comprise two or more types of modified internucleotidic linkages. In some embodiments, a provided oligonucleotide comprises a non-negatively charged internucleotidic linkage. In some embodiments, a non-negatively charged internucleotidic linkage is a neutral internucleotidic linkage. In some embodiments, a neutral internucleotidic linkage comprises a triazole, alkyne, or cyclic guanidine moiety. Such moieties an optionally substituted. In some embodiments, a provided oligonucleotide comprises a neutral internucleotidic linkage and another internucleotidic linkage which is not a neutral backbone. In some embodiments, a provided oligonucleotide comprises a neutral internucleotidic linkage and a phosphorothioate internucleotidic linkage. In some embodiments, provided oligonucleotide compositions comprising a plurality of oligonucleotides are chirally controlled and level of the plurality of oligonucleotides in the composition is controlled or pre-determined, and oligonucleotides of the plurality share a common stereochemistry configuration at one or more chiral internucleotidic linkages. For example, in some embodiments, oligonucleotides of a plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp; in some embodiments, oligonucleotides of a plurality share a common stereochemistry configuration at each chiral internucleotidic linkages. In some embodiments, a chiral internucleotidic linkage where a controlled level of oligonucleotides of a composition share a common stereochemistry configuration (independently in the Rp or Sp configuration) is referred to as a chirally controlled internucleotidic linkage. In some embodiments, a modified internucleotidic linkage is a non-negatively charged (neutral or cationic) internucleotidic linkage in that at a pH, (e.g., human physiological pH (˜7.4), pH of a delivery site (e.g., an organelle, cell, tissue, organ, organism, etc.), etc.), it largely (e.g., at least 5%, 10%, 20%, 30%, 40%, 50%, 60%, 70%, 80%, 90%, etc.; in some embodiments, at least 30%; in some embodiments, at least 40%; in some embodiments, at least 50%; in some embodiments, at least 60%; in some embodiments, at least 70%; in some embodiments, at least 80%; in some embodiments, at least 90%; in some embodiments, at least 99%; etc.;) exists as a neutral or cationic form (as compared to an anionic form (e.g., —O—P(O)(O⁻)—O— (the anionic form of natural phosphate linkage), —O—P(O)(S⁻)—O— (the anionic form of phosphorothioate linkage), etc.)), respectively. In some embodiments, a modified internucleotidic linkage is a neutral internucleotidic linkage in that at a pH, it largely exists as a neutral form. In some embodiments, a modified internucleotidic linkage is a cationic internucleotidic linkage in that at a pH, it largely exists as a cationic form. In some embodiments, a pH is human physiological pH (˜7.4). In some embodiments, a modified internucleotidic linkage is a neutral internucleotidic linkage in that at pH 7.4 in a water solution, at least 90% of the internucleotidic linkage exists as its neutral form. In some embodiments, a modified internucleotidic linkage is a neutral internucleotidic linkage in that in a water solution of the oligonucleotide, at least 50%, 60%, 70%, 80%, 90%, 95%, or 99% of the internucleotidic linkage exists in its neutral form. In some embodiments, the percentage is at least 90%. In some embodiments, the percentage is at least 95%. In some embodiments, the percentage is at least 99%. In some embodiments, a non-negatively charged internucleotidic linkage, e.g., a neutral internucleotidic linkage, when in its neutral form has no moiety with a pKa that is less than 8, 9, 10, 11, 12, 13, or 14. In some embodiments, pKa of an internucleotidic linkage in the present disclosure can be represented by pKa of CH₃—the internucleotidic linkage-CH₃ (i.e., replacing the two nucleoside units connected by the internucleotidic linkage with two —CH₃ groups). Without wishing to be bound by any particular theory, in at least some cases, a neutral internucleotidic linkage in an oligonucleotide can provide improved properties and/or activities, e.g., improved delivery, improved resistance to exonucleases and endonucleases, improved cellular uptake, improved endosomal escape and/or improved nuclear uptake, etc., compared to a comparable nucleic acid which does not comprises a neutral internucleotidic linkage.

In some embodiments, a non-negatively charged internucleotidic linkage has the structure of e.g., of formula I-n-1, I-n-2, I-n-3, II, II-a-1, II-a-2, II-b-1, II-b-2, II-c-1, II-c-2, II-d-1, II-d-2, etc. In some embodiments, a non-negatively charged internucleotidic linkage comprises a triazole or alkyne moiety. In some embodiments, a non-negatively charged internucleotidic linkage comprises a cyclic guanidine moiety. In some embodiments, a modified internucleotidic linkage comprising a cyclic guanidine moiety has the structure of:

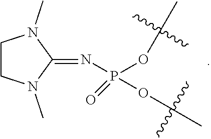 [SMILES: *OP(=O)(N=C1N(C)CCN1C)O*]

In some embodiments, a neutral internucleotidic linkage comprising a cyclic guanidine moiety is chirally controlled. In some embodiments, the present disclosure pertains to a composition comprising an oligonucleotide comprising at least one neutral internucleotidic linkage and at least one phosphorothioate internucleotidic linkage.

In some embodiments, the present disclosure pertains to a composition comprising an oligonucleotide comprising at least one neutral internucleotidic linkage and at least one phosphorothioate internucleotidic linkage, wherein the phosphorothioate internucleotidic linkage is a chirally controlled internucleotidic linkage in the Sp configuration.

In some embodiments, the present disclosure pertains to a composition comprising an oligonucleotide comprising at least one neutral internucleotidic linkage and at least one phosphorothioate internucleotidic linkage, wherein the phosphorothioate is a chirally controlled internucleotidic linkage in the Rp configuration.

In some embodiments, the present disclosure pertains to a composition comprising an oligonucleotide comprising at least one neutral internucleotidic linkage selected from a neutral internucleotidic linkage comprising an optionally substituted triazolyl group, a neutral internucleotidic linkage comprising an optionally substituted alkynyl group, and a neutral internucleotidic linkage comprising a Tmg group

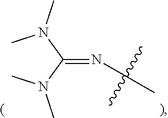 [SMILES: *N=C(N(C)C)N(C)C]

and at least one phosphorothioate.

In some embodiments, the present disclosure pertains to a composition comprising an oligonucleotide comprising at least one neutral internucleotidic linkage selected from a neutral internucleotidic linkage comprising an optionally substituted triazolyl group, a neutral internucleotidic linkage comprising an optionally substituted alkynyl group, and a neutral internucleotidic linkage comprising a Tmg group, and at least one phosphorothioate, wherein the phosphorothioate is a chirally controlled internucleotidic linkage in the Sp configuration.

In some embodiments, the present disclosure pertains to a composition comprising an oligonucleotide comprising at least one neutral internucleotidic linkage selected from a neutral internucleotidic linkage comprising an optionally substituted triazolyl group, a neutral internucleotidic linkage comprising an optionally substituted alkynyl group, and a neutral internucleotidic linkage comprising a Tmg group, and at least one phosphorothioate, wherein the phosphorothioate is a chirally controlled internucleotidic linkage in the Rp configuration.

Various types of internucleotidic linkages differ in properties. Without wishing to be bound by any theory, the present disclosure notes that a natural phosphate linkage (phosphodiester internucleotidic linkage) is anionic and may be unstable when used by itself without other chemical modifications in vivo; a phosphorothioate internucleotidic linkage is anionic, generally more stable in vivo than a natural phosphate linkage, and generally more hydrophobic; a neutral internucleotidic linkage such as one exemplified in the present disclosure comprising a cyclic guanidine moiety is neutral at physiological pH, can be more stable in vivo than a natural phosphate linkage, and more hydrophobic.

In some embodiments, a chirally controlled neutral internucleotidic linkage sis neutral at physiological pH, chirally controlled, stable in vivo, hydrophobic, and may increase endosomal escape.

In some embodiments, provided oligonucleotides comprise one or more regions, e.g., a block, wing, core, 5′-end, 3′-end, middle, seed, post-seed region, etc. In some embodiments, a region (e.g., a block, wing, core, 5′-end, 3′-end, middle region, etc.) comprises a non-negatively charged internucleotidic linkage, e.g., of formula I-n-1, I-n-2, I-n-3, II, II-a-1, II-a-2, II-b-1, II-b-2, II-c-1, II-c-2, II-d-1, II-d-2, etc. In some embodiments, a region comprises a neutral internucleotidic linkage. In some embodiments, a region comprises an internucleotidic linkage which comprises a triazole or alkyne moiety. In some embodiments, a region comprises an internucleotidic linkage which comprises a cyclic guanidine guanidine. In some embodiments, a region comprises an internucleotidic linkage which comprises a cyclic guanidine moiety. In some embodiments, a region comprises an internucleotidic linkage having the structure of

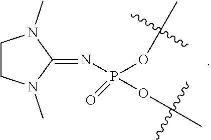 [SMILES: *OP(=O)(N=C1N(C)CCN1C)O*]

In some embodiments, such internucleotidic linkages are chirally controlled.

In some embodiments, the present disclosure provides an oligonucleotide composition comprising a first plurality of oligonucleotides which have a common base sequence and comprise one or more internucleotidic linkage, sugar, and/or base modifications.

In some embodiments, the present disclosure provides an oligonucleotide composition capable of directing single-stranded RNA interference and comprising a first plurality of oligonucleotides which have a common base sequence and comprise one or more internucleotidic linkage, and/or one or more sugar, and/or one or more base modifications. In some embodiments, an oligonucleotide or oligonucleotide composition is also capable of directing RNase H-mediated knockdown of a target gene RNA.

In some embodiments, a nucleotide is a natural nucleotide. In some embodiments, a nucleotide is a modified nucleotide. In some embodiments, a nucleotide is a nucleotide analog. In some embodiments, a base is a modified base. In some embodiments, a base is protected nucleobase, such as a protected nucleobase used in oligonucleotide synthesis. In some embodiments, a base is a base analog. In some embodiments, a sugar is a modified sugar. In some embodiments, a sugar is a sugar analog. In some embodiments, an internucleotidic linkage is a modified internucleotidic linkage. In some embodiments, a nucleotide comprises a base, a sugar, and an internucleotidic linkage, wherein each of the base, the sugar, and the internucleotidic linkage is independently and optionally naturally-occurring or non-naturally occurring. In some embodiments, a nucleoside comprises a base and a sugar, wherein each of the base and the sugar is independently and optionally naturally-occurring or non-naturally occurring. Non-limiting examples of nucleotides include DNA (2′-deoxy) and RNA (2′-OH) nucleotides; and those which comprise one or more modifications at the base, sugar and/or internucleotidic linkage. Non-limiting examples of sugars include ribose and deoxyribose; and ribose and deoxyribose with 2′-modifications, including but not limited to 2′-F, LNA, 2′-OMe, and 2′-MOE modifications. In some embodiments, an internucleotidic linkage can have a structure of Formula I as described in the present disclosure. In some embodiments, an internucleotidic linkage is a moiety which does not a comprise a phosphorus but serves to link two natural or non-natural sugars.

In some embodiments, the present disclosure provides chirally controlled oligonucleotide compositions that have an improved property and/or activity when compared to a reference condition, e.g., absence of the composition, or presence of a reference composition (e.g., a stereorandom composition of oligonucleotides having the same base sequence and chemical modifications).

In some embodiments, the present disclosure provides a chirally controlled oligonucleotide composition that directs a greater decrease of the expression, activity and/or level of a gene or a gene product thereof, single-stranded RNA interference and/or RNase H-mediated knockdown, when compared to a reference condition, e.g., absence of the composition, or presence of a reference composition (e.g., a stereorandom composition of oligonucleotides having the same base sequence and chemical modifications).

In some embodiments, an oligonucleotide composition comprising a plurality of oligonucleotides is stereorandom in that oligonucleotides of the plurality do not share a common stereochemistry at any chiral internucleotidic linkage. In some embodiments, an oligonucleotide composition comprising a plurality of oligonucleotides is chirally controlled in that oligonucleotides of the plurality share a common stereochemistry at one or more chiral internucleotidic linkages. In some embodiments, an oligonucleotide composition comprising a first plurality of oligonucleotides which is chirally controlled has a decreased susceptibility to endo- and exo-nucleases relative to an oligonucleotide composition comprising a first plurality of oligonucleotides which is stereorandom.

In some embodiments, an oligonucleotide composition is capable of directing a decrease in the expression and/or level of a target gene or its gene product. In some embodiments, an oligonucleotide composition is capable of directing single-stranded RNA interference. In some embodiments, an oligonucleotide composition is capable of directing RNase H-mediated knockdown. In some embodiments, an oligonucleotide composition is capable of directing RNase H-mediated knockdown and RNA interference of a target gene RNA. In some embodiments, an oligonucleotide composition is capable of directing RNase H-mediated knockdown of a first RNA target and RNA interference of a second RNA target, wherein the first and second RNA target are the same or different.

In some embodiments, a composition comprises a multimer of two or more of any: oligonucleotides of a first plurality and/or oligonucleotides of a second plurality, wherein the oligonucleotides of the first and second plurality can independently direct knockdown of the same or different targets independently via RNA interference and/or RNase H-mediated knockdown.

In some embodiments, the present disclosure provides an oligonucleotide composition comprising a first plurality of oligonucleotides which share:
1) a common base sequence;
2) a common pattern of backbone linkages;
3) common stereochemistry independently at at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 chiral internucleotidic linkages (“chirally controlled internucleotidic linkages”);
which composition is chirally controlled in that level of the first plurality of oligonucleotides in the composition is predetermined.

In some embodiments, an oligonucleotide composition comprising a plurality of oligonucleotides (e.g., a first plurality of oligonucleotides) is chirally controlled in that oligonucleotides of the plurality share a common stereochemistry independently at one or more chiral internucleotidic linkages. In some embodiments, oligonucleotides of the plurality share a common stereochemistry configuration at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chiral internucleotidic linkages, each of which is independently Rp or Sp In some embodiments, oligonucleotides of the plurality share a common stereochemistry configuration at each chiral internucleotidic linkages. In some embodiments, a chiral internucleotidic linkage where a predetermined level of oligonucleotides of a composition share a common stereochemistry configuration (independently Rp or Sp) is referred to as a chirally controlled internucleotidic linkage.

In some embodiments, a predetermined level of oligonucleotides of a provided composition, e.g., a first plurality of oligonucleotides of certain example compositions, comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 or more chirally controlled internucleotidic linkages.

In some embodiments, at least 5 internucleotidic linkages are chirally controlled; in some embodiments, at least 10 internucleotidic linkages are chirally controlled; in some embodiments, at least 15 internucleotidic linkages are chirally controlled; in some embodiments, each chiral internucleotidic linkage is chirally controlled.

In some embodiments, 1%-100% of chiral internucleotidic linkages are chirally controlled. In some embodiments, at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of chiral internucleotidic linkages are chirally controlled.

In some embodiments, the present disclosure provides an oligonucleotide composition comprising a first plurality of oligonucleotides which share:
1) a common base sequence;
2) a common pattern of backbone linkages; and
3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers.

In some embodiments, the common pattern of backbone chiral centers comprises at least one internucleotidic linkage comprising a chirally controlled chiral center.

In some embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition. In some embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence. In some embodiments, all oligonucleotides in a provided composition that are of or comprise a common base sequence are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In some embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, base modification, sugar modification and/or modified internucleotidic linkage. In some embodiments, all oligonucleotides in a provided composition that are of or comprise a common base sequence, base modification, sugar modification and/or modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In some embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that are of or comprise a common base sequence, pattern of base modification, pattern of sugar modification, and/or pattern of modified internucleotidic linkage. In some embodiments, all oligonucleotides in a provided composition that are of or comprise a common base sequence, pattern of base modification, pattern of sugar modification, and/or pattern of modified internucleotidic linkage are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition. In some embodiments, a predetermined level of oligonucleotides is at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in a provided composition that share a common base sequence, a common pattern of base modification, a common pattern of sugar modification, and/or a common pattern of modified internucleotidic linkages. In some embodiments, all oligonucleotides in a provided composition that share a common base sequence, a common pattern of base modification, a common pattern of sugar modification, and/or a common pattern of modified internucleotidic linkages are at least 1%, 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% of all oligonucleotides in the composition.

In some embodiments, a predetermined level is 1-100%. In some embodiments, a predetermined level is at least 1%. In some embodiments, a predetermined level is at least 5%. In some embodiments, a predetermined level is at least 10%. In some embodiments, a predetermined level is at least 20%. In some embodiments, a predetermined level is at least 30%. In some embodiments, a predetermined level is at least 40%. In some embodiments, a predetermined level is at least 50%. In some embodiments, a predetermined level is at least 60%. In some embodiments, a predetermined level is at least 10%. In some embodiments, a predetermined level is at least 70%. In some embodiments, a predetermined level is at least 80%. In some embodiments, a predetermined level is at least 90%. In some embodiments, a predetermined level is at least 5*(½^{g}), wherein g is the number of chirally controlled internucleotidic linkages. In some embodiments, a predetermined level is at least 10*(½^{g}), wherein g is the number of chirally controlled internucleotidic linkages. In some embodiments, a predetermined level is at least 100*(½^{g}), wherein g is the number of chirally controlled internucleotidic linkages. In some embodiments, a predetermined level is at least (0.80)^{g}, wherein g is the number of chirally controlled internucleotidic linkages. In some embodiments, a predetermined level is at least (0.80)^{g}, wherein g is the number of chirally controlled internucleotidic linkages. In some embodiments, a predetermined level is at least (0.80)^{g}, wherein g is the number of chirally controlled internucleotidic linkages. In some embodiments, a predetermined level is at least (0.85)^{g}, wherein g is the number of chirally controlled internucleotidic linkages. In some embodiments, a predetermined level is at least (0.90)^{g}, wherein g is the number of chirally controlled internucleotidic linkages. In some embodiments, a predetermined level is at least (0.95)^{g}, wherein g is the number of chirally controlled internucleotidic linkages. In some embodiments, a predetermined level is at least (0.96)^{g}, wherein g is the number of chirally controlled internucleotidic linkages. In some embodiments, a predetermined level is at least (0.97)^{g}, wherein g is the number of chirally controlled internucleotidic linkages. In some embodiments, a predetermined level is at least (0.98)^{g}, wherein g is the number of chirally controlled internucleotidic linkages. In some embodiments, a predetermined level is at least (0.99)^{g}, wherein g is the number of chirally controlled internucleotidic linkages. In some embodiments, to determine level of oligonucleotides having g chirally controlled internucleotidic linkages in a composition, product of diastereopurity of each of the g chirally controlled internucleotidic linkages: (diastereopurity of chirally controlled internucleotidic linkage 1)*(diastereopurity of chirally controlled internucleotidic linkage 2)* . . . *(diastereopurity of chirally controlled internucleotidic linkage g) is utilized as the level, wherein diastereopurity of each chirally controlled internucleotidic linkage is independently represented by diastereopurity of a dimer comprising the same internucleotidic linkage and nucleosides flanking the internucleotidic linkage and prepared under comparable methods as the oligonucleotides (e.g., comparable or preferably identical oligonucleotide preparation cycles, including comparable or preferably identical reagents and reaction conditions).

In some embodiments, levels of oligonucleotides and/or diastereopurity can be determined by analytical methods, e.g., chromatographic, spectrometric, spectroscopic methods or any combinations thereof.

Among other things, the present disclosure encompasses the recognition that stereorandom oligonucleotide preparations contain a plurality of distinct chemical entities that differ from one another, e.g., in the stereochemical structure (or stereochemistry) of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence and/or chemical modifications, they are different chemical entities at least due to their different backbone stereochemistry, and they can have, as demonstrated herein, different properties, e.g., sensitivity to nucleases, activities, distribution, etc. In some embodiments, a particular stereoisomer may be defined, for example, by its base sequence, its length, its pattern of backbone linkages, and its pattern of backbone chiral centers. In some embodiments, the present disclosure demonstrates that improvements in properties and activities achieved through control of stereochemistry within an oligonucleotide can be comparable to, or even better than those achieved through use of chemical modification.

In some embodiments, a provided oligonucleotide, e.g., an oligonucleotide capable of directing a decrease in the expression and/or level of a target gene or its gene product, an oligonucleotide capable of directing single-stranded RNA interference (e.g., a single-stranded RNAi agent, ssRNA or ssRNAi), or an oligonucleotide capable of directing single-stranded RNA interference and RNase H-mediated knockdown, etc, is represented by the structure: 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{mz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, wherein each variable is independently as described in the present disclosure.

In some embodiments, PX0 is a 5′-end structure. In some embodiments, each of PX1 to PX26 is independently an internucleotidic linkage. In some embodiments, PX27 is an internucleotidic linkage or OH. In some embodiments, N1 to N27 independently represent a nucleoside. In some embodiments, N1-PX1 to N27-PX27 independently represent a nucleotide. Any nucleoside can be the same or different than any adjacent nucleoside. Any nucleotide can be the same or different than any adjacent nucleotide. In some embodiments, wherein any of mz to yz>1, each base of N18 to N27 can be the same or different; and/or each nucleoside of N18 to N27 can be the same or different and can independently comprise the same or different modification (e.g., 2′-modification). In some embodiments, each of mz, nz, pz, rz, sz, tz, vz and wz can independently be 0 to 10. In some embodiments: if, in -(N18-PX18)_{mz}-, mz>1, then each N18 can be the same or different and/or each PX18 can be the same or different; if, in -(N19-PX19)_{nz}-, nz>1, each N19 can be the same or different and/or each PX19 can be the same or different; if, in -(N20-PX20)_{pz}-, pz>1, then each N20 can be the same or different and/or each PX20 can be the same or different; if, in -(N21-PX21)_{rz}-, rz>1, then each N21 can be the same or different and/or each PX21 can be the same or different; if, in -(N22-PX22)_{sz}-, sz>1, then each N22 can be the same or different and/or each PX22 can be the same or different; if, in -(N23-PX23)_{tz}-, tz>1, then each N23 can be the same or different and/or each PX23 can be the same or different; if, in -(N24-PX24)_{vz}-, vz>1, then each N24 can be the same or different and/or each PX24 can be the same or different; if, in -(N25-PX25)_{vz}-, wz>1, then each N25 can be the same or different and/or each PX26 can be the same or different; if, in -(N26-PX26-N27-PX27)_{yz}-, yz>1, then each N26 can be the same or different and each N27 can be the same or different, and each N26 and N27 can be the same or different and/or each PX26 can be the same or different and/or each PX27 can be the same or different and/or each PX26 and PX27 can be the same or different; etc.

In some embodiments, each N or PX can independently and optionally further comprise one or more additional chemical moiety, e.g., a targeting moiety, a carbohydrate moiety, a GalNAc moiety, a lipid moiety, etc.

In some embodiments, each of PX1 to PX27 independently represents an internucleotidic linkage, wherein each of PX1 to PX27 can be the same or different. In some embodiments, each of PX1 to PX27 independently represents an internucleotidic linkage which is a phosphorodiester, a phosphorothioate, a phosphorothioate in the Sp configuration, a phosphorothioate in the Rp configuration, an internucleotidic linkage, an internucleotidic linkage in the Sp configuration, or an internucleotidic linkage in the Rp configuration, wherein each of PX1 to PX27 can be the same or different.

In some embodiments, a 3′-end region is represented by: -(N26-PX26-N27-PX27)_{yz}-(CAP)_{zz}, wherein yz=1 and zz=0, or yz=0 and zz=1, or yz=1 and zz=1; -(N26-PX26-N27-PX27)_{yz}, wherein yz=1; -(CAP)_{zz}, wherein zz=1; or -(N26-PX26-N27-PX27)_{yz}-(CAP)_{zz}, wherein yz=1 and zz=1.

In some embodiments, PX27 represents an internucleotidic linkage, or —OH. In some embodiments, zz=0, yz=1 and PX27 is —OH. In some embodiments, zz=1 and yz=1, and PX27 is an internucleotidic linkage.

In some embodiments, PX27 represents an internucleotidic linkage which is a phosphorodiester, a phosphorothioate, a phosphorothioate in the Sp configuration, a phosphorothioate in the Rp configuration, an internucleotidic linkage, an internucleotidic linkage in the Sp configuration, or an internucleotidic linkage in the Rp configuration. In some embodiments, PX27 is —OH.

In some embodiments, wherein zz=1 (e.g., in the presence of a CAP), PX27 represents an an internucleotidic linkage which is a phosphorodiester, a phosphorothioate, a phosphorothioate in the Sp configuration, a phosphorothioate in the Rp configuration, an internucleotidic linkage, an internucleotidic linkage in the Sp configuration, or an internucleotidic linkage in the Rp configuration.

In some embodiments, an oligonucleotide comprises, in 5′ to 3′ order, a 5′-end region, a seed region, a post-seed region, and a 3′-end region, optionally further comprising an additional chemical moiety.

In some embodiments, a 5′-end region is the entire portion of an oligonucleotide which is 5′ to the seed region. In some embodiments, a 3′-end region is the entire portion of an oligonucleotide which is 3′ to the post-seed region.

In some embodiments, a 5′-end region is represented by any of: PX0-, PX0-N1-, PX0-N1-PX1-, PX0-N1-PX1-N2-, PX0-N1-PX1-N2-PX2-, PX0-N1-PX1-N2-PX2-N3-, or PX0-N1-PX1-N2-PX2-N3-PX3-.

In some embodiments, a 5′-end region is represented by any of: PX0-, PX0-N1-, or PX0-N1-PX1-.

In some embodiments, a 5′-end structure is represented by any of: PX0-, PX0-N1-, or PX0-N1-PX1-.

In some embodiments, a 5′-end structure is represented by PX0-.

In some embodiments, a 5′-end structure is a 5′-end group.

In some embodiments, a 5′-end structure comprises a 5′-end group.

In some embodiments, —N1-PX1- represents a 5′ nucleotide moiety. In some embodiments, —N1- represents a 5′ nucleoside.

In some embodiments, a 5′-end nucleoside is —N1-.

In some embodiments, a 5′-end nucleotide is —N1-PX1-.

In some embodiments, a provided oligonucleotide can comprise a 5′-end region, 5′-end structure, 5′-end group, 5′-end nucleoside, or 5′-end nucleotide described herein or known in the art.

In some embodiments, —PX0-, —PX0-N1-, and —PX0-N1-PX1- is represented by a structure described herein of a 5′-end structure, 5′-end region, 5′-nucleotide, modified 5′-nucleotide, 5′-nucleotide analog, or 5′-nucleoside, modified 5′-nucleoside or 5′-nucleoside analog.

In some embodiments, —N2-PX2-N3-PX3-N4-PX4-N5-PX5-N6-PX6-N7-PX7-represents a seed region. In some embodiments, —N2-PX2-N3-PX3-N4-PX4-N5-PX5-N6-PX6-represents a seed region. In some embodiments, —N3-PX3-N4-PX4-N5-PX5-N6-PX6-N7-PX7-represents a seed region. In some embodiments, —N2-PX2-N3-PX3-N4-PX4-N5-PX5-N6-PX6-N7-PX7-N8-PX8- represents a seed region. In some embodiments, —N3-PX3-N4-PX4-N5-PX5-N6-PX6-N7-PX7-N8-PX8- represents a seed region. In some embodiments, —N3-PX3-N4-PX4-N5-PX5-N6-PX6- represents a seed region. In some embodiments, —N4-PX4-N5-PX5-N6-PX6-N7-PX7-N8-PX8- represents a seed region.

In some embodiments, —N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}- represents a post-seed region (e.g., a region between a seed region and 3′-end region).

In some embodiments, —N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}- represents a post-seed region.

In some embodiments, —N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}- represents a post-seed region.

In some embodiments, —N2-PX2-N3-PX3-N4-PX4-N5-PX5-N6-PX6-N7-PX7-represents a seed region, and —N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}- represents a post-seed region.

In some embodiments, —N2-PX2-N3-PX3-N4-PX4-N5-PX5-N6-PX6- represents a seed region, and —N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{tz}-(N22-PX22)_{sz}-(N23-PX23)_{tz}-(N24-PX24)_{vz}-(N25-PX25)_{wz}- represents a post-seed region.

In some embodiments, —N3-PX3-N4-PX4-N5-PX5-N6-PX6-N7-PX7- represents a seed region, and —N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}4N23-PX23)_{tz}-(N24-PX24)_{vz}-(N25-PX25)_{wz}- represents a post-seed region.

In some embodiments, —N2-PX2-N3-PX3-N4-PX4-N5-PX5-N6-PX6-N7-PX7-N8-PX8-represents a seed region, and —N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{tz}-(N22-PX22)_{sz}4N23-PX23)_{tz}-(N24-PX24)_{vz}-(N25-PX25)_{wz}- represents a post-seed region.

In some embodiments, —N3-PX3-N4-PX4-N5-PX5-N6-PX6-N7-PX7-N8-PX8-represents a seed region, and —N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{tz}-(N22-PX22)_{sz}4N23-PX23)_{tz}-(N24-PX24)_{vz}-(N25-PX25)_{wz}- represents a post-seed region.

In some embodiments, —N3-PX3-N4-PX4-N5-PX5-N6-PX6- represents a seed region, and —N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}4N23-PX23)_{tz}-(N24-PX24)_{vz}-(N25-PX25)_{wz}- represents a post-seed region.

In some embodiments, —N4-PX4-N5-PX5-N6-PX6-N7-PX7-N8-PX8- represents a seed region, and —N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{tz}-(N22-PX22)_{sz}-(N23-PX23)_{tz}-(N24-PX24)_{vz}-(N25-PX25)_{wz}- represents a post-seed region.

In some embodiments, 5′-PX0-N1-PX1- represents a 5′-end region; —N2-PX2-N3-PX3-N4-PX4-N5-PX5-N6-PX6-N7- represents a seed region; -PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}- represents a post-seed region; and -(N26-PX26-N27-PX27)_{yz}-(CAP)_{zz}-3′ represents a 3′-end region.

In some embodiments, 5′-PX0-N1-PX1- represents a 5′-end region; —N2-PX2-N3-PX3-N4-PX4-N5-PX5-N6-PX6- represents a seed region; —N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz} represents a post-seed region; and -(N26-PX26-N27-PX27)_{yz}-(CAP)_{zz}-3′ represents a 3′-end region.

In some embodiments, 5′-PX0-N1-PX1-N2-PX2- represents a 5′-end region; —N3-PX3-N4-PX4-N5-PX5-N6-PX6-N7-PX7- represents a seed region; —N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz} represents a post-seed region; and -(N26-PX26-N27-PX27)_{yz}-(CAP)_{zz}-3′ represents a 3′-end region.

In some embodiments, 5′-PX0-N1-PX1-N2-PX2- represents a 5′-end region; —N3-PX3-N4-PX4-N5-PX5-N6-PX6-N7-PX7-N8-PX8- represents a seed region; —N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz} represents a post-seed region; and -(N26-PX26-N27-PX27)_{yz}-(CAP)_{zz}-3′ represents a 3′-end region.

In some embodiments, 5′-PX0-N1-PX1- represents a 5′-end region; —N2-PX2-N3-PX3-N4-PX4-N5-PX5-N6-PX6-N7-PX7-N8- represents a seed region; -PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz} represents a post-seed region; and -(N26-PX26-N27-PX27)_{yz}-(CAP)_{zz}-3′ represents a 3′-end region.

In some embodiments, 5′-PX0-N1-PX1-N2-PX2- represents a 5′-end region; —N3-PX3-N4-PX4-N5-PX5-N6-PX6- represents a seed region; —N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz} represents a post-seed region; and -(N26-PX26-N27-PX27)_{yz}-(CAP)_{zz}-3′ represents a 3′-end region.

In some embodiments, each of mz, nz, pz, rz, sz, tz, vz and wz is independently 0 to 10.

In some embodiments, each of mz, nz, pz, rz, sz, tz, vz and wz can independently be 0 to 10, and the total length of the oligonucleotide is no more than about 49 nucleotides. In some embodiments, each of mz, nz, pz, rz, sz, tz, vz and wz can independently be 0 to 10, and the total length of the oligonucleotide is no more than about 45 nucleotides. In some embodiments, each of mz, nz, pz, rz, sz, tz, vz and wz can independently be 0 to 10, and the total length of the oligonucleotide is no more than about 40 nucleotides. In some embodiments, each of mz, nz, pz, rz, sz, tz, vz and wz can independently be 0 to 10, and the total length of the oligonucleotide is no more than about 35 nucleotides. In some embodiments, each of mz, nz, pz, rz, sz, tz, vz and wz can independently be 0 to 10, and the total length of the oligonucleotide is no more than about 30 nucleotides. In some embodiments, each of mz, nz, pz, rz, sz, tz, vz and wz can independently be 0 to 10, and the total length of the oligonucleotide is no more than about 25 nucleotides. In some embodiments, each of mz, nz, pz, rz, sz, tz, vz and wz can independently be 0 to 10, and the total length of the oligonucleotide is no more than about 23 nucleotides. In some embodiments, each of mz, nz, pz, rz, sz, tz, vz and wz can independently be 0 to 10, and the total length of the oligonucleotide is no more than about 21 nucleotides.

In some embodiments, -(N26-PX26-N27-PX27)_{yz}-represents a 3′-terminal dinucleotide, wherein yz is 1. When yz=0, the 3′-terminal dinucleotide is absent. When yz=1, the 3′-terminal dinucleotide is present.

In some embodiments, -(CAP)_{zz}- represents an optional 3′-end cap, wherein zz is 0 or 1. When _{zz}=0, the CAP is absent. When _{zz}=1, the CAP is present.

In some embodiments, if yz=1, then zz=0. In some embodiments, if yz=0, zz=1. In some embodiments, yz=1 and zz=1, indicating that the molecule comprises both a 3′-terminal dinucleotide and a CAP.

In some embodiments, a 5′-end structure, a 5′-end region, 5′ nucleotide moiety, seed region, post-seed region, 3′-terminal dinucleotide and/or 3′-end cap independently have any structure described herein or known in the art. In some embodiments, any structure for a 5′-end described herein or known in the art and/or any structure for a 5′ nucleotide moiety described herein or known in the art and/or any structure for a seed region described herein or known in the art and/or any structure for a post-seed region described herein or known in the art and/or any structure for a 3′-terminal dinucleotide described herein or known in the art and/or any structure for a 3′-end cap described herein or known in the art can be combined.

In some embodiments, a provided oligonucleotide comprises one or more blocks. In some embodiments, a provided oligonucleotide comprise one or more blocks, wherein a block comprises one or more consecutive nucleosides, and/or nucleotides, and/or sugars, or bases, and/or internucleotidic linkages. In some embodiments, a block encompasses an entire seed region or a portion thereof. In some embodiments, a block encompasses an entire post-seed region or a portion thereof.

In some embodiments, provided oligonucleotides are blockmers.

In some embodiments, provided oligonucleotide are altmers.

In some embodiments, provided oligonucleotides are altmers comprising alternating blocks. In some embodiments, a blockmer or an altmer can be defined by chemical modifications (including presence or absence), e.g., base modifications, sugar modification, internucleotidic linkage modifications, stereochemistry, etc., or patterns thereof.

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides comprise one or more blocks comprising two or more different internucleotidic linkages. In some embodiments, provided oligonucleotides comprise one or more blocks comprising two or more total modified internucleotidic linkages and natural phosphate linkages. In some embodiments, provided oligonucleotides comprise one or more blocks comprising two or more different modified internucleotidic linkages. In some embodiments, provided oligonucleotides comprise alternating blocks comprising two or more different internucleotidic linkages. In some embodiments, provided oligonucleotides comprise alternating blocks comprising two or more total modified internucleotidic linkages and natural phosphate linkages. In some embodiments, provided oligonucleotides comprise alternating blocks comprising two or more different modified internucleotidic linkages. In some embodiments, a block comprising modified internucleotidic linkages have pattern of backbone chiral centers as described herein. In some embodiments, each block comprising modified internucleotidic linkages has the same pattern of backbone chiral centers. In some embodiments, blocks comprising modified internucleotidic linkages have different patterns of backbone chiral centers. In some embodiments, blocks comprising modified internucleotidic linkages have different length and/or modifications. In some embodiments, blocks comprising modified internucleotidic linkages have the same length and/or modifications. In some embodiments, blocks comprising modified internucleotidic linkages have the same length. In some embodiments, blocks comprising modified internucleotidic linkages have the same internucleotidic linkages.

In some embodiments, provided oligonucleotides are capable of directing single-stranded RNA interference and comprise a first block in the seed region (seed region-block), and a second block in the post-seed region (post-seed region-block), each of which independently comprise one or more modified internucleotidic linkages. In some embodiments, each of the seed region- and post-seed region-blocks independently comprises 2, 3, 4, 5, 6, 7 or more modified internucleotidic linkages. In some embodiments, a seed region-block comprises 4 or more modified internucleotidic linkages. In some embodiments, a seed region-block comprises 5 or more modified internucleotidic linkages. In some embodiments, a seed region-block comprises 6 or more modified internucleotidic linkages. In some embodiments, a seed region-block comprises 7 modified internucleotidic linkages. In some embodiments, a post-seed region-block comprises 4 or more modified internucleotidic linkages. In some embodiments, a post-seed region-block comprises 5 or more modified internucleotidic linkages. In some embodiments, a post-seed region-block comprises 6 or more modified internucleotidic linkages. In some embodiments, a post-seed region-block comprises 7 or more modified internucleotidic linkages. In some embodiments, each of the seed region- and post-seed region-blocks independently comprises at least 4 modified internucleotidic linkages. In some embodiments, each of the seed region- and post-seed region-blocks independently comprises at least 5 modified internucleotidic linkages. In some embodiments, each of the seed region- and post-seed region-blocks independently comprises at least 6 modified internucleotidic linkages. In some embodiments, each of the seed region- and post-seed region-blocks independently comprises at least 7 modified internucleotidic linkages. In some embodiments, modified internucleotidic linkages within a block are consecutive. In some embodiments, each linkage of the seed region-block is independently a phosphorothioate linkage. In some embodiments, each linkage of the seed region-block is independently chirally controlled. In some embodiments, each linkage of the seed region-block is Sp. In some embodiments, each linkage of the post-seed region-block is independently a modified internucleotidic linkage. In some embodiments, each linkage of the post-seed region-block is independently a phosphorothioate linkage. In some embodiments, each linkage of the post-seed region-block is independently chirally controlled. In some embodiments, each linkage of the post-seed region-block is Sp.

In some embodiments, provided oligonucleotides comprise one or more sugar modifications. In some embodiments, a sugar modification is at the 2′-position. In some embodiments, a sugar modification is selected from: 2′-F, 2′-OMe, and 2′-MOE. 2′-F is also designated 2′ Fluoro. 2′-OMe is also designated 2′-O-Methyl. 2′-MOE is also designated 2′-Methoxyethyl or MOE.

In some embodiments, provided oligonucleotides are capable of directing single-stranded RNA interference and comprise a first block in the seed region (seed region-block), and a second block in the post-seed region (post-seed region-block), each of which independently comprise one or more 2′-F. In some embodiments, each of the seed region- and post-seed region-blocks independently comprises 2, 3, 4, 5, 6, 7 or more 2′-F. In some embodiments, each of the seed region- and post-seed region-blocks independently comprises 2, 3, 4, 5, 6, 7 or more consecutive 2′-F. In some embodiments, a seed region-block comprises 4 or more 2′-F. In some embodiments, a seed region-block comprises 5 or more 2′-F. In some embodiments, a seed region-block comprises 6 or more 2′-F. In some embodiments, a seed region-block comprises 7 2′-F. In some embodiments, a post-seed region-block comprises 4 or more 2′-F. In some embodiments, a post-seed region-block comprises 5 or more 2′-F. In some embodiments, a post-seed region-block comprises 6 or more 2′-F. In some embodiments, a post-seed region-block comprises 7 or more 2′-F. In some embodiments, each of the seed region- and post-seed region-blocks independently comprises at least 4 2′-F. In some embodiments, each of the seed region- and post-seed region-blocks independently comprises at least 5 2′-F. In some embodiments, each of the seed region- and post-seed region-blocks independently comprises at least 6 2′-F. In some embodiments, each of the seed region- and post-seed region-blocks independently comprises at least 7 2′-F. In some embodiments, 2′-F within a block are consecutive.

In some embodiments, an oligonucleotide comprises one, two, three, four, five, six, seven, eight, nine, ten, or more 2′-F. In some embodiments, an oligonucleotide comprises two, three, four, five, six, seven, eight, nine, ten, or more consecutive sugar moieties comprising 2′-F.

In some embodiments, an oligonucleotide comprises only two 2′-F. In some embodiments, an oligonucleotide comprises only two 2′-F, wherein the two nucleotides are at the 2nd and 14th positions. Non-limiting examples of such oligonucleotides include: oligonucleotides of Format 70, FIG. 1F, and WV-7540 and WV-7543.

In some embodiments, an oligonucleotide comprises only two 2′-F, wherein the two nucleotides are at the 2nd and 14th positions, and wherein the first nucleotide (5′-end nucleotide or —N1-PX1-) is 2′-deoxy. Non-limiting examples of such an oligonucleotide include: Format 70, FIG. 1F, and WV-7540 and WV-7543.

In some embodiments, an oligonucleotide comprises only two 2′-F, wherein the two nucleotides are at the 2nd and 14th positions, and wherein the first nucleotide (5′-end nucleotide or —N1-PX1-) is 2′-deoxy T. Non-limiting examples of such an oligonucleotide include: Format 70, FIG. 1F, and WV-7540 and WV-7543.

In some embodiments, an oligonucleotide comprises only two 2′-F, wherein the two nucleotides are at the 2nd and 14th positions, and wherein the first nucleotide (5′-end nucleotide or —N1-PX1-) is 2′-deoxy, and the 5′-end structure (PX0) is —OH. Non-limiting examples of such an oligonucleotide include: oligonucleotides of Format 70, FIG. 1F, and WV-7540 and WV-7543.

In some embodiments, an oligonucleotide comprises only two 2′-F, wherein the two nucleotides are at the 2nd and 14th positions, and wherein the first nucleotide (5′-end nucleotide or —N1-PX1-) is 2′-deoxy T, and the 5′-end structure (PX0) is —OH. Non-limiting examples of such an oligonucleotide include: Format 70, FIG. 1F, and WV-7540 and WV-7543.

In some embodiments herein, in reference to an oligonucleotide, “first” (e.g., first nucleotide) refers to the 5′ end of the oligonucleotide, and “last” or “end” (e.g., last nucleotide or end nucleotide) refers to the 3′ end.

In some embodiments, provided oligonucleotides comprise sugars with a particular modification which alternate with sugars with no modification or a different modification. In some embodiments, sugars with a particular modification appear in one or more blocks.

In some embodiments, provided oligonucleotides comprise one or more blocks comprising sugars with a particular 2′ modification which alternate with sugars which independently have no modification or have a different modification. In some embodiments, provided oligonucleotides comprise one or more blocks comprising sugars with a 2′-F modification which alternate with sugars which independently have no modification or have a different modification. In some embodiments, provided oligonucleotides comprise one or more blocks comprising sugars with a 2′-OMe modification which alternate with sugars which independently have no modification or a different modification. In some embodiments, provided oligonucleotides one or more blocks comprising sugars with a 2′-OMe modification which alternate with sugars with a 2′-F modification.

In some embodiments, a block of sugars has or comprises a pattern of 2′-modifications of any of: ff, fffm, fffmm, fffmmm, fffmmmm, fffmmmmm, fffmmmmmm, fffmmmmmmf, fffmmmmmmff, fffmmmmmmffm, fffmmmmmmffmm, fffmmmmmmffmmf, fffmmmmmmffmmfm, fffmmmmmmffmmfmf, fffmmmmmmffmmfmfm, fffmmmmmmffmmfmfmf, fffmmmmmmffmmfmfmfm, fffmmmmmmffmmfmfmfmm, fffmmmmmmffmmfmfmfmmm, ffmmffmm, ffmmmmmmffmmfmfmfmmm, fmfmfmfmfmfmfm, fmfmfmfmfmfmfmf, fmfmfmfmfmfmfmfm, fmfmfmfmfmfmfmfmf, fmfmfmfmfmfmfmfmfm, fmfmfmfmfmfmfmfmfmf, fmfmfmfmfmfmfmfmfmfm, fmfmfmfmfmfmfmfmfmfmm, fmfmfmfmfmfmfmfmfmm, fmfmfmfmfmfmfmfmm, fmfmfmfmfmfmfmm, fmfmfmfmfmfmmm, fmmffmm, fmmmmmmffmmfmfmfmmm, mff, mffm, mffmf, mffmff, mffmffm, mffmmffmm, mfmfm, mfmfmfmfmfffmfmfmfmmm, mfmfmfmfmfmfmfm, mfmfmfmfmfmfmfmfmfmm, mfmfmfmfmfmfmfmfmfmmm, mfmfmfmfmfmfmfmfmm, mfmfmfmfmfmfmfmm, mfmfmfmfmfmfmm, mfmfmfmfmfmfmmm, mfmfmfmfmfmmm, mfmfmfmfmfmmmfm, mfmfmfmfmfmmmmm, mfmfmfmfmmm, mfmfmfmfmmmfmfm, mfmfmfmfmmmfmmm, mfmfmfmfmmmmmfm, mfmfmfmmm, mfmfmfmmmfmfmfm, mfmfmfmmmfmfmmm, mfmfmfmmmfmmmfm, mfmfmfmmmmmfmfm, mfmfmmm, mfmfmmmfmfmfmfm, mfmfmmmfmfmfmmm, mfmfmmmfmfmmmfm, mfmfmmmfmmmfmfm, mfmfmmmmmfmfmfm, mfmmm, mfmmmfmfmfmfmfm, mfmmmfmfmfmfmmm, mfmmmfmfmfmmmfm, mfmmmfmfmmmfmfm, mfmmmfmmmfmfmfm, mfmmmfmmmfmfmfm, mfmmmmmfmfmfmfm, mmffm, mmffmm, mmffmm, mmffmmf, mmffmmff, mmffmmffm, mmffmmffmm, mmffmmfmfmfmmm, mmm, mmmffmmfmfmfmmm, mmmfmfmfmfmfmfm, mmmfmfmfmfmfmmm, mmmfmfmfmfmmmfm, mmmfmfmfmmmfmfm, mmmfmfmmmfmfmfm, mmmfmmmfmfmfmfm, mmmmffmmfmfmfmmm, mmm, mmmm, mmmmm, mmmmmffmmfmfmfmmm, mmmmmfmfmfmfmfm, mmmmmm, mmmmmmffmmfmfmfmmm, mfmf, mfmf, mfmfmf, fmfm, fmfmfm, fmfmfmf, dfdf, dfdfdf, dfdfdfdf, fdfd, fdfdfd, fdfdfdfd, dfdfmfmf, dfmfmf, mfdfmf, or dfmfdf, wherein m indicates a 2′-OMe, f indicates a 2′-F, and d indicates no substitution at 2′-position. In some embodiments, a seed region and/or post-seed region can comprise a block of sugar modifications.

In some embodiments, a block is a stereochemistry block. In some embodiments, a block is an Rp block in that each internucleotidic linkage of the block is Rp. In some embodiments, a seed region-block is an Rp block. In some embodiments, a post-seed region-block is an Rp block. In some embodiments, a block is an Sp block in that each internucleotidic linkage of the block is Sp. In some embodiments, a seed region-block is an Sp block. In some embodiments, a post-seed region-block is an Sp block. In some embodiments, provided oligonucleotides comprise both Rp and Sp blocks. In some embodiments, provided oligonucleotides comprise one or more Rp but no Sp blocks. In some embodiments, provided oligonucleotides comprise one or more Sp but no Rp blocks. In some embodiments, provided oligonucleotides comprise one or more PO blocks wherein each internucleotidic linkage of the block is a natural phosphate linkage.

In some embodiments, a seed region-block is an Sp block wherein each sugar moiety comprises a 2′-F modification. In some embodiments, a seed region-block is an Sp block wherein each of internucleotidic linkage is a modified internucleotidic linkage and each sugar moiety comprises a 2′-F modification. In some embodiments, a seed region-block is an Sp block wherein each of internucleotidic linkage is a phosphorothioate linkage and each sugar moiety comprises a 2′-F modification. In some embodiments, a seed region-block comprises 4 or more nucleoside units. In some embodiments, a nucleoside unit is a nucleoside. In some embodiments, a seed region-block comprises 5 or more nucleoside units. In some embodiments, a seed region-block comprises 6 or more nucleoside units. In some embodiments, a seed region-block comprises 7 or more nucleoside units. In some embodiments, a post-seed region-block is an Sp block wherein each sugar moiety comprises a 2′-F modification. In some embodiments, a post-seed region-block is an Sp block wherein each of internucleotidic linkage is a modified internucleotidic linkage and each sugar moiety comprises a 2′-F modification. In some embodiments, a post-seed region-block is an Sp block wherein each of internucleotidic linkage is a phosphorothioate linkage and each sugar moiety comprises a 2′-F modification. In some embodiments, a post-seed region-block comprises 4 or more nucleoside units. In some embodiments, a post-seed region-block comprises 5 or more nucleoside units. In some embodiments, a post-seed region-block comprises 6 or more nucleoside units. In some embodiments, a post-seed region-block comprises 7 or more nucleoside units. In some embodiments, a seed region and/or post-seed region can comprise a block. In some embodiments, a seed region and/or post-seed region comprises a stereochemistry block.

In some embodiments, a type of nucleoside in a region, a block, or an oligonucleotide is followed by a specific type of internucleotidic linkage, e.g., natural phosphate linkage, modified internucleotidic linkage, Rp chiral internucleotidic linkage, Sp chiral internucleotidic linkage, etc. In some embodiments, A is followed by a Sp internucleotidic linkage. In some embodiments, A is followed by a Rp internucleotidic linkage. In some embodiments, A is followed by a natural phosphate linkage (PO). In some embodiments, U is followed by a Sp internucleotidic linkage. In some embodiments, U is followed by a Rp internucleotidic linkage. In some embodiments, U is followed by a natural phosphate linkage (PO). In some embodiments, C is followed by a Sp internucleotidic linkage. In some embodiments, C is followed by a Rp internucleotidic linkage. In some embodiments, C is followed by a natural phosphate linkage (PO). In some embodiments, G is followed by a Sp internucleotidic linkage. In some embodiments, G is followed by a Rp internucleotidic linkage. In some embodiments, G is followed by a natural phosphate linkage (PO). In some embodiments, C and U are followed by Sp internucleotidic linkages. In some embodiments, C and U are followed by Rp internucleotidic linkages. In some embodiments, C and U are followed by natural phosphate linkages (PO). In some embodiments, A and G are followed by Sp internucleotidic linkages. In some embodiments, A and G are followed by Rp internucleotidic linkages. In some embodiments, A and G are followed by natural phosphate linkages (PO).

In some embodiments, provided oligonucleotides comprise alternating blocks comprising modified sugar moieties and unmodified sugar moieties. In some embodiments, modified sugar moieties comprise 2′-modifications. In some embodiments, provided oligonucleotides comprise alternating 2′-OMe modified sugar moieties and unmodified sugar moieties.

In some embodiments, provided oligonucleotides comprise one or more 2′-F modified sugar moieties whose 3′-internucleotidic linkages are modified internucleotidic linkages. In some embodiments, a modified internucleotidic linkage is phosphorothioate. In some embodiments, a modified internucleotidic linkage is chirally controlled and is Rp. In some embodiments, a modified internucleotidic linkage is chirally controlled and is Sp. In some embodiments, provided oligonucleotides comprise one or more 2′-OR′ modified sugar moieties whose 3′-internucleotidic linkages are natural phosphate linkages.

In some embodiments, a provided oligonucleotide has a pattern of backbone chiral centers which comprises or is (Sp)m(Rp)n, (Rp)n(Sp)m, (Np)t(Rp)n(Sp)m, or (Sp)t(Rp)n(Sp)m (unless otherwise specified, description of patterns of modifications and stereochemistry are from 5′ to 3′ as typically used in the art). In some embodiments, a provided pattern of backbone chiral centers comprises or is (Sp)m(Rp)n. In some embodiments, a provided pattern of backbone chiral centers comprises or is (Rp)n(Sp)m. In some embodiments, a provided pattern of backbone chiral centers comprises or is (Np)t(Rp)n(Sp)m. In some embodiments, a provided pattern of backbone chiral centers comprises or is (Np)tRp(Sp)m. In some embodiments, a provided pattern of backbone chiral centers comprises or is (Sp)tRp(Sp)m. In some embodiments, a provided pattern of backbone chiral centers comprises repeating (Sp)m(Rp)n, (Rp)n(Sp)m, (Np)t(Rp)n(Sp)m, or (Sp)t(Rp)n(Sp)m units. In some embodiments, a repeating unit is (Sp)m(Rp)n. In some embodiments, a repeating unit is SpRp. In some embodiments, a repeating unit is SpSpRp. In some embodiments, a repeating unit is SpRpRp. In some embodiments, a repeating unit is RpRpSp. In some embodiments, a repeating unit is (Rp)n(Sp)m. In some embodiments, a repeating unit is (Np)t(Rp)n(Sp)m. In some embodiments, a repeating unit is (Sp)t(Rp)n(Sp)m.

In some embodiments, t, n and m each are independently 1-20. In some embodiments, n is 1. In some embodiments, m is at least 2, 3, 4, 5, 6, 7, 8, 9, 10, 11, 12, 13, 14, or 15. In some embodiments, m is 2, 3, 4, 5, 6, 7, 8, 9, 10, 11, 12, 13, 14, or 15. In some embodiments, t is at least 2, 3, 4, 5, 6, 7, 8, 9, 10, 11, 12, 13, 14, or 15. In some embodiments, t is 2, 3, 4, 5, 6, 7, 8, 9, 10, 11, 12, 13, 14, or 15. In some embodiments, each of m and t is independently at least 2, 3, 4, 5, 6, 7, 8, 9, 10, 11, 12, 13, 14, or 15. In some embodiments, each of m and t is independently 2, 3, 4, 5, 6, 7, 8, 9, 10, 11, 12, 13, 14, or 15. In some embodiments, at least one of m and t is at least 5, 6, 7, 8, 9, 10, 11, 12, 13, 14, or 15. In some embodiments, m is at least 5, 6, 7, 8, 9, 10, 11, 12, 13, 14, or 15. In some embodiments, t is at least 5, 6, 7, 8, 9, 10, 11, 12, 13, 14, or 15.

In some embodiments, a region comprising a pattern, or a repeating pattern, of backbone chiral centers of (Sp)m(Rp)n, (Rp)n(Sp)m, (Np)t(Rp)n(Sp)m, or (Sp)t(Rp)n(Sp)m (structurally starting from the first, and ending at the last, internucleotidic linkage of the internucleotidic linkages which have the pattern, or the repeating pattern, of (Sp)m(Rp)n, (Rp)n(Sp)m, (Np)t(Rp)n(Sp)m, or (Sp)t(Rp)n(Sp)m, respectively; a “(repeating) (Sp)m(Rp)n region”, a “(repeating) (Rp)n(Sp)m region”, a “(repeating) (Np)t(Rp)n(Sp)m region”, or a “(repeating) (Sp)t(Rp)n(Sp)m region”, respectively, depending on repeating or not. For example, a (Sp)t(Rp)n(Sp)m region ((Sp)7(Rp)1(Sp)3) in WV-2555: mA*SmGmCmUmU*SC*ST*ST*SG*ST*SC*SC*RA*SG*SC*SmUmUmUmA*SmU (SEQ ID NO:1)) comprises no 2′-OR sugar modifications. In some embodiments, each sugar moieties in the region is —CH₂— at the 2′-position. In some embodiments, each sugar moieties in the region is an unmodified, natural, 2′-deoxyribose moiety of DNA. In some embodiments, a region comprising a pattern, or a repeating pattern, of backbone chiral centers which comprises or is (Sp)m(Rp)n, (Rp)n(Sp)m, (Np)t(Rp)n(Sp)m, or (Sp)t(Rp)n(Sp)m is flanked by a 5′-end region, which structurally ends with a nucleoside moiety (which nucleoside moiety, at its 3′-end, is connected to the first internucleotidic linkage of the region comprising a pattern, or a repeating pattern, of backbone chiral centers which comprises or is (Sp)m(Rp)n, (Rp)n(Sp)m, (Np)t(Rp)n(Sp)m, or (Sp)t(Rp)n(Sp)m. For example, a flanking 5′-end region in WV-2555: mA*SmGmCmUmU*SC*ST*ST*SG*ST*SC*SC*RA*SG*SC*SmUmUmUmA*SmU (SEQ ID NO:1)). In some embodiments, a region comprising a pattern, or a repeating pattern, of backbone chiral centers which comprises or is (Sp)m(Rp)n, (Rp)n(Sp)m, (Np)t(Rp)n(Sp)m, or (Sp)t(Rp)n(Sp)m is flanked by a 3′-end region, which structurally starts with a nucleoside moiety (which nucleoside moiety, at its 5′-end, is connected to the last internucleotidic linkage of the region comprising a pattern, or a repeating pattern, of backbone chiral centers which comprises or is (Sp)m(Rp)n, (Rp)n(Sp)m, (Np)t(Rp)n(Sp)m, or (Sp)t(Rp)n(Sp)m. For example, a flanking 3′-end region in WV-2555: mA*SmGmCmUmU*SC*ST*ST*SG*ST*SC*SC*RA*SG*SC*SmUmUmUmA*SmU (SEQ ID NO:1)). In some embodiments, a region comprising a pattern, or a repeating pattern, of backbone chiral centers which comprises or is (Sp)m(Rp)n, (Rp)n(Sp)m, (Np)t(Rp)n(Sp)m, or (Sp)t(Rp)n(Sp)m is flanked by a 5′-end and a 3′-end region. In some embodiments, the flanking 5′-end region and/or the 3′-end region comprise a modified internucleotidic linkage. In some embodiments, the flanking 5′-end region and/or the 3′-end region comprise a modified internucleotidic linkage comprising a Sp linkage phosphorus. In some embodiments, the flanking 5′-end region and/or the 3′-end region comprise a Sp phosphorothioate linkage. In some embodiments, the flanking 5′-end region and/or the 3′-end region comprise one or more natural phosphate linkages. In some embodiments, the flanking 5′-end region and/or the 3′-end region comprise one or more consecutive natural phosphate linkages. In some embodiments, the flanking 5′-end comprises only one modified internucleotidic linkage which is the 5′-end internucleotidic linkage, and one or more consecutive natural phosphate linkages (for example, in WV-2555: mA*SmGmCmUmU*SC*ST*ST*SG*ST*SC*SC*RA*SG*SC*SmUmUmUmA*SmU (SEQ ID NO:1) (SOOOSSSSSSSRSSSOOOS)). In some embodiments, the flanking 3′-end comprises only one modified internucleotidic linkage which is the 3′-end internucleotidic linkage, and one or more consecutive natural phosphate linkages (for example, in WV-2555: mA*SmGmCmUmU*SC*ST*ST*SG*ST*SC*SC*RA*SG*SC*SmUmUmUmA*SmU (SEQ ID NO:1) (SOOOSSSSSSSRSSSOOOS)). In some embodiments, the flanking 5′-end region and/or the 3′-end region comprise 2′-modified sugar units. In some embodiments, each sugar unit in the 5′-end region and/or the 3′-end region is independently modified. In some embodiments, each sugar unit in the 5′-end region and/or the 3′-end region independently comprises a 2′-modification (for example, in WV-2555: mA*SmGmCmUmU*SC*ST*ST*SG*ST*SC*SC*RA*SG*SC*SmUmUmUmA*SmU. In some embodiments, each sugar unit in the 5′-end region and/or the 3′-end region comprises the same 2′-modification. In some embodiments, a 2′-modification is 2′-OR, wherein R is optionally substituted C₁₋₆ aliphatic. In some embodiments, a 2′-modification is 2′-OMe. In some embodiments, a 2′-modification is 2′-MOE. In some embodiments, a 2′-modification is a LNA modification (which comprises a type of C2-C4 bridge).

In some embodiments, a provided pattern of backbone chiral centers comprises (Rp/Sp)-(All Rp or All Sp)-(Rp/Sp). In some embodiments, a provided pattern of backbone chiral centers comprises (Rp)-(All Sp)-(Rp). In some embodiments, a provided pattern of backbone chiral centers comprises (Sp)-(All Sp)-(Sp). In some embodiments, a provided pattern of backbone chiral centers comprises (Sp)-(All Rp)-(Sp). In some embodiments, a provided pattern of backbone chiral centers comprises (Rp/Sp)-(repeating (Sp)m(Rp)n)-(Rp/Sp). In some embodiments, a provided pattern of backbone chiral centers comprises (Rp/Sp)-(repeating SpSpRp)-(Rp/Sp).

In some embodiments, a provided pattern of backbone chiral centers is (Rp/Sp)-(All Rp or All Sp)-(Rp/Sp). In some embodiments, a provided pattern of backbone chiral centers is (Sp)-(All Sp)-(Sp). In some embodiments, each chiral internucleotidic linkage is Sp. In some embodiments, a provided pattern of backbone chiral centers is (Rp)-(All Sp)-(Rp). In some embodiments, a provided pattern of backbone chiral centers is (Sp)-(All Rp)-(Sp). In some embodiments, a provided pattern of backbone chiral centers is (Rp/Sp)-(repeating (Sp)m(Rp)n)-(Rp/Sp). In some embodiments, a provided pattern of backbone chiral centers is (Rp/Sp)-(repeating SpSpRp)-(Rp/Sp).

In some embodiments, a seed region and/or post-seed region, or any portion thereof, can comprise a pattern of backbone chiral centers.

In some embodiments, provided oligonucleotides comprise internucleotidic linkages of a particular type which alternate with internucleotidic linkages of a different type. In some embodiments, internucleotidic linkages of various types include, but are not limited to, phosphodiester, phosphorothioate, stereorandom phosphorothioate, stereocontrolled phosphorothioate (Rp or Sp), phosphodithioate, or any other type of internucleotidic linkage described herein or known in the art.

In some embodiments, the present disclosure provides an oligonucleotide composition comprising a first plurality of oligonucleotides which:
1) have a common base sequence; and
2) comprise one or more modified sugar moieties and modified internucleotidic linkages.

In some embodiments, the present disclosure provides an oligonucleotide composition comprising a first plurality of oligonucleotides capable of directing single-stranded RNA interference which:
1) have a common base sequence complementary to a target sequence in a transcript; and
2) comprise one or more modified sugar moieties and modified internucleotidic linkages.

In some embodiments, a reference condition is absence of the composition. In some embodiments, a reference condition is presence of a reference composition. Example reference compositions comprising a reference plurality of oligonucleotides are extensively described in this disclosure. In some embodiments, oligonucleotides of the reference plurality have a different structural elements (chemical modifications, stereochemistry, etc.) compared with oligonucleotides of the first plurality in a provided composition. In some embodiments, a provided oligonucleotide composition comprising a first plurality of oligonucleotide is chirally controlled in that the first plurality of oligonucleotides comprise one or more chirally controlled internucleotidic linkages. In some embodiments, a provided oligonucleotide composition comprising a first plurality of oligonucleotide is chirally controlled in that the first plurality of oligonucleotides comprise 1-20 chirally controlled internucleotidic linkages. In some embodiments, the first plurality of oligonucleotides comprise 2, 3, 4, 5, 6, 7, 8, 9, 10, 11, 12, 13, 14, or 15 chirally controlled internucleotidic linkages. In some embodiments, a reference composition is a stereorandom preparation of oligonucleotides having the same chemical modifications. In some embodiments, a reference composition is a mixture of stereoisomers while a provided composition is a single-stranded RNAi agent of one stereoisomer. In some embodiments, oligonucleotides of the reference plurality have the same base sequence as oligonucleotide of the first plurality in a provided composition. In some embodiments, oligonucleotides of the reference plurality have the same chemical modifications as oligonucleotide of the first plurality in a provided composition. In some embodiments, oligonucleotides of the reference plurality have the same sugar modifications as oligonucleotide of the first plurality in a provided composition. In some embodiments, oligonucleotides of the reference plurality have the same base modifications as oligonucleotide of the first plurality in a provided composition. In some embodiments, oligonucleotides of the reference plurality have the same internucleotidic linkage modifications as oligonucleotide of the first plurality in a provided composition. In some embodiments, oligonucleotides of the reference plurality have the same base sequence and the same chemical modifications as oligonucleotide of the first plurality in a provided composition. In some embodiments, oligonucleotides of the reference plurality have the same stereochemistry as oligonucleotide of the first plurality in a provided composition but different chemical modifications, e.g., base modification, sugar modification, internucleotidic linkage modifications, etc.

In some embodiments, the present disclosure provides a composition comprising an oligonucleotide, wherein the oligonucleotide is complementary or substantially complementary to a target RNA sequence, has a length of about 15 to about 49 total nucleotides, wherein the oligonucleotide comprises at least one non-natural base, sugar and/or internucleotidic linkage.

In some embodiments, the present disclosure provides an oligonucleotide composition comprising a single-stranded RNAi agent, wherein the single-stranded RNAi agent is complementary or substantially complementary to a target RNA sequence, has a length of about 15 to about 49 total nucleotides, and is capable of directing target-specific RNA interference, wherein the single-stranded RNAi agent comprises at least one non-natural base, sugar and/or internucleotidic linkage.

In some embodiments, the length is 15 to 49, about 17 to about 49, 17 to 49, about 19 to about 29, 19 to 29, about 19 to about 25, 19 to 25, about 19 to about 23, or 19 to 23 total nucleotides.

In some embodiments, the present disclosure provides an oligonucleotide composition comprising a first plurality of oligonucleotides which:
1) have a common base sequence complementary or substantially complementary to a target sequence in a transcript; and
2) comprise one or more modified sugar moieties and modified internucleotidic linkages,
the oligonucleotide composition being characterized in that, when it is contacted with the transcript, knockdown of the transcript is improved relative to that observed under reference conditions selected from the group consisting of absence of the composition, presence of a reference composition, and combinations thereof.

In some embodiments, the present disclosure provides an oligonucleotide composition comprising a first plurality of oligonucleotides capable of directing single-stranded RNA interference which:
1) have a common base sequence complementary to a target sequence in a transcript; and
2) comprise one or more modified sugar moieties and modified internucleotidic linkages,
the oligonucleotide composition being characterized in that, when it is contacted with the transcript in a RNA interference system, RNAi-mediated knockdown of the transcript is improved relative to that observed under reference conditions selected from the group consisting of absence of the composition, presence of a reference composition, and combinations thereof.

In some embodiments, the present disclosure provides an oligonucleotide composition comprising a first plurality of oligonucleotides, wherein oligonucleotides of the first plurality are of a particular oligonucleotide type defined by:
1) base sequence;
2) pattern of backbone linkages;
3) pattern of backbone chiral centers; and
4) pattern of backbone phosphorus modifications.

In some embodiments, the present disclosure provides an oligonucleotide composition comprising a first plurality of oligonucleotides capable of directing single-stranded RNA interference, wherein oligonucleotides of the first plurality are of a particular oligonucleotide type defined by:
1) base sequence;
2) pattern of backbone linkages;
3) pattern of backbone chiral centers; and
4) pattern of backbone phosphorus modifications.

In some embodiments, the present disclosure provides an oligonucleotide composition comprising a first plurality of oligonucleotides of an oligonucleotide type, wherein the oligonucleotide type is defined by:
1) base sequence;
2) pattern of backbone linkages;
3) pattern of backbone chiral centers; and
4) pattern of backbone phosphorus modifications, which composition is chirally controlled in that it is enriched, relative to a substantially racemic preparation of oligonucleotides having the same base sequence, for oligonucleotides of the particular oligonucleotide type,
the oligonucleotide composition being characterized in that, when it is contacted with the transcript, knockdown of the transcript is improved relative to that observed under reference conditions selected from the group consisting of absence of the composition, presence of a reference composition, and combinations thereof.

In some embodiments, the present disclosure provides an oligonucleotide composition comprising a first plurality of oligonucleotides which are capable of directing single-stranded RNA interference and are of an oligonucleotide type, wherein the oligonucleotide type is defined by:
1) base sequence;
2) pattern of backbone linkages;
3) pattern of backbone chiral centers; and
4) pattern of backbone phosphorus modifications, which composition is chirally controlled in that it is enriched, relative to a substantially racemic preparation of oligonucleotides having the same base sequence, for oligonucleotides of the particular oligonucleotide type,
the oligonucleotide composition being characterized in that, when it is contacted with the transcript in a RNA interference system, RNAi-mediated knockdown of the transcript is improved relative to that observed under reference conditions selected from the group consisting of absence of the composition, presence of a reference composition, and combinations thereof.

In some embodiments, a provided oligonucleotide has any of the Formats illustrated in FIG. 1, or any structural element illustrated in any of the Formats illustrated in FIG. 1.

In some embodiments, a provided single-stranded RNAi agent has any of the Formats illustrated in FIG. 1, or any structural element illustrated in any of the Formats illustrated in FIG. 1.

Among other things, the present disclosure presents data showing that various oligonucleotides of the disclosed Formats are capable of directing a decrease in the expression and/or level of a target gene or its gene product, when targeted against any of several different sequences, in any of several different genes. In some embodiments, the present disclosure presents data showing that various RNAi agents of the disclosed Formats are capable of directing RNA interference against any of many different sequences, in any of many different genes.

In some embodiments, an oligonucleotide is of Format 1. In some embodiments, an oligonucleotide is of Format 2. In some embodiments, an oligonucleotide is of Format 3. In some embodiments, an oligonucleotide is of Format 4. In some embodiments, an oligonucleotide is of Format 5. In some embodiments, an oligonucleotide is of Format 6. In some embodiments, an oligonucleotide is of Format 7. In some embodiments, an oligonucleotide is of Format 8. In some embodiments, an oligonucleotide is of Format 9. In some embodiments, an oligonucleotide is of Format 10. In some embodiments, an oligonucleotide is of Format 11. In some embodiments, an oligonucleotide is of Format 12. In some embodiments, an oligonucleotide is of Format 13. In some embodiments, an oligonucleotide is of Format 14. In some embodiments, an oligonucleotide is of Format 15. In some embodiments, an oligonucleotide is of Format 16. In some embodiments, an oligonucleotide is of Format 17. In some embodiments, an oligonucleotide is of Format 18. In some embodiments, an oligonucleotide is of Format 19. In some embodiments, an oligonucleotide is of Format 20. In some embodiments, an oligonucleotide is of Format 21. In some embodiments, an oligonucleotide is of Format 22. In some embodiments, an oligonucleotide is of Format 23. In some embodiments, an oligonucleotide is of Format 24. In some embodiments, an oligonucleotide is of Format 25. In some embodiments, an oligonucleotide is of Format 26. In some embodiments, an oligonucleotide is of Format 27. In some embodiments, an oligonucleotide is of Format 28. In some embodiments, an oligonucleotide is of Format 29. In some embodiments, an oligonucleotide is of Format 30. In some embodiments, an oligonucleotide is of Format 31. In some embodiments, an oligonucleotide is of Format 32. In some embodiments, an oligonucleotide is of Format 33. In some embodiments, an oligonucleotide is of Format 34. In some embodiments, an oligonucleotide is of Format 35. In some embodiments, an oligonucleotide is of Format 36. In some embodiments, an oligonucleotide is of Format 37. In some embodiments, an oligonucleotide is of Format 38. In some embodiments, an oligonucleotide is of Format 39. In some embodiments, an oligonucleotide is of Format 40. In some embodiments, an oligonucleotide is of Format 41. In some embodiments, an oligonucleotide is of Format 42. In some embodiments, an oligonucleotide is of Format 43. In some embodiments, an oligonucleotide is of Format 44. In some embodiments, an oligonucleotide is of Format 45. In some embodiments, an oligonucleotide is of Format 46. In some embodiments, an oligonucleotide is of Format 47. In some embodiments, an oligonucleotide is of Format 48. In some embodiments, an oligonucleotide is of Format 49. In some embodiments, an oligonucleotide is of Format 50. In some embodiments, an oligonucleotide is of Format 51. In some embodiments, an oligonucleotide is of Format 52. In some embodiments, an oligonucleotide is of Format 53. In some embodiments, an oligonucleotide is of Format 54. In some embodiments, an oligonucleotide is of Format 55. In some embodiments, an oligonucleotide is of Format 56. In some embodiments, an oligonucleotide is of Format 57. In some embodiments, an oligonucleotide is of Format 58. In some embodiments, an oligonucleotide is of Format 59. In some embodiments, an oligonucleotide is of Format 60. In some embodiments, an oligonucleotide is of Format 61. In some embodiments, an oligonucleotide is of Format 62. In some embodiments, an oligonucleotide is of Format 63. In some embodiments, an oligonucleotide is of Format 64. In some embodiments, an oligonucleotide is of Format 65. In some embodiments, an oligonucleotide is of Format 66. In some embodiments, an oligonucleotide is of Format 67. In some embodiments, an oligonucleotide is of Format 68. In some embodiments, an oligonucleotide is of Format 69. In some embodiments, an oligonucleotide is of Format 70. In some embodiments, an oligonucleotide is of Format 71. In some embodiments, an oligonucleotide is of Format 72. In some embodiments, an oligonucleotide is of Format 73. In some embodiments, an oligonucleotide is of Format 74. In some embodiments, an oligonucleotide is of Format 75. In some embodiments, an oligonucleotide is of Format 76. In some embodiments, an oligonucleotide is of Format 77. In some embodiments, an oligonucleotide is of Format 78. In some embodiments, an oligonucleotide is of Format 79. In some embodiments, an oligonucleotide is of Format 80. In some embodiments, an oligonucleotide is of Format 81. In some embodiments, an oligonucleotide is of Format 82. In some embodiments, an oligonucleotide is of Format 83. In some embodiments, an oligonucleotide is of Format 84. In some embodiments, an oligonucleotide is of Format 85. In some embodiments, an oligonucleotide is of Format 86. In some embodiments, an oligonucleotide is of Format 87. In some embodiments, an oligonucleotide is of Format 88. In some embodiments, an oligonucleotide is of Format 89. In some embodiments, an oligonucleotide is of Format 90. In some embodiments, an oligonucleotide is of Format 91. In some embodiments, an oligonucleotide is of Format 92. In some embodiments, an oligonucleotide is of Format 93. In some embodiments, an oligonucleotide is of Format 94. In some embodiments, an oligonucleotide is of Format 95. In some embodiments, an oligonucleotide is of Format 96. In some embodiments, an oligonucleotide is of Format 97. In some embodiments, an oligonucleotide is of Format 98. In some embodiments, an oligonucleotide is of Format 99. In some embodiments, an oligonucleotide is of Format 100. In some embodiments, an oligonucleotide is of Format 101. In some embodiments, an oligonucleotide is of Format 102. In some embodiments, an oligonucleotide is of Format 103. In some embodiments, an oligonucleotide is of Format 104. In some embodiments, an oligonucleotide is of Format 105. In some embodiments, an oligonucleotide is of Format 106. In some embodiments, an oligonucleotide is of Format 107. Various non-limiting examples of formats of stereocontrolled (chirally controlled) oligonucleotides are shown in Tables 71A to 71C. In some embodiments, an oligonucleotide is of Format S1. In some embodiments, an oligonucleotide is of Format S2. In some embodiments, an oligonucleotide is of Format S3. In some embodiments, an oligonucleotide is of Format S4. In some embodiments, an oligonucleotide is of Format S5. In some embodiments, an oligonucleotide is of Format S6. In some embodiments, an oligonucleotide is of Format S7. In some embodiments, an oligonucleotide is of Format S8. In some embodiments, an oligonucleotide is of Format S9. In some embodiments, an oligonucleotide is of Format S10. In some embodiments, an oligonucleotide is of Format S11. In some embodiments, an oligonucleotide is of Format S12. In some embodiments, an oligonucleotide is of Format S13. In some embodiments, an oligonucleotide is of Format S14. In some embodiments, an oligonucleotide is of Format S15. In some embodiments, an oligonucleotide is of Format S16. In some embodiments, an oligonucleotide is of Format S17. In some embodiments, an oligonucleotide is of Format 518. In some embodiments, an oligonucleotide is of Format 519. In some embodiments, an oligonucleotide is of Format S20. In some embodiments, an oligonucleotide is of Format 521. In some embodiments, an oligonucleotide is of Format S22. In some embodiments, an oligonucleotide is of Format S23. In some embodiments, an oligonucleotide is of Format S24. In some embodiments, an oligonucleotide is of Format S25. In some embodiments, an oligonucleotide is of Format S26. In some embodiments, an oligonucleotide is of Format S27. In some embodiments, an oligonucleotide is of Format S28. In some embodiments, an oligonucleotide is of Format S29. In some embodiments, an oligonucleotide is of Format S30. In some embodiments, an oligonucleotide is of Format 531. In some embodiments, an oligonucleotide is of Format S32. In some embodiments, an oligonucleotide is of Format S33. In some embodiments, an oligonucleotide is of Format S34. In some embodiments, an oligonucleotide is of Format S35. In some embodiments, an oligonucleotide is of Format S36. In some embodiments, an oligonucleotide is of Format S37. In some embodiments, an oligonucleotide is of Format S38. In some embodiments, an oligonucleotide is of Format S39. In some embodiments, an oligonucleotide is of Format S40. In some embodiments, an oligonucleotide is of Format 541. In some embodiments, an oligonucleotide is of Format S42. In some embodiments, an oligonucleotide is of Format S43. In some embodiments, an oligonucleotide is of Format S44.

In some embodiments, an oligonucleotide having any of the structures described and/or illustrated herein is capable of directing RNA interference. In some embodiments, an oligonucleotide having any of the structures described and/or illustrated herein is capable of directing RNase H-mediated knockdown. In some embodiments, an oligonucleotide having any of the structures described and/or illustrated herein is capable of directing RNA interference and/or RNase H-mediated knockdown. In some embodiments, an oligonucleotide comprises any structural element of any oligonucleotide described herein, or any Format described herein or illustrated in FIG. 1. In some embodiments, an oligonucleotide comprises any structural element of any oligonucleotide described herein, or any Format described herein or illustrated in FIG. 1 and is capable of directing RNA interference. In some embodiments, an oligonucleotide comprises any structural element of any oligonucleotide described herein, or any Format described herein or illustrated in FIG. 1 and is capable of directing RNase H-mediated knockdown. In some embodiments, an oligonucleotide comprises any structural element of any oligonucleotide described herein, or any Format described herein or illustrated in FIG. 1 and is capable of directing RNA interference and/or RNase H-mediated knockdown.

In some embodiments, a RNAi agent comprises any one or more of: a 5′-end structure, a 5′-end region, a seed region, a post-seed region, and a 3′-end region, and an optional additional chemical moiety. In some embodiments, a seed region is any seed region described herein or known in the art. In some embodiments, a post-seed region can be any region between a seed region and a 3′-end region described herein or known in the art. In some embodiments, a 3′-end region can be any 3′-end region described herein or known in the art. In some embodiments, any optional additional chemical moiety can be any optional additional chemical moiety described herein or known in the art. Any individual 5′-end structure, 5′-end region, seed region, post-seed region, 3′-end region, and optional additional chemical moiety described herein or known in the art can be combined, independently, with any other 5′-end structure, 5′-end region, seed region, post-seed region, 3′-end region, and optional additional chemical moiety described herein or known in the art. In some embodiments, as non-limiting examples, a region of a single-stranded RNAi agent is a 5′-end structure, a 5′-end region, a seed region, a post-seed region, a portion of a seed region, a portion of a post-seed region, or a 3′-terminal dinucleotide.

In some embodiments, the base sequence of a provided oligonucleotide consists of the base sequence of any oligonucleotide disclosed herein. In some embodiments, the base sequence of a provided oligonucleotide comprises the base sequence of any oligonucleotide disclosed herein. In some embodiments, the base sequence of a provided oligonucleotide comprises a sequence comprising the sequence of 15 contiguous bases of the base sequence of any oligonucleotide disclosed herein. In some embodiments, the base sequence of a provided oligonucleotide comprises a sequence comprising the sequence of 20 contiguous bases, with up to 5 mismatches, of the base sequence of any oligonucleotide disclosed herein.

In some embodiments, a provided oligonucleotide is capable of directing a decrease in the expression and/or level of a target gene or its gene product. In some embodiments, a provided oligonucleotide is capable of directing single-stranded RNAi interference. In some embodiments, a provided oligonucleotide is capable of directing RNase H-mediated knockdown. In some embodiments, a provided oligonucleotide is capable of directing single-stranded RNA interference and RNase H-mediated knockdown. In some embodiments, an oligonucleotide comprises a sequence which targets any transcript or gene targeted by an oligonucleotide disclosed herein.

In some embodiments, provided oligonucleotides target ACVR2B or MSTN-R. In some embodiments, provided oligonucleotides target APOB. In some embodiments, provided oligonucleotides target APOC3. In some embodiments, provided oligonucleotides target FXI (Factor XI). In some embodiments, provided oligonucleotides target KRT14. In some embodiments, provided oligonucleotides target MSTN. In some embodiments, provided oligonucleotides target PCSK9.

In some embodiments, provided oligonucleotides target PNPLA3.

In some embodiments, provided oligonucleotides can be used to decrease or inhibit the activity, level and/or expression of a gene or its gene product. In some embodiments, provided oligonucleotides can be used to decrease or inhibit the activity, level and/or expression of a gene or its gene product, wherein abnormal or excessive activity, level and/or expression of, a deleterious mutation in, or abnormal tissue or inter- or intracellular distribution of a gene or its gene product is related to, causes and/or is associated with a disorder. In some embodiments, provided oligonucleotides can be used to treat a disorder and/or to manufacture a medicament for the treatment of a disorder related to, caused and/or associated with the abnormal or excessive activity, level and/or expression or abnormal distribution of a gene or its gene product.

In some embodiments, provided oligonucleotides can be used to treat or used to manufacture a medicament for treatment of a disorder related to a specific gene or gene product. In some embodiments, the present disclosure pertains to methods of using oligonucleotides disclosed herein which are capable of targeting ACVR2B and useful for treating and/or manufacturing a treatment for a ACVR2B-related disorder. In some embodiments, the present disclosure pertains to methods of using oligonucleotides disclosed herein which are capable of targeting APOB and useful for treating and/or manufacturing a treatment for a APOB-related disorder.

In some embodiments, the present disclosure pertains to methods of using oligonucleotides disclosed herein which are capable of targeting APOC3 and useful for treating and/or manufacturing a treatment for a APOC3-related disorder.

In some embodiments, the present disclosure pertains to methods of using oligonucleotides disclosed herein which are capable of targeting FXI (Factor XI) and useful for treating and/or manufacturing a treatment for a FXI (Factor XI)-related disorder. In some embodiments, the present disclosure pertains to methods of using oligonucleotides disclosed herein which are capable of targeting KRT or KRT14 and useful for treating and/or manufacturing a treatment for a KRT or KRT14-related disorder. In some embodiments, the present disclosure pertains to methods of using oligonucleotides disclosed herein which are capable of targeting myostatin (MSTN) and useful for treating and/or manufacturing a treatment for a myostatin (MSTN)-related disorder. In some embodiments, the present disclosure pertains to methods of using oligonucleotides disclosed herein which are capable of targeting PCSK9 and useful for treating and/or manufacturing a treatment for a PCSK9-related disorder.

In some embodiments, the present disclosure pertains to methods of using oligonucleotides disclosed herein which are capable of targeting PNPLA3 and useful for treating and/or manufacturing a treatment for a PNPLA3-related disorder.

In some embodiments, an oligonucleotide capable of targeting a gene comprises a base sequence which is a portion of or complementary or substantially complementary to a portion of the base sequence of the target gene. In some embodiments, a portion is at least 15 bases long. In some embodiments, a base sequence of a single-stranded RNAi agent can comprise or consist of a base sequence which has a specified maximum number of mismatches from a specified base sequence.

In some embodiments, a mismatch is a difference between the base sequence or length when two sequences are maximally aligned and compared. As a non-limiting example, a mismatch is counted if a difference exists between the base at a particular location in one sequence and the base at the corresponding position in another sequence. Thus, a mismatch is counted, for example, if a position in one sequence has a particular base (e.g., A), and the corresponding position on the other sequence has a different base (e.g., G, C or U). A mismatch is also counted, e.g., if a position in one sequence has a base (e.g., A), and the corresponding position on the other sequence has no base (e.g., that position is an abasic nucleotide which comprises a phosphate-sugar backbone but no base) or that position is skipped. A single-stranded nick in either sequence (or in the sense or antisense strand) may not be counted as mismatch, for example, no mismatch would be counted if one sequence comprises the sequence 5′-AG-3′, but the other sequence comprises the sequence 5′-AG-3′ with a single-stranded nick between the A and the G. A base modification is generally not considered a mismatch, for example, if one sequence comprises a C, and the other sequence comprises a modified C (e.g., 5mC) at the same position, no mismatch may be counted. In some embodiments, for purposes of counting mismatches, substitution of a T for U or vice versa is not considered a mismatch.

In some embodiments, an oligonucleotide is complementary or totally or 100% complementary to a target sequence (e.g., a RNA, such as a mRNA), meaning that the base sequence of the oligonucleotide has no mismatches with a sequence which is fully complementary (e.g., base-pairs via Watson-Crick basepairing) to the target sequence. Without wishing to be bound by any particular theory, the disclosure notes that, for a single-stranded RNAi agent, it is not necessary for the 5′-end nucleotide moiety or the 3′-terminal dinucleotide to base-pair with the target. These may be mismatches. In addition, an antisense oligonucleotide or single-stranded RNAi agent can have a small number of internal mismatches and still direct a decrease in the expression and/or level of a target gene or its gene product and/or direct RNase H-mediated knockdown and/or RNA interference. If a first base sequence of an oligonucleotide, (e.g., antisense oligonucleotide or single-stranded RNAi agent) has a small number of mismatches from a reference base sequence which is 100% complementary to a target sequence, then the first base sequence is substantially complementary to the target sequence. In some embodiments, an oligonucleotide, (e.g., antisense oligonucleotide or single-stranded RNAi agent) can have a base sequence which is complementary or substantially complementary to a target sequence. In some embodiments, complementarity is determined based on Watson-Crick base pairs (guanine-cytosine and adenine-thymine/uracil), wherein guanine, cytosine, adenine, thymine, uracil may be optionally and independently modified but maintains their pairing hydrogen-bonding patters as unmodified. In some embodiments, a sequence complementary to another sequence comprises at least 5, 6, 7, 8, 9, 10, 11, 12, 13, 14, 15, 16, 17, 18, 19, 20 bases.

In some embodiments, an oligonucleotide, oligonucleotide composition or oligonucleotide type has a common pattern of backbone linkages. In some embodiments, a common pattern of backbone linkages comprises at least 10 modified internucleotidic linkages.

In some embodiments, a common pattern of backbone linkages comprises at least 10, 11, 12, 13, 14, 15, 16, 17, 18, 19 or 20 modified internucleotidic linkages. In some embodiments, a common pattern of backbone linkages comprises at least 15 modified internucleotidic linkages. In some embodiments, a common pattern of backbone linkages comprises at least 19 modified internucleotidic linkages. In some embodiments, a common pattern of backbone linkages comprises no more than 10, 11, 12, 13, 14, 15, 16, 17, 18, 19 or 20 modified internucleotidic linkages. In some embodiments, a common pattern of backbone linkages comprises no more than 19 modified internucleotidic linkages. In some embodiments, a common pattern of backbone linkages comprises no more than 15 modified internucleotidic linkages. In some embodiments, a common pattern of backbone linkages comprises 11 to 21 modified internucleotidic linkages. In some embodiments, a common pattern of backbone linkages comprises 0 phosphodiesters. In some embodiments, a common pattern of backbone linkages comprises 1 phosphodiester. In some embodiments, a common pattern of backbone linkages comprises 2, 3, 4, 5, 6, 7, 8, 9, 10, 11, 12, 13, 14, 15, 16, 17, 18 or 19 phosphodiesters. In some embodiments, a common pattern of backbone linkages comprises at least 2 to 19 phosphodiesters. In some embodiments, the phosphodiesters are optionally contiguous or not contiguous. In some embodiments, the modified internucleotidic linkages are optionally contiguous or not contiguous.

In some embodiments, a common pattern of backbone linkages comprises at least 10 phosphorothioate linkages. In some embodiments, a common pattern of backbone linkages comprises at least 11 phosphorothioate linkages. In some embodiments, a common pattern of backbone linkages comprises at least 12 to 19 phosphorothioate linkages. In some embodiments, a common pattern of backbone linkages comprises at least 12, 13, 14, 15, 16, 17, 18, 19 or 20 phosphorothioate linkages. In some embodiments, a common pattern of backbone linkages comprises 0 phosphodiesters. In some embodiments, a common pattern of backbone linkages comprises 1 to 6 phosphodiesters and 13 to 19 phosphorothioate linkages. In some embodiments, the phosphodiesters are optionally contiguous or not contiguous. In some embodiments, the phosphorothioate linkages are optionally contiguous or not contiguous.

In some embodiments, an oligonucleotide, oligonucleotide composition or oligonucleotide type has a common pattern of backbone chiral centers. In some embodiments, a common pattern of backbone chiral centers comprises at least 1 internucleotidic linkage in the Sp configuration. In some embodiments, a common pattern of backbone chiral centers comprises at least 1 internucleotidic linkage which is phosphorothioate in the Sp configuration.

In some embodiments, a common pattern of backbone chiral centers comprises at least 5 internucleotidic linkages in the Sp configuration. In some embodiments, a common pattern of backbone chiral centers comprises at least 6 to 19 internucleotidic linkages in the Sp configuration. In some embodiments, a common pattern of backbone chiral centers comprises at least 6, 7, 8, 9, 10, 11, 12, 13, 14, 15, 16, 17, 18, 19 or 20 internucleotidic linkages in the Sp configuration. In some embodiments, a common pattern of backbone chiral centers comprises no more than 8 internucleotidic linkages in the Rp configuration. In some embodiments, a common pattern of backbone chiral centers comprises no more than 1 to 7 internucleotidic linkages in the Rp configuration. In some embodiments, a common pattern of backbone chiral centers comprises no more than 8 internucleotidic linkages which are not chiral (as a non-limiting example, a phosphodiester). In some embodiments, a common pattern of backbone chiral centers comprises no more than 1, 2, 3, 4, 5, 6, or 7 internucleotidic linkages which are not chiral. In some embodiments, a common pattern of backbone chiral centers comprises at least 10 internucleotidic linkages in the Sp configuration, and no more than 8 internucleotidic linkages which are not chiral. In some embodiments, a common pattern of backbone chiral centers comprises at least 11 internucleotidic linkages in the Sp configuration, and no more than 7 internucleotidic linkages which are not chiral. In some embodiments, a common pattern of backbone chiral centers comprises at least 12 internucleotidic linkages in the Sp configuration, and no more than 6 internucleotidic linkages which are not chiral. In some embodiments, a common pattern of backbone chiral centers comprises at least 13 internucleotidic linkages in the Sp configuration, and no more than 6 internucleotidic linkages which are not chiral. In some embodiments, a common pattern of backbone chiral centers comprises at least 14 internucleotidic linkages in the Sp configuration, and no more than 5 internucleotidic linkages which are not chiral. In some embodiments, a common pattern of backbone chiral centers comprises at least 15 internucleotidic linkages in the Sp configuration, and no more than 4 internucleotidic linkages which are not chiral. In some embodiments, the internucleotidic linkages in the Sp configuration are optionally contiguous or not contiguous. In some embodiments, the internucleotidic linkages in the Rp configuration are optionally contiguous or not contiguous. In some embodiments, the internucleotidic linkages which are not chiral are optionally contiguous or not contiguous.

In some embodiments, oligonucleotides in provided compositions have a common pattern of backbone phosphorus modifications. In some embodiments, a provided composition is an oligonucleotide composition that is chirally controlled in that the composition contains a predetermined level of oligonucleotides of an individual oligonucleotide type, wherein an oligonucleotide type is defined by:
1) base sequence;
2) pattern of backbone linkages;
3) pattern of backbone chiral centers; and
4) pattern of backbone phosphorus modifications.

As noted above and understood in the art, in some embodiments, base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, a particular oligonucleotide type may be defined by
1A) base identity;
1B) pattern of base modification;
1C) pattern of sugar modification;
2) pattern of backbone linkages;
3) pattern of backbone chiral centers; and
4) pattern of backbone phosphorus modifications.

Thus, in some embodiments, oligonucleotides of a particular type may share identical bases but differ in their pattern of base modifications and/or sugar modifications. In some embodiments, oligonucleotides of a particular type may share identical bases and pattern of base modifications (including, e.g., absence of base modification), but differ in pattern of sugar modifications.

In some embodiments, oligonucleotides of a particular type are chemically identical in that they have the same base sequence (including length), the same pattern of chemical modifications to sugar and base moieties, the same pattern of backbone linkages (e.g., pattern of natural phosphate linkages, non-negatively charged linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as -S⁻, and -L-R¹ of Formula I).

In some embodiments, the present disclosure provides chirally controlled oligonucleotide compositions of oligonucleotides comprising multiple (e.g., more than 5, 6, 7, 8, 9, or 10) internucleotidic linkages, and particularly for oligonucleotides comprising multiple (e.g., more than 5, 6, 7, 8, 9, or 10) chiral internucleotidic linkages. In some embodiments, in a stereorandom or racemic preparation of oligonucleotides, at least one chiral internucleotidic linkage is formed with less than 90:10, 95:5, 96:4, 97:3, or 98:2 diastereoselectivity. In some embodiments, for a stereoselective or chirally controlled preparation of oligonucleotides, each chiral internucleotidic linkage is formed with greater than 90:10, 95:5, 96:4, 97:3, or 98:2 diastereoselectivity. In some embodiments, for a stereoselective or chirally controlled preparation of oligonucleotides, each chiral internucleotidic linkage is formed with greater than 95:5 diastereoselectivity. In some embodiments, for a stereoselective or chirally controlled preparation of oligonucleotides, each chiral internucleotidic linkage is formed with greater than 96:4 diastereoselectivity. In some embodiments, for a stereoselective or chirally controlled preparation of oligonucleotides, each chiral internucleotidic linkage is formed with greater than 97:3 diastereoselectivity. In some embodiments, for a stereoselective or chirally controlled preparation of oligonucleotides, each chiral internucleotidic linkage is formed with greater than 98:2 diastereoselectivity. In some embodiments, for a stereoselective or chirally controlled preparation of oligonucleotides, each chiral internucleotidic linkage is formed with greater than 99:1 diastereoselectivity. In some embodiments, diastereoselectivity of a chiral internucleotidic linkage in an oligonucleotide may be measured through a model reaction, e.g. formation of a dimer under essentially the same or comparable conditions wherein the dimer has the same internucleotidic linkage as the chiral internucleotidic linkage, the 5′-nucleoside of the dimer is the same as the nucleoside to the 5′-end of the chiral internucleotidic linkage, and the 3′-nucleoside of the dimer is the same as the nucleoside to the 3′-end of the chiral internucleotidic linkage. In some embodiments, a chirally controlled internucleotidic linkage has 90%-100% (e.g., 90%, 91%, 92%, 93%, 94%, 95%, 96%, 97%, 98%, 99%, or 99.5%) diastereopurity at the linkage phosphorus. In some embodiments, a chirally controlled internucleotidic linkage has 95%-100% (e.g., 95%, 96%, 97%, 98%, 99%, or 99.5%) diastereopurity at the linkage phosphorus. In some embodiments, a chirally controlled internucleotidic linkage has 97%-100% (e.g., 97%, 98%, 99%, or 99.5%) diastereopurity at the linkage phosphorus. In some embodiments, a chirally controlled internucleotidic linkage has at least 97% diastereopurity. In some embodiments, a chirally controlled internucleotidic linkage has at least 98% diastereopurity. In some embodiments, a chirally controlled internucleotidic linkage has at least 99% diastereopurity. In some embodiments, a non-chirally controlled (racemic/stereorandom) internucleotidic linkage has less than 90% diastereopurity.

Among other things, the present disclosure provides oligonucleotide compositions and technologies for optimizing properties.

Among other things, the present disclosure provides oligonucleotide compositions and technologies for optimizing properties, e.g., improved single-stranded RNA interference, RNase H-mediated knockdown, etc. In some embodiments, the present disclosure provides methods for lowering immune response associated with administration of oligonucleotides and compositions thereof (i.e., of administering oligonucleotide compositions so that undesirable immune responses to oligonucleotides in the compositions are reduced, for example relative to those observed with a reference composition of nucleotides of comparable or identical nucleotide sequence). In some embodiments, the present disclosure provides methods for increasing binding to certain proteins by oligonucleotides and compositions thereof. In some embodiments, the present disclosure provides methods for increasing binding to certain proteins by oligonucleotides and compositions thereof. In some embodiments, the present disclosure provides methods for enhancing delivery of oligonucleotides and compositions thereof. Among other things, the present disclosure encompasses the recognition that optimal delivery of oligonucleotides to their targets, in some embodiments, involves balance of oligonucleotides binding to certain proteins so that oligonucleotides can be transported to the desired locations, and oligonucleotide release so that oligonucleotides can be properly released from certain proteins to perform their desired functions, for example, hybridization with their targets, cleavage of their targets, inhibition of translation, modulation of transcript processing, etc. As exemplified in this disclosure, the present disclosure recognizes, among other things, that improvement of oligonucleotide properties can be achieved through chemical modifications and/or stereochemistry.

In some embodiments, the present disclosure provides a method for treating or preventing a disease, comprising administering to a subject an oligonucleotide composition described herein.

In some embodiments, a disease is one in which, after administering a provided composition, knocking down a target nucleic acid via single-stranded RNA interference can repair, restore or introduce a new beneficial function.

In some embodiments, a disease is cancer.

In some embodiments, a common sequence comprises a sequence selected from Table 1A. In some embodiments, a common sequence is a sequence selected from Table 1A. In some embodiments, a pattern of backbone chiral centers is selected from those described in Table 1A.

In some embodiments, the present disclosure provides a method comprising administering a composition comprising a first plurality of oligonucleotides, which composition displays improved delivery as compared with a reference composition comprising a plurality of oligonucleotides, each of which also has the common base sequence but which differs structurally from the oligonucleotides of the first plurality in that:
individual oligonucleotides within the reference plurality differ from one another in stereochemical structure; and/or
at least some oligonucleotides within the reference plurality have a structure different from a structure represented by the plurality of oligonucleotides of the composition.

In some embodiments, the present disclosure provides a method of administering an oligonucleotide composition comprising a first plurality of oligonucleotides capable of directing a decrease in the expression and/or level of a target gene or its gene product and having a common nucleotide sequence, the improvement that comprises:
administering an oligonucleotide comprising a first plurality of oligonucleotides that is characterized by improved delivery relative to a reference oligonucleotide composition of the same common nucleotide sequence.

In some embodiments, the present disclosure provides a method of administering an oligonucleotide composition comprising a first plurality of oligonucleotides capable of directing single-stranded RNA interference and having a common nucleotide sequence, the improvement that comprises:
administering an oligonucleotide comprising a first plurality of oligonucleotides that is characterized by improved delivery relative to a reference oligonucleotide composition of the same common nucleotide sequence.

In some embodiments, the present disclosure provides a single-stranded RNAi agent of an oligonucleotide selected from any of the Tables, including but not limited to Table 1A, or otherwise disclosed herein. In some embodiments, the present disclosure provides a single-stranded RNAi agent of an oligonucleotide selected from any of the Tables, including but not limited to Table 1A, or otherwise disclosed herein, wherein the oligonucleotide is conjugated to a lipid moiety.

In some embodiments, the oligonucleotide is no more than 25 bases long. In some embodiments, the oligonucleotide is no more than 30 bases long. n some embodiments, the oligonucleotide is no more than 35 bases long. In some embodiments, the oligonucleotide is no more than 40 bases long. In some embodiments, the oligonucleotide is no more than 45 bases long. In some embodiments, the oligonucleotide is no more than 50 bases long. In some embodiments, the oligonucleotide is no more than 55 bases long. In some embodiments, the oligonucleotide is no more than 60 bases long.

In some embodiments, a provided oligonucleotide comprises a lipid moiety. In some embodiments, a lipid moiety is incorporated by conjugation with a lipid. In some embodiments, a lipid moiety is a fatty acid. In some embodiments, an oligonucleotide is conjugated to a fatty acid. In some embodiments, a provided single-stranded RNAi agent further comprises a lipid. In some embodiments, a provided single-stranded RNAi agent comprises a lipid moiety conjugated at the 9^{th} or 11^{th} nucleotide (counting from the 5′-end). In some embodiments, an oligonucleotide is conjugated at the base to a fatty acid. In some embodiments, a provided single-stranded RNAi agent comprises a lipid moiety. In some embodiments, a provided single-stranded RNAi agent comprises a lipid moiety conjugated at the base at the 9^{th} or 11^{th} nucleotide (counting from the 5′-end).

In some embodiments, the present disclosure provides a compound, e.g., an oligonucleotide, having the structure of formula O-I:

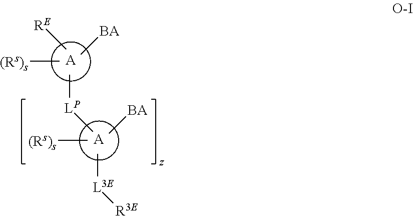

or a salt thereof, wherein each variable is independently as described in the present disclosure.

In some embodiments, the present disclosure provides a compound of structure:

A^{c}-[-L^{M}-(R^{D})_{a]b},[(A^{c})ₐ-L^{M}]_{b}-R^{D},(A^{c})ₐ-L^{M}-(A^{c})_{b}, or (A^{c})ₐ-L^{M}-(R^{D})_{b},

or a salt thereof, wherein each variable is independently as described in the present disclosure.

In some embodiments, each A^{c} is independently an oligonucleotide moiety of an oligonucleotide of formula O-I or a salt thereof (e.g., [H]ₐ-A^{c} or [H]_{b}-A^{c} is an oligonucleotide of formula O-I or a salt thereof). In some embodiments, the present disclosure provides an oligonucleotide having the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, the present disclosure provides oligonucleotide compositions comprising oligonucleotides having the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, the present disclosure provides oligonucleotide compositions comprising predetermined levels (as described in the present disclosure) of oligonucleotides having the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, the present disclosure provides chirally controlled oligonucleotide compositions comprising oligonucleotides having the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, oligonucleotides of a plurality (e.g., a first plurality), or oligonucleotides of an oligonucleotide type, are oligonucleotides having the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, oligonucleotides in provided compositions, e.g., provided chirally controlled oligonucleotide compositions, have the structure of A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or a salt thereof. In some embodiments, the structure is A^{c}-[-L^{M}-(R^{D})ₐ]_{b} or a salt thereof. In some embodiments, the structure is [(A^{c})ₐ-L^{M}]_{b}-R^{D} or a salt thereof. In some embodiments, the structure is (A^{c})ₐ-L^{M}-(A^{c})b or a salt thereof. In some embodiments, the structure is A^{c}-[-L^{M}-(R^{D})ₐ]_{b} or a salt thereof.

In some embodiments, each A^{c} is independently an oligonucleotide moiety of an oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, or a salt thereof.

In some embodiments, a conjugate has the structure of A^{c}-[L^{LD}-(R^{LD})ₐ]_{b}, wherein each variable is independently as described in the present disclosure.

In some embodiments, the present disclosure provides an oligonucleotide composition comprising a plurality of oligonucleotides having the structure of:

A^{c}-[-L^{LD}-(R^{LD})ₐ]_{b}, or [(A^{c})ₐ-L^{LD}]_{b}-R^{LD},
wherein:
each A^{c} is independently an oligonucleotide moiety (e.g., [H]ₐ-A^{c} or [H]_{b}-A^{c} is an oligonucleotide);
a is 1-1000;
b is 1-1000;
each L^{LD} is independently a linker moiety; and
each R^{LD} is independently a lipid moiety or a targeting moiety.

In some embodiments, the present disclosure provides an oligonucleotide composition comprising a plurality of oligonucleotides having the structure of:

A^{c}-[L^{LD}-(R^{LD})ₐ]_{b}, or [(A^{c})ₐ-L^{LD}]_{b}-R^{LD},
wherein:
each A^{c} is independently an oligonucleotide moiety (e.g., [H]ₐ-A^{c} or [H]_{b}-A^{c} is an oligonucleotide);
a is 1-1000;
b is 1-1000;
each L^{LD} is independently a covalent bond or an optionally substituted, C₁-C₈₀ saturated or partially unsaturated aliphatic group, wherein one or more methylene units are optionally and independently replaced by T^{LD} or an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
each R^{LD} is independently hydrogen, or an optionally substituted, C₁-C₈₀ saturated or partially unsaturated aliphatic group, wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, and —C(O)O—;
T^{LD} has the structure of Formula I′:

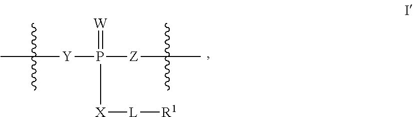

W is O, S or Se;
each of X, Y and Z is independently —O—, —S—, —N(-L-R¹)—, or L;
L is a covalent bond or an optionally substituted, linear or branched C₁-C₁₀ aliphatic, wherein one or more methylene units of L are optionally and independently replaced by an optionally substituted group selected from C1-C6 aliphatic moiety, C1-C6 alkenylene, —C≡C—, a C₁-C₆ heteroaliphatic moiety, —C(R′)₂—, -Cy-, —B(R′)—, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C1-C6 alkylene, C1-C6 alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two or more R′ are taken together with their intervening atoms to form an optionally substituted C₃-C₁₄ group selected from aryl, carbocyclyl, heterocyclyl, and heteroaryl;
-Cy- is an optionally substituted bivalent ring selected from phenylene, C₃-C₁₄ carbocyclylene, C₁₀-C₁₄ arylene, C₅-C₁₄ heteroarylene, and C₃-C₁₄ heterocyclylene; and
each R is independently hydrogen, or an optionally substituted group selected from C₁-C₂₀ aliphatic, C₃-C₂₀ carbocyclyl, C₆-C₂₀ aryl, C₅-C₂₀ heteroaryl, and C₃-C₂₀ heterocyclyl.

In some embodiments, [H]ₐ-A^{c} or [H]_{b}-A^{c} is an oligonucleotide having the structure of formula O-I, or a salt thereof. In some embodiments, [H]ₐ-A^{c} or [H]_{b}-A^{c} is an oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}(N19-PX19)_{nz}-(N20-PX20)_{pz}-(N21-PX21)_{tz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3 or a salt thereof.

In some embodiments, P in T^{LD} is P*. In some embodiments, a conjugate has the structure of [(A^{c})ₐ-L^{LD}]_{b}-R^{LD}. In some embodiments, a conjugate has the structure of (A^{c})ₐ-L^{LD}-R^{LD}.

In some embodiments, a is 1-100. In some embodiments, a is 1-50. In some embodiments, a is 1-40. In some embodiments, a is 1-30. In some embodiments, a is 1-20. In some embodiments, a is 1-15. In some embodiments, a is 1-10. In some embodiments, a is 1-9. In some embodiments, a is 1-8. In some embodiments, a is 1-7. In some embodiments, a is 1-6. In some embodiments, a is 1-5. In some embodiments, a is 1-4. In some embodiments, a is 1-3. In some embodiments, a is 1-2. In some embodiments, a is 1. In some embodiments, a is 2. In some embodiments, a is 3. In some embodiments, a is 4. In some embodiments, a is 5. In some embodiments, a is 6. In some embodiments, a is 7. In some embodiments, a is 8. In some embodiments, a is 9. In some embodiments, a is 10. In some embodiments, a is more than 10. In some embodiments, b is 1-100. In some embodiments, b is 1-50. In some embodiments, b is 1-40. In some embodiments, b is 1-30. In some embodiments, b is 1-20. In some embodiments, b is 1-15. In some embodiments, b is 1-10. In some embodiments, b is 1-9. In some embodiments, b is 1-8. In some embodiments, b is 1-7. In some embodiments, b is 1-6. In some embodiments, b is 1-5. In some embodiments, b is 1-4. In some embodiments, b is 1-3. In some embodiments, b is 1-2. In some embodiments, b is 1. In some embodiments, b is 2. In some embodiments, b is 3. In some embodiments, b is 4. In some embodiments, b is 5. In some embodiments, b is 6. In some embodiments, b is 7. In some embodiments, b is 8. In some embodiments, b is 9. In some embodiments, b is 10. In some embodiments, b is more than 10. In some embodiments, a conjugate has the structure of A^{c}-L^{LD}-R^{LD}. In some embodiments, A^{c} is conjugated through one or more of its sugar, base and/or internucleotidic linkage moieties. In some embodiments, A^{c} is conjugated through its 5′-OH (5′-O—). In some embodiments, A^{c} is conjugated through its 3′-OH (3′-O—). In some embodiments, A^{c} is conjugated through an internucleotidic linkage. In some embodiments, A^{c} is conjugated through a nucleobase. In some embodiments, A^{c} is conjugated through a sugar. In some embodiments, before conjugation, A^{c}-(H)_{b} (b is an integer of 1-1000 depending on valency of A^{c}) is an oligonucleotide as described herein, for example, one of those described in any one of the Tables. In some embodiments, L^{LD} is -L-. In some embodiments, L^{LD} comprises a phosphorothioate group. In some embodiments, L^{LD} is —C(O)NH—(CH₂)₆-OP(═O)(S⁻)—O—. In some embodiments, the —C(O)NH end is connected to R^{LD}, and the —O— end is connected to the oligonucleotide, e.g., through 5′- or 3′-end. In some embodiments, R^{LD} is optionally substituted C₁₀, C₁₅, C₁₆, C₁₇, C₁₈, C₁₉, C₂₀, C₂₁, C₂₂, C₂₃, C₂₄, or C₂₅ to C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₆₀, C₇₀, or C₈₀ aliphatic. In some embodiments, R^{LD} is optionally substituted C₁₀₋₈₀ aliphatic. In some embodiments, R^{LD} is optionally substituted C20-80 aliphatic. In some embodiments, R^{LD} is optionally substituted C₁₀₋₇₀ aliphatic. In some embodiments, R^{LD} is optionally substituted C20-70 aliphatic. In some embodiments, R^{LD} is optionally substituted C10-60 aliphatic. In some embodiments, R^{LD} is optionally substituted C20-60 aliphatic. In some embodiments, R^{LD} is optionally substituted C₁₀₋₅₀ aliphatic. In some embodiments, R^{LD} is optionally substituted C20-50 aliphatic. In some embodiments, R^{LD} is optionally substituted C₁₀₋₄₀ aliphatic. In some embodiments, R^{LD} is optionally substituted C₂₀₋₄₀ aliphatic. In some embodiments, R^{LD} is optionally substituted C₁₀₋₃₀ aliphatic. In some embodiments, R^{LD} is optionally substituted C₂₀₋₃₀ aliphatic. In some embodiments, R^{LD} is unsubstituted C₁₀, C₁₅, C₁₆, C₁₇, C₁₈, C₁₉, C₂₀, C₂₁, C₂₂, C₂₃, C₂₄, or C₂₅ to C₂₀, C₂₁, C₂₂, C₂₃, C₂₄, C₂₅, C₂₆, C₂₇, C₂₈, C₂₉, C₃₀, C₃₅, C₄₀, C₄₅, C₅₀, C₆₀, C₇₀, or C₈₀ aliphatic. In some embodiments, R^{LD} is unsubstituted C₁₀₋₈₀ aliphatic. In some embodiments, R^{LD} is unsubstituted C₂₀₋₈₀ aliphatic. In some embodiments, R^{LD} is unsubstituted C₁₀₋₇₀ aliphatic. In some embodiments, R^{LD} is unsubstituted C₂₀₋₇₀ aliphatic. In some embodiments, R^{LD} is unsubstituted C₁₀₋₆₀ aliphatic. In some embodiments, R^{LD} is unsubstituted C₂₀₋₆₀ aliphatic. In some embodiments, R^{LD} is unsubstituted C₁₀₋₅₀ aliphatic. In some embodiments, R^{LD} is unsubstituted C₂₀₋₅₀ aliphatic. In some embodiments, R^{LD} is unsubstituted C₁₀₋₄₀ aliphatic. In some embodiments, R^{LD} is unsubstituted C₂₀₋₄₀ aliphatic. In some embodiments, R^{LD} is unsubstituted C₁₀₋₃₀ aliphatic. In some embodiments, R^{LD} is unsubstituted C₂₀₋₃₀ aliphatic.

In some embodiments, R^{LD} is not hydrogen. In some embodiments, R^{LD} is a lipid moiety. In some embodiments, R^{LD} is a targeting moiety. In some embodiments, R^{LD} is a targeting moiety comprising a carbohydrate moiety. In some embodiments, R^{LD} is a GalNAc moiety.

In some embodiments, a single-stranded RNAi agent is any one of the preceding compositions, further comprising one or more additional components.

In some embodiments, a provided oligonucleotide is capable of degrading a target transcript, e.g., RNA, through both a RNase H mechanism and a RNAi mechanism.

In some embodiments, conjugation of a lipid moiety to an oligonucleotide improves at least one property of the oligonucleotide. In some embodiments, improved properties include increased activity (e.g., increased ability to direct a decrease in the expression and/or level of a target gene or its gene product and/or direct single-stranded RNA interference and/or direct RNase H-mediated knockdown) and/or improved distribution to a tissue. In some embodiments, a tissue is muscle tissue. In some embodiments, a tissue is skeletal muscle, gastrocnemius, triceps, heart or diaphragm. In some embodiments, improved properties include reduced hTLR9 agonist activity. In some embodiments, improved properties include hTLR9 antagonist activity. In some embodiments, improved properties include increased hTLR9 antagonist activity.

In general, properties of oligonucleotide compositions as described herein can be assessed using any appropriate assay.

Those of skill in the art will be aware of and/or will readily be able to develop appropriate assays for particular oligonucleotide compositions.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an alkyl group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, an alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an alkyl group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value). In some embodiments, use of the term “about” in reference to dosages means±5 mg/kg/day.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the phrase a “characteristic portion” of a protein or polypeptide is one that contains a continuous stretch of amino acids, or a collection of continuous stretches of amino acids, that together are characteristic of a protein or polypeptide. Each such continuous stretch generally will contain at least two amino acids. Furthermore, those of ordinary skill in the art will appreciate that typically at least 5, 10, 15, 20 or more amino acids are required to be characteristic of a protein. In general, a characteristic portion is one that, in addition to the sequence identity specified above, shares at least one functional characteristic with the relevant intact protein.

Characteristic structural element: The term “characteristic structural element” or “structural element” refers to a distinctive structural element that is found in all members of a family of polypeptides, small molecules, or nucleic acids, and therefore can be used by those of ordinary skill in the art to define members of the family. In some embodiments, a structural element of a single-stranded RNAi agent includes, but is not limited to: a 5′-end structure, a 5′-end region, a 5′ nucleotide moiety, a seed region, a post-seed region, a 3′-end region, a 3′-terminal dinucleotide, a 3′ cap, a pattern of modifications, a pattern of stereochemistry in the backbone, additional chemical moieties, etc.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Dosing regimen: As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regime comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Equivalent agents: Those of ordinary skill in the art, reading the present disclosure, will appreciate that the scope of useful agents in the context of the present disclosure is not limited to those specifically mentioned or exemplified herein. In particular, those skilled in the art will recognize that active agents typically have a structure that consists of a core and attached pendant moieties, and furthermore will appreciate that simple variations of such core and/or pendant moieties may not significantly alter activity of the agent. For example, in some embodiments, substitution of one or more pendant moieties with groups of comparable three-dimensional structure and/or chemical reactivity characteristics may generate a substituted compound or portion equivalent to a parent reference compound or portion. In some embodiments, addition or removal of one or more pendant moieties may generate a substituted compound equivalent to a parent reference compound. In some embodiments, alteration of core structure, for example by addition or removal of a small number of bonds (typically not more than 5, 4, 3, 2, or 1 bonds, and often only a single bond) may generate a substituted compound equivalent to a parent reference compound. In many embodiments, equivalent compounds may be prepared by methods illustrated in general reaction schemes as, for example, described below, or by modifications thereof, using readily available starting materials, reagents and conventional or provided synthesis procedures. In these reactions, it is also possible to make use of variants, which are in themselves known, but are not mentioned here.

Equivalent Dosage: The term “equivalent dosage” is used herein to compare dosages of different pharmaceutically active agents that effect the same biological result. Dosages of two different agents are considered to be “equivalent” to one another in accordance with the present disclosure if they achieve a comparable level or extent of the biological result. In some embodiments, equivalent dosages of different pharmaceutical agents for use in accordance with the present disclosure are determined using in vitro and/or in vivo assays as described herein. In some embodiments, one or more lysosomal activating agents for use in accordance with the present disclosure is utilized at a dose equivalent to a dose of a reference lysosomal activating agent; in some such embodiments, the reference lysosomal activating agent for such purpose is selected from the group consisting of small molecule allosteric activators (e.g., pyrazolpyrimidines), imminosugars (e.g., isofagomine), antioxidants (e.g., n-acetyl-cysteine), and regulators of cellular trafficking (e.g., Rab1a polypeptide).

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted form thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.).

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Intraperitoneal: The phrases “intraperitoneal administration” and “administered intraperitonealy” as used herein have their art-understood meaning referring to administration of a compound or composition into the peritoneum of a subject.

In vitro: As used herein, the term “in vitro” refers to events that occur in an artificial environment, e.g., in a test tube or reaction vessel, in cell culture, etc., rather than within an organism (e.g., animal, plant and/or microbe).

In vivo: As used herein, the term “in vivo” refers to events that occur within an organism (e.g., animal, plant and/or microbe).

Lower alkyl: The term “lower alkyl” refers to a C₁₋₄ straight or branched alkyl group. Example lower alkyl groups are methyl, ethyl, propyl, isopropyl, butyl, isobutyl, and tert-butyl.

Lower haloalkyl: The term “lower haloalkyl” refers to a C₁₋₄ straight or branched alkyl group that is substituted with one or more halogen atoms.

Optionally Substituted: As described herein, compounds, e.g., oligonucleotides, of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable atom, e.g., a suitable carbon atom, are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o})C(O)NR^{o}₂; —N(R^{o}C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR, —SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)₀₋₄S(O)₂R^{o}; —(CH₂)₀₋₄S(O)₂OR^{o}; —(CH₂)₀₋₄OS(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —Si(R^{o})₃; —OSi(R^{o})₃; —B(R^{o})₂; —OB(R^{o})₂; —OB(OR^{o})₂; —P(R^{o})₂; —P(OR^{o})₂; —OP(R^{o})₂; —OP(OR^{o})₂; —P(O)(R^{o})₂; —P(O)(OR^{o})₂; —OP(O)(R^{o})₂; —OP(O)(OR^{o})₂; —OP(O)(OR^{o})(SR^{o}); —SP(O)(R^{o})₂; —SP(O)(OR^{o})₂; —N(R^{o})P(O)(R^{o})₂; —N(R^{o})P(O)(OR^{o})₂; —P(R^{o})₂[B(R^{o})₃]; —P(OR^{o})₂[B(R^{o})₃]; —OP(R^{o})₂[B(R^{o})₃]; —OP(OR^{o})₂[B(R^{o})₃]; —(C₁₋₄ straight or branched)alkylene)O—N(R^{o})₂; or —(C₁₋₄ straight or branched)alkylene)C(O)O—N(R^{o})₂, wherein each R^{o} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{o}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{o} (or the ring formed by taking two independent occurrences of R^{o} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{o} include ═O and ═S.

Suitable divalent substituents, e.g., on a suitable carbon atom, are independently the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur.

Suitable substituents on the aliphatic group of R* are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated. As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, a provided oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen in the acidic groups are replaced with cations. In some embodiments, a pharmaceutically acceptable salt is a sodium salt of a provided oligonucleotide. In some embodiments, a pharmaceutically acceptable salt is a sodium salt of a provided oligonucleotide, wherein each acidic phosphate group exists as a salt form (all sodium salt); for example, a sodium salt of WV-2555 containing 19 Na⁺, or a sodium salt of WV-2555 containing 23 Na⁺.

Prodrug: A general, a “prodrug,” as that term is used herein and as is understood in the art, is an entity that, when administered to an organism, is metabolized in the body to deliver an active (e.g., therapeutic or diagnostic) agent of interest. Typically, such metabolism involves removal of at least one “prodrug moiety” so that the active agent is formed. Various forms of “prodrugs” are known in the art. For examples of such prodrug moieties, see:
a) Design of Prodrugs, edited by H. Bundgaard, (Elsevier, 1985) and Methods in Enzymology, 42:309-396, edited by K. Widder, et al. (Academic Press, 1985);
b) Prodrugs and Targeted Delivery, edited by by J. Rautio (Wiley, 2011);
c) A Textbook of Drug Design and Development, edited by Krogsgaard-Larsen;
d) Bundgaard, Chapter 5 “Design and Application of Prodrugs”, by H. Bundgaard, p. 113-191 (1991);
e) Bundgaard, Advanced Drug Delivery Reviews, 8:1-38 (1992);
f) Bundgaard, et al., Journal of Pharmaceutical Sciences, 77:285 (1988); and
g) Kakey a, et al., Chem. Pharm. Bull., 32:692 (1984).

As with other compounds described herein, prodrugs may be provided in any of a variety of forms, e.g., crystal forms, salt forms etc. In some embodiments, prodrugs are provided as pharmaceutically acceptable salts thereof.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethyl propynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxy acetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, Np-methoxybenzylideneamine, N-diphenylmethyleneamine, N-[(2-pyridyl)mesityl]methyleneamine, N-(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethyl silyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), di ethylisopropyl silyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-m ethylphenoxy acetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, tri ethyl silyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t- butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group.

In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano- 1,1-dimethylethyl, 4-N-methyl aminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Protein: As used herein, the term “protein” refers to a polypeptide (i.e., a string of at least two amino acids linked to one another by peptide bonds). In some embodiments, proteins include only naturally-occurring amino acids. In some embodiments, proteins include one or more non-naturally-occurring amino acids (e.g., moieties that form one or more peptide bonds with adjacent amino acids). In some embodiments, one or more residues in a protein chain contain a non-amino-acid moiety (e.g., a glycan, etc). In some embodiments, a protein includes more than one polypeptide chain, for example linked by one or more disulfide bonds or associated by other means. In some embodiments, proteins contain L-amino acids, D-amino acids, or both; in some embodiments, proteins contain one or more amino acid modifications or analogs known in the art. Useful modifications include, e.g., terminal acetylation, amidation, methylation, etc. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof and/or characteristic portions thereof.

RNA interference: As used herein, the terms “RNA interference” or “RNAi” refer to a post-transcriptional, targeted gene-silencing process involving the RISC (RNA-induced silencing complex). A process of RNAi reportedly naturally occurs when ribonuclease III (Dicer) cleaves a longer dsRNA into shorter fragments called siRNAs. A naturally-produced siRNA (small interfering RNA) is typically about 21 to 23 nucleotides long with an about 19 basepair duplex and two single-stranded overhangs and is typically RNA. These RNA segments then reportedly direct the degradation of the target nucleic acid, such as a mRNA or pre-mRNA. Dicer has reportedly also been implicated in the excision of 21- and 22-nucleotide small temporal RNAs (stRNAs) from precursor RNA of conserved structure that are implicated in translational control. Hutvagner et al. 2001, Science, 293, 834. Those skilled in the art are aware that RNAi can be mediated by a single-stranded or a double-stranded oligonucleotide that includes a sequence complementary or substantially complementary to a target sequence (e.g., in a target mRNA). Thus, in some embodiments of the present disclosure, a single-stranded oligonucleotide as described herein may act as an RNAi agent; in some embodiments, a double-stranded oligonucleotide as described herein may act as an RNAi agent. In some embodiments, an RNAi response involves an endonuclease complex, commonly referred to as an RNA-induced silencing complex (RISC), which directs cleavage of single-stranded mRNA complementary to the antisense strand of the siRNA. In some embodiments, RISC directs cleavage of target RNA complementary to provided oligonucleotides which can function as single-stranded RNAi agent. In some embodiments, cleavage of a target RNA takes place in the middle of the region complementary to the antisense strand of a siRNA duplex or single-stranded RNAi agent. In some embodiments, RNA interference is directed by a single-stranded oligonucleotide which acts as a single-stranded RNAi agent that can direct RNA interference in a mechanism involving the RISC pathway.

RNAi agent: As used herein, the term “RNAi agent,” “iRNA agent”, and the like, refer to an oligonucleotide that, when administered to a system in which a target gene product (e.g., a transcript, such as a pre-mRNA or a mRNA, of a target gene) is being or has been expressed, reduces level and/or activity (e.g., translation) of that target gene product. In some embodiments, an RNAi agent may be or comprise a single-stranded oligonucleotide or a double-stranded oligonucleotide. In some embodiments, an RNAi agent may have a structure recognized in the art as a siRNA (short inhibitory RNA), shRNA (short or small hairpin RNA), dsRNA (double-stranded RNA), microRNA, etc. In some embodiments, an RNAi agent may specifically bind to a RNA target (e.g., a transcript of a target gene). In some embodiments, upon binding to its target, and RNAi agent is loaded to the RISC (RNA-induced silencing complex). In some embodiments, an RNAi agent directs degradation of, and/or inhibits translation of, its target, in some embodiments via a mechanism involving the RISC (RNA-induced silencing complex) pathway. In some embodiments, an RNAi agent is an oligonucleotide that activates the RISC complex/pathway. In some embodiments, an RNAi agent comprises an antisense strand sequence. In some embodiments, an RNAi agent includes only one oligonucleotide strand (e.g., is a single-stranded oligonucleotide). In some embodiments, a single-stranded RNAi agent oligonucleotide can be or comprise a sense or antisense strand sequence, as described by Sioud 2005 J. Mol. Biol. 348: 1079-1090. In some embodiments, a RNAi agent is a compound capable of directing RNA interference. In some embodiments, a RNAi agent may have a structure or format as is found in “canonical” siRNA structure). In some embodiments, an RNAi agent may have a structure that differs from a “canonical” siRNA structure. To give but a few examples, in some embodiments, an RNAi agent can be longer or shorter than the canonical, can be blunt-ended, and/or can comprise one or more modifications, mismatches, gaps and/or nucleotide replacements. In some embodiments, an RNAi agent contains a 3′-end cap as described in the present disclosure. Without wishing to be bound by any particular theory, Applicant proposes that, in some embodiments, a 3′-end cap can allow both of two functions: (1) allowing RNA interference; and (2) increasing duration of activity and/or biological half-life, which may be accomplished, for example, by increased binding to the PAZ domain of Dicer and/or one or more Ago proteins and/or reducing or preventing degradation of the RNAi agent (e.g., by nucleases such as those in the serum or intestinal fluid). In some embodiments, a RNAi agent of the present disclosure targets (e.g., binds to, anneals to, etc.) a target mRNA. In some embodiments, exposure of a RNAi agent to its target results in a decrease of activity, level and/or expression, e.g., a “knock-down” or “knock-out” of the target. Particularly, in some embodiments, in the case of a disease, disorder and/or condition characterized by over-expression and/or hyper-activity of a target gene, administration of a RNAi agent to a cell, tissue, or subject knocks down the target gene enough to restore a normal level of activity, or to reduce activity to a level that can alleviate, ameliorate, relieve, inhibit, prevent, delay onset of, reduce severity of, and/or reduce incidence of one or more symptoms or features of a disease, disorder, and/or condition of the disease, disorder, and/or condition. In some embodiments, a RNAi agent is double-stranded comprising an antisense strand which is a single-stranded RNAi agent as described herein, which, in combination with a sense strand, can direct RNA interference.

Sample: A “sample” as used herein is a specific organism or material obtained therefrom. In some embodiments, a sample is a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, a sample is an organism. In some embodiments, a sample is a plant. In some embodiments, a sample is an animal. In some embodiments, a sample is a human. In some embodiments, a sample is an organism other than a human.

Single-stranded RNA interference: As used herein, the phrases “single-stranded RNAi” or “single-stranded RNA interference” or the like refer to a process or method of gene silencing directed at least in part by administration of a single-stranded RNAi agent to a system (e.g., cells, tissues, organs, subjects, etc.) where RNAi is to be directed by the agent, and which requires the RISC pathway. The terms may be utilized herein in certain instances to distinguish from “double-stranded RNAi” or “double-stranded RNA interference”, in which a double-stranded RNAi agent is administered to a system, and may be further processed, for example so that one of its two strands is loaded to RISC to, e.g., suppress translation, cleave target RNA, etc.

Single-stranded RNA1 agent: As used herein, the phrase “single-stranded RNAi agent” refers to a single-stranded oligonucleotide that can direct single-stranded RNA interference (RNAi or iRNA) or gene silencing via the RISC pathway. A single-stranded RNAi agent can comprise a polymer of one or more single-stranded nucleotides.

Subject: As used herein, the term “subject” or “test subject” refers to any organism to which a provided compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject may be suffering from and/or susceptible to a disease, disorder and/or condition.

Substantially: As used herein, the term “substantially” refers to the qualitative condition of exhibiting total or near-total extent or degree of a characteristic or property of interest. A base sequence which is substantially complementary to a second sequence is not identical to the second sequence, but is mostly or nearly identical to the second sequence. In addition, one of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and/or chemical phenomena.

Suffering from: An individual who is “suffering from” a disease, disorder and/or condition has been diagnosed with and/or displays one or more symptoms of a disease, disorder and/or condition.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Tautomeric forms: The phrase “tautomeric forms,” as used herein, is used to describe different isomeric forms of organic compounds that are capable of facile interconversion. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present disclosure. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the disclosure, chemical compositions may be provided that are or include pure preparations of a single tautomeric form of a compound. In some embodiments of the disclosure, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the disclosure, chemical compositions may contain all tautomeric forms of a compound. In some embodiments of the disclosure, chemical compositions may contain less than all tautomeric forms of a compound. In some embodiments of the disclosure, chemical compositions may contain one or more tautomeric forms of a compound in amounts that vary over time as a result of interconversion. In some embodiments of the disclosure, the tautomerism is keto-enol tautomerism. One of skill in the chemical arts would recognize that a keto-enol tautomer can be “trapped” (i.e., chemically modified such that it remains in the “enol” form) using any suitable reagent known in the chemical arts in to provide an enol derivative that may subsequently be isolated using one or more suitable techniques known in the art. Unless otherwise indicated, the present disclosure encompasses all tautomeric forms of relevant compounds, whether in pure form or in admixture with one another.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Wild-type: As used herein, the term “wild-type” has its art-understood meaning that refers to an entity having a structure and/or activity as found in nature in a “normal” (as contrasted with mutant, diseased, altered, etc) state or context. Those of ordinary skill in the art will appreciate that wild type genes and polypeptides often exist in multiple different forms (e.g., alleles).

Nucleic acid: The term “nucleic acid”, as used herein, includes any nucleotides and polymers thereof. The term “polynucleotide”, as used herein, refers to a polymeric form of nucleotides of any length, either ribonucleotides (RNA) or deoxyribonucleotides (DNA). These terms refer to the primary structure of the molecules and, thus, include double- and single-stranded DNA, and double- and single-stranded RNA. These terms include, as equivalents, analogs of either RNA or DNA made from modified nucleotides and/or modified polynucleotides, such as, though not limited to, methylated, protected and/or capped nucleotides or polynucleotides. The terms encompass poly- or oligo-ribonucleotides (RNA) and poly- or oligo-deoxyribonucleotides (DNA); RNA or DNA derived from N-glycosides or C-glycosides of nucleobases and/or modified nucleobases; nucleic acids derived from sugars and/or modified sugars; and nucleic acids derived from phosphate bridges and/or modified internucleotide linkages. The term encompasses nucleic acids containing any combinations of nucleobases, modified nucleobases, sugars, modified sugars, phosphate bridges or modified internucleotidic linkages. Examples include, and are not limited to, nucleic acids containing ribose moieties, nucleic acids containing deoxy-ribose moieties, nucleic acids containing both ribose and deoxyribose moieties, nucleic acids containing ribose and modified ribose moieties. Unless otherwise specified, the prefix poly- refers to a nucleic acid containing 2 to about 10,000 nucleotide monomer units and wherein the prefix oligo- refers to a nucleic acid containing 2 to about 200 nucleotide monomer units.

Nucleotide: The term “nucleotide” as used herein refers to a monomeric unit of a polynucleotide that consists of a heterocyclic base, a sugar, and one or more internucleotidic linkages. The naturally occurring bases (guanine, (G), adenine, (A), cytosine, (C), thymine, (T), and uracil (U)) are derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleotides are linked via internucleotidic linkages to form nucleic acids, or polynucleotides. Many internucleotidic linkages are known in the art (such as, though not limited to, phosphate, phosphorothioates, boranophosphates and the like). Artificial nucleic acids include PNAs (peptide nucleic acids), phosphotriesters, phosphorothionates, H-phosphonates, phosphoramidates, boranophosphates, methylphosphonates, phosphonoacetates, thiophosphonoacetates and other variants of the phosphate backbone of native nucleic acids, such as those described herein. In some embodiments, a natural nucleotide comprises a naturally occurring base, sugar and internucleotidic linkage. As used herein, the term “nucleotide” also encompasses structural analogs used in lieu of natural or naturally-occurring nucleotides, such as modified nucleotides and nucleotide analogs.

Modified nucleotide: The term “modified nucleotide” includes any chemical moiety which differs structurally from a natural nucleotide but is capable of performing at least one function of a natural nucleotide. In some embodiments, a modified nucleotide comprises a modification at a sugar, base and/or internucleotidic linkage. In some embodiments, a modified nucleotide comprises a modified sugar, modified nucleobase and/or modified internucleotidic linkage. In some embodiments, a modified nucleotide is capable of at least one function of a nucleotide, e.g., forming a subunit in a polymer capable of base-pairing to a nucleic acid comprising an at least complementary sequence of bases.

Analog: The term “analog” includes any chemical moiety which differs structurally from a reference chemical moiety or class of moieties, but which is capable of performing at least one function of such a reference chemical moiety or class of moieties. As non-limiting examples, a nucleotide analog differs structurally from a nucleotide but performs at least one function of a nucleotide; a nucleobase analog differs structurally from a nucleobase but performs at least one function of a nucleobase; etc.

Nucleoside: The term “nucleoside” refers to a moiety wherein a nucleobase or a modified nucleobase is covalently bound to a sugar or modified sugar.

Modified nucleoside: The term “modified nucleoside” refers to a moiety derived from or chemically similar to a natural nucleoside, but which comprises a chemical modification which differentiates it from a natural nucleoside. Non-limiting examples of modified nucleosides include those which comprise a modification at the base and/or the sugar. Non-limiting examples of modified nucleosides include those with a 2′ modification at a sugar. Non-limiting examples of modified nucleosides also include abasic nucleosides (which lack a nucleobase). In some embodiments, a modified nucleoside is capable of at least one function of a nucleoside, e.g., forming a moiety in a polymer capable of base-pairing to a nucleic acid comprising an at least complementary sequence of bases.

Nucleoside analog: The term “nucleoside analog” refers to a chemical moiety which is chemically distinct from a natural nucleoside, but which is capable of performing at least one function of a nucleoside. In some embodiments, a nucleoside analog comprises an analog of a sugar and/or an analog of a nucleobase. In some embodiments, a modified nucleoside is capable of at least one function of a nucleoside, e.g., forming a moiety in a polymer capable of base-pairing to a nucleic acid comprising a complementary sequence of bases.

Sugar: The term “sugar” refers to a monosaccharide or polysaccharide in closed and/or open form. In some embodiments, sugars are monosaccharides. In some embodiments, sugars are polysaccharides. Sugars include, but are not limited to, ribose, deoxyribose, pentofuranose, pentopyranose, and hexopyranose moieties. As used herein, the term “sugar” also encompasses structural analogs used in lieu of conventional sugar molecules, such as glycol, polymer of which forms the backbone of the nucleic acid analog, glycol nucleic acid (“GNA”), etc. As used herein, the term “sugar” also encompasses structural analogs used in lieu of natural or naturally-occurring nucleotides, such as modified sugars and nucleotide sugars.

Modified sugar: The term “modified sugar” refers to a moiety that can replace a sugar. A modified sugar mimics the spatial arrangement, electronic properties, or some other physicochemical property of a sugar.

Nucleobase: The term “nucleobase” refers to the parts of nucleic acids that are involved in the hydrogen-bonding that binds one nucleic acid strand to another complementary strand in a sequence specific manner. The most common naturally-occurring nucleobases are adenine (A), guanine (G), uracil (U), cytosine (C), and thymine (T). In some embodiments, the naturally-occurring nucleobases are modified adenine, guanine, uracil, cytosine, or thymine. In some embodiments, the naturally-occurring nucleobases are methylated adenine, guanine, uracil, cytosine, or thymine. In some embodiments, a nucleobase is a “modified nucleobase,” e.g., a nucleobase other than adenine (A), guanine (G), uracil (U), cytosine (C), and thymine (T). In some embodiments, the modified nucleobases are methylated adenine, guanine, uracil, cytosine, or thymine. In some embodiments, the modified nucleobase mimics the spatial arrangement, electronic properties, or some other physicochemical property of the nucleobase and retains the property of hydrogen-bonding that binds one nucleic acid strand to another in a sequence specific manner. In some embodiments, a modified nucleobase can pair with all of the five naturally occurring bases (uracil, thymine, adenine, cytosine, or guanine) without substantially affecting the melting behavior, recognition by intracellular enzymes or activity of the oligonucleotide duplex. As used herein, the term “nucleobase” also encompasses structural analogs used in lieu of natural or naturally-occurring nucleotides, such as modified nucleobases and nucleobase analogs.

Modified nucleobase: The terms “modified nucleobase”, “modified base” and the like refer to a chemical moiety which is chemically distinct from a nucleobase, but which is capable of performing at least one function of a nucleobase. In some embodiments, a modified nucleobase is a nucleobase which comprises a modification. In some embodiments, a modified nucleobase is capable of at least one function of a nucleobase, e.g., forming a moiety in a polymer capable of base-pairing to a nucleic acid comprising an at least complementary sequence of bases.

3′-end cap: The term “3′-end cap” refers to a non-nucleotidic chemical moiety bound to the 3′-end of an oligonucleotide, e.g., a RNAi agent. In some embodiments, a 3′-end cap replaces a 3′-terminal dinucleotide. In some embodiments, a 3′-end cap of an oligonucleotide performs at least one of the following functions: allowing RNA interference directed by the oligonucleotide, protecting the oligonucleotide from degradation or reducing the amount or rate of degradation of the oligonucleotide (e.g., by nucleases), reducing the off-target effects of a sense strand, or increasing the activity, duration or efficacy of RNA interference directed by the oligonucleotide. By describing a 3′-end cap as “non-nucleotidic”, it is meant that a 3′-end cap is not a nucleotidic moiety, or oligonucleotide moiety, connected to a sugar moiety of the rest of an oligonucleotide as it would do if it is part of an oligonucleotide chain. Certain example 3′-end caps are described herein. A person having ordinary skill understands that others 3′-end caps known in the art can be utilized in accordance in the present disclosure.

Blocking group: The term “blocking group” refers to a group that masks the reactivity of a functional group. The functional group can be subsequently unmasked by removal of the blocking group. In some embodiments, a blocking group is a protecting group.

Moiety: The term “moiety” refers to a specific segment or functional group of a molecule. Chemical moieties are often recognized chemical entities embedded in or appended to a molecule.

Solid support: The term “solid support” refers to any support which enables synthesis of nucleic acids. In some embodiments, the term refers to a glass or a polymer, that is insoluble in the media employed in the reaction steps performed to synthesize nucleic acids, and is derivatized to comprise reactive groups. In some embodiments, the solid support is Highly Cross-linked Polystyrene (HCP) or Controlled Pore Glass (CPG). In some embodiments, the solid support is Controlled Pore Glass (CPG). In some embodiments, the solid support is hybrid support of Controlled Pore Glass (CPG) and Highly Cross-linked Polystyrene (HCP).

Linker or Linking moiety: The terms “linker”, “linking moiety” and the like refer to any chemical moiety which connects one chemical moiety to another. In some embodiments, a linker is a moiety which connects one oligonucleotide to another oligonucleotide in a multimer. In some embodiments, a linker is a moiety optionally positioned between the terminal nucleoside and the solid support or between the terminal nucleoside and another nucleoside, nucleotide, or nucleic acid.

Gene: The terms “gene,” “recombinant gene” and “gene construct” as used herein, refer to a DNA molecule, or portion of a DNA molecule, that encodes a protein or a portion thereof. The DNA molecule can contain an open reading frame encoding the protein (as exon sequences) and can further include intron sequences. The term “intron” as used herein, refers to a DNA sequence present in a given gene which is not translated into protein and is found in some, but not all cases, between exons. It can be desirable for the gene to be operably linked to, (or it can comprise), one or more promoters, enhancers, repressors and/or other regulatory sequences to modulate the activity or expression of the gene, as is well known in the art.

Complementary DNA: As used herein, a “complementary DNA” or “cDNA” includes recombinant polynucleotides synthesized by reverse transcription of mRNA and from which intervening sequences (introns) have been removed.

Homology: “Homology” or “identity” or “similarity” refers to sequence similarity between two nucleic acid molecules. Homology and identity can each be determined by comparing a position in each sequence which can be aligned for purposes of comparison. When an equivalent position in the compared sequences is occupied by the same base, then the molecules are identical at that position; when the equivalent site occupied by the same or a similar nucleic acid residue (e.g., similar in steric and/or electronic nature), then the molecules can be referred to as homologous (similar) at that position. Expression as a percentage of homology/similarity or identity refers to a function of the number of identical or similar nucleic acids at positions shared by the compared sequences. A sequence which is “unrelated” or “non-homologous” shares less than 40% identity, less than 35% identity, less than 30% identity, or less than 25% identity with a sequence described herein. In comparing two sequences, the absence of residues (amino acids or nucleic acids) or presence of extra residues also decreases the identity and homology/similarity.

In some embodiments, the term “homology” describes a mathematically based comparison of sequence similarities which is used to identify genes with similar functions or motifs. The nucleic acid sequences described herein can be used as a “query sequence” to perform a search against public databases, for example, to identify other family members, related sequences or homologs. In some embodiments, such searches can be performed using the NBLAST and) (BLAST programs (version 2.0) of Altschul, et al. (1990) J. Mol. Biol. 215:403-10. In some embodiments, BLAST nucleotide searches can be performed with the NBLAST program, score=100, wordlength=12 to obtain nucleotide sequences homologous to nucleic acid molecules of the disclosure. In some embodiments, to obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g.,)(BLAST and BLAST) can be used (See www.ncbi.nlm.nih.gov).

Identity: As used herein, “identity” means the percentage of identical nucleotide residues at corresponding positions in two or more sequences when the sequences are aligned to maximize sequence matching, i.e., taking into account gaps and insertions. Identity can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Altschul, S. F. et al., J. Molec. Biol. 215: 403-410 (1990) and Altschul et al. Nuc. Acids Res. 25: 3389-3402 (1997)). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well-known Smith Waterman algorithm can also be used to determine identity.

Oligonucleotide: The term “oligonucleotide” refers to a polymer or oligomer of nucleotides, and may contain any combination of natural and non-natural nucleobases, sugars, and internucleotidic linkages.

Oligonucleotides can be single-stranded or double-stranded. As used herein, the term “oligonucleotide strand” encompasses a single-stranded oligonucleotide. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. In some embodiments, oligonucleotides are capable of directing a decrease in the expression and/or level of a target gene or its gene product. In some embodiments, oligonucleotides are capable of directing a decrease in the expression and/or level of a target gene or its gene product via RNA interference. In some embodiments, oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, oligonucleotides are capable of directing a decrease in the expression and/or level of a target gene or its gene product via RNA interference and/or RNase H-mediated knockdown. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, Ul adaptors, triplex-forming oligonucleotides, G-quadruplex oligonucleotides, RNA activators, immuno-stimulatory oligonucleotides, and decoy oligonucleotides.

Double-stranded and single-stranded oligonucleotides that are effective in inducing RNA interference are also referred to as a RNAi agent or iRNA agent, herein. In some embodiments, these RNA interference inducing oligonucleotides associate with a cytoplasmic multi-protein complex known as RNAi-induced silencing complex (RISC). In many embodiments, double-stranded RNAi agents are sufficiently long that they can be cleaved by an endogenous molecule, e.g., by Dicer, to produce smaller oligonucleotides that can enter the RISC machinery and participate in RISC mediated cleavage and/or translation suppression of a target sequence, e.g. a target mRNA sequence.

Oligonucleotides of the present disclosure can be of various lengths. In particular embodiments, oligonucleotides can range from about 2 to about 200 nucleotides in length. In various related embodiments, oligonucleotides, single-stranded, double-stranded, and triple-stranded, can range in length from about 4 to about 10 nucleotides, from about 10 to about 50 nucleotides, from about 20 to about 50 nucleotides, from about 15 to about 30 nucleotides, or from about 20 to about 30 nucleotides in length. In some embodiments, an oligonucleotide is from about 10 to about 40 nucleotides in length. In some embodiments, an oligonucleotide is from about 9 to about 39 nucleotides in length. In some embodiments, the oligonucleotide is at least 4 nucleotides in length. In some embodiments, the oligonucleotide is at least 5 nucleotides in length. In some embodiments, the oligonucleotide is at least 6 nucleotides in length. In some embodiments, the oligonucleotide is at least 7 nucleotides in length. In some embodiments, the oligonucleotide is at least 8 nucleotides in length. In some embodiments, the oligonucleotide is at least 9 nucleotides in length. In some embodiments, the oligonucleotide is at least 10 nucleotides in length. In some embodiments, the oligonucleotide is at least 11 nucleotides in length. In some embodiments, the oligonucleotide is at least 12 nucleotides in length. In some embodiments, the oligonucleotide is at least 15 nucleotides in length. In some embodiments, the oligonucleotide is at least 20 nucleotides in length. In some embodiments, the oligonucleotide is at least 25 nucleotides in length. In some embodiments, the oligonucleotide is at least 30 nucleotides in length. In some embodiments, the oligonucleotide is a duplex of complementary strands of at least 18 nucleotides in length. In some embodiments, the oligonucleotide is a duplex of complementary strands of at least 21 nucleotides in length. In some embodiments, each nucleotide counted in a length independently comprises an optionally substituted nucleobase selected from adenine, cytosine, guanosine, thymine, and uracil.

Internucleotidic linkage: As used herein, the phrase “internucleotidic linkage” refers generally to a linkage linking nucleoside units of an oligonucleotide or a nucleic acid. In some embodiments, an internucleotidic linkage is a phosphodiester linkage, as found in naturally occurring DNA and RNA molecules (natural phosphate linkage). In some embodiments, the term “internucleotidic linkage” includes a modified internucleotidic linkage. In some embodiments, an internucleotidic linkage is a “modified internucleotidic linkage” wherein each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described in the present disclosure. In some embodiments, an internucleotidic linkage is a phosphotriester linkage, phosphorothioate diester linkage

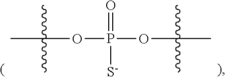 [SMILES: *OP(=O)([S-])O*]

or modified phosphorothioate triester linkage.

In some embodiments, an internucleotidic linkage is one of, e.g., PNA (peptide nucleic acid) or PMO (phosphorodiamidate Morpholino oligomer) linkage.

It is understood by a person of ordinary skill in the art that an internucleotidic linkage may exist as an anion or cation at a given pH due to the existence of acid or base moieties in the linkage.

Unless otherwise specified, when used with an oligonucleotide sequence, each of s, s1, s2, s3, s4, s5, s6 and s7 independently represents the following modified internucleotidic linkage as illustrated below:

| Sym- | |
|---|---|
| bol | Modified Internucleotidic Linkage |
| s | 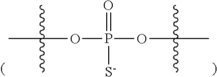 [SMILES: *OP(=O)([S-])O*] |
| | phosphorothioate |
| s1 | 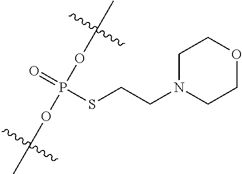 [SMILES: *OP(=O)(O*)SCCN1CCOCC1] |
| s2 | 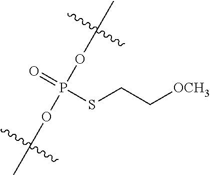 [SMILES: *OP(=O)(O*)SCCOC] |
| s3 | 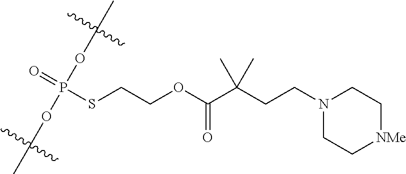 [SMILES: *OP(=O)(O*)SCCOC(=O)C(C)(C)CCN1CCN(C)CC1] |
| s4 | 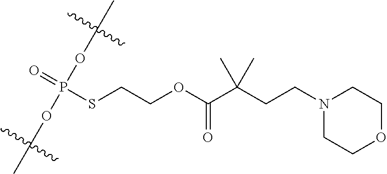 [SMILES: *OP(=O)(O*)SCCOC(=O)C(C)(C)CCN1CCOCC1] |
| s5 | 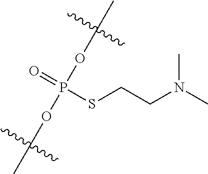 [SMILES: *OP(=O)(O*)SCCN(C)C] |
| s6 | 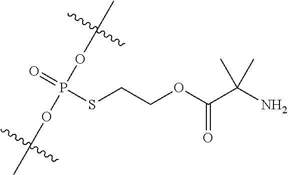 [SMILES: *OP(=O)(O*)SCCOC(=O)C(C)(C)N] |
| s7 | 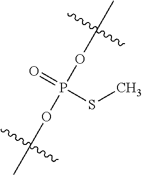 [SMILES: *OP(=O)(O*)SC] |
| s8 | 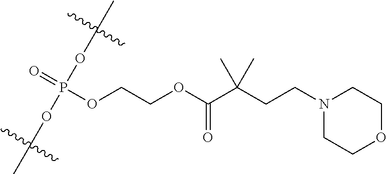 [SMILES: *OP(=O)(O*)OCCOC(=O)C(C)(C)CCN1CCOCC1] |
| s9 | 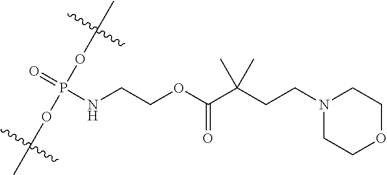 [SMILES: *OP(=O)(NCCOC(=O)C(C)(C)CCN1CCOCC1)O*] |
| s10 | 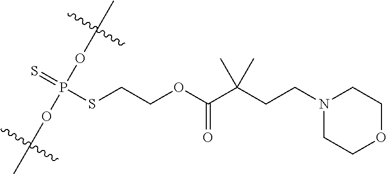 [SMILES: *OP(=S)(O*)SCCOC(=O)C(C)(C)CCN1CCOCC1] |
| s11 | 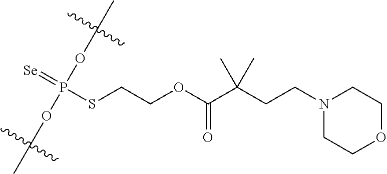 [SMILES: *OP(=[Se])(O*)SCCOC(=O)C(C)(C)CCN1CCOCC1] |
| s12 | 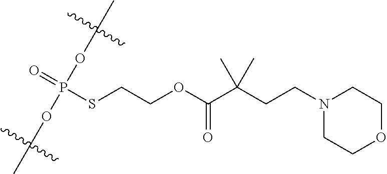 [SMILES: *OP(=O)(O*)SCCOC(=O)C(C)(C)CCN1CCOCC1] |
| s13 | 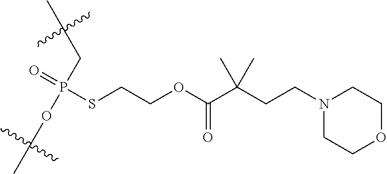 [SMILES: *CP(=O)(O*)SCCOC(=O)C(C)(C)CCN1CCOCC1] |
| s14 | 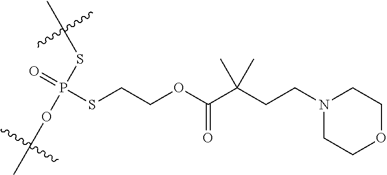 [SMILES: *OP(=O)(S*)SCCOC(=O)C(C)(C)CCN1CCOCC1] |
| s15 | 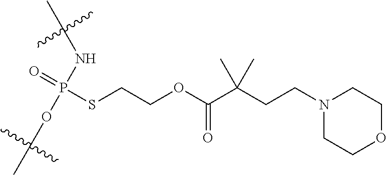 [SMILES: *NP(=O)(O*)SCCOC(=O)C(C)(C)CCN1CCOCC1] |
| s16 | 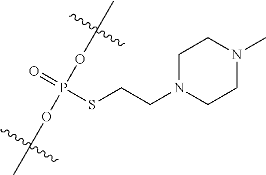 [SMILES: *OP(=O)(O*)SCCN1CCN(C)CC1] |
| s17 | 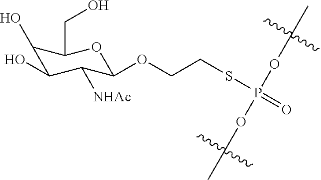 [SMILES: *OP(=O)(O*)SCCOC1OC(CO)C(O)C(O)C1NC(C)=O] |
| s18 | 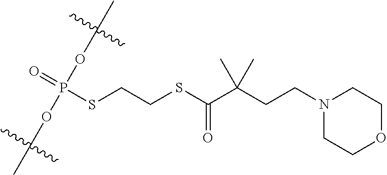 [SMILES: *OP(=O)(O*)SCCSC(=O)C(C)(C)CCN1CCOCC1] |

For instance, (Rp, Sp)-ATsCs1GA has 1) a phosphorothioate internucleotidic linkage

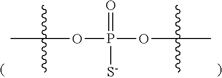 [SMILES: *OP(=O)([S-])O*]

between T and C; and 2) a phosphorothioate triester internucleotidic linkage having the structure of

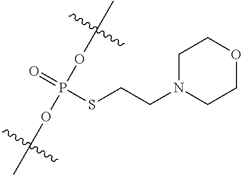 [SMILES: *OP(=O)(O*)SCCN1CCOCC1]

between C and G. Unless otherwise specified, the Rp/Sp designations preceding an oligonucleotide sequence describe the configurations of chiral linkage phosphorus atoms in the internucleotidic linkages sequentially from 5′ to 3′ of the oligonucleotide sequence. For instance, in (Rp, Sp)-ATsCs1GA, the phosphorus in the “s” linkage between T and C has Rp configuration and the phosphorus in “s1” linkage between C and G has Sp configuration.

In some embodiments, “All-(Rp)” or “All-(Sp)” is used to indicate that all chiral linkage phosphorus atoms in oligonucleotide have the same Rp or Sp configuration, respectively.

Oligonucleotide type: As used herein, the phrase “oligonucleotide type” is used to define an oligonucleotide that has a particular base sequence, pattern of backbone linkages (i.e., pattern of internucleotidic linkage types, for example, phosphate, phosphorothioate, etc.), pattern of backbone chiral centers (i.e. pattern of linkage phosphorus stereochemistry (Rp/Sp)), and pattern of backbone phosphorus modifications (e.g., pattern of “—XLR¹” groups in formula I). In some embodiments, oligonucleotides of a common designated “type” are structurally identical to one another.

One of skill in the art will appreciate that synthetic methods of the present disclosure provide for a degree of control during the synthesis of an oligonucleotide strand such that each nucleotide unit of the oligonucleotide strand can be designed and/or selected in advance to have a particular stereochemistry at the linkage phosphorus and/or a particular modification at the linkage phosphorus, and/or a particular base, and/or a particular sugar. In some embodiments, an oligonucleotide strand is designed and/or selected in advance to have a particular combination of stereocenters at the linkage phosphorus. In some embodiments, an oligonucleotide strand is designed and/or determined to have a particular combination of modifications at the linkage phosphorus. In some embodiments, an oligonucleotide strand is designed and/or selected to have a particular combination of bases. In some embodiments, an oligonucleotide strand is designed and/or selected to have a particular combination of one or more of the above structural characteristics. In some embodiments, the present disclosure provides compositions comprising or consisting of a plurality of oligonucleotide molecules (e.g., chirally controlled oligonucleotide compositions). In some embodiments, all such molecules are of the same type (i.e., are structurally identical to one another). In many embodiments, however, provided compositions comprise a plurality of oligonucleotides of different types, typically in pre-determined relative amounts.

Chiral control: As used herein, “chiral control” refers to control of the stereochemical designation of a chiral linkage phosphorus in a chiral internucleotidic linkage within an oligonucleotide. In some embodiments, a control is achieved through a chiral element that is absent from the sugar and base moieties of an oligonucleotide, for example, in some embodiments, a control is achieved through use of one or more chiral auxiliaries during oligonucleotide preparation as exemplified in the present disclosure, which chiral auxiliaries often are part of chiral phosphoramidites used during oligonucleotide preparation. In contrast to chiral control, a person having ordinary skill in the art appreciates that conventional oligonucleotide synthesis which does not use chiral auxiliaries cannot control stereochemistry at a chiral internucleotidic linkage if such conventional oligonucleotide synthesis is used to form the chiral internucleotidic linkage. In some embodiments, the stereochemical designation of each chiral linkage phosphorus in a chiral internucleotidic linkage within an oligonucleotide is controlled.

Chirally controlled oligonucleotide composition: The terms “chirally controlled oligonucleotide composition”, “chirally controlled nucleic acid composition”, and the like, as used herein, refers to a composition that comprises a plurality of oligonucleotides (or nucleic acids) which share 1) a common base sequence, 2) a common pattern of backbone linkages, and 3) a common pattern of backbone phosphorus modifications, wherein the plurality of oligonucleotides (or nucleic acids) share the same stereochemistry at one or more chiral internucleotidic linkages (chirally controlled internucleotidic linkages), and the level of the plurality of oligonucleotides (or nucleic acids) in the composition is pre-determined (e.g., through chirally controlled oligonucleotide preparation to form one or more chiral internucleotidic linkages). In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence are oligonucleotides of the plurality. In some embodiments, about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a chirally controlled oligonucleotide composition that share the common base sequence, the common pattern of backbone linkages, and the common pattern of backbone phosphorus modifications are oligonucleotides of the plurality. In some embodiments, a predetermined level is be about 1%-100%, (e.g., about 5%-100%, 10%-100%, 20%-100%, 30%-100%, 40%-100%, 50%-100%, 60%-100%, 70%-100%, 80-100%, 90-100%, 95-100%, 50%-90%, or about 5%, 10%, 20%, 30%, 40%, 50%, 60%, 70%, 80%, 85%, 90%, 91%, 92%, 93%, 94%, 95%, 96%, 97%, 98%, or 99%, or at least 5%, 10%, 20%, 30%, 40%, 50%, 60%, 70%, 80%, 85%, 90%, 91%, 92%, 93%, 94%, 95%, 96%, 97%, 98%, or 99%) of all oligonucleotides in a composition, or of all oligonucleotides in a composition that share a common base sequence (e.g., of a plurality of oligonucleotide or an oligonucleotide type), or of all oligonucleotides in a composition that share a common base sequence, a common pattern of backbone linkages, and a common pattern of backbone phosphorus modifications are oligonucleotides of the plurality, or of all oligonucleotides in a composition that share a common base sequence, a common patter of base modifications, a common pattern of sugar modifications, a common pattern of internucleotidic linkage types, and/or a common pattern of internucleotidic linkage modifications. In some embodiments, the plurality of oligonucleotides share the same stereochemistry at about 1-50 (e.g., about 1-10, 1-20, 5-10, 5-20, 10-15, 10-20, 10-25, 10-30, or about 1, 2, 3, 4, 5, 6, 7, 8, 9, 10, 11, 12, 13, 14, 15, 16, 17, 18, 19, or 20, or at least 1, 2, 3, 4, 5, 6, 7, 8, 9, 10, 11, 12, 13, 14, 15, 16, 17, 18, 19, or 20) chiral internucleotidic linkages. In some embodiments, the plurality of oligonucleotides share the same stereochemistry at about 1%-100% (e.g., about 5%-100%, 10%-100%, 20%-100%, 30%-100%, 40%-100%, 50%-100%, 60%-100%, 70%-100%, 80-100%, 90-100%, 95-100%, 50%-90%, about 5%, 10%, 15%, 20%, 25%, 30%, 35%, 40%, 45%, 50%, 55%, 60%, 65%, 70%, 75%, 80%, 85%, 90%, 95%, or 100%, or at least 5%, 10%, 15%, 20%, 25%, 30%, 35%, 40%, 45%, 50%, 55%, 60%, 65%, 70%, 75%, 80%, 85%, 90%, 95%, or 99%) of chiral internucleotidic linkages. In some embodiments, each chiral internucleotidic linkage is a chiral controlled internucleotidic linkage, and the composition is a completely chirally controlled oligonucleotide composition. In some embodiments, not all chiral internucleotidic linkages are chiral controlled internucleotidic linkages, and the composition is a partially chirally controlled oligonucleotide composition. In some embodiments, a chirally controlled oligonucleotide composition comprises predetermined levels of individual oligonucleotide or nucleic acids types. For instance, in some embodiments a chirally controlled oligonucleotide composition comprises one oligonucleotide type. In some embodiments, a chirally controlled oligonucleotide composition comprises more than one oligonucleotide type. In some embodiments, a chirally controlled oligonucleotide composition comprises multiple oligonucleotide types. In some embodiments, a chirally controlled oligonucleotide composition is a composition of oligonucleotides of an oligonucleotide type, which composition comprises a predetermined level of a plurality of oligonucleotides of the oligonucleotide type.

Chirally uniform: as used herein, the phrase “chirally uniform” is used to describe an oligonucleotide molecule or type in which all nucleotide units have the same stereochemistry at the linkage phosphorus. For instance, an oligonucleotide whose nucleotide units all have Rp stereochemistry at the linkage phosphorus is chirally uniform. Likewise, an oligonucleotide whose nucleotide units all have Sp stereochemistry at the linkage phosphorus is chirally uniform.

Predetermined: By predetermined (or pre-determined) is meant deliberately selected, for example as opposed to randomly occurring or achieved without control. Those of ordinary skill in the art, reading the present specification, will appreciate that the present disclosure provides technologies that permit selection of particular chemistry and/or stereochemistry features to be incorporated into oligonucleotide compositions, and further permits controlled preparation of oligonucleotide compositions having such chemistry and/or stereochemistry features. Such provided compositions are “predetermined” as described herein. Compositions that may contain certain oligonucleotides because they happen to have been generated through a process that are not controlled to intentionally generate the particular chemistry and/or stereochemistry features is not a “predetermined” composition. In some embodiments, a predetermined composition is one that can be intentionally reproduced (e.g., through repetition of a controlled process). In some embodiments, a predetermined level of a plurality of oligonucleotides in a composition means that the absolute amount, and/or the relative amount (ratio, percentage, etc.) of the plurality of oligonucleotides in the composition is controlled. In some embodiments, a predetermined level of a plurality of oligonucleotides in a composition is achieved through chirally controlled oligonucleotide preparation.

Linkage phosphorus: as defined herein, the phrase “linkage phosphorus” is used to indicate that the particular phosphorus atom being referred to is the phosphorus atom present in the internucleotidic linkage, which phosphorus atom corresponds to the phosphorus atom of a phosphodiester of an internucleotidic linkage as occurs in naturally occurring DNA and RNA. In some embodiments, a linkage phosphorus atom is in a modified internucleotidic linkage, wherein each oxygen atom of a phosphodiester linkage is optionally and independently replaced by an organic or inorganic moiety. In some embodiments, a linkage phosphorus atom is P L of Formula I. In some embodiments, a linkage phosphorus atom is chiral.

P-modification: as used herein, the term “P-modification” refers to any modification at the linkage phosphorus other than a stereochemical modification. In some embodiments, a P-modification comprises addition, substitution, or removal of a pendant moiety covalently attached to a linkage phosphorus. In some embodiments, the “P-modification” is —X-L-R¹ wherein each of X, L and R¹ is independently as defined and described in the present disclosure.

Blockmer: the term “blockmer,” as used herein, refers to an oligonucleotide strand whose pattern of structural features characterizing each individual nucleotide unit is characterized by the presence of at least two consecutive nucleotide units sharing a common structural feature at the internucleotidic phosphorus linkage. By common structural feature is meant common stereochemistry at the linkage phosphorus or a common modification at the linkage phosphorus. In some embodiments, the at least two consecutive nucleotide units sharing a common structure feature at the internucleotidic phosphorus linkage are referred to as a “block”. In some embodiments, a provided oligonucleotide is a blockmer.

In some embodiments, a blockmer is a “stereoblockmer,” e.g., at least two consecutive nucleotide units have the same stereochemistry at the linkage phosphorus. Such at least two consecutive nucleotide units form a “stereoblock.”

In some embodiments, a blockmer is a “P-modification blockmer,” e.g., at least two consecutive nucleotide units have the same modification at the linkage phosphorus. Such at least two consecutive nucleotide units form a “P-modification block”. For instance, (Rp, Sp)-ATsCsGA is a P-modification blockmer because at least two consecutive nucleotide units, the Ts and the Cs, have the same P-modification (i.e., both are a phosphorothioate diester). In the same oligonucleotide of (Rp, Sp)-ATsCsGA, TsCs forms a block, and it is a P-modification block.

In some embodiments, a blockmer is a “linkage blockmer,” e.g., at least two consecutive nucleotide units have identical stereochemistry and identical modifications at the linkage phosphorus. At least two consecutive nucleotide units form a “linkage block”. For instance, (Rp, Rp)-ATsCsGA is a linkage blockmer because at least two consecutive nucleotide units, the Ts and the Cs, have the same stereochemistry (both Rp) and P-modification (both phosphorothioate). In the same oligonucleotide of (Rp, Rp)-ATsCsGA, TsCs forms a block, and it is a linkage block.

In some embodiments, a blockmer comprises one or more blocks independently selected from a stereoblock, a P-modification block and a linkage block. In some embodiments, a blockmer is a stereoblockmer with respect to one block, and/or a P-modification blockmer with respect to another block, and/or a linkage blockmer with respect to yet another block.

Altmer: the term “altmer,” as used herein, refers to an oligonucleotide strand whose pattern of structural features characterizing each individual nucleotide unit is characterized in that no two consecutive nucleotide units of the oligonucleotide strand share a particular structural feature at the internucleotidic phosphorus linkage. In some embodiments, an altmer is designed such that it comprises a repeating pattern. In some embodiments, an altmer is designed such that it does not comprise a repeating pattern. In some embodiments, a provided oligonucleotide is a altmer.

In some embodiments, an altmer is a “stereoaltmer,” e.g., no two consecutive nucleotide units have the same stereochemistry at the linkage phosphorus.

In some embodiments, an altmer is a “P-modification altmer” e.g., no two consecutive nucleotide units have the same modification at the linkage phosphorus. For instance, All-(Sp)-CAs1GsT, in which each linkage phosphorus has a different P-modification than the others.

In some embodiments, an altmer is a “linkage altmer,” e.g., no two consecutive nucleotide units have identical stereochemistry or identical modifications at the linkage phosphorus.

Unimer: the term “unimer,” as used herein, refers to an oligonucleotide strand whose pattern of structural features characterizing each individual nucleotide unit is such that all nucleotide units within the strand share at least one common structural feature at the internucleotidic phosphorus linkage. By common structural feature is meant common stereochemistry at the linkage phosphorus or a common modification at the linkage phosphorus. In some embodiments, a provided oligonucleotide is a unimer.

In some embodiments, a unimer is a “stereounimer,” e.g., all nucleotide units have the same stereochemistry at the linkage phosphorus.

In some embodiments, a unimer is a “P-modification unimer”, e.g., all nucleotide units have the same modification at the linkage phosphorus.

In some embodiments, a unimer is a “linkage unimer,” e.g., all nucleotide units have the same stereochemistry and the same modifications at the linkage phosphorus.

Gapmer: as used herein, the term “gapmer” refers to an oligonucleotide strand characterized in that at least one internucleotidic phosphorus linkage of the oligonucleotide strand is a phosphate diester linkage, for example such as those found in naturally occurring DNA or RNA. In some embodiments, more than one internucleotidic phosphorus linkage of the oligonucleotide strand is a phosphate diester linkage such as those found in naturally occurring DNA or RNA. In some embodiments, a provided oligonucleotide is a gapmer.

Skipmer: as used herein, the term “skipmer” refers to a type of gapmer in which every other internucleotidic phosphorus linkage of the oligonucleotide strand is a phosphate diester linkage, for example such as those found in naturally occurring DNA or RNA, and every other internucleotidic phosphorus linkage of the oligonucleotide strand is a modified internucleotidic linkage. In some embodiments, a provided oligonucleotide is a skipmer.

For purposes of this disclosure, the chemical elements are identified in accordance with the Periodic Table of the Elements, CAS version, Handbook of Chemistry and Physics, 67th Ed., 1986-87, inside cover.

The methods and structures described herein relating to compounds and compositions of the disclosure also apply to the pharmaceutically acceptable acid or base addition salts and all stereoisomeric forms of these compounds and compositions.

### BRIEF DESCRIPTION OF THE DRAWINGS

FIG. 1, including FIG. 1A to 1L, presents cartoons of various ssRNAi formats and hybrid formats.

FIG. 2 presents cartoons of various antisense oligonucleotide formats.

FIG. 3A shows example multimer formats. Oligonucleotides can be joined directly and/or through linkers. As illustrated, a multimer can comprise oligonucleotide monomers of the same or different structures/types. In some embodiments, a monomer of a multimer is an ssRNAi agent. In some embodiments, a monomer of a multimer is a RNase H-dependent antisense oligonucleotide (ASO). Monomers can be joined through various positions, for example, the 5′-end, the 3′-end, or positions in between. FIG. 3B shows example chemistry approaches for joining monomers, which monomers may perform their functions through various pathways, to form multimers.

### DETAILED DESCRIPTION OF CERTAIN EMBODIMENTS

Synthetic oligonucleotides provide useful molecular tools in a wide variety of applications. For example, oligonucleotides are useful in therapeutic, diagnostic, research, and new nanomaterials applications. The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modifications, e.g., base modifications, sugar modifications, backbone modifications, etc., which, among other things, render these molecules less susceptible to degradation and improve other properties of oligonucleotides. From a structural point of view, modifications to internucleotide phosphate linkages can introduce chirality, and certain properties of oligonucleotides may be affected by configurations of phosphorus atoms that form the backbone of oligonucleotides. For example, in vitro studies have shown that properties of antisense oligonucleotides, such as binding affinity, sequence specific binding to complementary RNA, stability to nucleases, are affected by, inter alia, chirality of backbone phosphorus atoms.

Among other things, the present disclosure encompasses the recognition that structural elements of oligonucleotides, such as chemical modifications (e.g., modifications of sugar, base, and/or internucleotidic linkages) or patterns thereof, conjugation to lipids or other moieties, and/or stereochemistry [e.g., stereochemistry of backbone chiral centers (chiral internucleotidic linkages), and/or patterns thereof], can have significant impact on properties and activities (e.g., stability, specificity, selectivity, activities to reduce levels of products (transcripts and/or protein) of target genes, etc.). In some embodiments, oligonucleotide properties can be adjusted by optimizing chemical modifications (modifications of base, sugar, and/or internucleotidic linkage moieties), patterns of chemical modifications, stereochemistry and/or patterns of stereochemistry.

In some embodiments, the present disclosure demonstrates that oligonucleotide compositions comprising oligonucleotides with controlled structural elements, e.g., controlled chemical modifications and/or controlled backbone stereochemistry patterns, provide unexpected properties and activities, including but not limited to those described herein. In some embodiments, provided compositions comprising oligonucleotides having chemical modifications (e.g., base modifications, sugar modification, internucleotidic linkage modifications, etc.) or patterns thereof have improved properties and activities. Non-limiting examples of such improved properties include: directing a decrease in the expression and/or level of a target gene or its gene product; and/or directing RNA interference; and/or directing RNase H-mediated knockdown. In some embodiments, the present disclosure provides technologies (e.g., oligonucleotides, compositions, methods, etc.) for single-stranded RNAi. In some embodiments, a provided oligonucleotide is a ssRNAi agent.

In some embodiments, RNA interference is reportedly a post-transcriptional, targeted gene-silencing technique that uses an RNAi agent to target a RNA, e.g., a gene transcript such as a messenger RNA (mRNA), comprising a sequence complementary to the RNAi agent, for cleavage mediated by the RISC (RNA-induced silencing complex) pathway. In nature, a type of RNAi reportedly occurs when ribonuclease III (Dicer) cleaves a long dsRNA (double-stranded RNA) (e.g., a foreign dsRNA introduced into a mammalian cell) into shorter fragments called siRNAs. siRNAs (small interfering RNAs or short inhibitory RNAs) are typically about 21 to 23 nucleotides long and comprise about 19 base pair duplexes. The smaller RNA segments then reportedly mediate the degradation of the target mRNA. The RNAi response also reportedly features an endonuclease complex, commonly referred to as an RNA-induced silencing complex (RISC), which directs cleavage of single-stranded mRNA complementary to the antisense strand of the siRNA. Cleavage of the target RNA reportedly takes place in the middle of the region complementary to the antisense strand of the siRNA duplex. The use of the RNAi agent to a target transcript reportedly results in a decrease of gene activity, level and/or expression, e.g., a “knock-down” or “knock-out” of the target gene or target sequence. Artificial siRNAs are useful both as therapeutics and for experimental use.

In one aspect, an RNA interference agent includes a single stranded RNA that interacts with a target RNA sequence to direct the cleavage of the target RNA. Without wishing to be bound by theory, long double stranded RNA introduced into plants and invertebrate cells is reportedly broken down into siRNA by a Type III endonuclease known as Dicer (Sharp et al., Genes Dev. 2001, 15:485). Dicer, a ribonuclease-III-like enzyme, reportedly processes the dsRNA into 19-23 base pair short interfering RNAs with characteristic two base 3′ overhangs (Bernstein, et al., (2001) Nature 409:363). The siRNAs are reportedly then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15: 188). Thus, in one aspect the disclosure relates to a single stranded RNA that promotes the formation of a RISC complex to effect silencing of a target gene.

In some embodiments, a suitable RNAi agent can be selected by any processes known in the art or conceivable by one of ordinary skill in the art in accordance with the present disclosure. For example, the selection criteria can include one or more of the following steps: initial analysis of the target gene sequence and design of RNAi agents; this design can take into consideration sequence similarity across species (human, cynomolgus, mouse, etc.) and dissimilarity to other (non-target) genes; screening of RNAi agents in vitro (e.g., at 10 nM in cells expressing the target transcript); determination of EC50 or IC50 in cells; determination of viability of cells treated with RNAi agents, wherein it is desired, in some embodiments, that the RNAi agent to the target not inhibit the viability of these cells; testing with human PBMC (peripheral blood mononuclear cells), e.g., to test levels of TNF-alpha to estimate immunogenicity, wherein immunostimulatory sequences are usually less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein immunostimulatory sequences are usually less desired; determination of gene knockdown in vivo using cells or tumors in test animals; and optimization of specific modifications of the RNAi agents.

The so-called canonical siRNA structure is reportedly a double-stranded RNA molecule, wherein each strand is about 21 nucleotides long. The two strands are reportedly an antisense (or “guide”) strand, which recognizes and binds to a complementary sequence in the target transcript, and a sense (or “passenger”) strand, which is complementary to the antisense strand. The sense and antisense strands are reportedly largely complementary, typically forming two 3′ overhangs of 2 nucleotides on both ends.

While a canonical siRNA structure is reportedly double-stranded, RNAi agent can also be single-stranded. In some embodiments, a single-stranded RNAi agent corresponds to an antisense strand of a double-stranded siRNA, and the single-stranded RNAi agent lacks a corresponding passenger strand.

However, it has been reported that not all tested structural elements for single-stranded RNAi agents are effective; introduction of some structural elements into an oligonucleotide can reportedly interference with single-stranded RNA interference activity.

In some embodiments, the present disclosure provides oligonucleotides and compositions useful as RNAi agent. In some embodiments, the present disclosure provides oligonucleotides and compositions useful as single-stranded RNAi agent. The present disclosure, among other things, provides novel structures of single-stranded oligonucleotides capable of directing RNA interference. Without wishing to be bound by any particular theory, this disclosure notes that single-stranded RNAi agents have advantages over double-stranded RNAi agents. For example, single-stranded RNAi agents have a lower cost of goods, as the construction of only one strand is required. Additionally or alternatively, only one strand (the antisense strand) is administered to target a target transcript. A source of off-target effects directed by dsRNA is loading of the sense strand into RISC and binding to and knockdown of undesired targets (Jackson et al. 2003 Nat. Biotech. 21: 635-637), a single-stranded RNAi agent can elicit fewer off-target effects than a corresponding double-stranded RNAi agent. In addition, some single-stranded RNAi agents, including some disclosed herein, can target particular sequences which have not previously been successfully targeted with double-stranded RNAi agents (for example, they can reduce levels of the sequences, and/or products (transcripts and/or proteins) of the sequences, significantly more than double-stranded RNAi agents). The present disclosure, among other things, provides novel formats (modifications, stereochemistry, combinations thereof, etc.) for oligonucleotides which can direct single-stranded RNA interference.

### Oligonucleotides

In some embodiments, provided oligonucleotides can direct a decrease in the expression and/or level of a target gene or its gene product. In some embodiments, provided oligonucleotides can direct a decrease in levels of target products. In some embodiments, provided oligonucleotide can reduce levels of transcripts of target genes. In some embodiments, provided oligonucleotide can reduce levels of mRNA of target genes. In some embodiments, provided oligonucleotide can reduce levels of proteins encoded by target genes. In some embodiments, provided oligonucleotides can direct a decrease in the expression and/or level of a target gene or its gene product via RNA interference. In some embodiments, provided oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides can direct a decrease in the expression and/or level of a target gene or its gene product via RNA interference and/or RNase H-mediated knockdown. In some embodiments, provided oligonucleotides can direct a decrease in the expression and/or level of a target gene or its gene product by sterically blocking translation after binding to a target gene mRNA, and/or by altering or interfering with mRNA splicing and/or exon inclusion or exclusion. In some embodiments, provided oligonucleotides comprise one or more structural elements described herein or known in the art in accordance with the present disclosure, e.g., base sequences; modifications; stereochemistry; patterns of internucleotidic linkages; GC contents; long GC stretches; patterns of backbone linkages; patterns of backbone chiral centers; patterns of backbone phosphorus modifications; additional chemical moieties, including but not limited to, one or more targeting moieties, lipid moieties, and/or carbohydrate moieties, etc.; seed regions; post-seed regions; 5′-end structures; 5′-end regions; 5′ nucleotide moieties; 3′-end regions; 3′-terminal dinucleotides; 3′-end caps; etc. In some embodiments, a seed region of an oligonucleotide is or comprises the second to eighth, second to seventh, second to sixth, third to eighth, third to seventh, third to seven, or fourth to eighth or fourth to seventh nucleotides, counting from the 5′ end; and the post-seed region of the oligonucleotide is the region immediately 3′ to the seed region, and interposed between the seed region and the 3′ end region.

In some embodiments, a provided composition comprises an oligonucleotide. In some embodiments, a provided composition comprises one or more lipid moieties, one or more carbohydrate moieties (unless otherwise specified, other than sugar moieties of nucleoside units that form oligonucleotide chain with internucleotidic linkages), and/or one or more targeting components.

In some embodiments, the present disclosure provides a provided compound, e.g., an oligonucleotide of a provided composition, having the structure of formula O-I:

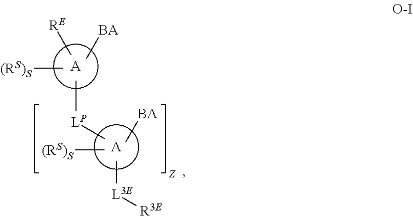

or a salt thereof, wherein:
R^{E} is a 5′-end group;
each BA is independently an optionally substituted group selected from C₁₋₃₀ cycloaliphatic, C₆₋₃₀ aryl, C₅₋₃₀ heteroaryl having 1-10 heteroatoms independently selected from oxygen, nitrogen, sulfur, phosphorus and silicon, C₃₋₃₀ heterocyclyl having 1-10 heteroatoms independently selected from oxygen, nitrogen, sulfur, phosphorus, boron and silicon, a natural nucleobase moiety, and a modified nucleobase moiety;
each R^{s} is independently —F, —Cl, —Br, —I, —CN, —N₃, —NO, —NO₂, -L-R′, -L-OR′, -L-SR′, -L-N(R′)₂, —O-L-OR′, —O-L-SR′, or —O-L-N(R′)₂;
s is 0-20;
each L is independently a covalent bond, or a bivalent, optionally substituted, linear or branched group selected from a C₁₋₃₀ aliphatic group and a C₁₋₃₀ heteroaliphatic group having 1-10 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};
each Cy^{L} is independently an optionally substituted tetravalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus, boron and silicon;
each Ring A is independently an optionally substituted 3-20 membered monocyclic, bicyclic or polycyclic ring having 0-10 heteroatoms independently selected from oxygen, nitrogen, sulfur, phosphorus and silicon;
each L^{P} is independently an internucleotidic linkage;
z is 1-1000;
L^{3E} is L or -L-L-;
R^{3E} is —R′, -L-R′, —OR′, or a solid support;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms independently selected from oxygen, nitrogen, sulfur, phosphorus and silicon.

In some embodiments, each L^{P} independently has the structure of formula I:

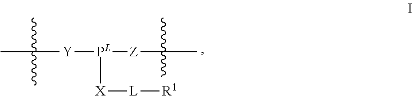

or a salt form thereof, wherein:
P^{L} is P(═W), P, or P→B(R′)₃;
W is O, S or Se;
R¹ is -L-R, halogen, —CN, —NO₂, —Si(R)₃, —OR, —SR, or —N(R)₂;
each of X, Y and Z is independently —O—, —S—, —N(-L-R¹)—, or L;
wherein each variable is independently as described in the present disclosure.

In some embodiments, each L^{P} independently has the structure of formula I, and R^{E} is —C(R^{5s})₃, -L-P^{DB}, —C(R^{5s})₂OH, -L-R^{5s}, or -L-P^{5s}-L-R^{5s}, or a salt form thereof, wherein each variable is independently as described in the present disclosure.

In some embodiments, R^{E} is —C(R^{5s})₃, -L-P^{DB}, —C(R^{5s})₂OH, -L-R^{5s}, or -L-P^{5s}-L-R^{5s}, or a salt form thereof;
each BA is independently an optionally substituted group selected from C₅₋₃₀ heteroaryl having 1-10 heteroatoms independently selected from oxygen, nitrogen, sulfur, phosphorus and silicon, and C₃₋₃₀ heterocyclyl having 1-10 heteroatoms independently selected from oxygen, nitrogen, sulfur, phosphorus, boron and silicon;
each Ring A is independently an optionally substituted 3-20 membered monocyclic, bicyclic or polycyclic ring having 0-10 heteroatoms independently selected from oxygen, nitrogen, sulfur, phosphorus and silicon; and
each L^{P} independently has the structure of formula I, wherein each variable is independently as described in the present disclosure.

In some embodiments, R^{E} is —C(R^{5s})₃, -L-P^{DB}, —C(R^{5s})₂OH, -L-R^{5s}, or -L-P^{5s}-L-R^{5s}, or a salt form thereof;
each BA is independently an optionally substituted C₅₋₃₀ heteroaryl having 1-10 heteroatoms independently selected from oxygen, nitrogen, sulfur, phosphorus and silicon, wherein the heteroaryl comprises one or more heteroatoms selected from oxygen and nitrogen;
each Ring A is independently an optionally substituted 5-10 membered monocyclic or bicyclic saturated ring having 0-5 heteroatoms independently selected from oxygen, nitrogen, sulfur, phosphorus and silicon, wherein the ring comprises at least one oxygen atom; and
each L^{P} independently has the structure of formula I, wherein each variable is independently as described in the present disclosure.

In some embodiments, R^{E} is —C(R^{5s})₃, -L-P^{DB}, —C(R^{5s})₂OH, -L-R^{5s}, or -L-P^{5s}-L-R^{5s}, or a salt form thereof;
each BA is independently an optionally substituted or protected nucleobase selected from adenine, cytosine, guanosine, thymine, and uracil;
each Ring A is independently an optionally substituted 5-7 membered monocyclic or bicyclic saturated ring having one or more oxygen atoms; and
each L^{P} independently has the structure of formula I, wherein each variable is independently as described in the present disclosure.

In some embodiments, R^{E} is a 5′-end group as described herein. In some embodiments, R^{E} is —C(R^{5s})₃, -L-P^{DB}, —C(R^{5s})₂OH, -L-R^{5s}, or -L-P^{5s}-L-R^{5s}, or a salt form thereof, wherein each variable is independently as described in the present disclosure. In some embodiments, R^{E} is —CH₂OH. In some embodiments, R^{E} is —CH₂OP(O)(OR)₂ or a salt form thereof, wherein each R is independently as described in the present disclosure. In some embodiments, R^{E} is —CH₂OP(O)(OH)₂ or a salt form thereof. In some embodiments, R^{E} is —CH₂OP(O)(OR)(SR) or a salt form thereof, wherein each R is independently as described in the present disclosure. In some embodiments, R^{E} is —CH₂OP(O)(SH)(OH) or a salt form thereof. In some embodiments, R^{E} is (E)-CH═CHP(O)(OR)₂ or a salt form thereof, wherein each R is independently as described in the present disclosure. In some embodiments, R^{E} is (E)-CH═CHP(O)(OH)₂.

In some embodiments, the present disclosure provides multimers of oligonucleotides. In some embodiments, a multimer is a multimer of the same oligonucleotides. In some embodiments, a multimer is a multimer of structurally different oligonucleotides. In some embodiments, each oligonucleotide of a multimer performs its functions independently through its own pathways, e.g., RNAi, RNase-H dependent, etc. In some embodiments, provided oligonucleotides exist in an oligomeric or polymeric form, in which one or more oligonucleotide moieties are linked together by linkers, e.g., L, L^{M}, etc., through nucleobases, sugars, and/or internucleotidic linkages of the oligonucleotide moieties. For example, in some embodiments, a provided multimer compound has the structure of (A^{c})ₐ-L^{M}-(A^{c})_{b}, wherein each variable is independently as described in the present disclosure. Example multimer technologies include those illustrated in FIG. 89, wherein each oligonucleotide can independently function through different pathways, e.g., RNAi, and/or RNase-H dependent.

In some embodiments, a provided compound, e.g., an oligonucleotide of a provided composition, has the structure of:

A^{c}-[-L^{M}-(R^{D})ₐ]_{b},[(A^{c})ₐ-L^{M}]_{b}-R^{D},(A^{c})ₐ-L^{M}-(A^{c})_{b}, or (A^{c})ₐ-L^{M}-(R^{D})_{b},

or a salt thereof, wherein:
each A^{c} is independently an oligonucleotide moiety (e.g., [H]ₐ-A^{c} or [H]_{b}-A^{c} is an oligonucleotide);
a is 1-1000;
b is 1-1000;
L^{M} is a multivalent linker; and
each R^{D} is independently a lipid moiety, a carbohydrate moiety, or a targeting moiety.

In some embodiments, a provided compound, e.g., an oligonucleotide of a provided composition, have the structure of:

A^{c}-[-L^{M}-(R^{D})ₐ]_{b},[(A^{c})ₐ-L^{M}]_{b}-R^{D},(A^{c})ₐ-L^{M}-(A^{c})_{b}, or (A^{c})ₐ-L^{M}-(R^{D})_{b},

or a salt thereof, wherein:
each A^{c} is independently an oligonucleotide moiety (e.g., [H]ₐ-A^{c} or [H]_{b}-A^{c} is an oligonucleotide);
a is 1-1000;
b is 1-1000;
each R^{D} is independently R^{LD}, R^{CD} or R^{TD};
R^{CD} is an optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};
R^{LD} is an optionally substituted, linear or branched group selected from a C₁₋₁₀₀ aliphatic group wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};
R^{TD} is a targeting moiety;
each L^{M} is independently a covalent bond, or a bivalent or multivalent, optionally substituted, linear or branched group selected from a C₁₋₁₀₀ aliphatic group and a C₁₋₁₀₀ heteroaliphatic group having 1-30 heteroatoms independently selected from oxygen, nitrogen, sulfur, phosphorus, boron and silicon, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};
each Cy^{L} is independently an optionally substituted tetravalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus, boron and silicon;
each R′ is independently —R, —C(O)R, —C(O)OR, or —S(O)₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms independently selected from oxygen, nitrogen, sulfur, phosphorus and silicon.

In some embodiments, R^{E} is —CH₂OH. In some embodiments, R^{E} is —CH₂OP(O)(R)₂ or a salt form thereof, wherein each R is independently as described in the present disclosure. In some embodiments, R^{E} is —CH₂OP(O)(OR)₂ or a salt form thereof, wherein each R is independently as described in the present disclosure. In some embodiments, R^{E} is —CH₂OP(O)(OH)₂ or a salt form thereof. In some embodiments, R^{E} is —CH₂OP(O)(OR)(SR) or a salt form thereof, wherein each R is independently as described in the present disclosure. In some embodiments, R^{E} is —CH₂OP(O)(SH)(OH) or a salt form thereof. In some embodiments, R^{E} is (E)-CH═CHP(O)(OR)₂ or a salt form thereof, wherein each R is independently as described in the present disclosure. In some embodiments, R^{E} is (E)-CH═CHP(O)(OH)₂.

In some embodiments, R^{E} is —CH(R^{5s})—OH, wherein R^{5s} is as described in the present disclosure. In some embodiments, R^{E} is —CH(R^{5s})—OP(O)(R)₂ or a salt form thereof, wherein each R^{5s} and R is independently as described in the present disclosure. In some embodiments, R^{E} is —CH(R^{5s})—OP(O)(OR)₂ or a salt form thereof, wherein each R^{5s} and R is independently as described in the present disclosure. In some embodiments, R^{E} is —CH(R^{5s})—OP(O)(OH)₂ or a salt form thereof. In some embodiments, R^{E} is —CH(R^{5s})—OP(O)(OR)(SR) or a salt form thereof. In some embodiments, R^{E} is —CH(R^{5s})—OP(O)(OH)(SH) or a salt form thereof. In some embodiments, R^{E} is —(R)—CH(R^{5s})—OH, wherein R^{5s} is as described in the present disclosure. In some embodiments, R^{E} is —(R)—CH(R^{5s})—OP(O)(R)₂ or a salt form thereof, wherein each R^{5s} and R is independently as described in the present disclosure. In some embodiments, R^{E} is —(R)—CH(R^{5s})—OP(O)(OR)₂ or a salt form thereof, wherein each R^{5s} and R is independently as described in the present disclosure. In some embodiments, R^{E} is —(R)—CH(R^{5s})—OP(O)(OH)₂ or a salt form thereof. In some embodiments, R^{E} is —(R)—CH(R^{5s})—OP(O)(OR)(SR) or a salt form thereof. In some embodiments, R^{E} is —(R)—CH(R^{5s})—OP(O)(OH)(SH) or a salt form thereof. In some embodiments, R^{E} is —(S)—CH(R^{5s})—OH, wherein R^{5s} is as described in the present disclosure. In some embodiments, R^{E} is —(S)—CH(R^{5s})—OP(O)(R)₂ or a salt form thereof, wherein each R^{5s} and R is independently as described in the present disclosure. In some embodiments, R^{E} is —(S)—CH(R^{5s})—OP(O)(OR)₂ or a salt form thereof, wherein each R^{5s} and R is independently as described in the present disclosure. In some embodiments, R^{E} is —(S)—CH(R^{5s})—OP(O)(OH)₂ or a salt form thereof. In some embodiments, R^{E} is —(S)—CH(R^{5s})—OP(O)(OR)(SR) or a salt form thereof. In some embodiments, R^{E} is —(S)—CH(R^{5s})—OP(O)(OH)(SH) or a salt form thereof. In some embodiments, R^{5s} is optionally substituted C₁, C₂, C₃, or C₄ aliphatic. In some embodiments, R^{5s} is C₁, C₂, C₃, or C₄ aliphatic or haloaliphatic. In some embodiments, R^{5s} is optionally substituted —CH₃. In some embodiments, R^{5s} is —CH₃.

In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, has the structure of formula O-I. In some embodiments, a compound of formula O-I is an oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′. In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3 has the structure of A^{c}-[-L^{M}-(R^{D})_{a]b}, [(A^{c})ₐ-L^{M}]_{b}-R^{D}, (A^{c})ₐ-L^{M}-(A^{c})_{b}, or (A^{c})ₐ-L^{M}-(R^{D})_{b}. In some embodiments, a compound of A^{c}-[-L^{M}-(R^{D})_{a]b}, [(A^{c})ₐ-L^{M}]_{b}-R^{D}, (A^{c})ₐ-L^{M}-(A^{c})_{b}, or (A^{c})ₐ-L^{M}-(R^{D})_{b} is an oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′. In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, has the structure of A^{c}-[-L^{M}-(R^{D})_{a]b}. In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, has the structure of [(A^{c})ₐ-L^{M}]_{b}-R^{D}. In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, has the structure of (A^{c})ₐ-L^{M}-(A^{c})_{b}. In some embodiments, a provided oligonucleotide, for example, a provided oligonucleotide having the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, has the structure of (A^{c})ₐ-L^{M}-(R^{D})_{b}. In some embodiments, such a provided oligonucleotide, or a provided composition comprising such an oligonucleotide, functions in a system (e.g., a biochemical assay, a cell, a tissue, an organ, an organism, a subject, etc.) through a RNase H pathway to, for example, reduce levels of a nucleic acid or a product encoded thereof. In some embodiments, such a provided oligonucleotide, or a provided composition comprising such an oligonucleotide, functions in a system through a RNAi pathway to, for example, reduce levels of a nucleic acid or a product encoded thereof. In some embodiments, a provided oligonucleotide is single-stranded. In some embodiments, a provided oligonucleotide is administered to a system single-stranded.

In some embodiments, a provided compound, e.g., oligonucleotide of formula O-I, A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)4N19-PX19)_{nz}-(N20-PX20)_{pz}-(N21-PX21)_{tz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, may exist as a salt. In some embodiments, the present disclosure provides salts of oligonucleotides, and pharmaceutical compositions thereof. In some embodiments, a salt is a pharmaceutically acceptable salt. In some embodiments, each hydrogen ion that may be donated to a base (e.g., under conditions of an aqueous solution, a pharmaceutical composition, etc.) is replaced by a non-H⁺ cation. For example, in some embodiments, a pharmaceutically acceptable salt of an oligonucleotide is an all-metal ion salt, wherein each hydrogen ion (for example, of —OH, —SH, etc.) of each internucleotidic linkage (e.g., a natural phosphate linkage, a phosphorothioate diester linkage, etc.) is replaced by a metal ion. In some embodiments, a provided salt is an all-sodium salt. In some embodiments, a provided pharmaceutically acceptable salt is an all-sodium salt. In some embodiments, a provided salt is an all-sodium salt, wherein each internucleotidic linkage which is a natural phosphate linkage (acid form —O—P(O)(OH)—O—), if any, exists as its sodium salt form (—O—P(O)(ONa)—O—), and each internucleotidic linkage which is a phosphorothioate diester linkage (acid form —O—P(O)(SH)—O—), if any, exists as its sodium salt form (—O—P(O)(SNa)—O—).

In some embodiments, where presence and/or activity of a particular allele (and/or its one or more products (e.g., RNA and/or protein products)) (a disease-associated allele) is associated (e.g., correlated) with presence, incidence and/or severity of one or more diseases and/or conditions, a different allele of the same sequence (e.g. gene) exists and is not so associated, or is associated to a lesser extent (e.g., shows less significant, or statistically insignificant correlation). In some such embodiments, oligonucleotides and methods thereof as described herein may preferentially or specifically target the associated allele relative to the one or more less-associated/unassociated allele(s).

In some embodiments, a target sequence is a sequence to which an oligonucleotide as described herein binds. In many embodiments, a target sequence is identical to, or is an exact complement of, a sequence of a provided oligonucleotide, or of consecutive residues therein (e.g., a provided oligonucleotide includes a target-binding sequence that is identical to, or an exact complement of, a target sequence). In some embodiments, a small number of differences/mismatches is tolerated between (a relevant portion of) an oligonucleotide and its target sequence. In many embodiments, a target sequence is present within a target gene. In many embodiments, a target sequence is present within a transcript (e.g., an mRNA and/or a pre-mRNA) produced from a target gene.

In some embodiments, a target sequence includes one or more allelic sites (i.e., positions within a target gene at which allelic variation occurs). In some such embodiments, a provided oligonucleotide binds to one allele preferentially or specifically relative to one or more other alleles. In some embodiments, a target-binding sequence is identical to, or is an exact complement of, a target sequence of one allele. In some embodiments, a target-binding sequence is identical to a target sequence of one allele. In some embodiments, a target-binding sequence is an exact complement of a target sequence of one allele. In some embodiments, a provided oligonucleotide binds preferentially to a disease-associated allele. In some embodiments, a provided oligonucleotide binds preferentially to a disease-associated allele, and comprises a target-binding sequence which is identical to, or is an exact complement of, a target sequence of a disease-associated allele but not other allele(s). For example, in some embodiments, an oligonucleotide (or a target-binding sequence portion thereof) provided herein has a sequence that is identical to, or an exact complement of a particular allelic version of a target sequence. In some embodiments, a target sequence is a sequence of a particular allele. In some embodiments, an oligonucleotide (or a target-binding sequence portion thereof) provided herein has a sequence that is identical to, or an exact complement of an allelic site of a disease-associated allele.

A target-binding sequence, and/or a target sequence that it is identical to, or an exact complement of, can be of various lengths. In some embodiments, a length is 2-30 bases or longer. In some embodiments, a length is 5-20 bases. In some embodiments, a length is 10-20 bases. In some embodiments, a length is 2 bases. In some embodiments, a length is 3 bases. In some embodiments, a length is 4 bases. In some embodiments, a length is 5 bases. In some embodiments, a length is 6 bases. In some embodiments, a length is 7 bases. In some embodiments, a length is 8 bases. In some embodiments, a length is 9 bases. In some embodiments, a length is 10 bases. In some embodiments, a length is 11 bases. In some embodiments, a length is 12 bases. In some embodiments, a length is 13 bases. In some embodiments, a length is 14 bases. In some embodiments, a length is 15 bases. In some embodiments, a length is 16 bases. In some embodiments, a length is 17 bases. In some embodiments, a length is 18 bases. In some embodiments, a length is 19 bases. In some embodiments, a length is 20 bases. In some embodiments, a base is optionally substituted adenine, cytosine, guanosine, thymine, or uracil. In some embodiments, complementarity is determined based on A=T/U and G≡C.

As appreciated by those skilled in the art, various allelic sites can be included in a target sequence in accordance with the present disclosure. In some embodiments, a target sequence comprises a SNP. In some embodiments, a target sequence comprises a mutation. In some embodiments, a SNP is a SNP in PNPLA3.

Various linker, lipid moieties, carbohydrate moieties and targeting moieties, including many known in the art, can be utilized in accordance with the present disclosure. In some embodiments, a lipid moiety is a targeting moiety. In some embodiments, a carbohydrate moiety is a targeting moiety. In some embodiments, a targeting moiety is a lipid moiety. In some embodiments, a targeting moiety is a carbohydrate moiety. As readily appreciated by those skilled in the art, various linkers, including those described in the present disclosure, can be utilized in accordance with the present disclosure to link two moieties, for example, a lipid/carbohydrate/targeting component with an oligonucleotide moiety. As readily appreciated by those skilled in the art, linkers described for linking two moieties can also be used to link other moieties, for example, linkers for linking a lipid and an oligonucleotide moiety can also be used to link a carbohydrate or target moiety with an oligonucleotide moiety and vice versa.

In some embodiments, A^{c} is of such an structure that its corresponding oligonucleotide (e.g., [H]ₐ-A^{c} or [H]_{b}-A^{c}) is an oligonucleotide described in the present disclosure, optionally comprising a lipid moiety, a carbohydrate moiety, and/or a targeting moiety. In some embodiments, A^{c} contains no lipid moieties, no carbohydrate moieties, and no targeting moieties. In some embodiments, A^{c} contains no lipid moieties and no carbohydrate moieties. In some embodiments, A^{c} contains no lipid moieties. In some embodiments, A^{c} contains no carbohydrate moieties. In some embodiments, A′ contains no targeting moieties. In some embodiments, A′ is a monovalent, bivalent, or multivalent oligonucleotide moiety of an oligonucleotide having the structure of formula O-I.

In some embodiments, the present disclosure provides oligonucleotides and oligonucleotide compositions that are chirally controlled. For instance, in some embodiments, a provided composition contains predetermined levels of one or more individual oligonucleotide types, wherein an oligonucleotide type is defined by: 1) base sequence; 2) pattern of backbone linkages; 3) pattern of backbone chiral centers; and 4) pattern of backbone P-modifications. In some embodiments, a particular oligonucleotide type may be defined by 1A) base identity; 1B) pattern of base modification; 1C) pattern of sugar modification; 2) pattern of backbone linkages; 3) pattern of backbone chiral centers; and 4) pattern of backbone P-modifications. In some embodiments, oligonucleotides of the same oligonucleotide type are identical. In some embodiments, the present disclosure provides chirally controlled oligonucleotide compositions of oligonucleotides, wherein the composition comprises a predetermined level of a plurality of oligonucleotides, wherein oligonucleotides of the plurality share a common base sequence, and comprise the same configuration of linkage phosphorus at at least 1, 2, 3, 4, 5, 6, 7, 8, 9, 10, 11, 12, 13, 14, 15, 16, 17, 18, 19, 20, 21, 22, 23, 24, or 25 chiral internucleotidic linkages (chirally controlled internucleotidic linkages).

In some embodiments, oligonucleotides of a predetermined level and/or a provided plurality, e.g., those of formula O-I, A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{tz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, comprise 1-30 chirally controlled internucleotidic linkages.

In some embodiments, provided oligonucleotides comprise 2-30 chirally controlled internucleotidic linkages. In some embodiments, provided oligonucleotides comprise 5-30 chirally controlled internucleotidic linkages. In some embodiments, provided oligonucleotides comprise 10-30 chirally controlled internucleotidic linkages. In some embodiments, provided oligonucleotides comprise 1 chirally controlled internucleotidic linkage. In some embodiments, provided oligonucleotides comprise 2 chirally controlled internucleotidic linkages. In some embodiments, provided oligonucleotides comprise 3 chirally controlled internucleotidic linkages. In some embodiments, provided oligonucleotides comprise 4 chirally controlled internucleotidic linkages. In some embodiments, provided oligonucleotides comprise 5 chirally controlled internucleotidic linkages. In some embodiments, provided oligonucleotides comprise 6 chirally controlled internucleotidic linkages. In some embodiments, provided oligonucleotides comprise 7 chirally controlled internucleotidic linkages. In some embodiments, provided oligonucleotides comprise 8 chirally controlled internucleotidic linkages. In some embodiments, provided oligonucleotides comprise 9 chirally controlled internucleotidic linkages. In some embodiments, provided oligonucleotides comprise 10 chirally controlled internucleotidic linkages. In some embodiments, provided oligonucleotides comprise 11 chirally controlled internucleotidic linkages. In some embodiments, provided oligonucleotides comprise 12 chirally controlled internucleotidic linkages. In some embodiments, provided oligonucleotides comprise 13 chirally controlled internucleotidic linkages. In some embodiments, provided oligonucleotides comprise 14 chirally controlled internucleotidic linkages. In some embodiments, provided oligonucleotides have 15 chirally controlled internucleotidic linkages. In some embodiments, provided oligonucleotides have 16 chirally controlled internucleotidic linkages. In some embodiments, provided oligonucleotides have 17 chirally controlled internucleotidic linkages. In some embodiments, provided oligonucleotides have 18 chirally controlled internucleotidic linkages. In some embodiments, provided oligonucleotides have 19 chirally controlled internucleotidic linkages. In some embodiments, provided oligonucleotides have 20 chirally controlled internucleotidic linkages.

In some embodiments, about 1-100% of all internucleotidic linkages are chirally controlled internucleotidic linkages. In some embodiments, a percentage is about 5%-100%. In some embodiments, a percentage is at least 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%. In some embodiments, a percentage is about 5%, 10%, 15%, 20%, 25%, 30%, 35%, 40%, 45%, 50%, 55%, 60%, 65%, 70%, 75%, 80%, 85%, 90%, 95%, 965, 96%, 98%, or 99%.

In some embodiments, a provided oligonucleotide is a unimer. In some embodiments, a provided oligonucleotide is a P-modification unimer. In some embodiments, a provided oligonucleotide is a stereounimer. In some embodiments, a provided oligonucleotide is a stereounimer of configuration Rp. In some embodiments, a provided oligonucleotide is a stereounimer of configuration Sp.

In some embodiments, a provided oligonucleotide is an altmer. In some embodiments, a provided oligonucleotide is a P-modification altmer. In some embodiments, a provided oligonucleotide is a stereoaltmer.

In some embodiments, a provided oligonucleotide is a blockmer. In some embodiments, a provided oligonucleotide is a P-modification blockmer. In some embodiments, a provided oligonucleotide is a stereoblockmer.

In some embodiments, a provided oligonucleotide is a gapmer.

In some embodiments, a provided oligonucleotide is a skipmer.

In some embodiments, a provided oligonucleotide is a hemimer. In some embodiments, a hemimer is an oligonucleotide wherein the 5′-end or the 3′-end region has a sequence that possesses a structure feature that the rest of the oligonucleotide does not have. In some embodiments, the 5′-end or the 3′-end region has or comprises 2 to 20 nucleotides. In some embodiments, a structural feature is a base modification. In some embodiments, a structural feature is a sugar modification. In some embodiments, a structural feature is a P-modification. In some embodiments, a structural feature is stereochemistry of the chiral internucleotidic linkage. In some embodiments, a structural feature is or comprises a base modification, a sugar modification, a P-modification, or stereochemistry of the chiral internucleotidic linkage, or combinations thereof. In some embodiments, a hemimer is an oligonucleotide in which each sugar moiety of the 5′-end region shares a common modification. In some embodiments, a hemimer is an oligonucleotide in which each sugar moiety of the 3′-end region shares a common modification. In some embodiments, a common sugar modification of the 5′ or 3′-end region is not shared by any other sugar moieties in the oligonucleotide. In some embodiments, an example hemimer is an oligonucleotide comprising a sequence of substituted or unsubstituted 2′-O-alkyl sugar modified nucleosides, bicyclic sugar modified nucleosides, β-D-ribonucleosides or β-D-deoxyribonucleosides (for example 2′-MOE modified nucleosides, and LNA™ or ENA™ bicyclic sugar modified nucleosides) at one terminus region and a sequence of nucleosides with a different sugar moiety (such as a substituted or unsubstituted 2′-O-alkyl sugar modified nucleosides, bicyclic sugar modified nucleosides or natural ones) at the other terminus region. In some embodiments, a provided oligonucleotide is a combination of one or more of unimer, altmer, blockmer, gapmer, hemimer and skipmer. In some embodiments, a provided oligonucleotide is a combination of one or more of unimer, altmer, blockmer, gapmer, and skipmer. For instance, in some embodiments, a provided oligonucleotide is both an altmer and a gapmer. In some embodiments, a provided nucleotide is both a gapmer and a skipmer. One of skill in the chemical and synthetic arts will recognize that numerous other combinations of patterns are available and are limited only by the commercial availability and/or synthetic accessibility of constituent parts required to synthesize a provided oligonucleotide in accordance with methods of the present disclosure. In some embodiments, a hemimer structure provides advantageous benefits. In some embodiments, provided oligonucleotides are 5′-hemimers that comprises modified sugar moieties in a 5′-end sequence. In some embodiments, provided oligonucleotides are 5′-hemimers that comprises modified 2′-sugar moieties in a 5′-end sequence.

In some embodiments, a provided oligonucleotide comprises one or more optionally substituted nucleotides. In some embodiments, a provided oligonucleotide comprises one or more modified nucleotides. In some embodiments, a provided oligonucleotide comprises one or more optionally substituted nucleosides. In some embodiments, a provided oligonucleotide comprises one or more modified nucleosides. In some embodiments, a provided oligonucleotide comprises one or more optionally substituted LNAs.

In some embodiments, a provided oligonucleotide comprises one or more optionally substituted nucleobases. In some embodiments, a provided oligonucleotide comprises one or more optionally substituted natural nucleobases. In some embodiments, a provided oligonucleotide comprises one or more optionally substituted modified nucleobases. In some embodiments, a provided oligonucleotide comprises one or more 5-methylcytidine; 5-hydroxymethylcytidine, 5-formylcytosine, or 5-carboxylcytosine. In some embodiments, a provided oligonucleotide comprises one or more 5-methylcytidine.

In some embodiments, each nucleobase of a provided oligonucleotide, e.g., one of formula O-I, A^{c}-[-L^{M}-(R^{D})ₐ]_{b}, [(A^{c})ₐ-L^{M}]_{b}-R^{D}, (A^{c})ₐ-L^{M}-(A^{c})_{b}, or (A^{c})ₐ-L^{M}-(R^{D})_{b}, or 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{mz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′, is independently an optionally substituted or protected nucleobase of adenine, cytosine, guanosine, thymine, or uracil.

In some embodiments, each base (BA) is independently an optionally substituted or protected nucleobase of adenine, cytosine, guanosine, thymine, or uracil. As appreciated by those skilled in the art, various protected nucleobases, including those widely known in the art, for example, those used in oligonucleotide preparation (e.g., protected nucleobases of WO/2010/064146, WO/2011/005761, WO/2013/012758, WO/2014/010250, US2013/0178612, WO/2014/012081, WO/2015/107425, WO2017/015555, and WO2017/062862, protected nucleobases of each of which are incorporated herein by reference), and can be utilized in accordance with the present disclosure.

In some embodiments, a provided oligonucleotide comprises one or more optionally substituted sugars. In some embodiments, a provided oligonucleotide comprises one or more optionally substituted sugars found in naturally occurring DNA and RNA. In some embodiments, a provided oligonucleotide comprises one or more optionally substituted ribose or deoxyribose. In some embodiments, a provided oligonucleotide comprises one or more optionally substituted ribose or deoxyribose, wherein one or more hydroxyl groups of the ribose or deoxyribose moiety is optionally and independently replaced by halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen, R′, —N(R′)₂, —OR′, or —SR′, wherein each R′ is independently as defined above and described herein. In some embodiments, a provided oligonucleotide comprises one or more optionally substituted deoxyribose, wherein the 2′ position of the deoxyribose is optionally and independently substituted with halogen. In some embodiments, a provided oligonucleotide comprises one or more optionally substituted deoxyribose, wherein the 2′ position of the deoxyribose is optionally and independently substituted with one or more —F. halogen. In some embodiments, a provided oligonucleotide comprises one or more optionally substituted deoxyribose, wherein the 2′ position of the deoxyribose is optionally and independently substituted with —OR′, wherein each R′ is independently as defined above and described herein. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ aliphatic. In some embodiments, a provided oligonucleotide comprises one or more optionally substituted deoxyribose, wherein the 2′ position of the deoxyribose is optionally and independently substituted with —OR′, wherein each R′ is independently an optionally substituted C₁-C₆ alkyl. In some embodiments, a provided oligonucleotide comprises one or more optionally substituted deoxyribose, wherein the 2′ position of the deoxyribose is optionally and independently substituted with —OMe. In some embodiments, a provided oligonucleotide comprises one or more optionally substituted deoxyribose, wherein the 2′ position of the deoxyribose is optionally and independently substituted with —O-methoxyethyl.

In some embodiments, a provided oligonucleotide is single-stranded oligonucleotide.

In some embodiments, a provided oligonucleotide is a hybridized oligonucleotide strand. In certain embodiments, a provided oligonucleotide is a partially hybridized oligonucleotide strand. In certain embodiments, a provided oligonucleotide is a completely hybridized oligonucleotide strand. In certain embodiments, a provided oligonucleotide is a double-stranded oligonucleotide. In certain embodiments, a provided oligonucleotide is a triple-stranded oligonucleotide (e.g., a triplex).

In some embodiments, a provided oligonucleotide is chimeric. For example, in some embodiments, a provided oligonucleotide is DNA-RNA chimera, DNA-LNA chimera, etc.

In some embodiments, any one of the structures comprising an oligonucleotide depicted in WO2012/030683 can be modified in accordance with methods of the present disclosure to provide chirally controlled compositions thereof. For example, in some embodiments, chirally controlled composition comprises a stereochemical control at any one or more of chiral linkage phosphorus atoms, optionally through incorporation of one or more P-modifications described in WO2012/030683 or the present disclosure. For example, in some embodiments, a particular nucleotide unit of an oligonucleotide of WO2012/030683 is preselected to be provided with chiral control at the linkage phosphorus of that nucleotide unit and/or to be P-modified with chiral control at the linkage phosphorus of that nucleotide unit.

In some embodiments, a provided oligonucleotide comprises a nucleic acid analog, e.g., GNA, LNA, PNA, TNA, F-HNA (F-THP or 3′-fluoro tetrahydropyran), MNA (mannitol nucleic acid, e.g., Leumann 2002 Bioorg. Med. Chem. 10: 841-854), ANA (anitol nucleic acid), and Morpholino.

In some embodiments, a provided oligonucleotide is characterized as having the ability to indirectly or directly increase or decrease activity of a protein or inhibition or promotion of the expression of a protein. In some embodiments, a provided oligonucleotide is characterized in that it is useful in the control of cell proliferation, viral replication, and/or any other cell signaling process.

In some embodiments, a provided oligonucleotide is about 2-500 nucleotide units in length. In some embodiments, a provided oligonucleotide is about 5-500 nucleotide units in length. In some embodiments, a provided oligonucleotide is about 10-50 nucleotide units in length. In some embodiments, a provided oligonucleotide is about 15-50 nucleotide units in length. In some embodiments, each nucleotide unit independently comprises a heteroaryl nucleobase unit (e.g., adenine, cytosine, guanosine, thymine, or uracil, each of which is optionally and independently substituted or protected), a sugar unit comprising a 5-10 membered heterocyclyl ring, and an internucleotidic linkage having the structure of formula I. In some embodiments, a provided oligonucleotide is from about 2 to about 200 nucleotide units in length. In some embodiments, a provided oligonucleotide is from about 2 to about 180 nucleotide units in length. In some embodiments, a provided oligonucleotide is from about 2 to about 160 nucleotide units in length. In some embodiments, a provided oligonucleotide is from about 2 to about 140 nucleotide units in length. In some embodiments, a provided oligonucleotide is from about 2 to about 120 nucleotide units in length. In some embodiments, a provided oligonucleotide is from about 2 to about 100 nucleotide units in length. In some embodiments, a provided oligonucleotide is from about 2 to about 90 nucleotide units in length. In some embodiments, a provided oligonucleotide is from about 2 to about 80 nucleotide units in length. In some embodiments, a provided oligonucleotide is from about 2 to about 70 nucleotide units in length. In some embodiments, a provided oligonucleotide is from about 2 to about 60 nucleotide units in length. In some embodiments, a provided oligonucleotide is from about 2 to about 50 nucleotide units in length. In some embodiments, a provided oligonucleotide is from about 2 to about 40 nucleotide units in length. In some embodiments, a provided oligonucleotide is from about 2 to about 30 nucleotide units in length. In some embodiments, a provided oligonucleotide is from about 2 to about 29 nucleotide units in length. In some embodiments, a provided oligonucleotide is from about 2 to about 28 nucleotide units in length. In some embodiments, a provided oligonucleotide is from about 2 to about 27 nucleotide units in length. In some embodiments, a provided oligonucleotide is from about 2 to about 26 nucleotide units in length. In some embodiments, a provided oligonucleotide is from about 2 to about 25 nucleotide units in length. In some embodiments, a provided oligonucleotide is from about 2 to about 24 nucleotide units in length. In some embodiments, a provided oligonucleotide is from about 2 to about 23 nucleotide units in length. In some embodiments, a provided oligonucleotide is from about 2 to about 22 nucleotide units in length. In some embodiments, a provided oligonucleotide is from about 2 to about 21 nucleotide units in length. In some embodiments, a provided oligonucleotide is from about 2 to about 20 nucleotide units in length.

In some embodiments, a provided oligonucleotide is from about 4 to about 200 nucleotide units in length. In some embodiments, a provided oligonucleotide is from about 4 to about 180 nucleotide units in length. In some embodiments, a provided oligonucleotide is from about 4 to about 160 nucleotide units in length. In some embodiments, a provided oligonucleotide is from about 4 to about 140 nucleotide units in length. In some embodiments, a provided oligonucleotide is from about 4 to about 120 nucleotide units in length. In some embodiments, a provided oligonucleotide is from about 4 to about 100 nucleotide units in length. In some embodiments, a provided oligonucleotide is from about 4 to about 90 nucleotide units in length. In some embodiments, a provided oligonucleotide is from about 4 to about 80 nucleotide units in length. In some embodiments, a provided oligonucleotide is from about 4 to about 70 nucleotide units in length. In some embodiments, a provided oligonucleotide is from about 4 to about 60 nucleotide units in length. In some embodiments, a provided oligonucleotide is from about 4 to about 50 nucleotide units in length. In some embodiments, a provided oligonucleotide is from about 4 to about 40 nucleotide units in length. In some embodiments, a provided oligonucleotide is from about 4 to about 30 nucleotide units in length. In some embodiments, a provided oligonucleotide is from about 4 to about 29 nucleotide units in length. In some embodiments, a provided oligonucleotide is from about 4 to about 28 nucleotide units in length. In some embodiments, a provided oligonucleotide is from about 4 to about 27 nucleotide units in length. In some embodiments, a provided oligonucleotide is from about 4 to about 26 nucleotide units in length. In some embodiments, a provided oligonucleotide is from about 4 to about 25 nucleotide units in length. In some embodiments, a provided oligonucleotide is from about 4 to about 24 nucleotide units in length. In some embodiments, a provided oligonucleotide is from about 4 to about 23 nucleotide units in length. In some embodiments, a provided oligonucleotide is from about 4 to about 22 nucleotide units in length. In some embodiments, a provided oligonucleotide is from about 4 to about 21 nucleotide units in length. In some embodiments, a provided oligonucleotide is from about 4 to about 20 nucleotide units in length.

In some embodiments, a provided oligonucleotide is from about 5 to about 10 nucleotide units in length. In some embodiments, a provided oligonucleotide is from about 10 to about 30 nucleotide units in length. In some embodiments, a provided oligonucleotide is from about 15 to about 25 nucleotide units in length. In some embodiments, a provided oligonucleotide is from about 5, 6, 7, 8, 9, 10, 11, 12, 13, 14, 15, 16, 17, 18, 19, 20, 21, 22, 23, 24, or 25 nucleotide units in length.

In some embodiments, an oligonucleotide is at least 2 nucleotide units in length. In some embodiments, an oligonucleotide is at least 2, 3, 4, 5, 6, 7, 8, 9, 10, 11, 12, 13, 14, 15, 16, 17, 18, 19, 20, 21, 22, 23, 24, or 25 nucleotide units in length. In some embodiments, an oligonucleotide is at least 5 nucleotide units in length. In some embodiments, an oligonucleotide is at least 10 nucleotide units in length. In some embodiments, an oligonucleotide is at least 15 nucleotide units in length. In some embodiments, an oligonucleotide is at least 16 nucleotide units in length. In some embodiments, an oligonucleotide is at least 17 nucleotide units in length. In some embodiments, an oligonucleotide is at least 18 nucleotide units in length. In some embodiments, an oligonucleotide is at least 19 nucleotide units in length. In some embodiments, an oligonucleotide is at least 20 nucleotide units in length. In some embodiments, an oligonucleotide is at least 21 nucleotide units in length. In some embodiments, an oligonucleotide is at least 22 nucleotide units in length. In some embodiments, an oligonucleotide is at least 23 nucleotide units in length. In some embodiments, an oligonucleotide is at least 24 nucleotide units in length. In some embodiments, an oligonucleotide is at least 25 nucleotide units in length. In some other embodiments, an oligonucleotide is at least 30 nucleotide units in length. In some other embodiments, an oligonucleotide is a duplex of complementary strands of at least 18 nucleotide units in length. In some other embodiments, an oligonucleotide is a duplex of complementary strands of at least 21 nucleotide units in length.

In some embodiments, the 5′-end and/or the 3′-end of a provided oligonucleotide is modified. In some embodiments, the 5′-end and/or the 3′-end of a provided oligonucleotide is modified with a terminal cap moiety. Examples of such modifications, including terminal cap moieties are extensively described herein and in the art, for example but not limited to those described in US Patent Application Publication US 2009/0023675A1.

In some embodiments, oligonucleotides of an oligonucleotide type characterized by 1) a common base sequence and length, 2) a common pattern of backbone linkages, and 3) a common pattern of backbone chiral centers, have the same chemical structure. For example, they have the same base sequence, the same pattern of nucleoside modifications, the same pattern of backbone linkages (i.e., pattern of internucleotidic linkage types, for example, phosphate, phosphorothioate, etc), the same pattern of backbone chiral centers (i.e. pattern of linkage phosphorus stereochemistry (Rp/Sp)), and the same pattern of backbone phosphorus modifications (e.g., pattern of “—XLR¹” groups in Formula I).

Single-Stranded RNAi Agents and Antisense Oligonucleotides

In some embodiments, the present disclosure provides oligonucleotides. In some embodiments, the present disclosure provides oligonucleotides which decrease the expression and/or level of a target gene or its gene product. Those of ordinary skill in the art, reading the present disclosure, will appreciate that, in some embodiments, provided oligonucleotides may act as RNAi agents. Alternatively or additionally, in some embodiments, provided oligonucleotides may act via an RNase H-dependent mechanism and/or another biochemical mechanism that does not involve RNA interference.

Among other things, the present disclosure defines certain structural attributes that may be particularly desirable and/or effective in an oligonucleotide. Among other things, the present disclosure defines certain structural attributes that may be particularly desirable and/or effective in an oligonucleotide that acts as an RNAi agent. In some embodiments, the present disclosure defines certain structural attributes that may be particularly desirable and/or effective in an oligonucleotide that acts via an RNase H-dependent mechanism and/or other biochemical mechanism. In some embodiments, the present disclosure defines certain structural attributes that may be particularly desirable and/or effective in a single-stranded ssRNAi agent (ssRNAi or ssRNAi agent); in some such embodiments, as described further herein below, such structural attributes may be distinct from those that are particularly desirable and/or effective in a corresponding strand of a double-stranded RNAi agent (dsRNAi or dsRNAi agent). In some embodiments, provided oligonucleotides are single-stranded RNAi agents (e.g., which can be loaded into RISC and/or can direct or enhance RISC-mediated target). In some embodiments, provided oligonucleotides are antisense oligonucleotides (e.g., which can be loaded into RNase H and/or direct or enhance RNase-H-mediated cleavage of a target and/or operate via a different biochemical mechanism).

In some embodiments (including in some single-stranded oligonucleotide embodiments), oligonucleotides that act as RNAi agents may have one or more different structural attributes and/or functional properties from those oligonucleotides that act via an RNase H-dependent mechanism. In some embodiments, an oligonucleotide can direct a decrease in the expression and/or level of a target gene or its gene product by sterically blocking translation after binding to a target gene mRNA, and/or by altering or interfering with mRNA splicing and/or exon inclusion or exclusion (e.g., skipping). In some embodiments, an oligonucleotide can perform a function, or a significant percentage of a function (for example, 10-100%, no less than 10%, 20%, 30%, 40%, 50%, 60%, 70%, 80%, 90% percent or more) independent of RNA interference or RISC.

In some embodiments, a provided oligonucleotide is an antisense oligonucleotide (ASO) which directs cleavage of a target RNA mediated by RNase H and not RISC (RNA interference silencing complex).

In some embodiments, a provided oligonucleotide is a single-stranded RNAi (ssRNAi) agent which directs cleavage of a target mRNA mediated by the RISC (RNA interference silencing complex) and not the enzyme RNase H. In some embodiments, an oligonucleotide can perform a function, or a significant percentage of a function (for example, 10-100%, no less than 10%, 20%, 30%, 40%, 50%, 60%, 70%, 80%, 90% percent or more) independent of RNase H.

A double-stranded RNAi agent can also direct cleavage of a target mRNA using RISC and not the enzyme RNase H. In some embodiments, a single-stranded RNAi agent differs from a double-stranded RNAi agent in that a ssRNAi agent includes only a single oligonucleotide strand and generally does not comprise a double-stranded region of significant length, and a dsRNAi agent comprises a double stranded region of significant length (e.g., at least about 15 bp, or about 19 bp in a “canonical” siRNA). In some embodiments, a dsRNAi comprises two separate, complementary strands (which are not covalently linked) which form a double-stranded region (e.g., in a “canonical” siRNA), or a long single strand which comprises two complementary sequences which together form a double-stranded region (e.g., in a shRNA or short hairpin RNA). In some embodiments of a dsRNAi, the passenger strand has a single-stranded nick, forming two strands. In some embodiments, the present disclosure demonstrates that sequences and/or structural elements (chemical modifications, stereochemistry, etc.) required for efficacious single-stranded RNAi agents may differ from those required for efficacious double-stranded RNAi agents.

Among other things, the present disclosure encompasses the recognition that certain designs (e.g., sequences and/or structural elements) which may be suitable for double-stranded RNAi agents may not be suitable for single-stranded RNAi agents (including single-stranded RNAi agents of provided formats described herein), and vice versa. In some embodiments, the present disclosure provides designs for effective ssRNAi. In some embodiments, the present disclosure demonstrates that certain base sequences, when combined with structural elements (modifications, stereochemistry, additional chemical moiety or moieties, etc.) in accordance with the present disclosure, can provided oligonucleotides having unexpectedly high activities, for example, when administered as ssRNAi agents, particularly in comparison with oligonucleotides comprising the same sequences but double-stranded and administered as dsRNAi agents. In some embodiments, the present disclosure demonstrates that certain base sequences, when combined with structural elements (modifications, stereochemistry, additional chemical moiety or moieties, etc.) in accordance with the present disclosure, can provided oligonucleotides having unexpectedly high activities, for example, the ability to decrease the expression and/or level of a target gene or its gene product.

Structural and Functional Differences Between Single-Stranded RNAi (ssRNAi) Agents, Double-Stranded RNAi (dsRNAi) Agents, and RNase H-Dependent Antisense Oligonucleotides (ASOs)

In some embodiments, single-stranded RNAi (ssRNAi) agents, double-stranded RNAi (dsRNAi) agents and RNase H-dependent antisense oligonucleotides (ASOs) all involve binding of an agent or oligonucleotide (or portion thereof) to a complementary (or substantially complementary) target RNA (e.g., a mRNA or pre-mRNA), followed by cleavage of the target RNA and/or a decrease the expression and/or level of a target gene or its gene product. In some embodiments, RNAi agents, whether double- or single-stranded, employ the RISC, or RNA interference silencing complex, which includes the enzyme Ago-2 (Argonaute-2). In some embodiments, RNase H-dependent antisense oligonucleotides are single-stranded and employ a different enzyme, RNase H. RNAse H is reportedly a cellular endonuclease which cleaves the RNA strand of an RNA:DNA duplex; see U.S. Pat. No. 7,919,472. See also, Saetrom (2004 Bioinformatics 20: 3055-3063); Kretschmer-Kazemi Far et al. (2003 Nucleic Acids 31: 4417-4424); Bertrand et al. (2002) Biochem. Biophys. Res. Comm. 296: 1000-1004); Vickers et al. (2003 J. Biol. Chem. 278: 7108). In some embodiments, oligonucleotides that can direct RNase H-mediated knockdown include, but are not limited to, those consisting of or comprising a region of consecutive 2′-deoxy nucleotide units which contain no 2′-modifications. In some embodiments, oligonucleotides that can direct RNase H-mediated knockdown are gap-widened oligonucleotides or gapmers. In some embodiments, a gapmer comprises an internal region comprises a plurality of nucleotides that supports RNase H cleavage and is positioned between external regions having a plurality of nucleotides that are chemically distinct from the nucleosides of the internal region. In some embodiments, a gapmer comprise a span of 2′-deoxy nucleotides containing no 2′-modifications, flanked or adjacent to one or two wings. In some embodiments, a gap directs RNase H cleavage of the corresponding RNA target. In some embodiments, the wings do not direct or act as substrates for RNase H cleavage. The wings can be of varying lengths (including, but not limited to, 1 to 8 nt) and can comprise various modifications or analogs (including, but not limited to, 2′-modifications, including, but not limited to, 2′-OMe and 2′-MOE). See, as non-limiting examples, U.S. Pat. Nos. 9,550,988; 7,919,472; 5,013,830; 5,149,797; 5,220,007; 5,256,775; 5,366,878; 5,403,711; 5,491,133; 5,565,350; 5,623,065; 5,652,355; 5,652,356; and 5,700,922. In some embodiments, presence of one or more such modifications or analogs may correlate with modified (e.g., increased, reduced, or altered) RNase H cleavage of a target.

In some embodiments, double-stranded RNAi agents, even the antisense strand thereof, differ structurally from a RNase H-dependent antisense oligonucleotide. In some embodiments, RNase H-dependent antisense oligonucleotides and siRNA oligonucleotides seem to have completely opposite characteristics, both regarding 5′-end structures and overall duplex stability.

Double-stranded RNAi agents can reportedly be naturally-produced in a cell by the Dicer enzyme, which cleaves larger RNA molecules, such as double-stranded RNA from invading viruses, into a dsRNA. The canonical structure of a dsRNA agent comprises two strands of RNA, each about 19 to 23 nt long, which are annealed to form an about 19-21 bp double-stranded region and two 3′ dinucleotide overhangs. For a double-stranded RNAi agent, the sense strand is reportedly unwound from the duplex before the antisense strand is incorporated into RISC. Aside from the natural separation of a double-stranded RNAi agent into antisense and sense strands, single-stranded RNAi agents have not been reported to be naturally produced in a human cell.

Among other things, the present disclosure provides the teaching that, in many cases, a single-stranded RNAi agent is not simply an isolated antisense strand of a double-stranded RNAi agent in that, for example, an antisense strand of an effective dsRNAi agent may be much less effective than the dsRNAi agent, and a ssRNAi agent, when formulated as a dsRNAi agent (for example, by annealing with a sense strand), may be much less effective than the ssRNAi agent. In some embodiments, double-stranded and single-stranded RNAi agents differ in many significant ways. Structural parameters of double-stranded RNAi agents are not necessarily reflected in single-stranded RNAi agents.

In some embodiments, the present disclosure teaches that target sequences which are suitable for double-stranded RNAi agents may not be suitable for single-stranded RNAi agents, and vice versa. For example, in at least some cases, single-stranded versions of double-stranded RNAi agents may not be efficacious. As a non-limiting example, Table 46A shows that several ssRNAi agents were constructed with sequences derived from dsRNAi. These ssRNAi based on dsRNAi were generally less efficacious than the corresponding dsRNAi.

In some embodiments, double-stranded and single-stranded RNAi agents also differ in their sensitivity to incorporation of chirally controlled internucleotidic linkages. For example, Matranga et al. (2005 Cell 123: 607-620) reported that introduction of a single Sp internucleotidic linkage (e.g., a single Sp PS) into the sense strand of a double-stranded RNAi agent greatly decreased RISC assembly and RNA interference activity. In contrast, in some embodiments, data shown herein demonstrate that, surprisingly, incorporation of a Sp internucleotidic linkage)(e.g., Sp PS) can perform two functions for a single-stranded RNAi agent: (a) it increases stability against nucleases; and (b) does not interfere with RNA interference activity. Many example oligonucleotides can perform as efficacious single-stranded RNAi agents comprising one or more chirally controlled internucleotidic linkages (e.g., Sp internucleotidic linkages, or Sp PS (phosphorothioate) are shown herein).

Alternatively or additionally, in some embodiments, double-stranded and single-stranded RNAi agents differ in their maximum GC content. In some embodiments, a double-stranded RNAi agent is more limited, for example, in its GC content, as too high a GC content interferes with duplex unwinding, as reported in, for example, U.S. Pat. No. 7,507,811 to Khvorova et al., which reports that a double-stranded RNAi agent can have a GC content up to 52%. In contrast, in some embodiments, data shown herein shows that efficacious single-stranded RNAi agents can have a GC content of up to 70%. Among other things, some target sequences with a GC content of up to 70% may be explorable for use as ssRNAi while not suitable for dsRNAi.

Alternatively or additionally, double-stranded and single-stranded RNAi agents can differ in the maximum length of a GC span. For example, Naito et al. 2004 Nucl. Acids Res. 32: W124-W129 reported a rule indicating that highly effective double-stranded RNAi agents should lack a GC stretch over 9 bp in length. In some embodiments, the present disclosure shows examples of efficacious single-stranded RNAi agents comprising GC stretches of up to 11 in length.

Alternatively or additionally, double-stranded and single-stranded RNAi agents can differ in immunogenicity. In some embodiments, some single-stranded RNAi agents are reportedly more immunogenic than double-stranded RNAi agents. Sioud J. Mol. Biol. (2005) 348, 1079-1090. In some embodiments, several double-stranded RNAi agents reportedly did not induce an immune response, whereas corresponding single-stranded RNAi agents did. In some embodiments, the present disclosure provides oligonucleotides with low immunogenicity. In some embodiments, such oligonucleotides can be utilized as ssRNAi reagent.

Among other things, the present disclosure encompasses the recognition that certain conventional designs of single-stranded RNAi agents, which derive single-stranded RNAi agents, including base sequences, from double-stranded RNAi agents, often fail to provide effective single-stranded RNAi agents. In some embodiments, the present disclosure demonstrates that, surprisingly, ssRNAi agents derived from base sequences of effective RNase H-dependent ASOs can produce efficacious ssRNAi agents (see Table 46A).

In some embodiments, the present disclosure provides oligonucleotides which can be utilized as efficacious RNase-H dependent ASOs, which comprise regions of 2′-deoxy nucleotides without 2′-modifications, and which are complementary or substantially complementary to RNA sequences or portions thereof. In some embodiments, a region can be, for example, a core sequence of about 10 nt flanked on one or both sides by wings, wherein the wings differ from the core in chemistry and can comprise, as non-limiting examples, 2′-modifications or internucleotidic linkage modifications.

Oligonucleotides

In some embodiments, provided oligonucleotides can direct a decrease in the expression and/or level of a target gene or its gene product. In some embodiments, provided oligonucleotides can direct a decrease in the expression and/or level of a target gene or its gene product via RNA interference. In some embodiments, provided oligonucleotides can direct a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides can direct a decrease in the expression and/or level of a target gene or its gene product via RNA interference and/or RNase H-mediated knockdown. In some embodiments, provided oligonucleotides can direct a decrease in the expression and/or level of a target gene or its gene product by sterically blocking translation after binding to a target gene mRNA, and/or by altering or interfering with mRNA splicing and/or exon inclusion or exclusion.

In some embodiments, a provided oligonucleotide has a structural element or format or portion thereof described herein.

In some embodiments, a provided oligonucleotide capable of directing a decrease in the expression and/or level of a target gene or its gene product has a structural element or format or portion thereof described herein.

In some embodiments, a provided oligonucleotide capable of directing a decrease in the expression and/or level of a target gene or its gene product has the format of any oligonucleotide disclosed herein, e.g., in Table 1A, or in the Figures or Tables, or otherwise disclosed herein.

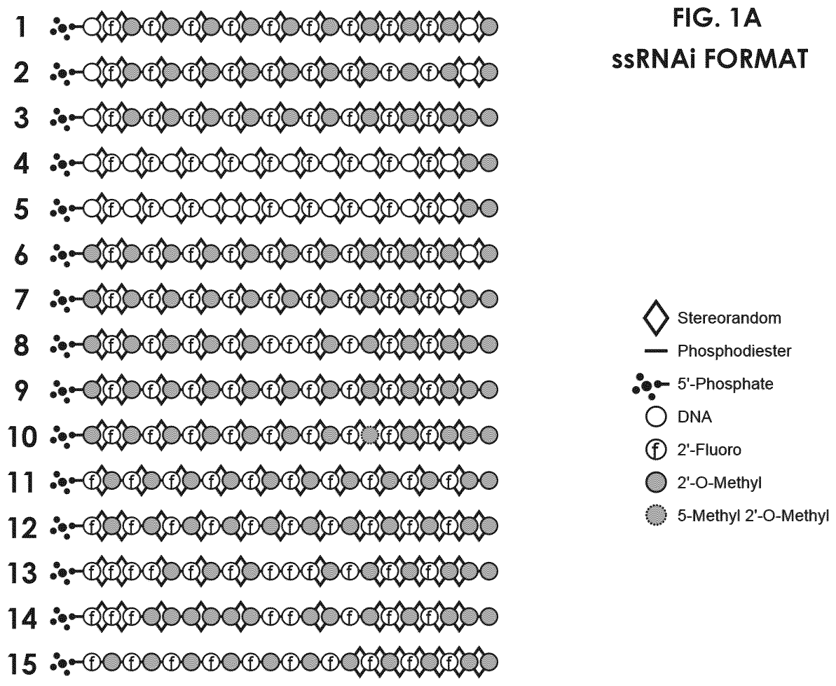

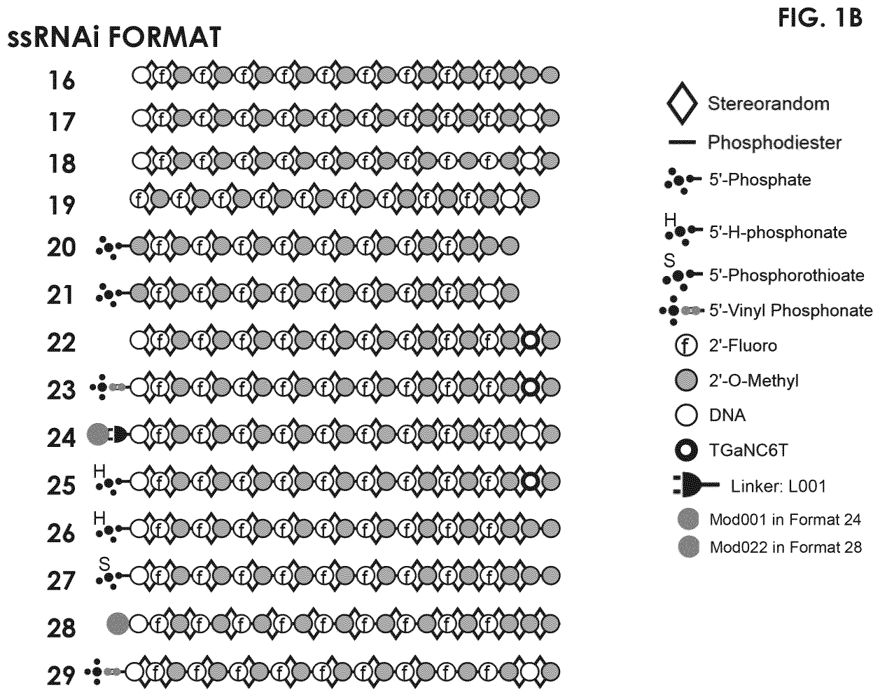

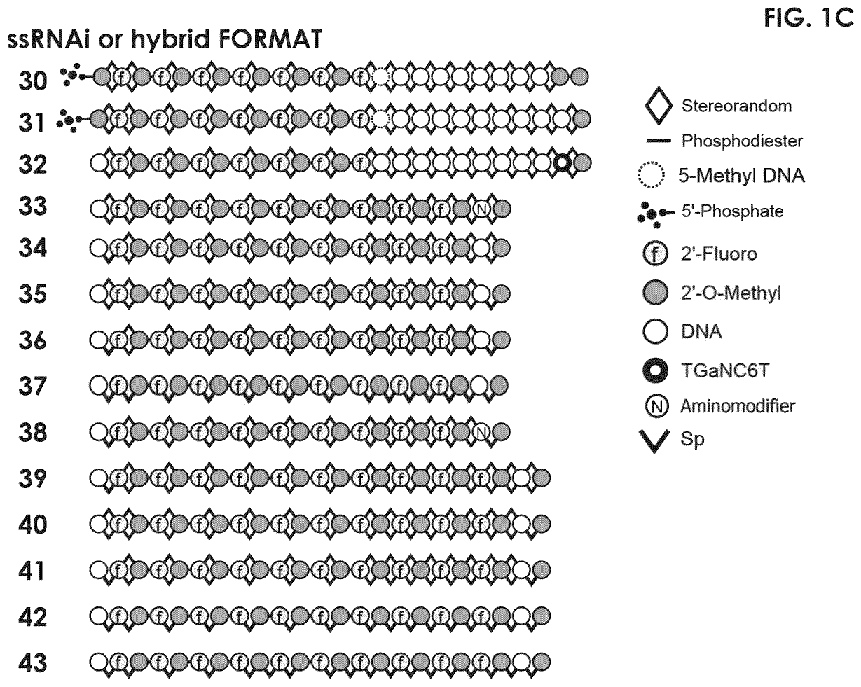

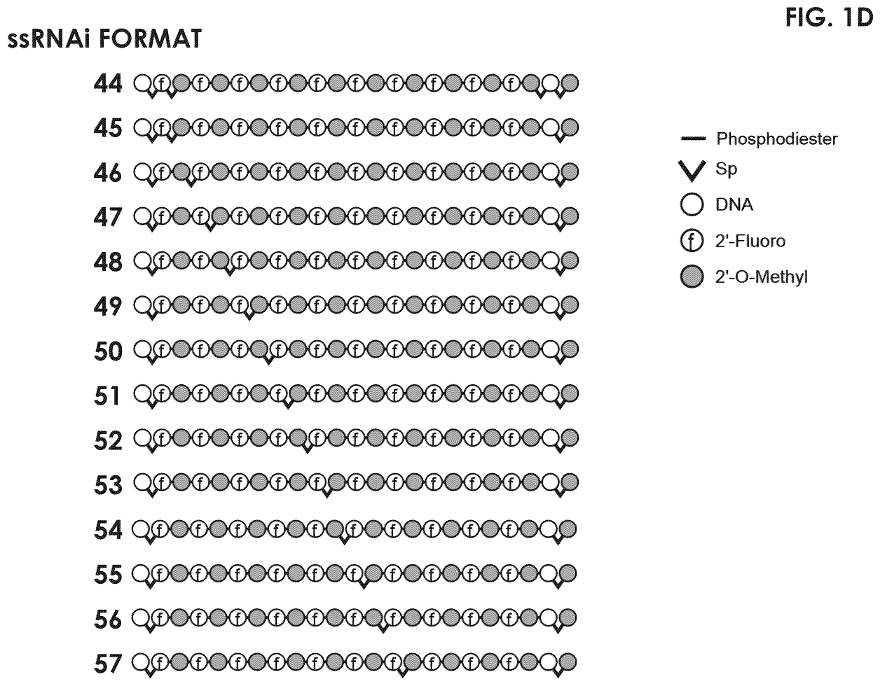

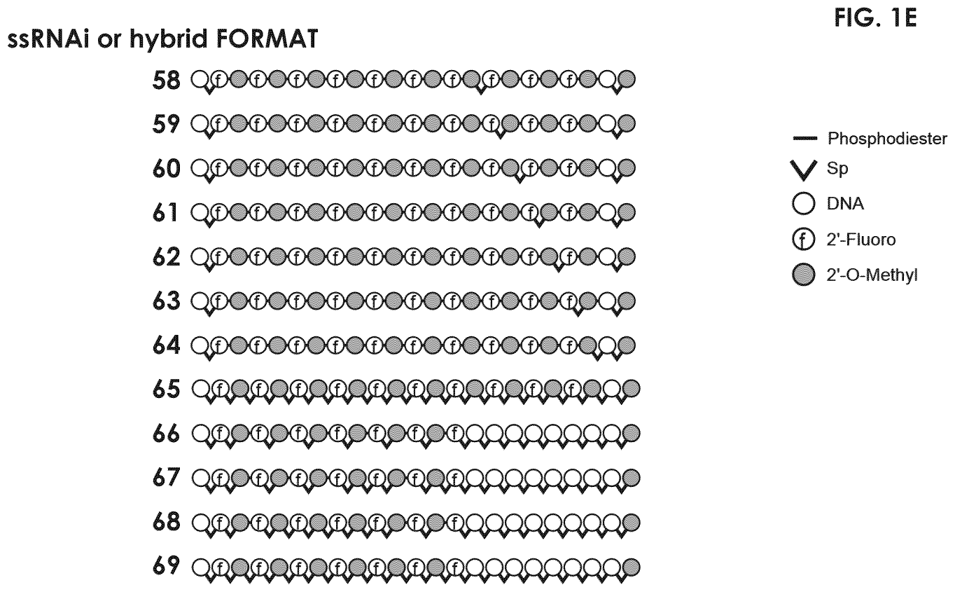

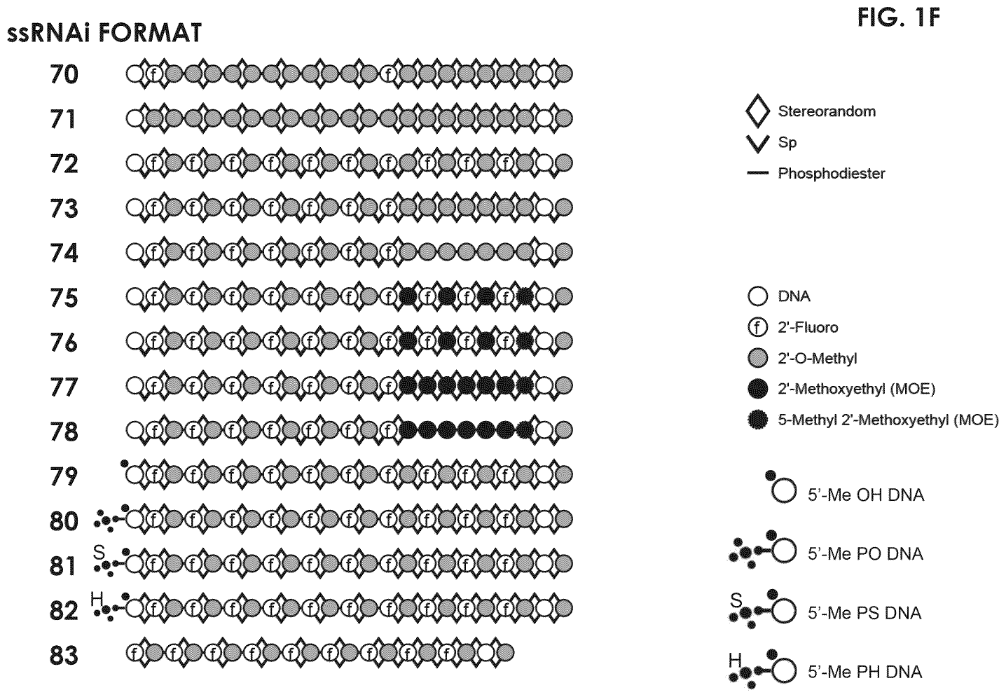

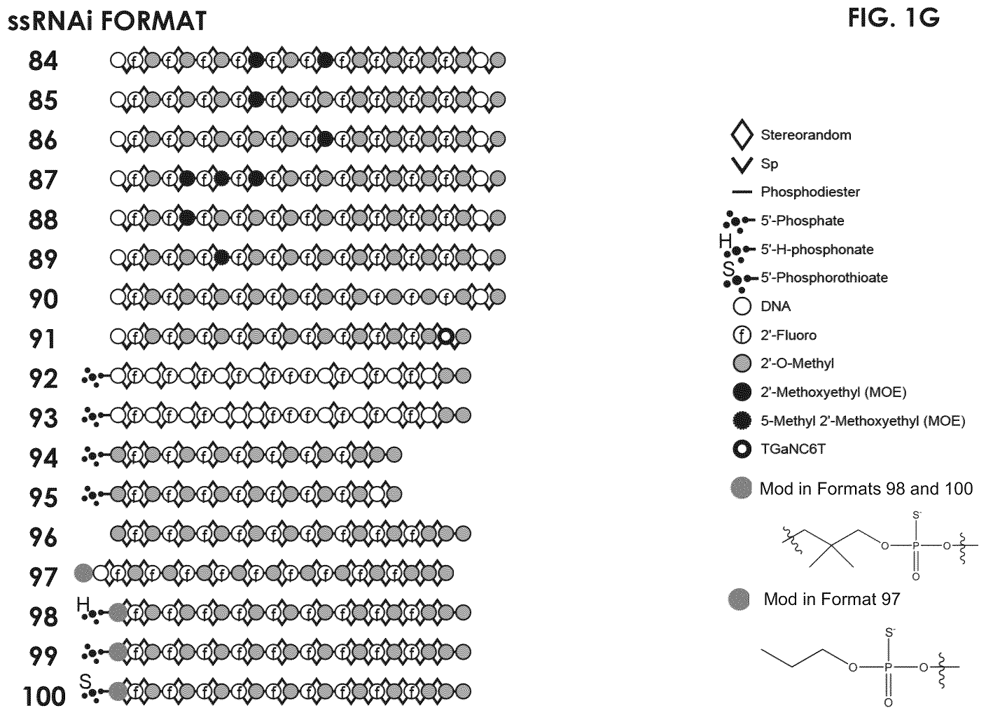

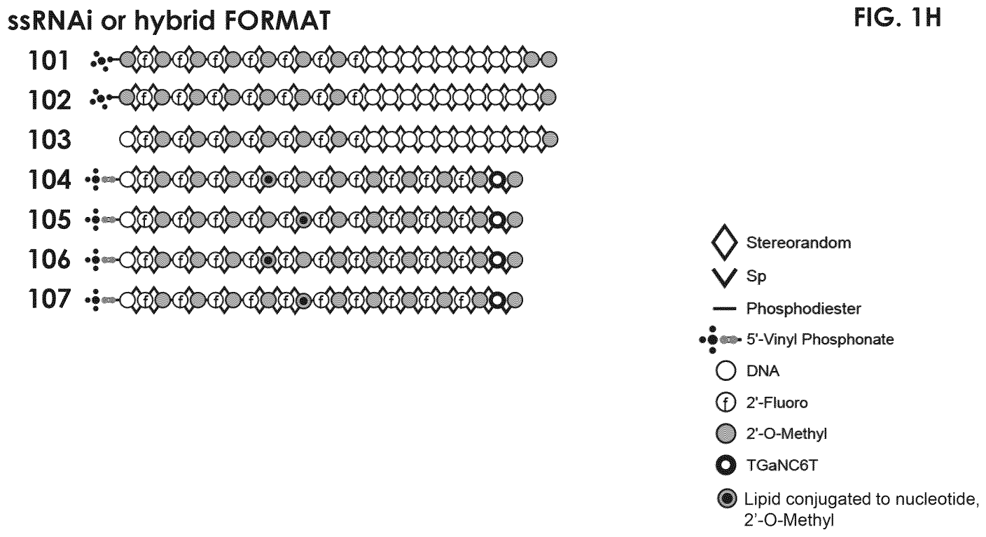

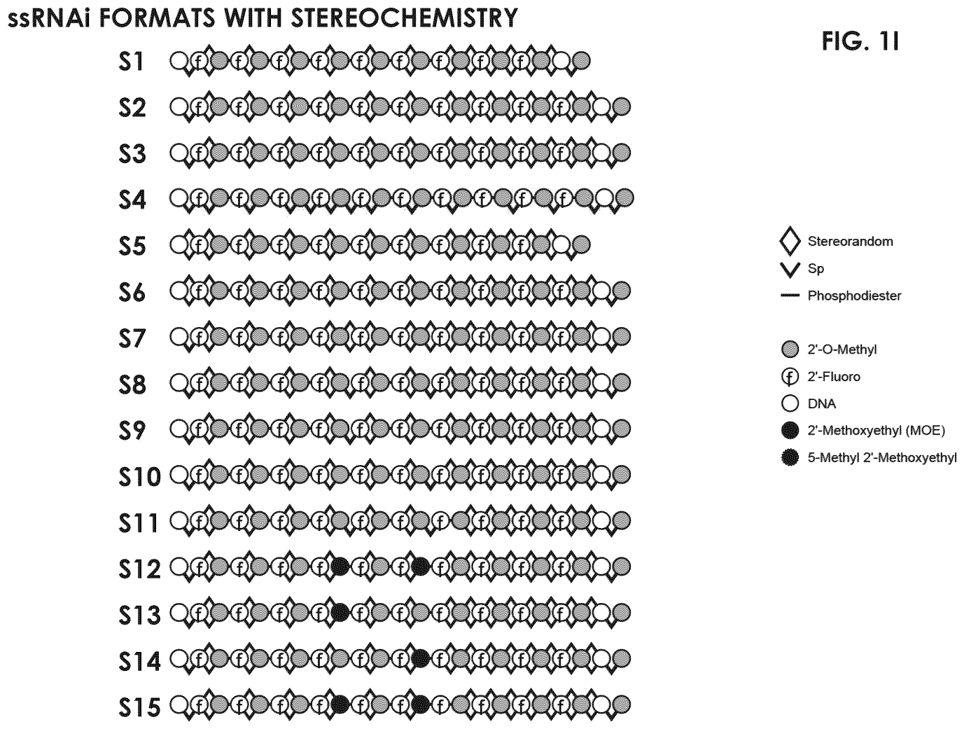

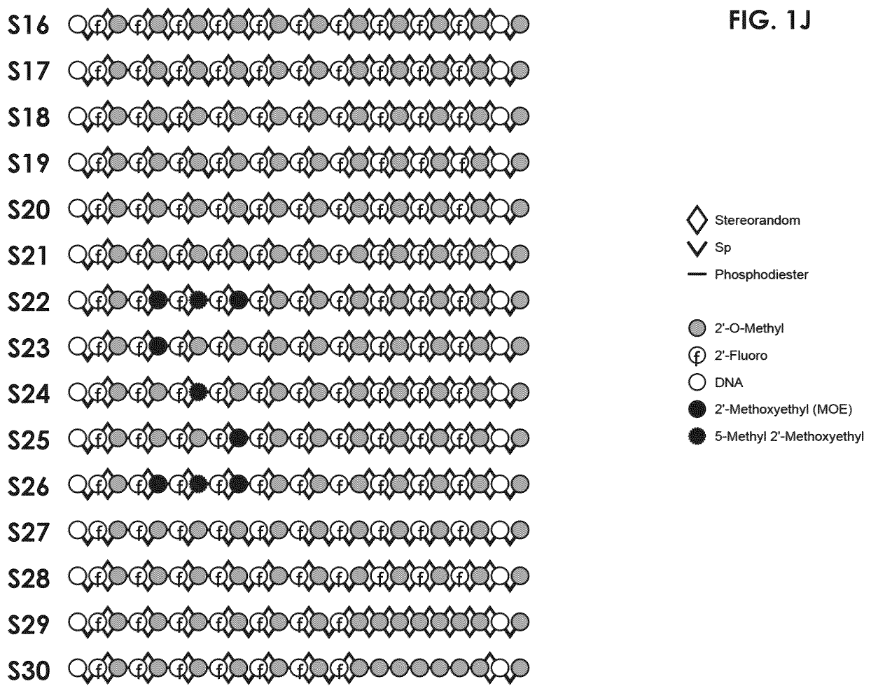

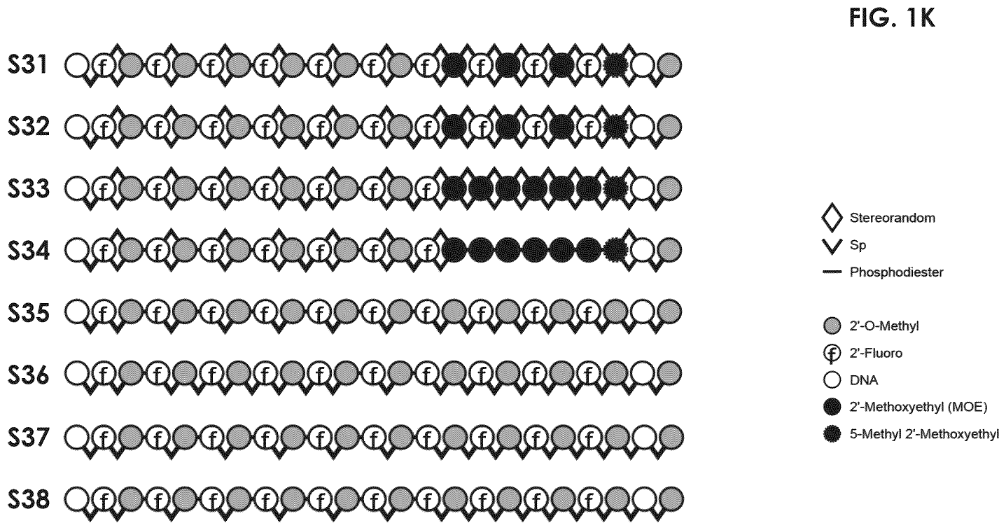

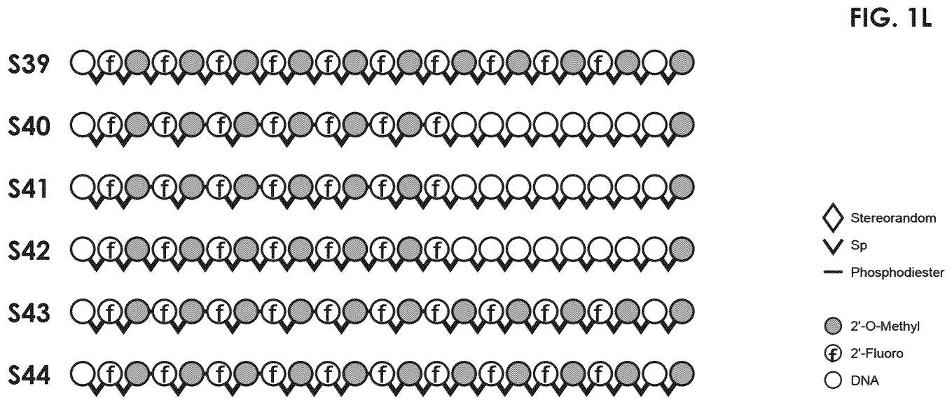

In some embodiments, a provided oligonucleotide has any of Formats illustrated in FIG. 1.

The present disclosure presents data showing that various oligonucleotides of various formats are capable of directing a decrease in the expression and/or level of a target gene or its gene product targeted against any of multiple different sequences, in multiple different genes, in multiple different species; additional data was generated supporting the efficacy of ssRNAi agents of the disclosed Formats and not shown.

In some embodiments, a provided oligonucleotide capable of directing RNase H-mediated knockdown has a structural element or format or portion thereof described herein.

In some embodiments, a provided oligonucleotide capable of directing RNase H-mediated knockdown has the format of any oligonucleotide disclosed herein, e.g., in Table 1A or in the Figures or Tables, or otherwise disclosed herein.

In some embodiments, a provided oligonucleotide has any of Formats illustrated in FIG. 1.

The present disclosure presents data showing that various oligonucleotides of various formats are capable of directing RNase H-mediated knockdown against any of multiple different sequences, in multiple different genes, in multiple different species; additional data was generated supporting the efficacy of ssRNAi agents of the disclosed Formats and not shown.

In some embodiments, a provided oligonucleotide capable of directing single-stranded RNA interference has a structural element or format or portion thereof described herein.

In some embodiments, a provided oligonucleotide capable of directing single-stranded RNA interference has the format of any oligonucleotide disclosed herein, e.g., in Table 1A or in the Figures or Tables, or otherwise disclosed herein.

In some embodiments, a provided single-stranded RNAi agent has any of the Formats illustrated in FIG. 1.

The present disclosure presents data showing that various RNAi agents of various formats are capable of directing RNA interference against any of multiple different sequences, in any of multiple different genes; additional data was generated supporting the efficacy of ssRNAi agents of the disclosed Formats and not shown.

In some embodiments, a target of RNAi is a transcript. In some embodiments, a transcript is pre-mRNA. In some embodiments, a transcript is mature RNA. In some embodiments, a transcript is mRNA. In some embodiments, a transcript comprises a mutation. In some embodiments, a mutation is a frameshift. In some embodiments, a transcript comprises a premature termination codon. In some embodiments, a target of RNAi is a RNA which is not a mRNA. In some embodiments, a target of RNAi is a non-coding RNA. In some embodiments, a target of RNAi is a long non-coding RNA. In some embodiments, provided oligonucleotides in provided compositions, e.g., oligonucleotides of a first plurality, comprise base modifications, sugar modifications, and/or internucleotidic linkage modifications. In some embodiments, provided oligonucleotides comprise base modifications and sugar modifications. In some embodiments, provided oligonucleotides comprise base modifications and internucleotidic linkage modifications. In some embodiments, provided oligonucleotides comprise sugar modifications and internucleotidic modifications. In some embodiments, provided compositions comprise base modifications, sugar modifications, and internucleotidic linkage modifications. Example chemical modifications, such as base modifications, sugar modifications, internucleotidic linkage modifications, etc. are widely known in the art including but not limited to those described in this disclosure. In some embodiments, a modified base is substituted A, T, C, G or U. In some embodiments, a sugar modification is 2′-modification. In some embodiments, a 2′-modification is 2-F modification. In some embodiments, a 2′-modification is 2′-OR′. In some embodiments, a 2′-modification is 2′-OR′, wherein R′ is optionally substituted alkyl. In some embodiments, a 2′-modification is 2′-OMe. In some embodiments, a 2′-modification is 2′-MOE. In some embodiments, a modified sugar moiety is a bridged bicyclic or polycyclic ring. In some embodiments, a modified sugar moiety is a bridged bicyclic or polycyclic ring having 5-20 ring atoms wherein one or more ring atoms are optionally and independently heteroatoms. Example ring structures are widely known in the art, such as those found in BNA, LNA, etc. In some embodiments, provided oligonucleotides comprise both one or more modified internucleotidic linkages and one or more natural phosphate linkages. In some embodiments, oligonucleotides comprising both modified internucleotidic linkage and natural phosphate linkage and compositions thereof provide improved properties, e.g., activities, etc. In some embodiments, a modified internucleotidic linkage is a chiral internucleotidic linkage. In some embodiments, a modified internucleotidic linkage is a phosphorothioate linkage. In some embodiments, a modified internucleotidic linkage is a substituted phosphorothioate linkage.

Among other things, the present disclosure encompasses the recognition that stereorandom oligonucleotide preparations contain a plurality of distinct chemical entities that differ from one another, e.g., in the stereochemical structure of individual backbone chiral centers within the oligonucleotide chain. Without control of stereochemistry of backbone chiral centers, stereorandom oligonucleotide preparations provide uncontrolled compositions comprising undetermined levels of oligonucleotide stereoisomers. Even though these stereoisomers may have the same base sequence, they are different chemical entities at least due to their different backbone stereochemistry, and they can have, as demonstrated herein, different properties, e.g., activities, etc. Among other things, the present disclosure provides new compositions that are or contain particular stereoisomers of oligonucleotides of interest. In some embodiments, a particular stereoisomer may be defined, for example, by its base sequence, its length, its pattern of backbone linkages, and its pattern of backbone chiral centers. As is understood in the art, in some embodiments, base sequence may refer to the identity and/or modification status of nucleoside residues (e.g., of sugar and/or base components, relative to standard naturally occurring nucleotides such as adenine, cytosine, guanosine, thymine, and uracil) in an oligonucleotide and/or to the hybridization character (i.e., the ability to hybridize with particular complementary residues) of such residues. In some embodiments, the present disclosure provide an oligonucleotide composition comprising a predetermined level of oligonucleotides of an individual oligonucleotide type which are chemically identical, e.g., they have the same base sequence, the same pattern of nucleoside modifications (modifications to sugar and base moieties, if any), the same pattern of backbone chiral centers, and the same pattern of backbone phosphorus modifications. The present disclosure demonstrates, among other things, that individual stereoisomers of a particular oligonucleotide can show different stability and/or activity from each other. In some embodiments, property improvements achieved through inclusion and/or location of particular chiral structures within an oligonucleotide can be comparable to, or even better than those achieved through use of particular backbone linkages, residue modifications, etc. (e.g., through use of certain types of modified phosphates [e.g., phosphorothioate, substituted phosphorothioate, etc.], sugar modifications [e.g., 2′-modifications, etc.], and/or base modifications [e.g., methylation, etc.]). Among other things, the present disclosure recognizes that, in some embodiments, properties (e.g., activities, etc.) of an oligonucleotide can be adjusted by optimizing its pattern of backbone chiral centers, optionally in combination with adjustment/optimization of one or more other features (e.g., linkage pattern, nucleoside modification pattern, etc.) of the oligonucleotide. As exemplified by various examples in the present disclosure, provided chirally controlled oligonucleotide compositions can demonstrate improved properties, e.g., improved single-stranded RNA interference activity, RNase H-mediated knockdown, improved delivery, etc.

In some embodiments, oligonucleotide properties can be adjusted by optimizing stereochemistry (pattern of backbone chiral centers) and chemical modifications (modifications of base, sugar, and/or internucleotidic linkage) or patterns thereof.

In some embodiments, a pattern of backbone chiral centers provides increased stability. In some embodiments, a pattern of backbone chiral centers provides surprisingly increased activity. In some embodiments, a pattern of backbone chiral centers provides increased stability and activity. In some embodiments, a pattern of backbone chiral centers provides surprisingly increased binding to certain proteins. In some embodiments, a pattern of backbone chiral centers provides surprisingly enhanced delivery. In some embodiments, a pattern of backbone chiral centers comprises or is (Sp)m(Rp)n, (Rp)n(Sp)m, (Np)t(Rp)n(Sp)m, or (Sp)t(Rp)n(Sp)m. In some embodiments, a pattern of backbone chiral centers comprises or is (Rp)n(Sp)m, (Np)t(Rp)n(Sp)m, or (Sp)t(Rp)n(Sp)m, wherein m>2. In some embodiments, a pattern of backbone chiral centers comprises or is (Rp)n(Sp)m, (Np)t(Rp)n(Sp)m, or (Sp)t(Rp)n(Sp)m, wherein n is 1, t>1, and m>2. In some embodiments, m>3. In some embodiments, m>4. In some embodiments, a pattern of backbone chiral centers comprises one or more achiral natural phosphate linkages.

In some embodiments, a pattern of backbone chiral centers comprises or is (Rp)n(Sp)m, (Sp)t(Rp)n, (Np)t(Rp)n(Sp)m, (Sp)t(Sp)m or (Sp)t(Rp)n(Sp)m. In some embodiments, a pattern of backbone chiral centers comprises or is (Rp)n(Sp)m, (Sp)t(Rp)n, (Np)t(Rp)n(Sp)m, or (Sp)t(Rp)n(Sp)m, and the oligonucleotides comprises one or more 2′-modifications as described herein. In some embodiments, a pattern of backbone chiral centers comprises or is (Rp)n(Sp)m, (Sp)t(Rp)n, (Np)t(Rp)n(Sp)m, or (Sp)t(Rp)n(Sp)m, and the oligonucleotides comprises one or more 2′-F modifications as described herein. In some embodiments, a pattern of backbone chiral centers comprises or is (Rp)n(Sp)m, (Sp)t(Rp)n, (Np)t(Rp)n(Sp)m, or (Sp)t(Rp)n(Sp)m, and the oligonucleotides comprises one or more 2′-OR modifications as described herein. In some embodiments, a pattern of backbone chiral centers comprises or is (Rp)n(Sp)m. In some embodiments, a pattern of backbone chiral centers comprises or is (Sp)t(Rp)n. In some embodiments, a pattern of backbone chiral centers comprises or is (Np)t(Rp)n(Sp)m. In some embodiments, a pattern of backbone chiral centers comprises or is (Sp)t(Sp)m, optionally with n achiral phosphate diester internucleotidic linkages and/or stereorandom (non-chirally controlled) chiral internucleotidic linkages between the section having (Sp)t and the section having (Sp)m. In some embodiments, there are n achiral phosphate diester internucleotidic linkages in between. In some embodiments, there are n stereorandom chiral internucleotidic linkages in between. In some embodiments, a pattern of backbone chiral centers comprises or is (Sp)t(Rp)n(Sp)m. In some embodiments, each oft and m is independently equal to or greater than 1, 2, 3, 4, 5, 6, 7, 8, 9, 10, 11, 12, 13, 14, 15, 16, 17, 18, 19 or 20.

In some embodiments, a common pattern of backbone chiral centers (e.g., a pattern of backbone chiral centers in a single-stranded RNAi agent) comprises a pattern of i^{o}-i^{s}-i^{o}-i^{s}-i^{o}, i^{o}-i^{s}-i^{s}-i^{s}-i^{o}, i^{o}-i^{s}-i^{s}-i^{s}-i^{o}-i^{s}, i^{s}-i^{o}-i^{s}-i^{o}, i^{s}-i^{o}-i^{s}-i^{o}, i^{s}-i^{o}-i^{s}-i^{o}-i^{s}, i^{s}-i^{o}-i^{s}-i^{o}-i^{s}-i^{o}, i^{s}-i^{o}-i^{s}-i^{o}-i^{s}-i^{o}-i^{s}-i^{o}, i^{s}-i^{o}-i^{s}-i^{s}-i^{s}-i^{o}, i^{s}-i^{s}-i^{o}-i^{s}-i^{s}-i^{s}-i^{o}-i^{s}-i^{s}, i^{s}-i^{s}-i^{s}-i^{o}-i^{s}-i^{o}-i^{s}-i^{s}-i^{s}, i^{s}-i^{s}-i^{s}-i^{s}, i^{s}-i^{s}-i^{s}-i^{s}-i^{s}-i^{s}-i^{s}-i^{s}, i^{s}-i^{s}-i^{s}-i^{s}-i^{s}-i^{s}-i^{s}-i^{s}-i^{s}, or i^{r}- i^{r}-i^{r}, wherein is represents an internucleotidic linkage in the Sp configuration; i^{o} represents an achiral internucleotidic linkage; and i^{r} represents an internucleotidic linkage in the Rp configuration.

In some embodiments, a common pattern of backbone chiral centers (e.g., a pattern of backbone chiral centers in a single-stranded RNAi agent) comprises a pattern of OSOSO, OSSSO, OSSSOS, SOSO, SOSO, SOSOS, SOSOSO, SOSOSOSO, SOSSSO, SSOSSSOSS, SSSOSOSSS, SSSSOSOSSSS, SSSSS, SSSSSS, SSSSSSS, SSSSSSSS, SSSSSSSSS, or RRR, wherein S represents a phosphorothioate in the Sp configuration, and O represents a phosphodiester. wherein R represents a phosphorothioate in the Rp configuration.

In some embodiments, the non-chiral center is a phosphodiester linkage. In some embodiments, the chiral center in a Sp configuration is a phosphorothioate linkage. In some embodiments, the non-chiral center is a phosphodiester linkage. In some embodiments, the chiral center in a Sp configuration is a phosphorothioate linkage.

In some embodiments, provided oligonucleotides comprise at least two pairs of alternating phosphodiester and phosphorothioate internucleotidic linkages. In some embodiments, provided oligonucleotides comprise at least 3 pairs of alternating phosphodiester and phosphorothioate internucleotidic linkages. In some embodiments, provided oligonucleotides comprise at least two pairs of alternating phosphodiester and phosphorothioate internucleotidic linkages; and further comprise a block comprising 5 or more consecutive phosphorothioate internucleotidic linkages, wherein at least one phosphorothioate linkage is chirally controlled. In some embodiments, provided oligonucleotides comprise at least two pairs of alternating phosphodiester and phosphorothioate internucleotidic linkages; and further comprise a block comprising 3, 4, 5, 6, 7 or more consecutive phosphorothioate internucleotidic linkages, wherein at least one phosphorothioate linkage is chirally controlled. In some embodiments, provided oligonucleotides comprise at least 5 pairs of alternating phosphodiester and phosphorothioate internucleotidic linkages; and further comprise a block comprising 3, 4, 5, 6, 7 or more consecutive phosphorothioate internucleotidic linkages, wherein at least one phosphorothioate linkage is chirally controlled. In some embodiments, provided oligonucleotides comprise at least 6 pairs of alternating phosphodiester and phosphorothioate internucleotidic linkages; and further comprise a block comprising 3, 4, 5, 6, 7 or more consecutive phosphorothioate internucleotidic linkages, wherein at least one phosphorothioate linkage is chirally controlled. In some embodiments, provided oligonucleotides comprise at least two pairs of alternating phosphodiester and phosphorothioate internucleotidic linkages; and further comprise a block comprising 5 or more consecutive phosphodiester internucleotidic linkages, wherein at least one phosphorothioate linkage is chirally controlled. In some embodiments, provided oligonucleotides comprise one or more natural phosphate linkages and one or more modified internucleotidic linkages. Provided oligonucleotides can comprise various number of natural phosphate linkages.

In some embodiments, provided oligonucleotides comprise no natural phosphate linkages. In some embodiments, provided oligonucleotides comprise one natural phosphate linkage. In some embodiments, provided oligonucleotides comprise 2 or more natural phosphate linkages. In some embodiments, provided oligonucleotides comprise 3 or more natural phosphate linkages. In some embodiments, provided oligonucleotides comprise 4 or more natural phosphate linkages. In some embodiments, provided oligonucleotides comprise 5 or more natural phosphate linkages. In some embodiments, provided oligonucleotides comprise 6 or more natural phosphate linkages. In some embodiments, provided oligonucleotides comprise 7 or more natural phosphate linkages. In some embodiments, provided oligonucleotides comprise 8 or more natural phosphate linkages. In some embodiments, provided oligonucleotides comprise 9 or more natural phosphate linkages. In some embodiments, provided oligonucleotides comprise 10 or more natural phosphate linkages. In some embodiments, provided oligonucleotides comprise 15 or more natural phosphate linkages. In some embodiments, provided oligonucleotides comprise 20 or more natural phosphate linkages. In some embodiments, provided oligonucleotides comprise 25 or more natural phosphate linkages. In some embodiments, provided oligonucleotides comprise 30 or more natural phosphate linkages. In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, 5% or more of the internucleotidic linkages of provided oligonucleotides are natural phosphate linkages. In some embodiments, 10% or more of the internucleotidic linkages of provided oligonucleotides are natural phosphate linkages. In some embodiments, 15% or more of the internucleotidic linkages of provided oligonucleotides are natural phosphate linkages. In some embodiments, 20% or more of the internucleotidic linkages of provided oligonucleotides are natural phosphate linkages. In some embodiments, 25% or more of the internucleotidic linkages of provided oligonucleotides are natural phosphate linkages. In some embodiments, 30% or more of the internucleotidic linkages of provided oligonucleotides are natural phosphate linkages. In some embodiments, 35% or more of the internucleotidic linkages of provided oligonucleotides are natural phosphate linkages. In some embodiments, 40% or more of the internucleotidic linkages of provided oligonucleotides are natural phosphate linkages

In some embodiments, 5% or more of the internucleotidic linkages of provided oligonucleotides are modified internucleotidic linkages. In some embodiments, 10% or more of the internucleotidic linkages of provided oligonucleotides are modified internucleotidic linkages. In some embodiments, 15% or more of the internucleotidic linkages of provided oligonucleotides are modified internucleotidic linkages. In some embodiments, 20% or more of the internucleotidic linkages of provided oligonucleotides are modified internucleotidic linkages. In some embodiments, 25% or more of the internucleotidic linkages of provided oligonucleotides are modified internucleotidic linkages. In some embodiments, 30% or more of the internucleotidic linkages of provided oligonucleotides are modified internucleotidic linkages. In some embodiments, 35% or more of the internucleotidic linkages of provided oligonucleotides are modified internucleotidic linkages. In some embodiments, 40% or more of the internucleotidic linkages of provided oligonucleotides are modified internucleotidic linkages. In some embodiments, provided oligonucleotides can bind to a transcript, and improve single-stranded RNA interference of the transcript. In some embodiments, provided oligonucleotides improve single-stranded RNA interference, with efficiency greater than a comparable oligonucleotide under one or more suitable conditions.

In some embodiments, a provided improved single-stranded RNA interference is at least 10%, 20%, 30%, 40%, 50%, 60%, 70%, 80%, 90%, 100%, 110%, 120%, 130%, 140%, 150%, 160%, 170%, 180%, 190% more than, or 2, 3, 4, 5, 6, 7, 8, 9, 10, 11, 12, 13, 14, 15, 16, 17, 18, 19, 20, 30, 40, 50 or more fold of, that of a comparable oligonucleotide under one or more suitable conditions.

In some embodiments, expression or level of a target gene or its gene product is decreased by at least about 10%, 15%, 20%, 25%, 30%, 35%, 40%, 45%, 50%, 60%, 70%, or 80% by administration of an oligonucleotide. In some embodiments, expression or level of a target gene or its gene product is decreased by at least about 10%, 15%, 20%, 25%, 30%, 35%, 40%, 45%, 50%, 60%, 70%, or 80% total by RNase H-mediated knockdown and/or RNA interference directed by an oligonucleotide. In some embodiments, expression or level of a target gene or its gene product is decreased by at least about 10%, 15%, 20%, 25%, 30%, 35%, 40%, 45%, 50%, 60%, 70%, or 80% total by RNase H-mediated knockdown and/or RNA interference directed by an oligonucleotide at a concentration of 5 nm or less in a cell(s) in vitro. In some embodiments, a cell(s) is a mammalian cell(s). In some embodiments, a cell(s) is a human cell(s). In some embodiments, a cell(s) is a hepatic cell(s). In some embodiments, a cell(s) is a Huh7 or Hep3B cell(s). In some embodiments, a single-stranded RNAi agent is capable of decreasing expression or level of a target gene or its gene product by at least about 20% in a cell(s) in vitro at a concentration of 25 nM or less. In some embodiments, a single-stranded RNAi agent is capable of decreasing expression or level of a target gene or its gene product by at least about 50% in a cell(s) in vitro at a concentration of 25 nM or less. In some embodiments, an oligonucleotide is capable of decreasing expression or level of a target gene or its gene product by at least about 20% in a cell(s) in vitro at a concentration of 25 nM or less. In some embodiments, an oligonucleotide is capable of decreasing expression or level of a target gene or its gene product by at least about 50% in a cell(s) in vitro at a concentration of 25 nM or less. In some embodiments, IC50 is inhibitory concentration to decrease expression or level or a target gene or its gene product by 50% in a cell(s) in vitro. In some embodiments, a single-stranded RNAi agent has an IC50 of no more than about 10 nM in a cell(s) in vitro. In some embodiments, a single-stranded RNAi agent has an IC50 of no more than about 2 nM in a cell(s) in vitro. In some embodiments, an oligonucleotide has an IC50 of no more than about 10 nM in a cell(s) in vitro. In some embodiments, an oligonucleotide has an IC50 of no more than about 2 nM in a cell(s) in vitro.

In some embodiments, the present disclosure provides an oligonucleotide, e.g., a single-stranded RNAi agent of an oligonucleotide type whose pattern of backbone chiral centers comprises (Sp)mRp or Rp(Sp)m. In some embodiments, the present disclosure provides a single-stranded RNAi agent of an oligonucleotide type whose pattern of backbone chiral centers comprises Rp(Sp)m. In some embodiments, the present disclosure provides a single-stranded RNAi agent of an oligonucleotide type whose pattern of backbone chiral centers comprises (Sp)mRp. In some embodiments, m is 2. In some embodiments, the present disclosure provides a single-stranded RNAi agent of an oligonucleotide type whose pattern of backbone chiral centers comprises Rp(Sp)₂. In some embodiments, the present disclosure provides a single-stranded RNAi agent of an oligonucleotide type whose pattern of backbone chiral centers comprises (Sp)₂Rp(Sp)₂. In some embodiments, the present disclosure provides a single-stranded RNAi agent of an oligonucleotide type whose pattern of backbone chiral centers comprises (Rp)₂Rp(Sp)₂. In some embodiments, the present disclosure provides a single-stranded RNAi agent of an oligonucleotide type whose pattern of backbone chiral centers comprises RpSpRp(Sp)₂. In some embodiments, the present disclosure provides a single-stranded RNAi agent of an oligonucleotide type whose pattern of backbone chiral centers comprises SpRpRp(Sp)₂. In some embodiments, the present disclosure provides a single-stranded RNAi agent of an oligonucleotide type whose pattern of backbone chiral centers comprises (Sp)₂Rp.

As defined herein, m is 1-50. In some embodiments, m is 1. In some embodiments, m is 2-50. In some embodiments, m is 2, 3, 4, 5, 6, 7 or 8. In some embodiments, m is 3, 4, 5, 6, 7 or 8. In some embodiments, m is 4, 5, 6, 7 or 8. In some embodiments, m is 5, 6, 7 or 8. In some embodiments, m is 6, 7 or 8. In some embodiments, m is 7 or 8. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16. In some embodiments, m is 17. In some embodiments, m is 18. In some embodiments, m is 19. In some embodiments, m is 20. In some embodiments, m is 21. In some embodiments, m is 22. In some embodiments, m is 23. In some embodiments, m is 24. In some embodiments, m is 25. In some embodiments, m is greater than 25.

In some embodiments, a provided oligonucleotide comprises any pattern of stereochemistry described herein. In some embodiments, a provided oligonucleotide comprises any pattern of stereochemistry described herein and is capable of directing RNA interference. In some embodiments, a provided oligonucleotide comprises any pattern of stereochemistry described herein and is capable of directing RNase H-mediated knockdown. In some embodiments, a provided oligonucleotide comprises any pattern of stereochemistry described herein and is capable of directing RNA interference and RNase H-mediated knockdown. In some embodiments, a provided oligonucleotide comprises any pattern of stereochemistry described herein and is capable of directing RNA interference, wherein the pattern of stereochemistry is in the seed and/or post-seed region. In some embodiments, a provided oligonucleotide comprises any pattern of stereochemistry described herein and is capable of directing RNA interference and RNase H-mediated knockdown, wherein the pattern of stereochemistry is in the seed and/or post-seed region.

In some embodiments, a provided oligonucleotide comprises any modification or pattern of modification described herein. In some embodiments, a provided oligonucleotide comprises any modification or pattern of modification described herein and is capable of directing RNA interference. In some embodiments, a provided oligonucleotide comprises any pattern of modification described herein and is capable of directing RNase H-mediated knockdown. In some embodiments, a provided oligonucleotide comprises any pattern of modification described herein and is capable of directing RNA interference and RNase H-mediated knockdown. In some embodiments, a provided oligonucleotide comprises any pattern of modification described herein and is capable of directing RNA interference, wherein the pattern of modification is in the seed and/or post-seed region. In some embodiments, a provided oligonucleotide comprises any pattern of modification described herein and is capable of directing RNA interference and RNase H-mediated knockdown, wherein the pattern of modification is in the seed and/or post-seed region. In some embodiments, a modification or pattern of modification is a modification or pattern of modifications at the 2′ position of a sugar. In some embodiments, a modification or pattern of modification is a modification or pattern of modifications of sugars, e.g., at the 2′ position of a sugar, including but not limited to, 2′-deoxy, 2′-F, 2′-OMe, 2′-MOE, and 2′-OR1, wherein R1 is optionally substituted C₁-6 alkyl.

In some embodiments, the present disclosure demonstrates that 2′-F modifications, among other things, can improve single-stranded RNA interference. In some embodiments, the present disclosure demonstrates that Sp internucleotidic linkages, among other things, at the 5′- and 3′-ends can improve oligonucleotide stability. In some embodiments, the present disclosure demonstrates that, among other things, natural phosphate linkages and/or Rp internucleotidic linkages can improve removal of oligonucleotides from a system. As appreciated by a person having ordinary skill in the art, various assays known in the art can be utilized to assess such properties in accordance with the present disclosure.

In some embodiments, provided oligonucleotides capable of directing single-stranded RNA interference comprise one or more modified sugar moieties. In some embodiments, 5% or more of the sugar moieties of provided oligonucleotides are modified.

In some embodiments, provided oligonucleotides comprise one or more modified sugar moieties. In some embodiments, 5%, 10%, 15%, 20%, 25%, 30%, 35%, 40%, 45%, 50%, 55%, 60%, 65%, 70%, 75%, 80%, 85%, 90%, 95% or more, or 100%, of the sugar moieties of provided oligonucleotides are modified. In some embodiments, a modified sugar moiety comprises a 2′-modification. In some embodiments, a modified sugar moiety comprises a 2′-modification. In some embodiments, a 2′-modification is 2′-OR¹. In some embodiments, a 2′-modification is a 2′-OMe. In some embodiments, a 2′-modification is a 2′-MOE. In some embodiments, a 2′-modification is an LNA sugar modification. In some embodiments, a 2′-modification is 2′-F. In some embodiments, each sugar modification is independently a 2′-modification. In some embodiments, each sugar modification is independently 2′-OR¹ or 2′-F. In some embodiments, each sugar modification is independently 2′-OR¹ or 2′-F, wherein R¹ is optionally substituted C₁₋₆ alkyl. In some embodiments, each sugar modification is independently 2′-OR¹ or 2′-F, wherein at least one is 2′-F. In some embodiments, each sugar modification is independently 2′-OR¹ or 2′-F, wherein R¹ is optionally substituted C₁₋₆ alkyl, and wherein at least one is 2′-OR¹. In some embodiments, each sugar modification is independently 2′-OR¹ or 2′-F, wherein at least one is 2′-F, and at least one is 2′-OR¹. In some embodiments, each sugar modification is independently 2′-OR¹ or 2′-F, wherein R¹ is optionally substituted C₁₋₆ alkyl, and wherein at least one is 2′-F, and at least one is 2′-OR¹.

In some embodiments, a nucleoside comprising a 2′-modification is followed by a modified internucleotidic linkage. In some embodiments, a nucleoside comprising a 2′-modification is preceded by a modified internucleotidic linkage. In some embodiments, a modified internucleotidic linkage is a chiral internucleotidic linkage. In some embodiments, a modified internucleotidic linkage is a phosphorothioate. In some embodiments, a chiral internucleotidic linkage is Sp. In some embodiments, a nucleoside comprising a 2′-modification is followed by an Sp chiral internucleotidic linkage. In some embodiments, a nucleoside comprising a 2′-F is followed by an Sp chiral internucleotidic linkage. In some embodiments, a nucleoside comprising a 2′-modification is preceded by an Sp chiral internucleotidic linkage. In some embodiments, a nucleoside comprising a 2′-F is preceded by an Sp chiral internucleotidic linkage. In some embodiments, a chiral internucleotidic linkage is Rp. In some embodiments, a nucleoside comprising a 2′-modification is followed by an Rp chiral internucleotidic linkage. In some embodiments, a nucleoside comprising a 2′-F is followed by an Rp chiral internucleotidic linkage. In some embodiments, a nucleoside comprising a 2′-modification is preceded by an Rp chiral internucleotidic linkage. In some embodiments, a nucleoside comprising a 2′-F is preceded by an Rp chiral internucleotidic linkage.

In some embodiments, the present disclosure provides an oligonucleotide composition comprising a first plurality of oligonucleotides, wherein:
oligonucleotides of the first plurality have the same base sequence; and
oligonucleotides of the first plurality comprise one or more modified sugar moieties, or comprise one or more natural phosphate linkages and one or more modified internucleotidic linkages.

In some embodiments, oligonucleotides of the first plurality comprise one or more modified sugar moieties. In some embodiments, provided oligonucleotides comprise one or more modified sugar moieties. In some embodiments, provided oligonucleotides comprise 2, 3, 4, 5, 6, 7, 8, 9, 10, 11, 12, 13, 14, 15, 16, 17, 18, 19, 20, 21, 22, 23, 24, or 35 modified sugar moieties.

In some embodiments, provided oligonucleotides comprise one or more 2′-F. In some embodiments, provided oligonucleotides comprise two or more 2′-F. In some embodiments, provided oligonucleotides comprise three or more 2′-F. In some embodiments, provided oligonucleotides comprise four or more 2′-F. In some embodiments, provided oligonucleotides comprise five or more 2′-F. In some embodiments, provided oligonucleotides comprise six or more 2′-F. In some embodiments, provided oligonucleotides comprise seven or more 2′-F. In some embodiments, provided oligonucleotides comprise eight or more 2′-F. In some embodiments, provided oligonucleotides comprise nine or more 2′-F. In some embodiments, provided oligonucleotides comprise ten or more 2′-F. In some embodiments, provided oligonucleotides comprise 11 or more 2′-F. In some embodiments, provided oligonucleotides comprise 12 or more 2′-F. In some embodiments, provided oligonucleotides comprise 13 or more 2′-F. In some embodiments, provided oligonucleotides comprise 14 or more 2′-F. In some embodiments, provided oligonucleotides comprise 15 or more 2′-F. In some embodiments, provided oligonucleotides comprise 16 or more 2′-F. In some embodiments, provided oligonucleotides comprise 17 or more 2′-F. In some embodiments, provided oligonucleotides comprise 18 or more 2′-F. In some embodiments, provided oligonucleotides comprise 19 or more 2′-F. In some embodiments, provided oligonucleotides comprise 20 or more 2′-F. In some embodiments, provided oligonucleotides comprise 21 or more 2′-F. In some embodiments, provided oligonucleotides comprise 22 or more 2′-F. In some embodiments, provided oligonucleotides comprise 23 or more 2′-F. In some embodiments, provided oligonucleotides comprise 24 or more 2′-F. In some embodiments, provided oligonucleotides comprise 25 or more 2′-F. In some embodiments, provided oligonucleotides comprise 30 or more 2′-F. In some embodiments, provided oligonucleotides comprise 35 or more 2′-F. In some embodiments, in provided oligonucleotides, a nucleoside comprising a 2′-modification is followed by a modified internucleotidic linkage. In some embodiments, in provided oligonucleotides, a nucleoside comprising a 2′-modification is preceded by a modified internucleotidic linkage. In some embodiments, a modified internucleotidic linkage is a chiral internucleotidic linkage. In some embodiments, a modified internucleotidic linkage is a phosphorothioate. In some embodiments, a chiral internucleotidic linkage is Sp. In some embodiments, in provided oligonucleotides, a nucleoside comprising a 2′-modification is followed by an Sp chiral internucleotidic linkage. In some embodiments, in provided oligonucleotides, a nucleoside comprising a 2′-F is followed by an Sp chiral internucleotidic linkage. In some embodiments, in provided oligonucleotides, a nucleoside comprising a 2′-modification is preceded by an Sp chiral internucleotidic linkage. In some embodiments, in provided oligonucleotides, a nucleoside comprising a 2′-F is preceded by an Sp chiral internucleotidic linkage. In some embodiments, a chiral internucleotidic linkage is Rp. In some embodiments, in provided oligonucleotides, a nucleoside comprising a 2′-modification is followed by an Rp chiral internucleotidic linkage. In some embodiments, in provided oligonucleotides, a nucleoside comprising a 2′-F is followed by an Rp chiral internucleotidic linkage. In some embodiments, in provided oligonucleotides, a nucleoside comprising a 2′-modification is preceded by an Rp chiral internucleotidic linkage. In some embodiments, in provided oligonucleotides, a nucleoside comprising a 2′-F is preceded by an Rp chiral internucleotidic linkage. In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, oligonucleotides of the first plurality comprise one or more natural phosphate linkages and one or more modified internucleotidic linkages.

Provided oligonucleotides can comprise various number of natural phosphate linkages. In some embodiments, provided oligonucleotides comprise no natural phosphate linkages. In some embodiments, provided oligonucleotides comprise one natural phosphate linkage. In some embodiments, provided oligonucleotides comprise 2 or more natural phosphate linkages. In some embodiments, provided oligonucleotides comprise about 5 or more consecutive modified sugar moieties. In some embodiments, provided oligonucleotides comprise no more than about 95% unmodified sugar moieties. In some embodiments, provided oligonucleotides comprise no more than about 50% unmodified sugar moieties. In some embodiments, each sugar moiety of the oligonucleotides of the first plurality is independently modified. Provided oligonucleotides can comprise various number of natural phosphate linkages. In some embodiments, provided oligonucleotides comprise no natural phosphate linkages. In some embodiments, provided oligonucleotides comprise one natural phosphate linkage. In some embodiments, provided oligonucleotides comprise 2 or more natural phosphate linkages. In some embodiments, provided oligonucleotides comprise 3 or more natural phosphate linkages. In some embodiments, provided oligonucleotides comprise 4 or more natural phosphate linkages. In some embodiments, provided oligonucleotides comprise 5 or more natural phosphate linkages. In some embodiments, provided oligonucleotides comprise 6 or more natural phosphate linkages. In some embodiments, provided oligonucleotides comprise 7 or more natural phosphate linkages. In some embodiments, provided oligonucleotides comprise 8 or more natural phosphate linkages. In some embodiments, provided oligonucleotides comprise 9 or more natural phosphate linkages. In some embodiments, provided oligonucleotides comprise 10 or more natural phosphate linkages. In some embodiments, provided oligonucleotides comprise 15 or more natural phosphate linkages. In some embodiments, provided oligonucleotides comprise 20 or more natural phosphate linkages. In some embodiments, provided oligonucleotides comprise 25 or more natural phosphate linkages. In some embodiments, provided oligonucleotides comprise 30 or more natural phosphate linkages. In some embodiments, provided oligonucleotides comprise about 25 or more consecutive modified sugar moieties. In some embodiments, provided oligonucleotides comprise about 20 or more consecutive modified sugar moieties. In some embodiments, provided oligonucleotides comprise about 15 or more consecutive modified sugar moieties. In some embodiments, provided oligonucleotides comprise about 10 or more consecutive modified sugar moieties. In some embodiments, provided oligonucleotides comprise about 9 or more consecutive modified sugar moieties. In some embodiments, provided oligonucleotides comprise about 8 or more consecutive modified sugar moieties. In some embodiments, provided oligonucleotides comprise about 7 or more consecutive modified sugar moieties. In some embodiments, provided oligonucleotides comprise about 6 or more consecutive modified sugar moieties. In some embodiments, provided oligonucleotides comprise about 5 or more consecutive modified sugar moieties. In some embodiments, provided oligonucleotides comprise about 4 or more consecutive modified sugar moieties. In some embodiments, provided oligonucleotides comprise about 3 or more consecutive modified sugar moieties. In some embodiments, provided oligonucleotides comprise about 2 or more consecutive modified sugar moieties. In some embodiments, provided oligonucleotides comprise about 25 or more consecutive modified sugar moieties. In some embodiments, provided oligonucleotides comprise about 20 or more consecutive modified sugar moieties. In some embodiments, provided oligonucleotides comprise about 15 or more consecutive modified sugar moieties. In some embodiments, provided oligonucleotides comprise about 10 or more consecutive modified sugar moieties. In some embodiments, provided oligonucleotides comprise about 5 or more consecutive modified sugar moieties. In some embodiments, provided oligonucleotides comprise no more than about 95% unmodified sugar moieties. In some embodiments, provided oligonucleotides comprise no more than about 90% unmodified sugar moieties. In some embodiments, provided oligonucleotides comprise no more than about 85% unmodified sugar moieties. In some embodiments, provided oligonucleotides comprise no more than about 80% unmodified sugar moieties. In some embodiments, provided oligonucleotides comprise no more than about 70% unmodified sugar moieties. In some embodiments, provided oligonucleotides comprise no more than about 60% unmodified sugar moieties. In some embodiments, provided oligonucleotides comprise no more than about 50% unmodified sugar moieties. In some embodiments, provided oligonucleotides comprise no more than about 40% unmodified sugar moieties. In some embodiments, provided oligonucleotides comprise no more than about 30% unmodified sugar moieties. In some embodiments, provided oligonucleotides comprise no more than about 20% unmodified sugar moieties. In some embodiments, provided oligonucleotides comprise no more than about 10% unmodified sugar moieties. In some embodiments, provided oligonucleotides comprise no more than about 5% unmodified sugar moieties. In some embodiments, each sugar moiety of the oligonucleotides of the first plurality is independently modified. In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown.

In some embodiments, provided compositions alter transcript single-stranded RNA interference so that an undesired target and/or biological function are suppressed. In some embodiments, in such cases provided composition can also induce cleavage of the transcript after hybridization.

In some embodiments, each oligonucleotide of the first plurality comprises one or more modified sugar moieties and/or one or more modified internucleotidic linkages.

In some embodiments, each oligonucleotide of the first plurality comprises no more than about 95% unmodified sugar moieties. In some embodiments, each oligonucleotide of the first plurality comprises no more than about 50% unmodified sugar moieties. In some embodiments, each oligonucleotide of the first plurality comprises no more than about 5% unmodified sugar moieties. In some embodiments, each sugar moiety of the oligonucleotides of the first plurality is independently modified.

In some embodiments, each oligonucleotide of the first plurality comprises two or more modified internucleotidic linkages. In some embodiments, each oligonucleotide of the first plurality comprises three or more modified internucleotidic linkages. In some embodiments, each oligonucleotide of the first plurality comprises four or more modified internucleotidic linkages. In some embodiments, each oligonucleotide of the first plurality comprises five or more modified internucleotidic linkages. In some embodiments, each oligonucleotide of the first plurality comprises ten or more modified internucleotidic linkages.

In some embodiments, each oligonucleotide of the first plurality comprises about 15 or more modified internucleotidic linkages. In some embodiments, each oligonucleotide of the first plurality comprises about 20 or more modified internucleotidic linkages. In some embodiments, each oligonucleotide of the first plurality comprises about 25 or more modified internucleotidic linkages.

In some embodiments, each oligonucleotide of the first plurality comprises no more than about 30% natural phosphate linkages. In some embodiments, each oligonucleotide of the first plurality comprises no more than about 20% natural phosphate linkages. In some embodiments, each oligonucleotide of the first plurality comprises no more than about 10% natural phosphate linkages. In some embodiments, each oligonucleotide of the first plurality comprises no more than about 5% natural phosphate linkages.

In some embodiments, compared to a reference condition, provided chirally controlled oligonucleotide compositions are surprisingly effective. In some embodiments, desired biological effects (e.g., as measured by increased levels of desired mRNA, proteins, etc., decreased levels of undesired mRNA, proteins, etc.) can be enhanced by more than 5, 10, 15, 20, 25, 30, 40, 50, or 100 fold. In some embodiments, a change is measured by increase of a desired mRNA level compared to a reference condition. In some embodiments, a change is measured by decrease of an undesired mRNA level compared to a reference condition. In some embodiments, a reference condition is absence of oligonucleotide treatment. In some embodiments, a reference condition is a stereorandom composition of oligonucleotides having the same base sequence and chemical modifications.

In some embodiments, provided oligonucleotides contain increased levels of one or more isotopes. In some embodiments, provided oligonucleotides are labeled, e.g., by one or more isotopes of one or more elements, e.g., hydrogen, carbon, nitrogen, etc. In some embodiments, provided oligonucleotides in provided compositions, e.g., oligonucleotides of a first plurality, comprise base modifications, sugar modifications, and/or internucleotidic linkage modifications, wherein the oligonucleotides contain an enriched level of deuterium. In some embodiments, provided oligonucleotides are labeled with deuterium (replacing —¹H with —²H) at one or more positions. In some embodiments, one or more ¹H of an oligonucleotide or any moiety conjugated to the oligonucleotide (e.g., a targeting moiety, lipid moiety, etc.) is substituted with ²H. Such oligonucleotides can be used in any composition or method described herein.

The present invention includes all pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I, ¹²⁴I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

Isotopically-labelled compounds can generally be prepared by conventional techniques known to those skilled in the art or by processes analogous to those described in the accompanying Examples and Preparations using an appropriate isotopically-labelled reagent in place of the non-labelled reagent previously employed.

In some embodiments, controlling structural elements of oligonucleotides, such as chemical modifications (e.g., modifications of a sugar, base and/or internucleotidic linkage) or patterns thereof, alterations in stereochemistry (e.g., stereochemistry of a backbone chiral internucleotidic linkage) or patterns thereof, substitution of an atom with an isotope of the same element, and/or conjugation with an additional chemical moiety (e.g., a lipid moiety, targeting moiety, etc.) can have a significant impact on a desired biological effect. In some embodiments, a desired biological effect is enhanced by more than 2 fold.

In some embodiments, a desired biological effect is enhanced by more than 3, 4, 5, 6, 7, 8, 9, 10, 11, 12, 13, 14, 15, 20, 25, 30, 40, 50, 60, 70, 80, 90, or 100 fold.

In some embodiments, a desired biological effect is directing a decrease in the expression and/or level of a target gene or its gene product. In some embodiments, a desired biological effect is improved single-stranded RNA interference. In some embodiments, a desired biological effect is improved RNase H-mediated knockdown. In some embodiments, a desired biological effect is improved single-stranded RNA interference and/or RNase H-mediated knockdown.

In some embodiments, the present disclosure provides an oligonucleotide composition comprising a first plurality of oligonucleotides which:
1) have a common base sequence complementary to a target sequence in a transcript; and
2) comprise one or more modified sugar moieties and modified internucleotidic linkages.

In some embodiments, a provided oligonucleotide composition is characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, the present disclosure provides an oligonucleotide composition comprising a first plurality of oligonucleotides capable of directing single-stranded RNA interference, wherein an oligonucleotide type is defined by:
1) base sequence;
2) pattern of backbone linkages;
3) pattern of backbone chiral centers; and
4) pattern of backbone phosphorus modifications,
   which composition is chirally controlled in that it is enriched, relative to a substantially racemic preparation of oligonucleotides having the same base sequence, for oligonucleotides of the particular oligonucleotide type,
the oligonucleotide composition being characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof.

In some embodiments, each of the consecutive nucleoside units is independently preceded and/or followed by a modified internucleotidic linkage. In some embodiments, each of the consecutive nucleoside units is independently preceded and/or followed by a phosphorothioate linkage. In some embodiments, each of the consecutive nucleoside units is independently preceded and/or followed by a chirally controlled modified internucleotidic linkage. In some embodiments, each of the consecutive nucleoside units is independently preceded and/or followed by a chirally controlled phosphorothioate linkage. In some embodiments, a modified internucleotidic linkage has a structure of Formula I. In some embodiments, a modified internucleotidic linkage has a structure of Formula I-a.

In some embodiments, the present disclosure provides a single-stranded RNAi agent comprising a predetermined level of a first plurality of oligonucleotides, wherein:
oligonucleotides of the first plurality have the same base sequence;
oligonucleotides of the first plurality comprise a seed region comprising 2, 3, 4, 5, 6, 7 or more consecutive Sp modified internucleotidic linkages, a post-seed region comprising 2, 3, 4, 5, 6, 7, 8, 9, 10 or more consecutive Sp modified internucleotidic linkages.

In some embodiments, a seed region comprises 2 or more consecutive Sp modified internucleotidic linkages. In some embodiments, a seed region comprises 3, 4, 5, 6, 7, 8, 9, 10 or more consecutive Sp modified internucleotidic linkages. In some embodiments, a seed region comprises 3 or more consecutive Sp modified internucleotidic linkages. In some embodiments, a seed region comprises 4 or more consecutive Sp modified internucleotidic linkages. In some embodiments, a post-seed region comprises 4 or more consecutive Sp modified internucleotidic linkages.

In some embodiments, the present disclosure provides a single-stranded RNAi agent comprising a predetermined level of a first plurality of oligonucleotides, wherein:
oligonucleotides of the first plurality have the same base sequence;
oligonucleotides of the first plurality comprise a seed region comprising 4, 5, 6, 7, 8 or more consecutive Sp modified internucleotidic linkages, a post-seed region comprising 4, 5, 6, 7, 8, 9, 10 or more consecutive Sp modified internucleotidic linkages, and a middle region between the seed region and the 3′-region comprising 1, 2, 3, 4, 5, 6, 7, 8, 9, 10 or more natural phosphate linkages.

In some embodiments, the present disclosure provides a single-stranded RNAi agent comprising a predetermined level of a first plurality of oligonucleotides, wherein:
oligonucleotides of the first plurality have the same base sequence;
oligonucleotides of the first plurality comprise a seed region comprising 5, 6, 7, 8 or more consecutive Sp modified internucleotidic linkages, a post-seed region comprising 5, 6, 7, 8, 9, 10 or more consecutive Sp modified internucleotidic linkages, and a middle region between the seed region and the 3′-region comprising 2, 3, 4, 5, 6, 7, 8 or more natural phosphate linkages.

In some embodiments, the present disclosure provides a single-stranded RNAi agent comprising a predetermined level of a first plurality of oligonucleotides, wherein:
oligonucleotides of the first plurality have the same base sequence;
oligonucleotides of the first plurality comprise a seed region comprising 6, 7 or more consecutive Sp modified internucleotidic linkages, a post-seed region comprising 6, 7, 8, 9, 10 or more consecutive Sp modified internucleotidic linkages.

In some embodiments, the present disclosure provides a single-stranded RNAi agent comprising a predetermined level of a first plurality of oligonucleotides, wherein:
oligonucleotides of the first plurality have the same base sequence;
oligonucleotides of the first plurality comprise a seed region comprising 6, 7, 8 or more consecutive Sp modified internucleotidic linkages, a post-seed region comprising 6, 7, 8, 9, 10 or more consecutive Sp modified internucleotidic linkages.

In some embodiments, a modified internucleotidic linkage has a structure of Formula I. In some embodiments, a modified internucleotidic linkage has a structure of Formula I-a.

As demonstrated in the present disclosure, in some embodiments, a provided oligonucleotide composition is characterized in that, when it is contacted with the transcript in a single-stranded RNA interference system, RNAi-mediated knockdown of the transcript is improved relative to that observed under reference conditions selected from the group consisting of absence of the composition, presence of a reference composition, and combinations thereof. In some embodiments, single-stranded RNA interference is increased 10%, 20%, 30%, 40%, 50%, 60%, 70%, 80%, 90%, 100%, or 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, 1000 fold or more. In some embodiments, as exemplified in the present disclosure, levels of the plurality of oligonucleotides, e.g., a first plurality of oligonucleotides, in provided compositions are pre-determined.

In some embodiments, the present disclosure provides an oligonucleotide composition comprising a first plurality of oligonucleotides defined by having:
1) a common base sequence and length;
2) a common pattern of backbone linkages; and
3) a common pattern of backbone chiral centers, which composition is a substantially pure preparation of a single oligonucleotide in that a predetermined level of the oligonucleotides in the composition have the common base sequence and length, the common pattern of backbone linkages, and the common pattern of backbone chiral centers.

In some embodiments, a common base sequence and length may be referred to as a common base sequence. In some embodiments, oligonucleotides having a common base sequence may have the same pattern of nucleoside modifications, e.g., sugar modifications, base modifications, etc. In some embodiments, a pattern of nucleoside modifications may be represented by a combination of locations and modifications. In some embodiments, a pattern of backbone linkages comprises locations and types (e.g., phosphate, phosphorothioate, substituted phosphorothioate, etc.) of each internucleotidic linkages. A pattern of backbone chiral centers of an oligonucleotide can be designated by a combination of linkage phosphorus stereochemistry (Rp/Sp) from 5′ to 3′. As exemplified above, locations of non-chiral linkages may be obtained, for example, from pattern of backbone linkages.

As understood by a person having ordinary skill in the art, a stereorandom or racemic preparation of oligonucleotides is prepared by non-stereoselective and/or low-stereoselective coupling of nucleotide monomers, typically without using any chiral auxiliaries, chiral modification reagents, and/or chiral catalysts. In some embodiments, in a substantially racemic (or chirally uncontrolled) preparation of oligonucleotides, all or most coupling steps are not chirally controlled in that the coupling steps are not specifically conducted to provide enhanced stereoselectivity. An example substantially racemic preparation of oligonucleotides is the preparation of phosphorothioate oligonucleotides through sulfurizing phosphite triesters from commonly used phosphoramidite oligonucleotide synthesis with either tetraethylthiuram disulfide or (TETD) or 3H-1, 2-bensodithiol-3-one 1, 1-dioxide (BDTD), a well-known process in the art. In some embodiments, substantially racemic preparation of oligonucleotides provides substantially racemic oligonucleotide compositions (or chirally uncontrolled oligonucleotide compositions).

In some embodiments, at least one coupling of a nucleotide monomer has a diastereoselectivity lower than about 60:40, 70:30, 80:20, 85:15, 90:10, 91:9, 92:8, 97:3, 98:2, or 99:1. In some embodiments, in a stereorandom or racemic preparations, at least one internucleotidic linkage has a diastereoselectivity lower than about 60:40, 70:30, 80:20, 85:15, 90:10, 91:9, 92:8, 97:3, 98:2, or 99:1.

In some embodiments, a chirally controlled internucleotidic linkage, such as those of oligonucleotides of chirally controlled oligonucleotide compositions, has a diastereoselectivity of 90:10, 91:9, 92:8, 93:7, 94:6, 95:5, 96:4, 97:3, 98:2, 99:1 or more.

As understood by a person having ordinary skill in the art, in some embodiments, diastereoselectivity of a coupling or a linkage can be assessed through the diastereoselectivity of a dimer formation under the same or comparable conditions, wherein the dimer has the same 5′- and 3′-nucleosides and internucleotidic linkage.

In some embodiments, the present disclosure provides chirally controlled (and/or stereochemically pure) oligonucleotide compositions comprising a first plurality of oligonucleotides defined by having:
1) a common base sequence and length;
2) a common pattern of backbone linkages; and
3) a common pattern of backbone chiral centers, which composition is a substantially pure preparation of a single oligonucleotide in that at least about 10% of the oligonucleotides in the composition have the common base sequence and length, the common pattern of backbone linkages, and the common pattern of backbone chiral centers.

In some embodiments, the present disclosure provides chirally controlled oligonucleotide composition of a first plurality of oligonucleotides in that the composition is enriched, relative to a substantially racemic preparation of the same oligonucleotides, for oligonucleotides of a single oligonucleotide type. In some embodiments, the present disclosure provides chirally controlled oligonucleotide composition of a first plurality of oligonucleotides in that the composition is enriched, relative to a substantially racemic preparation of the same oligonucleotides, for oligonucleotides of a single oligonucleotide type that share:
1) a common base sequence and length;
2) a common pattern of backbone linkages; and
3) a common pattern of backbone chiral centers.

In some embodiments, the present disclosure provides an oligonucleotide composition comprising a first plurality of oligonucleotides capable of directing single-stranded RNA interference, wherein oligonucleotides are of a particular oligonucleotide type characterized by:
1) a common base sequence and length;
2) a common pattern of backbone linkages; and
3) a common pattern of backbone chiral centers;
   which composition is chirally controlled in that it is enriched, relative to a substantially racemic preparation of oligonucleotides having the same base sequence and length, for oligonucleotides of the particular oligonucleotide type.

In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have identical structures.

In some embodiments, oligonucleotides of an oligonucleotide type have a common pattern of backbone phosphorus modifications and a common pattern of sugar modifications. In some embodiments, oligonucleotides of an oligonucleotide type have a common pattern of backbone phosphorus modifications and a common pattern of base modifications. In some embodiments, oligonucleotides of an oligonucleotide type have a common pattern of backbone phosphorus modifications and a common pattern of nucleoside modifications. In some embodiments, oligonucleotides of an oligonucleotide type are identical.

In some embodiments, at least about 20% of the oligonucleotides in the composition have a common base sequence and length, a common pattern of backbone linkages, and a common pattern of backbone chiral centers. In some embodiments, at least about 25% of the oligonucleotides in the composition have a common base sequence and length, a common pattern of backbone linkages, and a common pattern of backbone chiral centers. In some embodiments, at least about 30% of the oligonucleotides in the composition have a common base sequence and length, a common pattern of backbone linkages, and a common pattern of backbone chiral centers. In some embodiments, at least about 35% of the oligonucleotides in the composition have a common base sequence and length, a common pattern of backbone linkages, and a common pattern of backbone chiral centers. In some embodiments, at least about 40% of the oligonucleotides in the composition have a common base sequence and length, a common pattern of backbone linkages, and a common pattern of backbone chiral centers. In some embodiments, at least about 45% of the oligonucleotides in the composition have a common base sequence and length, a common pattern of backbone linkages, and a common pattern of backbone chiral centers. In some embodiments, at least about 50% of the oligonucleotides in the composition have a common base sequence and length, a common pattern of backbone linkages, and a common pattern of backbone chiral centers. In some embodiments, at least about 55% of the oligonucleotides in the composition have a common base sequence and length, a common pattern of backbone linkages, and a common pattern of backbone chiral centers. In some embodiments, at least about 60% of the oligonucleotides in the composition have a common base sequence and length, a common pattern of backbone linkages, and a common pattern of backbone chiral centers. In some embodiments, at least about 65% of the oligonucleotides in the composition have a common base sequence and length, a common pattern of backbone linkages, and a common pattern of backbone chiral centers. In some embodiments, at least about 70% of the oligonucleotides in the composition have a common base sequence and length, a common pattern of backbone linkages, and a common pattern of backbone chiral centers. In some embodiments, at least about 75% of the oligonucleotides in the composition have a common base sequence and length, a common pattern of backbone linkages, and a common pattern of backbone chiral centers. In some embodiments, at least about 80% of the oligonucleotides in the composition have a common base sequence and length, a common pattern of backbone linkages, and a common pattern of backbone chiral centers. In some embodiments, at least about 85% of the oligonucleotides in the composition have a common base sequence and length, a common pattern of backbone linkages, and a common pattern of backbone chiral centers. In some embodiments, at least about 90% of the oligonucleotides in the composition have a common base sequence and length, a common pattern of backbone linkages, and a common pattern of backbone chiral centers. In some embodiments, at least about 92% of the oligonucleotides in the composition have a common base sequence and length, a common pattern of backbone linkages, and a common pattern of backbone chiral centers. In some embodiments, at least about 94% of the oligonucleotides in the composition have a common base sequence and length, a common pattern of backbone linkages, and a common pattern of backbone chiral centers. In some embodiments, at least about 95% of the oligonucleotides in the composition have a common base sequence and length, a common pattern of backbone linkages, and a common pattern of backbone chiral centers. In some embodiments, at least about 91%, 92%, 93%, 94%, 95%, 96%, 97%, 98%, or 99% of the oligonucleotides in the composition have a common base sequence and length, a common pattern of backbone linkages, and a common pattern of backbone chiral centers. In some embodiments, greater than about 99% of the oligonucleotides in the composition have a common base sequence and length, a common pattern of backbone linkages, and a common pattern of backbone chiral centers. In some embodiments, purity of a single-stranded RNAi agent of an oligonucleotide can be expressed as the percentage of oligonucleotides in the composition that have a common base sequence and length, a common pattern of backbone linkages, and a common pattern of backbone chiral centers.

In some embodiments, oligonucleotides having a common base sequence and length, a common pattern of backbone linkages, and a common pattern of backbone chiral centers have a common pattern of backbone phosphorus modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers have a common pattern of backbone phosphorus modifications and a common pattern of sugar modifications. In some embodiments, oligonucleotides having a common base sequence and length, a common pattern of backbone linkages, and a common pattern of backbone chiral centers have a common pattern of backbone phosphorus modifications and a common pattern of base modifications. In some embodiments, oligonucleotides having a common base sequence and length, a common pattern of backbone linkages, and a common pattern of backbone chiral centers have a common pattern of backbone phosphorus modifications and a common pattern of nucleoside modifications. In some embodiments, oligonucleotides having a common base sequence and length, a common pattern of backbone linkages, and a common pattern of backbone chiral centers are identical.

In some embodiments, oligonucleotides in provided compositions have a common pattern of backbone phosphorus modifications. In some embodiments, a common base sequence is a base sequence of an oligonucleotide type. In some embodiments, a provided composition is an oligonucleotide composition that is chirally controlled in that the composition contains a predetermined level of a first plurality of oligonucleotides of an individual oligonucleotide type, wherein an oligonucleotide type is defined by:
1) base sequence;
2) pattern of backbone linkages;
3) pattern of backbone chiral centers; and
4) pattern of backbone phosphorus modifications.

As noted above and understood in the art, in some embodiments, the base sequence of an oligonucleotide may refer to the identity and/or modification status of nucleoside residues (e.g., of sugar and/or base components, relative to standard naturally occurring nucleotides such as adenine, cytosine, guanosine, thymine, and uracil) in the oligonucleotide and/or to the hybridization character (i.e., the ability to hybridize with particular complementary residues) of such residues.

In some embodiments, oligonucleotides of a particular type are identical in that they have the same base sequence (including length), the same pattern of chemical modifications to sugar and base moieties, the same pattern of backbone linkages (e.g., pattern of natural phosphate linkages, phosphorothioate linkages, phosphorothioate triester linkages, and combinations thereof), the same pattern of backbone chiral centers (e.g., pattern of stereochemistry (Rp/Sp) of chiral internucleotidic linkages), and the same pattern of backbone phosphorus modifications (e.g., pattern of modifications on the internucleotidic phosphorus atom, such as —S⁻, and -L-R¹ of Formula I).

In some embodiments, purity of a single-stranded RNAi agent of an oligonucleotide type is expressed as the percentage of oligonucleotides in the composition that are of the oligonucleotide type. In some embodiments, at least about 10% of the oligonucleotides in a single-stranded RNAi agent are of the same oligonucleotide type. In some embodiments, at least about 50% of the oligonucleotides in a single-stranded RNAi agent are of the same oligonucleotide type. In some embodiments, at least about 60% of the oligonucleotides in a single-stranded RNAi agent are of the same oligonucleotide type. In some embodiments, at least about 70% of the oligonucleotides in a single-stranded RNAi agent are of the same oligonucleotide type. In some embodiments, at least about 80% of the oligonucleotides in a single-stranded RNAi agent are of the same oligonucleotide type. In some embodiments, at least about 90% of the oligonucleotides in a single-stranded RNAi agent are of the same oligonucleotide type.

In some embodiments, purity of a single-stranded RNAi agent can be controlled by stereoselectivity of each coupling step in its preparation process. In some embodiments, a coupling step has a stereoselectivity (e.g., diastereoselectivity) of 60% (60% of the new internucleotidic linkage formed from the coupling step has the intended stereochemistry). After such a coupling step, the new internucleotidic linkage formed may be referred to have a 60% purity. In some embodiments, each coupling step has a stereoselectivity of at least 60%, 70%, 80%, or 90%. In some embodiments, each coupling step has a stereoselectivity of at least 95%. In some embodiments, each coupling step has a stereoselectivity of at least 96%. In some embodiments, each coupling step has a stereoselectivity of at least 97%. In some embodiments, each coupling step has a stereoselectivity of at least 98%. In some embodiments, each coupling step has a stereoselectivity of at least 99%. In some embodiments, each coupling step has a stereoselectivity of at least 99.5%. In some embodiments, each coupling step has a stereoselectivity of virtually 100%. In some embodiments, a coupling step has a stereoselectivity of virtually 100% in that all detectable product from the coupling step by an analytical method (e.g., NMR, HPLC, etc) has the intended stereoselectivity.

Among other things, the present disclosure recognizes that combinations of oligonucleotide structural elements (e.g., patterns of chemical modifications, backbone linkages, backbone chiral centers, and/or backbone phosphorus modifications) can provide surprisingly improved properties such as bioactivities.

In some embodiments, provided chirally controlled (and/or stereochemically pure) preparations are RNAi agent oligonucleotides.

In some embodiments, provided chirally controlled (and/or stereochemically pure) preparations are of oligonucleotides that include one or more modified backbone linkages, bases, and/or sugars.

In some embodiments, provided chirally controlled (and/or stereochemically pure) preparations are of oligonucleotides having a common base sequence of at least 8 bases. In some embodiments, provided chirally controlled (and/or stereochemically pure) preparations are of oligonucleotides having a common base sequence of at least 14 bases. In some embodiments, provided chirally controlled (and/or stereochemically pure) preparations are of oligonucleotides having a common base sequence of at least 15 to 25 bases. In some embodiments, provided chirally controlled (and/or stereochemically pure) preparations are of oligonucleotides having a common base sequence of at least 30, 35, 40, 45, 50, 55, 60, 65, 70, or 75 bases.

In some embodiments, provided compositions comprise oligonucleotides containing one or more residues which are modified at the sugar moiety. In some embodiments, provided compositions comprise oligonucleotides containing one or more residues which are modified at the 2′ position of the sugar moiety (referred to herein as a “2′-modification”). Examples of such modifications are described above and herein and include, but are not limited to, 2′-OMe, 2′-MOE, 2′-LNA, 2′-F, FRNA, FANA, S-cEt, etc. In some embodiments, provided compositions comprise oligonucleotides containing one or more residues which are 2′-modified. For example, in some embodiments, provided oligonucleotides contain one or more residues which are 2′-O-methoxyethyl (2′-MOE)-modified residues. In some embodiments, provided compositions comprise oligonucleotides which do not contain any 2′-modifications. In some embodiments, provided compositions are oligonucleotides which do not contain any 2′-MOE residues. That is, in some embodiments, provided oligonucleotides are not MOE-modified. Additional example sugar modifications are described in the present disclosure.

In some embodiments, one or more is one. In some embodiments, one or more is two. In some embodiments, one or more is three. In some embodiments, one or more is four. In some embodiments, one or more is five. In some embodiments, one or more is six. In some embodiments, one or more is seven. In some embodiments, one or more is eight. In some embodiments, one or more is nine. In some embodiments, one or more is ten. In some embodiments, one or more is at least one. In some embodiments, one or more is at least two. In some embodiments, one or more is at least three. In some embodiments, one or more is at least four. In some embodiments, one or more is at least five. In some embodiments, one or more is at least six. In some embodiments, one or more is at least seven. In some embodiments, one or more is at least eight. In some embodiments, one or more is at least nine. In some embodiments, one or more is at least ten.

In some embodiments, a 5′-internucleotidic linkage connected to a sugar moiety without a 2′-modification is a modified linkage. In some embodiments, a 5′-internucleotidic linkage connected to a sugar moiety without a 2′-modification is a linkage having the structure of Formula I. In some embodiments, a 5′-internucleotidic linkage connected to a sugar moiety without a 2′-modification is phosphorothioate linkage. In some embodiments, a 5′-internucleotidic linkage connected to a sugar moiety without a 2′-modification is a substituted phosphorothioate linkage. In some embodiments, a 5′-internucleotidic linkage connected to a sugar moiety without a 2′-modification is a phosphorothioate triester linkage. In some embodiments, each 5′-internucleotidic linkage connected to a sugar moiety without a 2′-modification is a modified linkage. In some embodiments, each 5′-internucleotidic linkage connected to a sugar moiety without a 2′-modification is a linkage having the structure of Formula I. In some embodiments, each 5′-internucleotidic linkage connected to a sugar moiety without a 2′-modification is phosphorothioate linkage. In some embodiments, each 5′-internucleotidic linkage connected to a sugar moiety without a 2′-modification is a substituted phosphorothioate linkage. In some embodiments, each 5′-internucleotidic linkage connected to a sugar moiety without a 2′-modification is a phosphorothioate triester linkage.

In some embodiments, a 3′-internucleotidic linkage connected to a sugar moiety without a 2′-modification is a modified linkage. In some embodiments, a 3′-internucleotidic linkage connected to a sugar moiety without a 2′-modification is a linkage having the structure of Formula I. In some embodiments, a 3′-internucleotidic linkage connected to a sugar moiety without a 2′-modification is phosphorothioate linkage. In some embodiments, a 3′-internucleotidic linkage connected to a sugar moiety without a 2′-modification is a substituted phosphorothioate linkage. In some embodiments, a 3′-internucleotidic linkage connected to a sugar moiety without a 2′-modification is a phosphorothioate triester linkage. In some embodiments, each 3′-internucleotidic linkage connected to a sugar moiety without a 2′-modification is a modified linkage. In some embodiments, each 3′-internucleotidic linkage connected to a sugar moiety without a 2′-modification is a linkage having the structure of Formula I. In some embodiments, each 3′-internucleotidic linkage connected to a sugar moiety without a 2′-modification is phosphorothioate linkage. In some embodiments, each 3′-internucleotidic linkage connected to a sugar moiety without a 2′-modification is a substituted phosphorothioate linkage. In some embodiments, each 3′-internucleotidic linkage connected to a sugar moiety without a 2′-modification is a phosphorothioate triester linkage.

In some embodiments, both internucleotidic linkages connected to a sugar moiety without a 2′-modification are modified linkages. In some embodiments, both internucleotidic linkages connected to a sugar moiety without a 2′-modification are linkage having the structure of Formula I. In some embodiments, both internucleotidic linkages connected to a sugar moiety without a 2′-modification are phosphorothioate linkages. In some embodiments, both internucleotidic linkages connected to a sugar moiety without a 2′-modification are substituted phosphorothioate linkages. In some embodiments, both internucleotidic linkages connected to a sugar moiety without a 2′-modification are phosphorothioate triester linkages. In some embodiments, each internucleotidic linkage connected to a sugar moiety without a 2′-modification is a modified linkage. In some embodiments, each internucleotidic linkage connected to a sugar moiety without a 2′-modification is a linkage having the structure of Formula I. In some embodiments, each internucleotidic linkage connected to a sugar moiety without a 2′-modification is phosphorothioate linkage. In some embodiments, each internucleotidic linkage connected to a sugar moiety without a 2′-modification is a substituted phosphorothioate linkage. In some embodiments, each internucleotidic linkage connected to a sugar moiety without a 2′-modification is a phosphorothioate triester linkage.

In some embodiments, a sugar moiety without a 2′-modification is a sugar moiety found in a natural DNA nucleoside.

A person of ordinary skill in the art understands that various regions of a target transcript can be targeted by provided compositions and methods. In some embodiments, a base sequence of provided oligonucleotides comprises an intron sequence. In some embodiments, a base sequence of provided oligonucleotides comprises an exon sequence. In some embodiments, a base sequence of provided oligonucleotides comprises an intron and an exon sequence.

As understood by a person having ordinary skill in the art, provided oligonucleotides and compositions, among other things, can target a great number of nucleic acid polymers. For instance, in some embodiments, provided oligonucleotides and compositions may target a transcript of a nucleic acid sequence, wherein a common base sequence of oligonucleotides (e.g., a base sequence of an oligonucleotide type) comprises or is a sequence complementary to a sequence of the transcript. In some embodiments, a common base sequence comprises a sequence complimentary to a sequence of a target. In some embodiments, a common base sequence is a sequence complimentary to a sequence of a target. In some embodiments, a common base sequence comprises or is a sequence 100% complimentary to a sequence of a target. In some embodiments, a common base sequence comprises a sequence 100% complimentary to a sequence of a target. In some embodiments, a common base sequence is a sequence 100% complimentary to a sequence of a target.

In some embodiments, as described in this disclosure, provided oligonucleotides and compositions may provide new cleavage patterns, higher cleavage rate, higher cleavage degree, higher cleavage selectivity, etc. In some embodiments, provided compositions can selectively suppress (e.g., cleave) a transcript from a target nucleic acid sequence which has one or more similar sequences exist within a subject or a population, each of the target and its similar sequences contains a specific nucleotidic characteristic sequence element that defines the target sequence relative to the similar sequences. In some embodiments, for example, a target sequence is a wild-type allele or copy of a gene, and a similar sequence is a sequence has very similar base sequence, e.g., a sequence having SNP, mutations, etc.

In some embodiments, a similar sequence has greater than 60%, 65%, 70%, 75%, 80%, 85%, 90%, 91%, 92%, 93%, 94%, 95%, 96%, 97%, 98% or 99% sequence homology with a target sequence. In some embodiments, a target sequence is a disease-causing copy of a nucleic acid sequence comprising one or more mutations and/or SNPs, and a similar sequence is a copy not causing the disease (wild type). In some embodiments, a target sequence comprises a mutation, wherein a similar sequence is the corresponding wild-type sequence. In some embodiments, a target sequence is a mutant allele, while a similar sequence is a wild-type allele. In some embodiments, a target sequence comprises an SNP that is associated with a disease-causing allele, while a similar sequence comprises the same SNP that is not associates with the disease-causing allele. In some embodiments, the region of a target sequence that is complementary to a common base sequence of a provided oligonucleotide composition has greater than 60%, 65%, 70%, 75%, 80%, 85%, 90%, 91%, 92%, 93%, 94%, 95%, 96%, 97%, 98% or 99% sequence homology with the corresponding region of a similar sequence. In some embodiments, the region of a target sequence that is complementary to a common base sequence of a provided oligonucleotide composition differs from the corresponding region of a similar sequence at less than 5, less than 4, less than 3, less than 2, or only 1 base pairs. In some embodiments, the region of a target sequence that is complementary to a common base sequence of a provided oligonucleotide composition differs from the corresponding region of a similar sequence only at a mutation site or SNP site. In some embodiments, the region of a target sequence that is complementary to a common base sequence of a provided oligonucleotide composition differs from the corresponding region of a similar sequence only at a mutation site. In some embodiments, the region of a target sequence that is complementary to a common base sequence of a provided oligonucleotide composition differs from the corresponding region of a similar sequence only at an SNP site.

In some embodiments, a common base sequence comprises or is a sequence complementary to a characteristic sequence element. In some embodiments, a common base sequence comprises a sequence complementary to a characteristic sequence element. In some embodiments, a common base sequence is a sequence complementary to a characteristic sequence element. In some embodiments, a common base sequence comprises or is a sequence 100% complementary to a characteristic sequence element. In some embodiments, a common base sequence comprises a sequence 100% complementary to a characteristic sequence element. In some embodiments, a common base sequence is a sequence 100% complementary to a characteristic sequence element. In some embodiments herein, a characteristic sequence element is, as non-limiting examples, a seed region, a post-seed region or a portion of a seed region, or a portion of a post-seed region or a 3′-terminal dinucleotide.

In some embodiments, a characteristic sequence element comprises or is a mutation. In some embodiments, a characteristic sequence element comprises a mutation. In some embodiments, a characteristic sequence element is a mutation. In some embodiments, a characteristic sequence element comprises or is a point mutation. In some embodiments, a characteristic sequence element comprises a point mutation. In some embodiments, a characteristic sequence element is a point mutation. In some embodiments, a characteristic sequence element comprises or is an SNP. In some embodiments, a characteristic sequence element comprises an SNP. In some embodiments, a characteristic sequence element is an SNP.

In some embodiments, a common base sequence 100% matches a target sequence, which it does not 100% match a similar sequence of the target sequence.

For example, in some embodiments, a common base sequence matches a mutation in the disease-causing copy or allele of a target nucleic acid sequence, but does not match a non-disease-causing copy or allele at the mutation site; in some other embodiments, a common base sequence matches an SNP in the disease-causing allele of a target nucleic acid sequence, but does not match a non-disease-causing allele at the corresponding site.

Among other things, the present disclosure recognizes that a base sequence may have impact on oligonucleotide properties. In some embodiments, a base sequence may have impact on cleavage pattern of a target when oligonucleotides having the base sequence are utilized for suppressing a target, e.g., through a pathway involving RNase H: for example, structurally similar (all phosphorothioate linkages, all stereorandom) oligonucleotides have different sequences may have different cleavage patterns.

In some embodiments, a common base sequence is a base sequence that comprises a SNP.

As a person having ordinary skill in the art understands, provided oligonucleotide compositions and methods have various uses as known by a person having ordinary skill in the art. Methods for assessing provided compositions, and properties and uses thereof, are also widely known and practiced by a person having ordinary skill in the art. Example properties, uses, and/or methods include but are not limited to those described in WO/2014/012081 and WO/2015/107425.

In some embodiments, a common base sequence comprises or is a sequence complementary to a nucleic acid sequence. In some embodiments, a common base sequence comprises or is a sequence 100% complementary to a nucleic acid sequence. In some embodiments, a common base sequence comprises or is a sequence complementary to a disease-causing or disease-related nucleic acid sequence. In some embodiments, a common base sequence comprises or is a sequence 100% complementary to a disease-causing nucleic acid sequence. In some embodiments, a common base sequence comprises or is a sequence complementary to a characteristic sequence element of disease-causing nucleic acid sequence, which characteristic sequences differentiate a disease-causing nucleic acid sequence from a non-diseasing-causing nucleic acid sequence. In some embodiments, a common base sequence comprises or is a sequence 100% complementary to a characteristic sequence element of disease-causing nucleic acid sequence, which characteristic sequences differentiate a disease-causing nucleic acid sequence from a non-diseasing-causing nucleic acid sequence. In some embodiments, a common base sequence comprises or is a sequence complementary to a disease-associated nucleic acid sequence. In some embodiments, a common base sequence comprises or is a sequence 100% complementary to a disease-associated nucleic acid sequence. In some embodiments, a common base sequence comprises or is a sequence complementary to a characteristic sequence element of disease-associated nucleic acid sequence, which characteristic sequences differentiate a disease-associated nucleic acid sequence from a non-diseasing-associated nucleic acid sequence. In some embodiments, a common base sequence comprises or is a sequence 100% complementary to a characteristic sequence element of disease-associated nucleic acid sequence, which characteristic sequences differentiate a disease-associated nucleic acid sequence from a non-diseasing-associated nucleic acid sequence.

In some embodiments, a common base sequence comprises or is a sequence complementary to a gene. In some embodiments, a common base sequence comprises or is a sequence 100% complementary to a gene. In some embodiments, a common base sequence comprises or is a sequence complementary to a characteristic sequence element of a gene, which characteristic sequences differentiate the gene from a similar sequence sharing homology with the gene. In some embodiments, a common base sequence comprises or is a sequence 100% complementary to a characteristic sequence element of a gene, which characteristic sequences differentiate the gene from a similar sequence sharing homology with the gene. In some embodiments, a common base sequence comprises or is a sequence complementary to characteristic sequence element of a target gene, which characteristic sequences comprises a mutation that is not found in other copies of the gene, e.g., the wild-type copy of the gene, another mutant copy the gene, etc. In some embodiments, a common base sequence comprises or is a sequence 100% complementary to characteristic sequence element of a target gene, which characteristic sequences comprises a mutation that is not found in other copies of the gene, e.g., the wild-type copy of the gene, another mutant copy the gene, etc.

In some embodiments, a chiral internucleotidic linkage has the structure of Formula I. In some embodiments, a chiral internucleotidic linkage is phosphorothioate. In some embodiments, each chiral internucleotidic linkage in a single oligonucleotide of a provided composition independently has the structure of Formula I. In some embodiments, each chiral internucleotidic linkage in a single oligonucleotide of a provided composition is a phosphorothioate.

In some embodiments, oligonucleotides of the present disclosure comprise one or more modified sugar moieties. In some embodiments, oligonucleotides of the present disclosure comprise one or more modified base moieties. As known by a person of ordinary skill in the art and described in the disclosure, various modifications can be introduced to a sugar and/or moiety. For example, in some embodiments, a modification is a modification described in U.S. Pat. No. 9,006,198, WO2014/012081 and WO/2015/107425, the sugar and base modifications of each of which are incorporated herein by reference.

In some embodiments, a sugar modification is a 2′-modification. Commonly used 2′-modifications include but are not limited to 2′-OR¹, wherein R¹ is not hydrogen. In some embodiments, a modification is 2′-OR, wherein R is optionally substituted aliphatic. In some embodiments, a modification is 2′-OMe. In some embodiments, a modification is 2′-O-MOE. In some embodiments, the present disclosure demonstrates that inclusion and/or location of particular chirally pure internucleotidic linkages can provide stability improvements comparable to or better than those achieved through use of modified backbone linkages, bases, and/or sugars. In some embodiments, a provided single oligonucleotide of a provided composition has no modifications on the sugars. In some embodiments, a provided single oligonucleotide of a provided composition has no modifications on 2′-positions of the sugars (i.e., the two groups at the 2′-position are either —H/—H or —H/—OH). In some embodiments, a provided single oligonucleotide of a provided composition does not have any 2′-MOE modifications.

In some embodiments, a 2′-modification is —O-L- or -L- which connects the 2′-carbon of a sugar moiety to another carbon of a sugar moiety. In some embodiments, a 2′-modification is —O-L- or -L- which connects the 2′-carbon of a sugar moiety to the 4′-carbon of a sugar moiety. In some embodiments, a 2′-modification is S-cEt. In some embodiments, a modified sugar moiety is an LNA moiety.

In some embodiments, a 2′-modification is —F. In some embodiments, a 2′-modification is FANA. In some embodiments, a 2′-modification is FRNA.

In some embodiments, a sugar modification is a 5′-modification, e.g., R-5′-Me, S-5′-Me, etc.

In some embodiments, a sugar modification changes the size of the sugar ring. In some embodiments, a sugar modification is the sugar moiety in FHNA.

In some embodiments, a sugar modification replaces a sugar moiety with another cyclic or acyclic moiety. Examples of such moieties are widely known in the art, including but not limited to those used in morpholino (optionally with its phosphorodiamidate linkage), glycol nucleic acids, etc.

In some embodiments, a single oligonucleotide in a provided composition has at least about 25% of its internucleotidic linkages in Sp configuration. In some embodiments, a single oligonucleotide in a provided composition has at least about 30% of its internucleotidic linkages in Sp configuration. In some embodiments, a single oligonucleotide in a provided composition has at least about 35% of its internucleotidic linkages in Sp configuration. In some embodiments, a single oligonucleotide in a provided composition has at least about 40% of its internucleotidic linkages in Sp configuration. In some embodiments, a single oligonucleotide in a provided composition has at least about 45% of its internucleotidic linkages in Sp configuration. In some embodiments, a single oligonucleotide in a provided composition has at least about 50% of its internucleotidic linkages in Sp configuration. In some embodiments, a single oligonucleotide in a provided composition has at least about 55% of its internucleotidic linkages in Sp configuration. In some embodiments, a single oligonucleotide in a provided composition has at least about 60% of its internucleotidic linkages in Sp configuration. In some embodiments, a single oligonucleotide in a provided composition has at least about 65% of its internucleotidic linkages in Sp configuration. In some embodiments, a single oligonucleotide in a provided composition has at least about 70% of its internucleotidic linkages in Sp configuration. In some embodiments, a single oligonucleotide in a provided composition has at least about 75% of its internucleotidic linkages in Sp configuration. In some embodiments, a single oligonucleotide in a provided composition has at least about 80% of its internucleotidic linkages in Sp configuration. In some embodiments, a single oligonucleotide in a provided composition has at least about 85% of its internucleotidic linkages in Sp configuration. In some embodiments, a single oligonucleotide in a provided composition has at least about 90% of its internucleotidic linkages in Sp configuration.

In some embodiments, an ssRNAi agent is or comprises an oligonucleotide selected from the group consisting of WV-1275, WV-1277, WV-1307, WV-1308, WV-1308, WV-1828, WV-1829, WV-1830, WV-1831, WV-2110, WV-2110, WV-2110, WV-2110, WV-2110, WV-2110, WV-2111, WV-2111, WV-2112, WV-2113, WV-2113, WV-2146, WV-2147, WV-2148, WV-2149, WV-2150, WV-2151, WV-2152, WV-2153, WV-2154, WV-2155, WV-2156, WV-2157, WV-2386, WV-2386, WV-2420, WV-2477, WV-2652, WV-2653, WV-2654, WV-2655, WV-2656, WV-2657, WV-2658, WV-2693, WV-2696, WV-2697, WV-2698, WV-2699, WV-2712, WV-2713, WV-2714, WV-2715, WV-2716, WV-2717, WV-2718, WV-2719, WV-2720, WV-2721, WV-2721, WV-2817, WV-2818, WV-3021, WV-3068, WV-3069, WV-3107, WV-3122, WV-3122, WV-3124 to WV-3127, WV-3133 to WV-3137, WV-3242, WV-3247, WV-3755, WV-3755 to WV-3764, WV-3756, WV-3757, WV-3758, WV-3759, WV-3760, WV-3761, WV-3762, WV-3763, WV-3764, WV-3981, WV-3981 to WV-3985, WV-3982, WV-3983, WV-3984, WV-3985, WV-4007, WV-4007 to WV-4011, WV-4008, WV-4009, WV-4010, WV-4011, WV-4012, WV-4012 to WV-4017, WV-4013, WV-4014, WV-4015, WV-4016, WV-4017, WV-4018, WV-4019, WV-4020, WV-4021, WV-4022, WV-4023, WV-4024, WV-4025, WV-4026, WV-4027, WV-4028, WV-4029, WV-4030, WV-4031, WV-4032, WV-4033, WV-4034, WV-4035, WV-4036, WV-4037, WV-4038, WV-4039, WV-4040, WV-4041, WV-4042, WV-4043, WV-4044, WV-4045, WV-4046, WV-4047, WV-4048, WV-4049, WV-4050, WV-4051, WV-4052, WV-4053, WV-4054, WV-4055, WV-4056, WV-4057, WV-4058, WV-4059, WV-4060, WV-4061, WV-4062, WV-4063, WV-4064, WV-4065, WV-4075, WV-4098, WV-4161, WV-4264, WV-4264 to WV-4267, WV-4265, WV-4266, WV-4267, WV-4268, WV-4268 to WV-4277, WV-4269, WV-4270, WV-4271, WV-4272, WV-4273, WV-4274, WV-4275, WV-4276, WV-4277, WV-5288, WV-5289, WV-5289, WV-5290, WV-5291, WV-5292, WV-5293, WV-5294, WV-5295, WV-5296, WV-5297, WV-5298, WV-5299, WV-5300, WV-5301, WV-6214, WV-6215, WV-6411 to 6430, WV-6431, WV-6431 to WV-6438, WV-6439, WV-6763, WV-6764, WV-6764, WV-6765, WV-6766, WV-7316, WV-7462, WV-7462, WV-7463, WV-7464, WV-7465, WV-7465, WV-7466, WV-7466, WV-7467, WV-7468, WV-7468, WV-7469, WV-7521, WV-7522, WV-7523, WV-7524, WV-7525, WV-7526, WV-7527, WV-7528, WV-7540 to WV-7544, WV-7542, WV-7542, WV-7635, WV-7636, WV-7637, WV-7638, WV-7639, WV-7640, WV-7641, WV-7642, WV-7643, WV-7644, WV-7645, WV-7646, WV-7647, WV-7648, WV-7649, WV-7650, WV-7672, WV-7673, WV-2111, WV-2113, WV-2114, WV-2148, WV-2149, WV-2152, WV-2153, WV-2156, WV-2157, WV-2387, WV-3069, WV-7523, WV-7524, WV-7525, WV-7526, WV-7527, WV-7528, and any ssRNAi of any format described in FIG. 1 or otherwise herein.

In some embodiments, an ssRNAi agent is or comprises an oligonucleotide selected from the group consisting of any ssRNAi of any format described in FIG. 1 or otherwise herein. Those skilled in the art, reading the present specification, will appreciate that the present disclosure specifically does not exclude the possibility that any oligonucleotide described herein which is labeled as a ssRNAi agent may also or alternatively operate through another mechanism (e.g., as an antisense oligonucleotide; mediating knock-down via a RNaseH mechanism; sterically hindering translation; or any other biochemical mechanism).

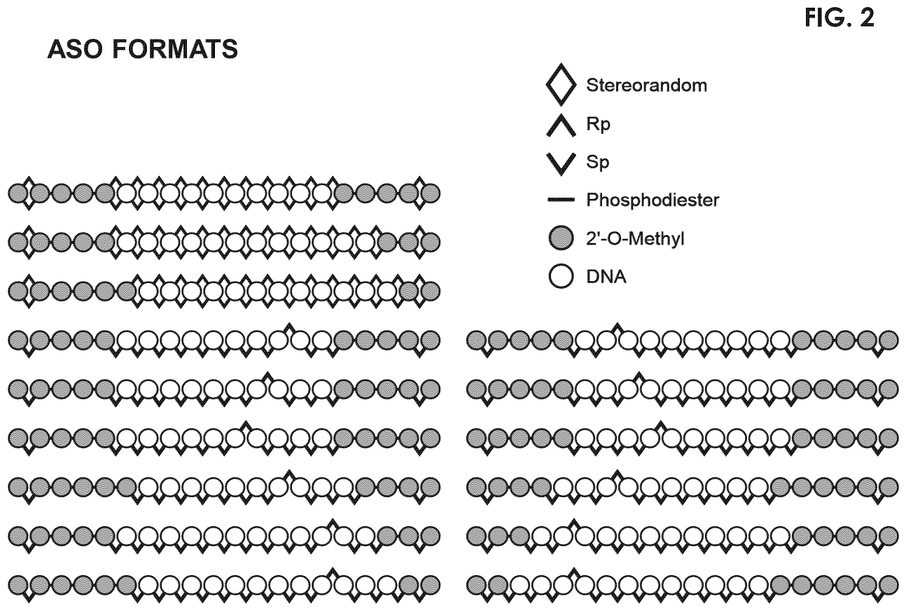

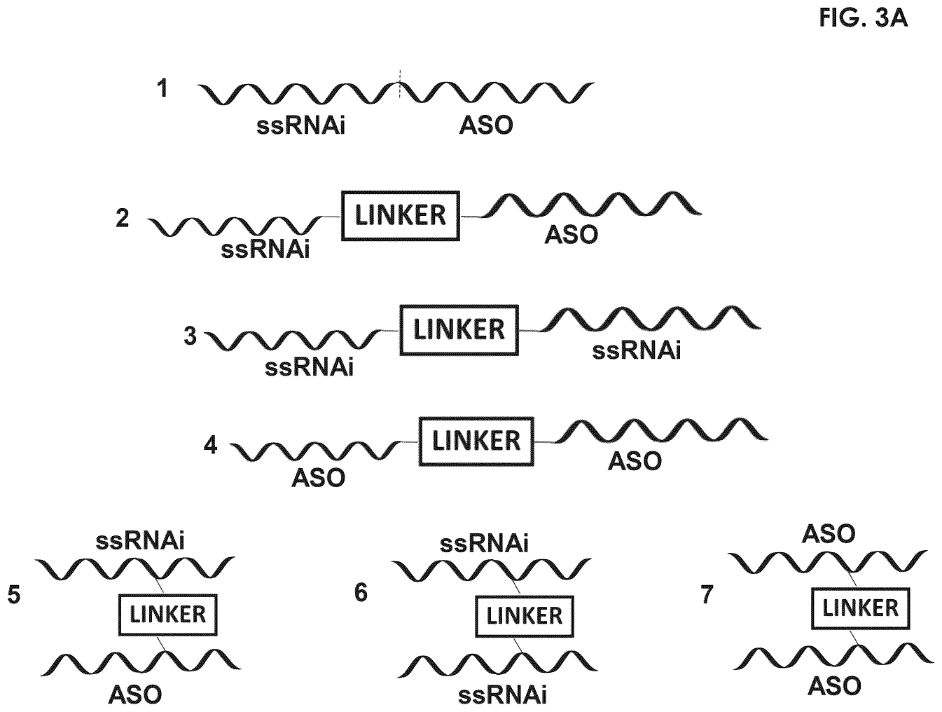

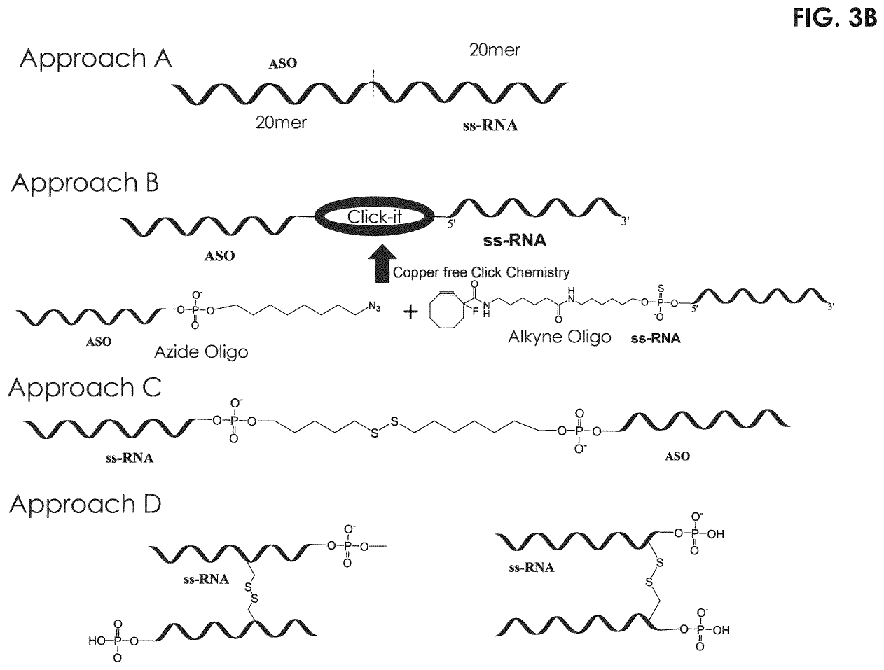

In some embodiments, an antisense oligonucleotide (ASO) is or comprises an oligonucleotide selected from the group consisting of: WV-1308, WV-1391 to WV-1481, WV-1422, WV-1434 WV-1436, WV-1441, WV-1443, WV-1452, WV-1850 to WV-1891, WV-1863, WV-1864, WV-1868, WV-1870 WV-1871, WV-1876, WV-1878, WV-1883, WV-1884, WV-1885, WV-1886, WV-1887, WV-2110, WV-2111, WV-2114, WV-2115 to WV-2124, WV-2126, WV-2128 to WV-2139, WV-2134, WV-2134, WV-2141 WV-2372, WV-2372, WV-2386, WV-2387, WV-2420 WV-2477 WV-2549, WV-2549 to WV-2554, WV-2549 to WV-2554, WV-2550, WV-2551, WV-2552, WV-2553, WV-2553, WV-2554, WV-2554, WV-2644, WV-2645, WV-2646, WV-2647, WV-2677, WV-2678, WV-2678, WV-2722, WV-2723, WV-2724, WV-2725, WV-2726, WV-2727, WV-3021, WV-3367 to WV-3380, WV-3380, WV-3381, WV-3381 to WV-3394, WV-3387 WV-3387, WV-3390, WV-3391, WV-3392, WV-3393, WV-3394, WV-3394, WV-3394, WV-3395 to WV-3408, WV-3398, WV-3399, WV-3399, WV-3402, WV-3404, WV-3408, WV-3409 to WV-3422, WV-3411, WV-3413, WV-3416, WV-3421, WV-3423 to WV-3436, WV-3433, WV-3437 to WV-3450, WV-3443, WV-3443, WV-3451, WV-3452, WV-3453, WV-3454, WV-3455, WV-3456, WV-3457, WV-3458, WV-3459, WV-3460, WV-3461, WV-3462, WV-3858 to WV-3864, WV-3860 to WV-3864, WV-3860 to WV-3864, WV-3968, WV-4054, WV-437, WV-6003, WV-6003, WV-6822, WV-6823, WV-6824, WV-6825, WV-692 to WV-777, WV-723, WV-737, WV-742, WV-744, WV-753, WV-7778 to WV-7793, WV-7794 to WV-7816, WV-779 to WV-787, WV-7804 to WV-7808, WV-7804 to WV-7808, WV-7817 to WV-7839, WV-7827 to WV-7831, WV-7827 to WV-7831, WV-7840 to WV-7862, WV-7850 to WV-7854, WV-7850 to WV-7854, WV-788 to WV-873, WV-8030, WV-8044, WV-8111, WV-8112, WV-819, WV-833, WV-838, WV-840, WV-849, WV-875 to WV-883, WV-993, WV-1868, WV-2134, WV-3367, WV-3368, WV-3369, WV-3370, WV-3371, WV-3372, WV-3373, WV-3374, WV-3375, WV-3376, WV-3377, WV-3378, WV-3379, WV-3380, WV-3387, WV-6825, WV-2111, WV-2113, WV-2114, WV-2148, WV-2149, WV-2152, WV-2153, WV-2156, WV-2157, WV-2387, WV-3069, WV-7523, WV-7524, WV-7525, WV-7526, WV-7527, WV-7528, and any oligonucleotide of any format described in FIG. 2.

In some embodiments, an antisense oligonucleotide (ASO) is or comprises an oligonucleotide selected from the group consisting of any oligonucleotide of any format described in FIG. 2. Those skilled in the art, reading the present specification, will appreciate that the present disclosure specifically does not exclude the possibility that any oligonucleotide described herein which is labeled as an antisense oligonucleotide (ASO) may also or alternatively operate through another mechanism (e.g., as a ssRNAi utilizing RISC); the disclosure also notes that various ASOs may operate via different mechanisms (utilizing RNaseH, sterically blocking translation or other post-transcriptional processes, changing the conformation of a target nucleic acid, etc.).

In some embodiments, a hybrid oligonucleotide is or comprises an oligonucleotide selected from the group consisting of: WV-2111, WV-2113, WV-2114, WV-2148, WV-2149, WV-2152, WV-2153, WV-2156, WV-2157, WV-2387, WV-3069, WV-7523, WV-7524, WV-7525, WV-7526, WV-7527, WV-7528, and any oligonucleotide of any of Formats S40 to S42 of FIG. 1L; or Formats 30-32, 66-69 or 101-103 of FIG. 1. Those skilled in the art, reading the present specification, will appreciate that the present disclosure specifically does not exclude the possibility that any oligonucleotide described herein which is labeled as a hybrid oligonucleotide may also or alternatively operate through another mechanism (e.g., as an antisense oligonucleotide; mediating knock-down via a RNaseH mechanism; sterically hindering translation; or any other biochemical mechanism).

### Chirally Controlled Oligonucleotides and Chirally Controlled Oligonucleotide Compositions

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides are chirally controlled.

The present disclosure provides chirally controlled oligonucleotides, and chirally controlled oligonucleotide compositions which are of high crude purity and of high diastereomeric purity. In some embodiments, the present disclosure provides chirally controlled oligonucleotides, and chirally controlled oligonucleotide compositions which are of high crude purity. In some embodiments, the present disclosure provides chirally controlled oligonucleotides, and chirally controlled oligonucleotide compositions which are of high diastereomeric purity.

In some embodiments, a single-stranded RNAi agent is a substantially pure preparation of an oligonucleotide type in that oligonucleotides in the composition that are not of the oligonucleotide type are impurities form the preparation process of said oligonucleotide type, in some case, after certain purification procedures.

In some embodiments, the present disclosure provides oligonucleotides comprising one or more diastereomerically pure internucleotidic linkages with respect to the chiral linkage phosphorus. In some embodiments, the present disclosure provides oligonucleotides comprising one or more diastereomerically pure internucleotidic linkages having the structure of Formula I. In some embodiments, the present disclosure provides oligonucleotides comprising one or more diastereomerically pure internucleotidic linkages with respect to the chiral linkage phosphorus, and one or more phosphate diester linkages. In some embodiments, the present disclosure provides oligonucleotides comprising one or more diastereomerically pure internucleotidic linkages having the structure of Formula I, and one or more phosphate diester linkages. In some embodiments, the present disclosure provides oligonucleotides comprising one or more diastereomerically pure internucleotidic linkages having the structure of Formula I-c, and one or more phosphate diester linkages. In some embodiments, such oligonucleotides are prepared by using stereoselective oligonucleotide synthesis, as described in this application, to form pre-designed diastereomerically pure internucleotidic linkages with respect to the chiral linkage phosphorus. Example internucleotidic linkages, including those having structures of Formula I, are further described below.

In some embodiments, the present disclosure provides a chirally controlled oligonucleotide, wherein at least two of the individual internucleotidic linkages within the oligonucleotide have different stereochemistry and/or different P-modifications relative to one another.

Internucleotidic Linkages

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any internucleotidic linkage described herein or known in the art.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any internucleotidic linkage described herein or known in the art.

A non-limiting example of an internucleotidic linkage or unmodified internucleotidic linkage is a phosphodiester; non-limiting examples of modified internucleotidic linkages include those in which one or more oxygen of a phosphodiester has been replaced by, as non-limiting examples, sulfur (as in a phosphorothioate), H, alkyl, or another moiety or element which is not oxygen. A non-limiting example of an internucleotidic linkage is a moiety which does not a comprise a phosphorus but serves to link two sugars. A non-limiting example of an internucleotidic linkage is a moiety which does not a comprise a phosphorus but serves to link two sugars in the backbone of an oligonucleotide. Disclosed herein are additional non-limiting examples of nucleotides, modified nucleotides, nucleotide analogs, internucleotidic linkages, modified internucleotidic linkages, bases, modified bases, and base analogs, sugars, modified sugars, and sugar analogs, and nucleosides, modified nucleosides, and nucleoside analogs.

In certain embodiments, a internucleotidic linkage has the structure of Formula I:

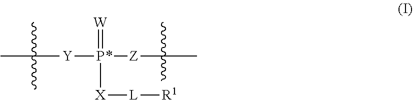

wherein each variable is as defined and described below. In some embodiments, a linkage of Formula I is chiral. In some embodiments, the present disclosure provides a chirally controlled oligonucleotide comprising one or more modified internucleotidic linkages of Formula I. In some embodiments, the present disclosure provides a chirally controlled oligonucleotide comprising one or more modified internucleotidic linkages of Formula I, and wherein individual internucleotidic linkages of Formula I within the oligonucleotide have different P-modifications relative to one another. In some embodiments, the present disclosure provides a chirally controlled oligonucleotide comprising one or more modified internucleotidic linkages of Formula I, and wherein individual internucleotidic linkages of Formula I within the oligonucleotide have different —X-L-R¹ relative to one another. In some embodiments, the present disclosure provides a chirally controlled oligonucleotide comprising one or more modified internucleotidic linkages of Formula I, and wherein individual internucleotidic linkages of Formula I within the oligonucleotide have different X relative to one another. In some embodiments, the present disclosure provides a chirally controlled oligonucleotide comprising one or more modified internucleotidic linkages of Formula I, and wherein individual internucleotidic linkages of Formula I within the oligonucleotide have different -L-R¹ relative to one another. In some embodiments, a chirally controlled oligonucleotide is an oligonucleotide in a provided composition that is of the particular oligonucleotide type. In some embodiments, a chirally controlled oligonucleotide is an oligonucleotide in a provided composition that has the common base sequence and length, the common pattern of backbone linkages, and the common pattern of backbone chiral centers. In some embodiments, a chirally controlled oligonucleotide is an oligonucleotide in a chirally controlled composition that is of the particular oligonucleotide type, and the chirally controlled oligonucleotide is of the type. In some embodiments, a chirally controlled oligonucleotide is an oligonucleotide in a provided composition that comprises a predetermined level of a plurality of oligonucleotides that share a common base sequence, a common pattern of backbone linkages, and a common pattern of backbone chiral centers, and the chirally controlled oligonucleotide shares the common base sequence, the common pattern of backbone linkages, and the common pattern of backbone chiral centers.

In some embodiments, a modified internucleotidic linkage is a non-negatively charged internucleotidic linkage. In some embodiments, a modified internucleotidic linkage is a neutral internucleotidic linkage. In some embodiments, provided oligonucleotides comprise one or more non-negatively charged internucleotidic linkages. In some embodiments, a non-negatively charged internucleotidic linkage is a positively charged internucleotidic linkage. In some embodiments, a non-negatively charged internucleotidic linkage is a neutral internucleotidic linkage. In some embodiments, a modified internucleotidic linkage (e.g., a non-negatively charged internucleotidic linkage) comprises optionally substituted triazolyl. In some embodiments, a modified internucleotidic linkage (e.g., a non-negatively charged internucleotidic linkage) comprises optionally substituted alkynyl. In some embodiments, a modified internucleotidic linkage comprises a triazole or alkyne moiety. In some embodiments, a triazole moiety, e.g., a triazolyl group, is optionally substituted. In some embodiments, a triazole moiety, e.g., a triazolyl group) is substituted. In some embodiments, a triazole moiety is unsubstituted. In some embodiments, a modified internucleotidic linkage comprises an optionally substituted cyclic guanidine moiety. In some embodiments, a modified internucleotidic linkage comprises an optionally substituted cyclic guanidine moiety and has the structure of:

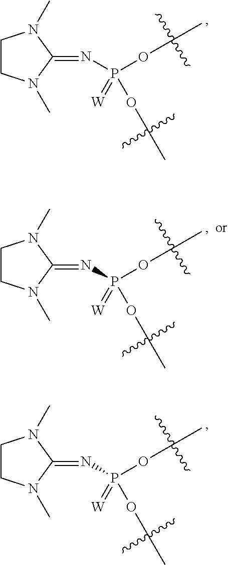

wherein W is O or S. In some embodiments, W is O. In some embodiments, W is S. In some embodiments, a non-negatively charged internucleotidic linkage is stereochemically controlled.

In some embodiments, an internucleotidic linkage comprising a triazole moiety (e.g., an optionally substituted triazolyl group) in a provided oligonucleotide, e.g., a DMD oligonucleotide, has the structure of:

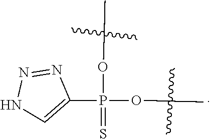 [SMILES: *OP(=S)(O*)c1c[nH]nn1]

In some embodiments an internucleotidic linkage comprising a triazole moiety has the formula of

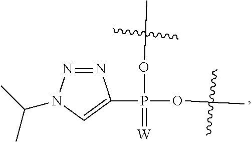

where W is O or S. In some embodiments, an internucleotidic linkage comprising an alkyne moiety (e.g., an optionally substituted alkynyl group) has the formula of:

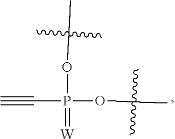

wherein W is O or S. In some embodiments, an internucleotidic linkage comprises a cyclic guanidine moiety. In some embodiments, an internucleotidic linkage comprising a cyclic guanidine moiety has the structure of:

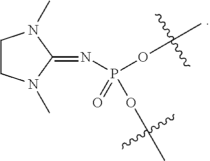 [SMILES: *OP(=O)(N=C1N(C)CCN1C)O*]

In some embodiments, a neutral internucleotidic linkage or internucleotidic linkage comprising a cyclic guanidine moiety is stereochemically controlled.

In some embodiments, a DMD oligonucleotide comprises a lipid moiety In some embodiments, an internucleotidic linkage comprises a Tmg group

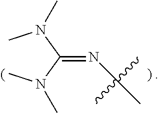 [SMILES: *N=C(N(C)C)N(C)C]

In some embodiments, an internucleotidic linkage comprises a Tmg group and has the structure of

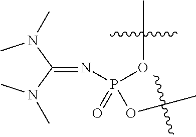 [SMILES: *OP(=O)(N=C(N(C)C)N(C)C)O*]

(the “Tmg internucleotidic linkage”). In some embodiments, neutral internucleotidic linkages include internucleotidic linkages of PNA and PMO, and an Tmg internucleotidic linkage.

In some embodiments, the present disclosure provides a composition comprising a plurality of oligonucleotides of a particular oligonucleotide type defined by:
1) base sequence;
2) pattern of backbone linkages;
3) pattern of backbone chiral centers; and
4) pattern of backbone phosphorus modifications,
   wherein:
oligonucleotides of the plurality comprise at least 1, 2, 3, 4, 5, 6, 7, 8, 9, 10, 11, 12, 13, 14, 15, 16, 17, 18, 19, or 20 chirally controlled internucleotidic linkages; and
oligonucleotides of the plurality comprise at least 1, 2, 3, 4, 5, 6, 7, 8, 9, 10, 11, 12, 13, 14, 15, 16, 17, 18, 19, or 20 non-negatively charged internucleotidic linkages.

In some embodiments, a modified internucleotidic linkage has the structure of formula I, I-a, I-b, I-c, I-n-1, I-n-2, I-n-3, II, II-a-1, II-a-2, II-b-1, II-b-2, II-c-1, II-c-2, II-d-1, II-d-2, etc., or a salt form thereof. In some embodiments, a modified internucleotidic linkage has a structure of formula I or a salt form thereof. In some embodiments, a modified internucleotidic linkage has a structure of formula I-a or a salt form thereof.

In some embodiments, a modified internucleotidic linkage is a non-negatively charged internucleotidic linkage. In some embodiments, a modified internucleotidic linkage is a positively-charged internucleotidic linkage. In some embodiments, a modified internucleotidic linkage is a neutral internucleotidic linkage. In some embodiments, a non-negatively charged internucleotidic linkage has the structure of formula I, I-a, I-b, I-c, I-n-1, I-n-2, I-n-3, II, II-a-1, II-a-2, II-b-1, II-b-2, II-c-1, II-c-2, II-d-1, II-d-2, etc., or a salt form thereof. In some embodiments, a non-negatively charged internucleotidic linkage comprises an optionally substituted 3-20 membered heterocyclyl or heteroaryl group having 1-10 heteroatoms. In some embodiments, a non-negatively charged internucleotidic linkage comprises an optionally substituted 3-20 membered heterocyclyl or heteroaryl group having 1-10 heteroatoms, wherein at least one heteroatom is nitrogen. In some embodiments, such a heterocyclyl or heteroaryl group is of a 5-membered ring. In some embodiments, such a heterocyclyl or heteroaryl group is of a 6-membered ring.

In some embodiments, a non-negatively charged internucleotidic linkage comprises an optionally substituted 5-20 membered heteroaryl group having 1-10 heteroatoms. In some embodiments, a non-negatively charged internucleotidic linkage comprises an optionally substituted 5-20 membered heteroaryl group having 1-10 heteroatoms, wherein at least one heteroatom is nitrogen. In some embodiments, a non-negatively charged internucleotidic linkage comprises an optionally substituted 5-6 membered heteroaryl group having 1-4 heteroatoms, wherein at least one heteroatom is nitrogen. In some embodiments, a non-negatively charged internucleotidic linkage comprises an optionally substituted 5-membered heteroaryl group having 1-4 heteroatoms, wherein at least one heteroatom is nitrogen. In some embodiments, a heteroaryl group is directly bonded to a linkage phosphorus. In some embodiments, a non-negatively charged internucleotidic linkage comprises an optionally substituted triazolyl group. In some embodiments, a non-negatively charged internucleotidic linkage comprises an unsubstituted triazolyl group, e.g.,

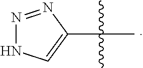 [SMILES: *c1c[nH]nn1]

In some embodiments, a non-negatively charged internucleotidic linkage comprises a substituted triazolyl group, e.g.,

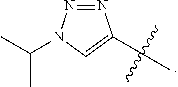 [SMILES: *c1cn(C(C)C)nn1]

In some embodiments, a non-negatively charged internucleotidic linkage comprises an optionally substituted 5-20 membered heterocyclyl group having 1-10 heteroatoms. In some embodiments, a non-negatively charged internucleotidic linkage comprises an optionally substituted 5-20 membered heterocyclyl group having 1-10 heteroatoms, wherein at least one heteroatom is nitrogen. In some embodiments, a non-negatively charged internucleotidic linkage comprises an optionally substituted 5-6 membered heterocyclyl group having 1-4 heteroatoms, wherein at least one heteroatom is nitrogen. In some embodiments, a non-negatively charged internucleotidic linkage comprises an optionally substituted 5-membered heterocyclyl group having 1-4 heteroatoms, wherein at least one heteroatom is nitrogen. In some embodiments, at least two heteroatoms are nitrogen. In some embodiments, a heterocyclyl group is directly bonded to a linkage phosphorus. In some embodiments, a heterocyclyl group is bonded to a linkage phosphorus through a linker, e.g., ═N— when the heterocyclyl group is part of a guanidine moiety who directed bonded to a linkage phosphorus through its ═N—. In some embodiments, a non-negatively charged internucleotidic linkage comprises an optionally substituted

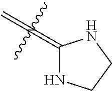 [SMILES: *=C1NCCN1]

group. In some embodiments, a non-negatively charged internucleotidic linkage comprises an substituted

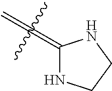 [SMILES: *=C1NCCN1]

group. In some embodiments, a non-negatively charged internucleotidic linkage comprises a

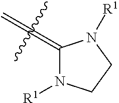

group. In some embodiments, each R¹ is independently optionally substituted C₁₋₆ alkyl. In some embodiments, each R¹ is independently methyl.

In some embodiments, a modified internucleotidic linkage, e.g., a non-negatively charged internucleotidic linkage, comprises a triazole or alkyne moiety, each of which is optionally substituted. In some embodiments, a modified internucleotidic linkage comprises a triazole moiety. In some embodiments, a modified internucleotidic linkage comprises a unsubstituted triazole moiety. In some embodiments, a modified internucleotidic linkage comprises a substituted triazole moiety. In some embodiments, a modified internucleotidic linkage comprises an alkyl moiety. In some embodiments, a modified internucleotidic linkage comprises an optionally substituted alkynyl group. In some embodiments, a modified internucleotidic linkage comprises an unsubstituted alkynyl group. In some embodiments, a modified internucleotidic linkage comprises a substituted alkynyl group. In some embodiments, an alkynyl group is directly bonded to a linkage phosphorus.

In some embodiments, an oligonucleotide comprises different types of internucleotidic phosphorus linkages. In some embodiments, a chirally controlled oligonucleotide comprises at least one natural phosphate linkage and at least one modified (non-natural) internucleotidic linkage. In some embodiments, an oligonucleotide comprises at least one natural phosphate linkage and at least one phosphorothioate. In some embodiments, an oligonucleotide comprises at least one non-negatively charged internucleotidic linkage. In some embodiments, an oligonucleotide comprises at least one natural phosphate linkage and at least one non-negatively charged internucleotidic linkage. In some embodiments, an oligonucleotide comprises at least one phosphorothioate internucleotidic linkage and at least one non-negatively charged internucleotidic linkage. In some embodiments, an oligonucleotide comprises at least one phosphorothioate internucleotidic linkage, at least one natural phosphate linkage, and at least one non-negatively charged internucleotidic linkage. In some embodiments, oligonucleotides comprise one or more, e.g., 1, 2, 3, 4, 5, 6, 7, 8, 9, 10, 11, 12, 13, 14, 15, 16, 17, 18, 19, 20 or more non-negatively charged internucleotidic linkages. In some embodiments, a non-negatively charged internucleotidic linkage is not negatively charged in that at a given pH in an aqueous solution less than 50%, 40%, 40%, 30%, 20%, 10%, 5%, or 1% of the internucleotidic linkage exists in a negatively charged salt form. In some embodiments, a pH is about pH 7.4. In some embodiments, a pH is about 4-9. In some embodiments, the percentage is less than 10%. In some embodiments, the percentage is less than 5%. In some embodiments, the percentage is less than 1%. In some embodiments, an internucleotidic linkage is a non-negatively charged internucleotidic linkage in that the neutral form of the internucleotidic linkage has no pKa that is no more than about 1, 2, 3, 4, 5, 6, or 7 in water. In some embodiments, no pKa is 7 or less. In some embodiments, no pKa is 6 or less. In some embodiments, no pKa is 5 or less. In some embodiments, no pKa is 4 or less. In some embodiments, no pKa is 3 or less. In some embodiments, no pKa is 2 or less. In some embodiments, no pKa is 1 or less. In some embodiments, pKa of the neutral form of an internucleotidic linkage can be represented by pKa of the neutral form of a compound having the structure of CH₃—the internucleotidic linkage-CH₃. For example, pKa of the neutral form of an internucleotidic linkage having the structure of formula I may be represented by the pKa of the neutral form of a compound having the structure of

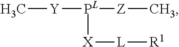

pKa of

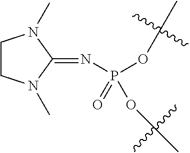 [SMILES: *OP(=O)(N=C1N(C)CCN1C)O*]

can be represented by pKa

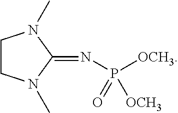

In some embodiments, a non-negatively charged internucleotidic linkage is a neutral internucleotidic linkage. In some embodiments, a non-negatively charged internucleotidic linkage is a positively-charged internucleotidic linkage. In some embodiments, a non-negatively charged internucleotidic linkage comprises a guanidine moiety. In some embodiments, a non-negatively charged internucleotidic linkage comprises a heteroaryl base moiety. In some embodiments, a non-negatively charged internucleotidic linkage comprises a triazole moiety. In some embodiments, a non-negatively charged internucleotidic linkage comprises an alkynyl moiety.

In some embodiments, a non-negatively charged internucleotidic linkage has the structure of formula I, I-a, I-b, I-c, I-n-1, I-n-2, I-n-3, II, II-a-1, II-a-2, II-b-1, II-b-2, II-c-1, II-c-2, II-d-1, II-d-2, or a salt form thereof (not negatively charged). In some embodiments, an internucleotidic linkage, e.g., a non-negatively charged internucleotidic linkage, has the structure of formula I-n-1 or a salt form thereof:

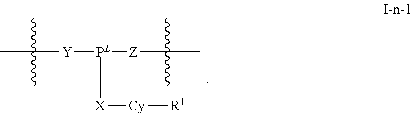

In some embodiments, X is a covalent bond and —X-Cy-R¹ is -Cy-R¹. In some embodiments, -Cy- is an optionally substituted bivalent group selected from a 5-20 membered heteroaryl ring having 1-10 heteroatoms, and a 3-20 membered heterocyclyl ring having 1-10 heteroatoms. In some embodiments, -Cy- is an optionally substituted bivalent 5-20 membered heteroaryl ring having 1-10 heteroatoms. In some embodiments, -Cy-R¹ is optionally substituted 5-20 membered heteroaryl ring having 1-10 heteroatoms, wherein at least one heteroatom is nitrogen. In some embodiments, -Cy-R¹ is optionally substituted 5-membered heteroaryl ring having 1-4 heteroatoms, wherein at least one heteroatom is nitrogen. In some embodiments, -Cy-R¹ is optionally substituted 6-membered heteroaryl ring having 1-4 heteroatoms, wherein at least one heteroatom is nitrogen. In some embodiments, -Cy-R¹ is optionally substituted triazolyl.

In some embodiments, an internucleotidic linkage, e.g., a non-negatively charged internucleotidic linkage, has the structure of formula I-n-2 or a salt form thereof:

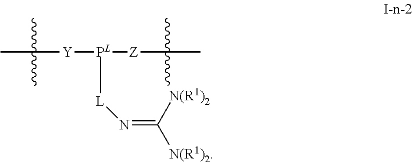

In some embodiments, R¹ is R′. In some embodiments, L is a covalent bond. In some embodiments, an internucleotidic linkage, e.g., a non-negatively charged internucleotidic linkage, has the structure of formula I-n-3 or a salt form thereof:

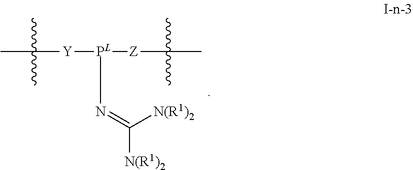

In some embodiments, two R′ on different nitrogen atoms are taken together to form a ring as described. In some embodiments, a formed ring is 5-membered. In some embodiments, a formed ring is 6-membered. In some embodiments, a formed ring is substituted. In some embodiments, the two R′ group that are not taken together to form a ring are each independently R. In some embodiments, the two R′ group that are not taken together to form a ring are each independently hydrogen or an optionally substituted C₁₋₆ aliphatic. In some embodiments, the two R′ group that are not taken together to form a ring are each independently hydrogen or an optionally substituted C₁₋₆ alkyl. In some embodiments, the two R′ group that are not taken together to form a ring are the same. In some embodiments, the two R′ group that are not taken together to form a ring are different. In some embodiments, both of them are —CH₃.

In some embodiments, a internucleotidic linkage, e.g., a non-negatively charged internucleotidic linkage, has the structure of formula II or a salt form thereof:

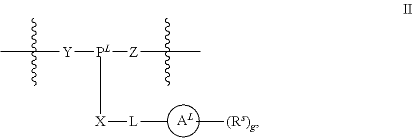

or a salt form thereof, wherein:
P^{L} is P(═W), P, or P→B(R′)₃;
W is O, N(-L-R⁵), S or Se;
   each of X, Y and Z is independently —O—, —S—, —N(-L-R⁵)—, or L;
Ring A^{L} is an optionally substituted 3-20 membered monocyclic, bicyclic or polycyclic ring having 0-10 heteroatoms;
each R^{s} is independently —H, halogen, —CN, —N₃, —NO, —NO₂, -L-R′, -L-Si(R)₃, -L-OR′, -L-SR′, -L-N(R′)₂, —O-L-R′, —O-L-Si(R)₃, —O-L-OR¹, —O-L-SR′, or —O-L-N(R′)₂;
g is 0-20;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced with C₁₋₆ alkylene, C₁₋₆ alkenylene, —C≡C—, a bivalent C₁-C₆ heteroaliphatic group having 1-5 heteroatoms, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—, and one or more CH or carbon atoms are optionally and independently replaced with Cy^{L};
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each Cy^{L} is independently an optionally substituted trivalent or tetravalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or,
two or more R groups on the same atom are optionally and independently taken together with the atom to form an optionally substituted, 3-30 membered, monocyclic, bicyclic or polycyclic ring having, in addition to the atom, 0-10 heteroatoms, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a internucleotidic linkage, e.g., a non-negatively charged internucleotidic linkage of formula II, has the structure of formula II-a-1 or a salt form thereof:

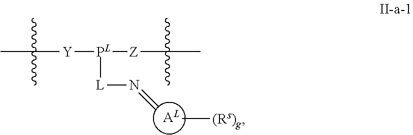

or a salt form thereof.

In some embodiments, a internucleotidic linkage, e.g., a non-negatively charged internucleotidic linkage of formula II, has the structure of formula II-a-2 or a salt form thereof:

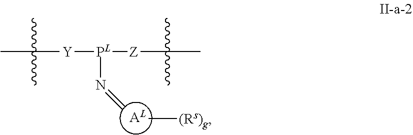

or a salt form thereof.

In some embodiments, A^{L} is bonded to —N═ or L through a carbon atom. In some embodiments, an internucleotidic linkage, e.g., a non-negatively charged internucleotidic linkage of formula II or II-a-1, II-a-2, has the structure of formula II-b-1 or a salt form thereof:

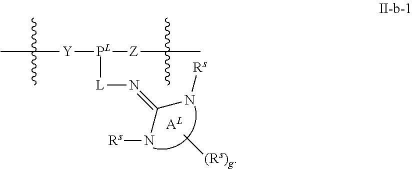

In some embodiments, a structure of formula II-a-1 or II-a-2 may be referred to a structure of formula II-a. In some embodiments, a structure of formula II-b-1 or II-b-2 may be referred to a structure of formula II-b. In some embodiments, a structure of formula II-c-1 or II-c-2 may be referred to a structure of formula II-c. In some embodiments, a structure of formula II-d-1 or II-d-2 may be referred to a structure of formula II-d.

In some embodiments, A^{L} is bonded to —N═ or L through a carbon atom. In some embodiments, an internucleotidic linkage, e.g., a non-negatively charged internucleotidic linkage of formula II or II-a-1, II-a-2, has the structure of formula II-b-2 or a salt form thereof:

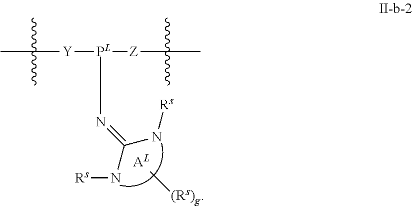

In some embodiments, Ring A^{L} is an optionally substituted 3-20 membered monocyclic ring having 0-10 heteroatoms (in addition to the two nitrogen atoms for formula II-b). In some embodiments, Ring A^{L} is an optionally substituted 5-membered monocyclic saturated ring.

In some embodiments, an internucleotidic linkage, e.g., a non-negatively charged internucleotidic linkage of formula II, II-a, or II-b, has the structure of formula II-c-1 or a salt form thereof:

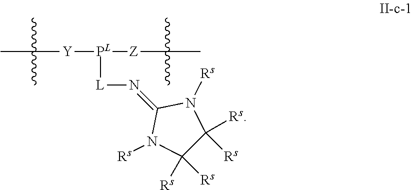

In some embodiments, an internucleotidic linkage, e.g., a non-negatively charged internucleotidic linkage of formula II, II-a, or II-b, has the structure of formula II-c-2 or a salt form thereof:

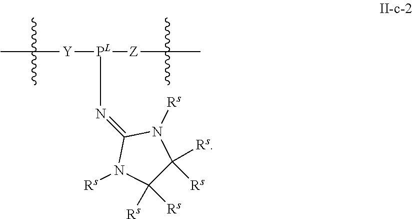

In some embodiments, an internucleotidic linkage, e.g., a non-negatively charged internucleotidic linkage of formula II, II-a, II-b, or II-c has the structure of formula II-d-1 or a salt form thereof:

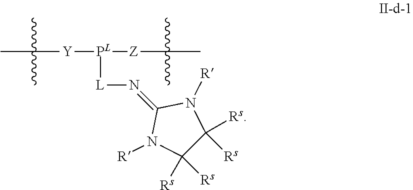

In some embodiments, an internucleotidic linkage, e.g., a non-negatively charged internucleotidic linkage of formula II, II-a, II-b, or II-c has the structure of formula II-d-2 or a salt form thereof:

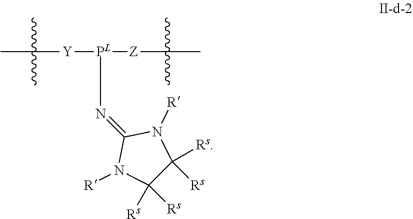

In some embodiments, each R′ is independently optionally substituted C₁₋₆ aliphatic. In some embodiments, each R′ is independently optionally substituted C₁₋₆ alkyl. In some embodiments, each R′ is independently —CH₃. In some embodiments, each R^{s} is —H.

In some embodiments, a non-negatively charged internucleotidic linkage has the structure of

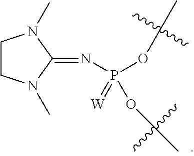

In some embodiments, a non-negatively charged internucleotidic linkage has the structure of

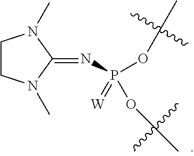

In some embodiments, a non-negatively charged internucleotidic linkage has the structure of

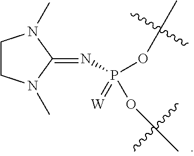

In some embodiments, a non-negatively charged internucleotidic linkage has the structure of

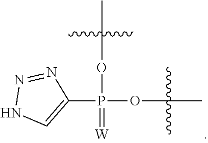

In some embodiments, a non-negatively charged internucleotidic linkage has the structure of

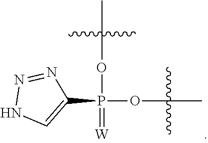

In some embodiments, a non-negatively charged internucleotidic linkage has the structure of

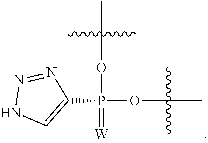

In some embodiments, a non-negatively charged internucleotidic linkage has the structure of

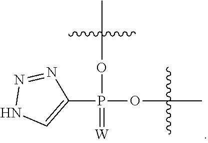

In some embodiments, a non-negatively charged internucleotidic linkage has the structure of

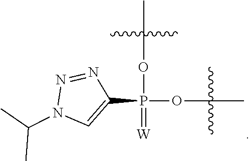

In some embodiments, a non-negatively charged internucleotidic linkage has the structure of

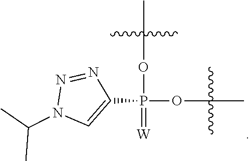

In some embodiments, a non-negatively charged internucleotidic linkage has the structure of

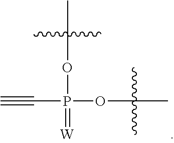

In some embodiments, a non-negatively charged internucleotidic linkage has the structure of

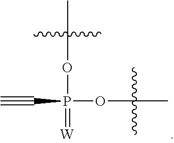

In some embodiments, a non-negatively charged internucleotidic linkage has the structure of

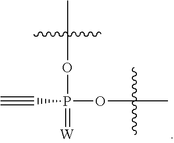

In some embodiments, W is O. In some embodiments, W is S.

In some embodiments, each L^{P} independently has the structure of formula I, I-a, I-b, I-c, I-n-1, I-n-2, I-n-3, II, II-a-1, II-a-2, II-b-1, II-b-2, II-c-1, II-c-2, II-d-1, II-d-2, or a salt form thereof.

In some embodiments, the present disclosure provides oligonucleotides comprising one or more non-negatively charged internucleotidic linkages. In some embodiments, a non-negatively charged internucleotidic linkage is a neutral internucleotidic linkage. In some embodiments, the present disclosure provides oligonucleotides comprising one or more neutral internucleotidic linkages. In some embodiments, a non-negatively charged internucleotidic linkage has the structure of formula I-n-1, I-n-2, I-n-3, II, II-a-1, II-a-2, II-b-1, II-b-2, II-c-1, II-c-2, II-d-1, II-d-2, or a salt form thereof.

In some embodiments, a non-negatively charged internucleotidic linkage comprises a triazole moiety. In some embodiments, a non-negatively charged internucleotidic linkage comprises an optionally substituted triazolyl group. In some embodiments, a non-negatively charged internucleotidic linkage has the structure of

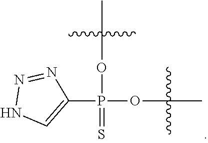 [SMILES: *OP(=S)(O*)c1c[nH]nn1]

In some embodiments, a non-negatively charged internucleotidic linkage has the structure of

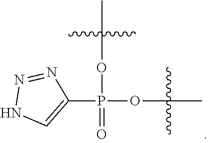 [SMILES: *OP(=O)(O*)c1c[nH]nn1]

In some embodiments, a non-negatively charged internucleotidic linkage comprises a substituted triazolyl group. In some embodiments, a non-negatively charged internucleotidic linkage has the structure of

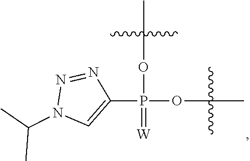

wherein W is O or S. In some embodiments, a non-negatively charged internucleotidic linkage comprises an optionally substituted alkynyl group. In some embodiments, a non-negatively charged internucleotidic linkage has the structure of

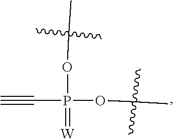

wherein W is O or S.

In some embodiments, the present disclosure provides oligonucleotides comprising an internucleotidic linkage, e.g., a non-negatively charged internucleotidic linkage, which comprises a cyclic guanidine moiety. In some embodiments, an internucleotidic linkage comprises a cyclic guanidine and has the structure of:

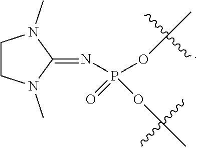 [SMILES: *OP(=O)(N=C1N(C)CCN1C)O*]

In some embodiments, an internucleotidic linkage, e.g., a non-negatively charged internucleotidic linkage, comprising a cyclic guanidine is stereochemically controlled.

In some embodiments, a non-negatively charged internucleotidic linkage, or a neutral internucleotidic linkage, is or comprising a structure selected from

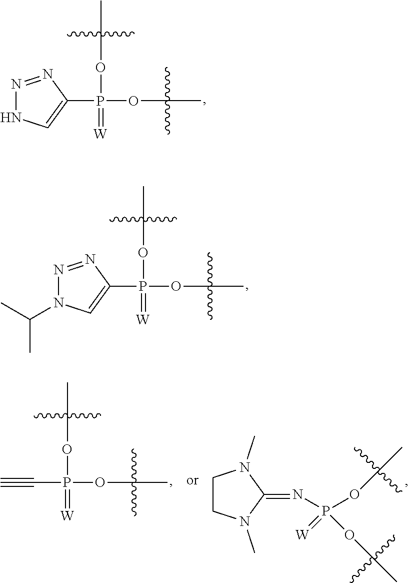

wherein W is O or S. In some embodiments, a non-negatively charged internucleotidic linkage is a chirally controlled internucleotidic linkage. In some embodiments, a neutral internucleotidic linkage is a chirally controlled internucleotidic linkage. In some embodiments, a nucleic acid or an oligonucleotide comprising a modified internucleotidic linkage comprising a cyclic guanidine moiety is a siRNA, double-straned siRNA, single-stranded siRNA, gapmer, skipmer, blockmer, antisense oligonucleotide, antagomir, microRNA, pre-microRNs, antimir, supermir, ribozyme, Ul adaptor, RNA activator, RNAi agent, decoy oligonucleotide, triplex forming oligonucleotide, aptamer or adjuvant.

In some embodiments, an oligonucleotide comprises a neutral internucleotidic linkage and a chirally controlled internucleotidic linkage. In some embodiments, an oligonucleotide comprises a neutral internucleotidic linkage and a chirally controlled internucleotidic linkage which is a phosphorothioate in the Rp or Sp configuration. In some embodiments, the present disclosure provides an oligonucleotide comprising one or more non-negatively charged internucleotidic linkages and one or more phosphorothioate internucleotidic linkage, wherein each phosphorothioate internucleotidic linkage in the oligonucleotide is independently a chirally controlled internucleotidic linkage. In some embodiments, the present disclosure provides an oligonucleotide comprising one or more neutral internucleotidic linkages and one or more phosphorothioate internucleotidic linkage, wherein each phosphorothioate internucleotidic linkage in the oligonucleotide is independently a chirally controlled internucleotidic linkage. In some embodiments, a provided oligonucleotide comprises at least 5, 6, 7, 8, 9, 10, 11, 12, 13, 14, 15, 16, 17, 18, 19, 20 or more chirally controlled phosphorothioate internucleotidic linkages.

Without wishing to be bound by any particular theory, the present disclosure notes that a neutral internucleotidic linkage can be more hydrophobic than a phosphorothioate internucleotidic linkage (PS), which is more hydrophobic than a phosphodiester linkage (natural phosphate linkage, PO). Typically, unlike a PS or PO, a neutral internucleotidic linkage bears less charge. Without wishing to be bound by any particular theory, the present disclosure notes that incorporation of one or more neutral internucleotidic linkages into an oligonucleotide may increase oligonucleotides' ability to be taken up by a cell and/or to escape from endosomes. Without wishing to be bound by any particular theory, the present disclosure notes that incorporation of one or more neutral internucleotidic linkages can be utilized to modulate melting temperature between an oligonucleotide and its target nucleic acid.

Without wishing to be bound by any particular theory, the present disclosure notes that incorporation of one or more non-negatively charged internucleotidic linkages, e.g., neutral internucleotidic linkages, into an oligonucleotide may be able to increase the oligonucleotide's ability to mediate a function such as exon skipping or gene knockdown. In some embodiments, an oligonucleotide capable of mediating knockdown of level of a nucleic acid or a product encoded thereby comprises one or more non-negatively charged internucleotidic linkages. In some embodiments, an oligonucleotide capable of mediating knockdown of expression of a target gene comprises one or more non-negatively charged internucleotidic linkages. In some embodiments, an oligonucleotide capable of mediating knockdown of expression of a target gene comprises one or more neutral internucleotidic linkages.

In some embodiments, a non-negatively charged internucleotidic linkage is not chirally controlled. In some embodiments, a non-negatively charged internucleotidic linkage is chirally controlled. In some embodiments, a non-negatively charged internucleotidic linkage is chirally controlled and its linkage phosphorus is Rp. In some embodiments, a non-negatively charged internucleotidic linkage is chirally controlled and its linkage phosphorus is Sp.

In some embodiments, a provided oligonucleotide comprises 1, 2, 3, 4, 5, 6, 7, 8, 9, 10, or more non-negatively charged internucleotidic linkages. In some embodiments, a provided oligonucleotide comprises 1, 2, 3, 4, 5, 6, 7, 8, 9, 10, or more neutral internucleotidic linkages. In some embodiments, each of non-negatively charged internucleotidic linkage and/or neutral internucleotidic linkages is optionally and independently chirally controlled. In some embodiments, each non-negatively charged internucleotidic linkage in an oligonucleotide is independently a chirally controlled internucleotidic linkage. In some embodiments, each neutral internucleotidic linkage in an oligonucleotide is independently a chirally controlled internucleotidic linkage. In some embodiments, at least one non-negatively charged internucleotidic linkage/neutral internucleotidic linkage has the structure of

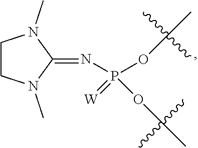

wherein W is O or S. In some embodiments, at least one non-negatively charged internucleotidic linkage/neutral internucleotidic linkage has the structure of

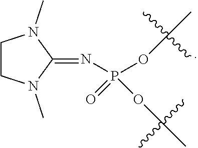 [SMILES: *OP(=O)(N=C1N(C)CCN1C)O*]

In some embodiments, at least one non-negatively charged internucleotidic linkage/neutral internucleotidic linkage has the structure of

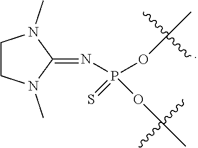 [SMILES: *OP(=S)(N=C1N(C)CCN1C)O*]

In some embodiments, at least one non-negatively charged internucleotidic linkage/neutral internucleotidic linkage has the structure of

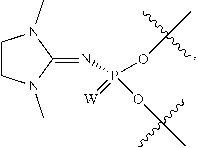

wherein W is O or S. In some embodiments, at least one non-negatively charged internucleotidic linkage/neutral internucleotidic linkage has the structure of

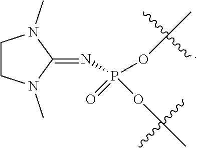

In some embodiments, at least one non-negatively charged internucleotidic linkage/neutral internucleotidic linkage has the structure of

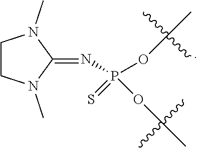

In some embodiments, at least one non-negatively charged internucleotidic linkage/neutral internucleotidic linkage has the structure of

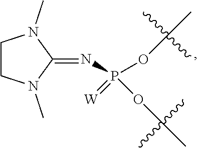

wherein W is O or S. In some embodiments, at least one non-negatively charged internucleotidic linkage/neutral internucleotidic linkage has the structure of

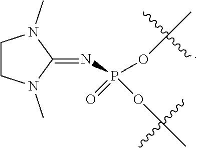

In some embodiments, at least one non-negatively charged internucleotidic linkage/neutral internucleotidic linkage has the structure of

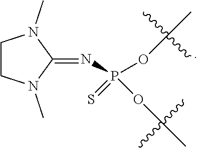

In some embodiments, a provided oligonucleotide comprises at least one non-negatively charged internucleotidic linkage wherein its linkage phosphorus is in Rp configuration, and at least one non-negatively charged internucleotidic linkage wherein its linkage phosphorus is in Sp configuration.

In some embodiments, the present disclosure provides a chirally controlled oligonucleotide, wherein at least two of the individual internucleotidic linkages within the oligonucleotide have different stereochemistry and/or different P-modifications relative to one another and the oligonucleotide has a structure represented by the following formula:

[S^{B}n1R^{B}n2S^{B}n3R^{B}n4 . . . S^{B}nxR^{B}ny]
wherein:
each R^{B} independently represents a block of nucleotide units having the R configuration at the linkage phosphorus;
each S^{B} independently represents a block of nucleotide units having the S configuration at the linkage phosphorus;
each of n1-ny is zero or an integer, with the requirement that at least one odd n and at least one even n must be non-zero so that the oligonucleotide includes at least two individual internucleotidic linkages with different stereochemistry relative to one another; and wherein the sum of n1-ny is between 2 and 200, and in some embodiments is between a lower limit selected from the group consisting of 2, 3, 4, 5, 6, 7, 8, 9, 10, 11, 12, 13, 14, 15, 16, 17, 18, 19, 20, 21, 22, 23, 24, 25 or more and an upper limit selected from the group consisting of 5, 10, 15, 20, 25, 30, 35, 40, 45, 50, 55, 60, 65, 70, 75, 80, 85, 90, 95, 100, 110, 120, 130, 140, 150, 160, 170, 180, 190, and 200, the upper limit being larger than the lower limit.

In some such embodiments, each n has the same value; in some embodiments, each even n has the same value as each other even n; in some embodiments, each odd n has the same value each other odd n; in some embodiments, at least two even ns have different values from one another; in some embodiments, at least two odd ns have different values from one another.

In some embodiments, at least two adjacent ns are equal to one another, so that a provided oligonucleotide includes adjacent blocks of S stereochemistry linkages and R stereochemistry linkages of equal lengths. In some embodiments, provided oligonucleotides include repeating blocks of S and R stereochemistry linkages of equal lengths. In some embodiments, provided oligonucleotides include repeating blocks of S and R stereochemistry linkages, where at least two such blocks are of different lengths from one another; in some such embodiments each S stereochemistry block is of the same length, and is of a different length from each R stereochemistry length, which may optionally be of the same length as one another.

In some embodiments, at least two skip-adjacent ns are equal to one another, so that a provided oligonucleotide includes at least two blocks of linkages of a first stereochemistry that are equal in length to one another and are separated by a block of linkages of the other stereochemistry, which separating block may be of the same length or a different length from the blocks of first stereochemistry.

In some embodiments, ns associated with linkage blocks at the ends of a provided oligonucleotide are of the same length. In some embodiments, provided oligonucleotides have terminal blocks of the same linkage stereochemistry. In some such embodiments, the terminal blocks are separated from one another by a middle block of the other linkage stereochemistry.

In some embodiments, a provided oligonucleotide of formula [S^{B}n1R^{B}n2S^{B}n3R^{B}n4 . . . S^{B}nxR^{B}ny] is a stereoblockmer. In some embodiments, a provided oligonucleotide of formula [S^{B}n1R^{B}n2S^{B}n3R^{B}n4 . . . S^{B}nxR^{B}ny] is a stereoskipmer. In some embodiments, a provided oligonucleotide of formula [S^{B}n1R^{B}n2S^{B}n3R^{B}n4 . . . S^{B}nxR^{B}ny] is a stereoaltmer. In some embodiments, a provided oligonucleotide of formula [S^{B}n1R^{B}n2S^{B}n3R^{B}n4 . . . S^{B}nxR^{B}ny] is a gapmer.

In some embodiments, a provided oligonucleotide of formula [S^{B}n1R^{B}n2S^{B}n3R^{B}n4 . . . S^{B}nxR^{B}ny] is of any of the above described patterns and further comprises patterns of P-modifications. For instance, in some embodiments, a provided oligonucleotide of formula [S^{B}n1R^{B}n2S^{B}n3R^{B}n4 . . . S^{B}nxR^{B}ny] and is a stereoskipmer and P-modification skipmer. In some embodiments, a provided oligonucleotide of formula [S^{B}n1R^{B}n2S^{B}n3R^{B}n4 . . . S^{B}nxR^{B}ny] and is a stereoblockmer and P-modification altmer. In some embodiments, a provided oligonucleotide of formula [S^{B}n1R^{B}n2S^{B}n3R^{B}n4 . . . S^{B}nxR^{B}ny] and is a stereoaltmer and P-modification blockmer.

In some embodiments, a provided oligonucleotide of formula [S^{B}n1R^{B}n2S^{B}n3R^{B}n4 . . . S^{B}nxR^{B}ny] is a chirally controlled oligonucleotide comprising one or more modified internucleotidic linkages independently having the structure of Formula I:

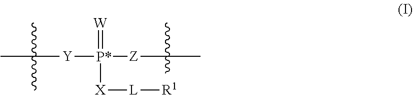

wherein:
P* is a symmetric phosphorus atom, or asymmetric phosphorus atom that is either Rp or Sp;
W is O, S or Se;
each of X, Y and Z is independently —O—, —S—, —N(-L-R¹)—, or L;
L is a covalent bond or an optionally substituted, linear or branched C₁-C₁₀ alkylene, wherein one or more methylene units of L are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
   two R′ are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene;
each R is independently hydrogen, or an optionally substituted group selected from C₁-C₆ aliphatic, carbocyclyl, aryl, heteroaryl, and heterocyclyl; and
each

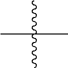

independently represents a connection to a nucleoside.

In some embodiments, L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene;
each R is independently hydrogen, or an optionally substituted group selected from C₁-C₆ aliphatic, phenyl, carbocyclyl, aryl, heteroaryl, and heterocyclyl; and
each

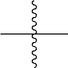

independently represents a connection to a nucleoside.

In some embodiments, a chirally controlled oligonucleotide comprises one or more modified internucleotidic phosphorus linkages. In some embodiments, a chirally controlled oligonucleotide comprises, e.g., a phosphorothioate or a phosphorothioate triester linkage. In some embodiments, a chirally controlled oligonucleotide comprises a phosphorothioate triester linkage. In some embodiments, a chirally controlled oligonucleotide comprises at least two phosphorothioate triester linkages. In some embodiments, a chirally controlled oligonucleotide comprises at least three phosphorothioate triester linkages. In some embodiments, a chirally controlled oligonucleotide comprises at least four phosphorothioate triester linkages. In some embodiments, a chirally controlled oligonucleotide comprises at least five phosphorothioate triester linkages. Examples of such modified internucleotidic phosphorus linkages are described further herein.

In some embodiments, a chirally controlled oligonucleotide comprises different internucleotidic phosphorus linkages. In some embodiments, a chirally controlled oligonucleotide comprises at least one phosphate diester internucleotidic linkage and at least one modified internucleotidic linkage. In some embodiments, a chirally controlled oligonucleotide comprises at least one phosphate diester internucleotidic linkage and at least one phosphorothioate triester linkage. In some embodiments, a chirally controlled oligonucleotide comprises at least one phosphate diester internucleotidic linkage and at least two phosphorothioate triester linkages. In some embodiments, a chirally controlled oligonucleotide comprises at least one phosphate diester internucleotidic linkage and at least three phosphorothioate triester linkages. In some embodiments, a chirally controlled oligonucleotide comprises at least one phosphate diester internucleotidic linkage and at least four phosphorothioate triester linkages. In some embodiments, a chirally controlled oligonucleotide comprises at least one phosphate diester internucleotidic linkage and at least five phosphorothioate triester linkages. Examples of such modified internucleotidic phosphorus linkages are described further herein.

In some embodiments, a phosphorothioate triester linkage comprises a chiral auxiliary, which, for example, is used to control the stereoselectivity of a reaction. In some embodiments, a phosphorothioate triester linkage does not comprise a chiral auxiliary. In some embodiments, a phosphorothioate triester linkage is intentionally maintained until and/or during the administration to a subject.

In some embodiments, a chirally controlled oligonucleotide is linked to a solid support. In some embodiments, a chirally controlled oligonucleotide is cleaved from a solid support.

In some embodiments, a chirally controlled oligonucleotide comprises at least one phosphate diester internucleotidic linkage and at least two consecutive modified internucleotidic linkages. In some embodiments, a chirally controlled oligonucleotide comprises at least one phosphate diester internucleotidic linkage and at least two consecutive phosphorothioate triester internucleotidic linkages.

In some embodiments, the present disclosure provides compositions comprising or consisting of a plurality of provided oligonucleotides (e.g., chirally controlled oligonucleotide compositions). In some embodiments, all such provided oligonucleotides are of the same type, i.e., all have the same base sequence, pattern of backbone linkages (i.e., pattern of internucleotidic linkage types, for example, phosphate, phosphorothioate, etc), pattern of backbone chiral centers (i.e. pattern of linkage phosphorus stereochemistry (Rp/Sp)), and pattern of backbone phosphorus modifications (e.g., pattern of “—XLR¹” groups in Formula I, disclosed herein). In some embodiments, all oligonucleotides of the same type are identical. In many embodiments, however, provided compositions comprise a plurality of oligonucleotides types, typically in pre-determined relative amounts.

In some embodiments, a provided oligonucleotide comprises a pattern of backbone linkages. In some embodiments, a pattern of backbone linkages is or comprises a sequence of any of: OOO, OOOO, OOOOO, OOOOOOO, OOOOOOO, OOOOOOOO, OOOOOOOOO, OOOOOOOOOO, OXOX, OXOX, OXXO, XOOX, XXOOXX, XOXOXOXX, OXOXOXOO, XXX, XXXX, XXXXX, XXXXXX, XXXXXXX, XXXXXXXX, XXXXXXXXX, XXXXXXXXXX, OOOOOOOOOOOOOOOOO, OOOOOOOOOOOOOOOOOO, OOOOOOOOOOOOOOOOOOO, OOOOOOOOOOOOOOOOOOOO, OOOOOOOOOOOOOOOOOOOOO, OOOOOOOOOOOOOOOOOOOOOO, XOXOXOXOOOXOOXXXXXO, XOXOXOXOXOXOOOOOOOOXX, XOXOXOXOXOXOOOOOOXX, XOXOXOXOXOXOOOOOXXX, XOXOXOXOXOXOXOOOOOOXX, XOXOXOXOXOXOXOOOOXX, XOXOXOXOXOXOXXXXXX, XOXOXOXOXOXOXXXXXXO, XOXOXOXOXOXOXXXXXXX, XOXOXOXOXOXOXXXXXXXXXXO, XOXOXOXOXOXOXXXXXXXXXXX, XXOXOXOXOOOXOOXXXXXO, XXOXOXOXOXOXOOOOOOOOXX, XXOXOXOXOXOXOOOOOOXX, XXOXOXOXOXOXOOOOOXXX, XXOXOXOXOXOXOXOOOOOOXX, XXOXOXOXOXOXOXOOOOXX, XXOXOXOXOXOXOXXXXXX, XXOXOXOXOXOXOXXXXXXO, XXOXOXOXOXOXOXXXXXXX, XXOXOXOXOXOXOXXXXXXXXXXO, XXOXOXOXOXOXOXXXXXXXXXXX, XXOXOXXXOOOXOOXXXXXO, XXOXOXXXOXOXOOOOOOOOXX, XXOXOXXXOXOXOOOOOOXX, XXOXOXXXOXOXOOOOOXXX, XXOXOXXXOXOXOXOOOOOOXX, XXOXOXXXOXOXOXOOOOXX, XXOXOXXXOXOXOXXXXXX, XXOXOXXXOXOXOXXXXXXO, XXOXOXXXOXOXOXXXXXXX, XXOXOXXXOXOXOXXXXXXXXXXO, XXOXOXXXOXOXOXXXXXXXXXXX, XXOXOXXXOXXXOOOOOOOOXX, XXOXOXXXOXXXOOOOOOXX, XXOXOXXXOXXXOOOOOXXX, XXOXOXXXOXXXOXOOOOOOXX, XXOXOXXXOXXXOXOOOOXX, XXOXOXXXOXXXOXXXXXX, XXOXOXXXOXXXOXXXXXXO, XXOXOXXXOXXXOXXXXXXX, XXOXOXXXOXXXOXXXXXXXXXXO, XXOXOXXXOXXXOXXXXXXXXXXX, XXOXOXXXXOOXOOXXXXXO, XXOXOXXXXXOXOOOOOOOOXX, XXOXOXXXXXOXOOOOOOXX, XXOXOXXXXXOXOOOOOXXX, XXOXOXXXXXOXOXOOOOOOXX, XXOXOXXXXXOXOXOOOOXX, XXOXOXXXXXOXOXXXXXX, XXOXOXXXXXOXOXXXXXXO, XXOXOXXXXXOXOXXXXXXX, XXOXOXXXXXOXOXXXXXXXXXXO, XXOXOXXXXXOXOXXXXXXXXXXX, XXOXXXOXOOOXOOXXXXXO, XXOXXXOXOXOXOOOOOOOOXX, XXOXXXOXOXOXOOOOOOXX, XXOXXXOXOXOXOOOOOXXX, XXOXXXOXOXOXOXOOOOOOXX, XXOXXXOXOXOXOXOOOOXX, XXOXXXOXOXOXOXXXXXX, XXOXXXOXOXOXOXXXXXXO, XXOXXXOXOXOXOXXXXXXX, XXOXXXOXOXOXOXXXXXXXXXXO, XXOXXXOXOXOXOXXXXXXXXXXX, XXOXXXOXOXXXOOOOOOOOXX, XXOXXXOXOXXXOOOOOOXX, XXOXXXOXOXXXOOOOOXXX, XXOXXXOXOXXXOXOOOOOOXX, XXOXXXOXOXXXOXOOOOXX, XXOXXXOXOXXXOXXXXXX, XXOXXXOXOXXXOXXXXXXO, XXOXXXOXOXXXOXXXXXXX, XXOXXXOXOXXXOXXXXXXXXXXO, XXOXXXOXOXXXOXXXXXXXXXXX, XXOXXXOXXOOXOOXXXXXO, XXOXXXOXXXOXOOOOOOOOXX, XXOXXXOXXXOXOOOOOOXX, XXOXXXOXXXOXOOOOOXXX, XXOXXXOXXXOXOXOOOOOOXX, XXOXXXOXXXOXOXOOOOXX, XXOXXXOXXXOXOXXXXXX, XXOXXXOXXXOXOXXXXXXO, XXOXXXOXXXOXOXXXXXXX, XXOXXXOXXXOXOXXXXXXXXXXO, XXOXXXOXXXOXOXXXXXXXXXXX, XXOXXXXXOOOXOOXXXXXO, XXOXXXXXOXOXOOOOOOOOXX, XXOXXXXXOXOXOOOOOOXX, XXOXXXXXOXOXOOOOOXXX, XXOXXXXXOXOXOXOOOOOOXX, XXOXXXXXOXOXOXOOOOXX, XXOXXXXXOXOXOXXXXXX, XXOXXXXXOXOXOXXXXXXO, XXOXXXXXOXOXOXXXXXXX, XXOXXXXXOXOXOXXXXXXXXXXO, XXOXXXXXOXOXOXXXXXXXXXXX, XXXOXOXOXOOOXOOXXXXXO, XXXOXOXOXOXOXOOOOOOOOXX, XXXOXOXOXOXOXOOOOOOXX, XXXOXOXOXOXOXOOOOOXXX, XXXOXOXOXOXOXOXOOOOOOXX, XXXOXOXOXOXOXOXOOOOXX, XXXOXOXOXOXOXOXXXXXX, XXXOXOXOXOXOXOXXXXXXO, XXXOXOXOXOXOXOXXXXXXX, XXXOXOXOXOXOXOXXXXXXXXXXO, XXXOXOXOXOXOXOXXXXXXXXXXX, XXXXOXOXOOOXOOXXXXXO, XXXXOXOXOXOXOOOOOOOOXX, XXXXOXOXOXOXOOOOOOXX, XXXXOXOXOXOXOOOOOXXX, XXXXOXOXOXOXOXOOOOOOXX, XXXXOXOXOXOXOXOOOOXX, XXXXOXOXOXOXOXXXXXX, XXXXOXOXOXOXOXXXXXXO, XXXXOXOXOXOXOXXXXXXX, XXXXOXOXOXOXOXXXXXXXXXXO, XXXXOXOXOXOXOXXXXXXXXXXX, XXXXOXOXOXXXOOOOOOOOXX, XXXXOXOXOXXXOOOOOOXX, XXXXOXOXOXXXOOOOOXXX, XXXXOXOXOXXXOXOOOOOOXX, XXXXOXOXOXXXOXOOOOXX, XXXXOXOXOXXXOXXXXXX, XXXXOXOXOXXXOXXXXXXO, XXXXOXOXOXXXOXXXXXXX, XXXXOXOXOXXXOXXXXXXXXXXO, XXXXOXOXOXXXOXXXXXXXXXXX, XXXXOXOXXOOXOOXXXXXO, XXXXOXOXXOOXOOXXXXXO, XXXXOXOXXXOXOOOOOOOOXX, XXXXOXOXXXOXOOOOOOXX, XXXXOXOXXXOXOOOOOXXX, XXXXOXOXXXOXOXOOOOOOXX, XXXXOXOXXXOXOXOOOOXX, XXXXOXOXXXOXOXXXXXX, XXXXOXOXXXOXOXXXXXXO, XXXXOXOXXXOXOXXXXXXX, XXXXOXOXXXOXOXXXXXXXXXXO, XXXXOXOXXXOXOXXXXXXXXXXX, XXXXOXOXXXXXOOOOOOOOXX, XXXXOXOXXXXXOOOOOOXX, XXXXOXOXXXXXOOOOOXXX, XXXXOXOXXXXXOXOOOOOOXX, XXXXOXOXXXXXOXOOOOXX, XXXXOXOXXXXXOXXXXXX, XXXXOXOXXXXXOXXXXXXO, XXXXOXOXXXXXOXXXXXXX, XXXXOXOXXXXXOXXXXXXXXXXO, XXXXOXOXXXXXOXXXXXXXXXXX, XXXXOXXXOOOXOOXXXXXO, XXXXOXXXOOOXOOXXXXXO, XXXXOXXXOXOXXOOOOOOOXX, XXXXOXXXOXOXXOOOOOXX, XXXXOXXXOXOXXOOOOXXX, XXXXOXXXOXOXXXOOOOOOXX, XXXXOXXXOXOXXXOOOOXX, XXXXOXXXOXOXXXXXXXX, XXXXOXXXOXOXXXXXXXXO, XXXXOXXXOXOXXXXXXXXX, XXXXOXXXOXOXXXXXXXXXXXXO, XXXXOXXXOXOXXXXXXXXXXXXX, XXXXOXXXOXXXOOOOOOOOXX, XXXXOXXXOXXXOOOOOOXX, XXXXOXXXOXXXOOOOOXXX, XXXXOXXXOXXXOXOOOOOOXX, XXXXOXXXOXXXOXOOOOXX, XXXXOXXXOXXXOXXXXXX, XXXXOXXXOXXXOXXXXXXO, XXXXOXXXOXXXOXXXXXXX, XXXXOXXXOXXXOXXXXXXXXXXO, XXXXOXXXOXXXOXXXXXXXXXXX, XXXXXXOXOOOXOOXXXXXO, XXXXXXOXOOOXOOXXXXXO, XXXXXXOXOXOXOOOOOOOOXX, XXXXXXOXOXOXOOOOOOXX, XXXXXXOXOXOXOOOOOXXX, XXXXXXOXOXOXOXOOOOOOXX, XXXXXXOXOXOXOXOOOOXX, XXXXXXOXOXOXOXXXXXX, XXXXXXOXOXOXOXXXXXXO, XXXXXXOXOXOXOXXXXXXX, XXXXXXOXOXOXOXXXXXXXXXXO, XXXXXXOXOXOXOXXXXXXXXXXX, XXXXXXOXOXXXOOOOOOOOXX, XXXXXXOXOXXXOOOOOOXX, XXXXXXOXOXXXOOOOOXXX, XXXXXXOXOXXXOXOOOOOOXX, XXXXXXOXOXXXOXOOOOXX, XXXXXXOXOXXXOXXXXXX, XXXXXXOXOXXXOXXXXXXO, XXXXXXOXOXXXOXXXXXXX, XXXXXXOXOXXXOXXXXXXXXXXO, XXXXXXOXOXXXOXXXXXXXXXXX, XXXXXXXXXXXXXXXXXXX, XXXXXXXXXXXXXXXXXXXX, XXXXXXXXXXXXXXXXXXXXX, XXXXXXXXXXXXXXXXXXXXXX, XXXXXXXXXXXXXXXXXXXXXXX, XXXXXXXXXXXXXXXXXXXXXXXX, XXXXXXXXXXXXXXXXXXXXXXXXX, XXXXXXXXXXXXXXXXXXXXXXXXXX, XXXXXXXXXXXXXXXXXXXXXXXXXXX, or XXXXXXXXXXXXXXXXXXXXXXXXXXXX, wherein O indicates a phosphodiester, and X indicates an internucleotidic linkage or modified internucleotidic linkage which is not phosphodiester; in some embodiments, a modified internucleotidic linkage is a phosphorothioate; in some embodiments, a modified internucleotidic linkage is chirally controlled; in some embodiments, a modified internucleotidic linkage is a chirally controlled phosphorothioate.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any internucleotidic linkage described herein or known in the art. In some embodiments, a moiety that binds ASPGR is, for example, a GalNAc moiety is any GalNAc, or variant or modification thereof, as described herein or known in the art. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any internucleotidic linkage described herein or known in the art in combination with any other structural element or modification described herein, including but not limited to, base sequence or portion thereof, sugar, base (nucleobase); stereochemistry or pattern thereof; additional chemical moiety, including but not limited to, a targeting moiety, a lipid moiety, a carbohydrate moiety, etc.; seed region; post-seed region; 5′-end structure; 5′-end region; 5′ nucleotide moiety; 3′-end region; 3′-terminal dinucleotide; 3′-end cap; length; GC content; additional chemical moiety, including but not limited to, a targeting moiety, lipid moiety, a GalNAc, etc.; format or any structural element thereof, and/or any other structural element or modification described herein; and in some embodiments, the present disclosure pertains to multimers of any such oligonucleotides.

In some embodiments, a chirally controlled oligonucleotide comprises one or more modified internucleotidic phosphorus linkages. In some embodiments, a chirally controlled oligonucleotide comprises, e.g., a phosphorothioate or a phosphorothioate triester linkage.

In some embodiments, a chirally controlled oligonucleotide comprises a phosphorothioate triester linkage. In some embodiments, a chirally controlled oligonucleotide comprises at least two phosphorothioate triester linkages. In some embodiments, a chirally controlled oligonucleotide comprises at least three phosphorothioate triester linkages. In some embodiments, a chirally controlled oligonucleotide comprises at least four phosphorothioate triester linkages. In some embodiments, a chirally controlled oligonucleotide comprises at least five phosphorothioate triester linkages. Example modified internucleotidic phosphorus linkages are described further herein. In some embodiments, a chirally controlled oligonucleotide comprises different internucleotidic phosphorus linkages. In some embodiments, a chirally controlled oligonucleotide comprises at least one phosphate diester internucleotidic linkage and at least one modified internucleotidic linkage. In some embodiments, a chirally controlled oligonucleotide comprises at least one phosphate diester internucleotidic linkage and at least one phosphorothioate triester linkage. In some embodiments, a chirally controlled oligonucleotide comprises at least one phosphate diester internucleotidic linkage and at least two phosphorothioate triester linkages. In some embodiments, a chirally controlled oligonucleotide comprises at least one phosphate diester internucleotidic linkage and at least three phosphorothioate triester linkages. In some embodiments, a chirally controlled oligonucleotide comprises at least one phosphate diester internucleotidic linkage and at least four phosphorothioate triester linkages.

In some embodiments, the present disclosure provides oligonucleotides comprising one or more modified internucleotidic linkages independently having the structure of Formula I:

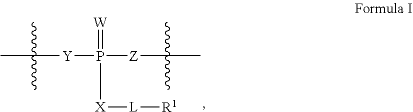

W is O, S or Se;
each of X, Y and Z is independently —O—, —S—, —N(-L-R¹)-, or L;
L is a covalent bond or an optionally substituted, linear or branched C₁-C₁₀ aliphatic, wherein one or more methylene units of L are optionally and independently replaced by an optionally substituted group selected from C₁-C₆ aliphatic moiety, C₁-C₆ alkenylene, —C≡C—, a C₁-C₆ heteroaliphatic moiety, —C(R′)₂—, -Cy-, —B(R′)—, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—;
R¹ is halogen, R, or an optionally substituted C₁-C₅₀ aliphatic wherein one or more methylene units are optionally and independently replaced by an optionally substituted group selected from C₁-C₆ alkylene, C₁-C₆ alkenylene, —C≡C—, a C₁-C₆ heteroaliphatic moiety, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂— —SC(O)—, —C(O)S—, —OC(O)—, and —C(O)O—
each R′ is independently —R, —C(O)R, —CO₂R, or —SO₂R, or:
two or more R′ are taken together with their intervening atoms to form an optionally substituted C₃-C₁₄ group selected from aryl, carbocyclyl, heterocyclyl, and heteroaryl;
-Cy- is an optionally substituted bivalent ring selected from phenylene, C₃-C₁₄ carbocyclylene, C₁₀-C₁₄ arylene, C₅-C₁₄ heteroarylene, and C₃-C₁₄ heterocyclylene; and
each R is independently hydrogen, or an optionally substituted group selected from C₁-C₂₀ aliphatic, C₃-C₂₀ carbocyclyl, C₆-C₂₀ aryl, C₅-C₂₀ heteroaryl, and C₃-C₂₀ heterocyclyl.

In some embodiments of Formula I, P in T^{LD} is P*. In some embodiments, P* is an asymmetric phosphorus atom and is either Rp or Sp. In some embodiments, P* is Rp. In other embodiments, P* is Sp. In some embodiments, an oligonucleotide comprises one or more internucleotidic linkages of Formula I wherein each P* is independently Rp or Sp. In some embodiments, an oligonucleotide comprises one or more internucleotidic linkages of Formula I wherein each P* is Rp. In some embodiments, an oligonucleotide comprises one or more internucleotidic linkages of Formula I wherein each P* is Sp. In some embodiments, an oligonucleotide comprises at least one internucleotidic linkage of Formula I wherein P* is Rp. In some embodiments, an oligonucleotide comprises at least one internucleotidic linkage of Formula I wherein P* is Sp. In some embodiments, an oligonucleotide comprises at least one internucleotidic linkage of Formula I wherein P* is Rp, and at least one internucleotidic linkage of Formula I wherein P* is Sp.

In some embodiments of Formula I, W is O, —S—, or Se. In some embodiments, W is O. In some embodiments, W is S. In some embodiments, W is Se. In some embodiments, an oligonucleotide comprises at least one internucleotidic linkage of Formula I wherein W is O. In some embodiments, an oligonucleotide comprises at least one internucleotidic linkage of Formula I wherein W is S. In some embodiments, an oligonucleotide comprises at least one internucleotidic linkage of Formula I wherein W is Se.

In some embodiments of Formula I, an oligonucleotide comprises at least one internucleotidic linkage of Formula I wherein W is O. In some embodiments, an oligonucleotide comprises at least one internucleotidic linkage of Formula I wherein W is S.

In some embodiments, each R is independently hydrogen, or an optionally substituted group selected from C₁-C₆ aliphatic, phenyl, carbocyclyl, aryl, heteroaryl, and heterocyclyl.

In some embodiments, R is hydrogen. In some embodiments, R is an optionally substituted group selected from C₁-C₆ aliphatic, phenyl, carbocyclyl, aryl, heteroaryl, and heterocyclyl.

In some embodiments, R is an optionally substituted C₁-C₆ aliphatic. In some embodiments, R is an optionally substituted C₁-C₆ alkyl. In some embodiments, R is optionally substituted, linear or branched hexyl. In some embodiments, R is optionally substituted, linear or branched pentyl. In some embodiments, R is optionally substituted, linear or branched butyl. In some embodiments, R is optionally substituted, linear or branched propyl. In some embodiments, R is optionally substituted ethyl. In some embodiments, R is optionally substituted methyl.

In some embodiments, R is optionally substituted phenyl. In some embodiments, R is substituted phenyl. In some embodiments, R is phenyl.

In some embodiments, R is optionally substituted carbocyclyl. In some embodiments, R is optionally substituted C₃-C₁₀ carbocyclyl. In some embodiments, R is optionally substituted monocyclic carbocyclyl. In some embodiments, R is optionally substituted cycloheptyl. In some embodiments, R is optionally substituted cyclohexyl. In some embodiments, R is optionally substituted cyclopentyl. In some embodiments, R is optionally substituted cyclobutyl. In some embodiments, R is an optionally substituted cyclopropyl. In some embodiments, R is optionally substituted bicyclic carbocyclyl.

In some embodiments, R is an optionally substituted aryl. In some embodiments, R is an optionally substituted bicyclic aryl ring.

In some embodiments, R is an optionally substituted heteroaryl. In some embodiments, R is an optionally substituted 5-6 membered monocyclic heteroaryl ring having 1-3 heteroatoms independently selected from nitrogen, sulfur, and oxygen. In some embodiments, R is a substituted 5-6 membered monocyclic heteroaryl ring having 1-3 heteroatoms independently selected from nitrogen, oxygen, and sulfur. In some embodiments, R is an unsubstituted 5-6 membered monocyclic heteroaryl ring having 1-3 heteroatoms independently selected from nitrogen, sulfur, and oxygen.

In some embodiments, R is an optionally substituted 5-membered monocyclic heteroaryl ring having 1-3 heteroatoms independently selected from nitrogen, sulfur, and oxygen. In some embodiments, R is an optionally substituted 6 membered monocyclic heteroaryl ring having 1-3 heteroatoms independently selected from nitrogen, oxygen, and sulfur.

In some embodiments, R is an optionally substituted 5-membered monocyclic heteroaryl ring having 1 heteroatom selected from nitrogen, oxygen, and sulfur. In some embodiments, R is selected from pyrrolyl, furanyl, and thienyl.

In some embodiments, R is an optionally substituted 5-membered heteroaryl ring having 2 heteroatoms independently selected from nitrogen, oxygen, and sulfur. In certain embodiments, R is an optionally substituted 5-membered heteroaryl ring having 1 nitrogen atom, and an additional heteroatom selected from sulfur and oxygen. Example R groups include optionally substituted pyrazolyl, imidazolyl, thiazolyl, isothiazolyl, oxazolyl or isoxazolyl.

In some embodiments, R is a 6-membered heteroaryl ring having 1-3 nitrogen atoms. In other embodiments, R is an optionally substituted 6-membered heteroaryl ring having 1-2 nitrogen atoms. In some embodiments, R is an optionally substituted 6-membered heteroaryl ring having 2 nitrogen atoms. In certain embodiments, R is an optionally substituted 6-membered heteroaryl ring having 1 nitrogen. Example R groups include optionally substituted pyridinyl, pyrimidinyl, pyrazinyl, pyridazinyl, triazinyl, or tetrazinyl.

In certain embodiments, R is an optionally substituted 8-10 membered bicyclic heteroaryl ring having 1-4 heteroatoms independently selected from nitrogen, oxygen, and sulfur. In some embodiments, R is an optionally substituted 5,6-fused heteroaryl ring having 1-4 heteroatoms independently selected from nitrogen, oxygen, and sulfur. In other embodiments, R is an optionally substituted 5,6-fused heteroaryl ring having 1-2 heteroatoms independently selected from nitrogen, oxygen, and sulfur. In certain embodiments, R is an optionally substituted 5,6-fused heteroaryl ring having 1 heteroatom independently selected from nitrogen, oxygen, and sulfur. In some embodiments, R is an optionally substituted indolyl. In some embodiments, R is an optionally substituted azabicyclo[3.2.1]octanyl. In certain embodiments, R is an optionally substituted 5,6-fused heteroaryl ring having 2 heteroatoms independently selected from nitrogen, oxygen, and sulfur. In some embodiments, R is an optionally substituted azaindolyl. In some embodiments, R is an optionally substituted benzimidazolyl. In some embodiments, R is an optionally substituted benzothiazolyl. In some embodiments, R is an optionally substituted benzoxazolyl. In some embodiments, R is an optionally substituted indazolyl. In certain embodiments, R is an optionally substituted 5,6-fused heteroaryl ring having 3 heteroatoms independently selected from nitrogen, oxygen, and sulfur.

In certain embodiments, R is an optionally substituted 6,6-fused heteroaryl ring having 1-4 heteroatoms independently selected from nitrogen, oxygen, and sulfur. In some embodiments, R is an optionally substituted 6,6-fused heteroaryl ring having 1-2 heteroatoms independently selected from nitrogen, oxygen, and sulfur. In other embodiments, R is an optionally substituted 6,6-fused heteroaryl ring having 1 heteroatom independently selected from nitrogen, oxygen, and sulfur. In some embodiments, R is an optionally substituted quinolinyl. In some embodiments, R is an optionally substituted isoquinolinyl. According to one aspect, R is an optionally substituted 6,6-fused heteroaryl ring having 2 heteroatoms independently selected from nitrogen, oxygen, and sulfur. In some embodiments, R is a quinazoline or a quinoxaline.

In some embodiments, R is an optionally substituted heterocyclyl. In some embodiments, R is an optionally substituted 3-7 membered saturated or partially unsaturated heterocyclic ring having 1-2 heteroatoms independently selected from nitrogen, oxygen, and sulfur. In some embodiments, R is a substituted 3-7 membered saturated or partially unsaturated heterocyclic ring having 1-2 heteroatoms independently selected from nitrogen, oxygen, and sulfur. In some embodiments, R is an unsubstituted 3-7 membered saturated or partially unsaturated heterocyclic ring having 1-2 heteroatoms independently selected from nitrogen, oxygen, and sulfur.

In some embodiments, R is an optionally substituted heterocyclyl. In some embodiments, R is an optionally substituted 6 membered saturated or partially unsaturated heterocyclic ring having 1-2 heteroatoms independently selected from nitrogen, oxygen, and sulfur. In some embodiments, R is an optionally substituted 6 membered partially unsaturated heterocyclic ring having 2 heteroatoms independently selected from nitrogen, oxygen, and sulfur. In some embodiments, R is an optionally substituted 6 membered partially unsaturated heterocyclic ring having 2 oxygen atom.

In certain embodiments, R is a 3-7 membered saturated or partially unsaturated heterocyclic ring having 1-2 heteroatoms independently selected from nitrogen, oxygen, and sulfur. In certain embodiments, R is oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothiophenyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothiophenyl, or tetrahydrothiopyranyl. In some embodiments, R is an optionally substituted 5-membered saturated or partially unsaturated heterocyclic ring having 1-2 heteroatoms independently selected from nitrogen, oxygen, and sulfur.

In certain embodiments, R is an optionally substituted 5-6 membered partially unsaturated monocyclic ring having 1-2 heteroatoms independently selected from nitrogen, oxygen, and sulfur. In certain embodiments, R is an optionally substituted tetrahydropyridinyl, dihydrothiazolyl, dihydrooxazolyl, or oxazolinyl group.

In some embodiments, R is an optionally substituted 8-10 membered bicyclic saturated or partially unsaturated heterocyclic ring having 1-4 heteroatoms independently selected from nitrogen, oxygen, and sulfur. In some embodiments, R is an optionally substituted indolinyl. In some embodiments, R is an optionally substituted isoindolinyl. In some embodiments, R is an optionally substituted 1, 2, 3, 4-tetrahydroquinoline. In some embodiments, R is an optionally substituted 1, 2, 3, 4-tetrahydroisoquinoline.

In some embodiments, each R′ is independently —R, —C(O)R, —CO₂R, or —SO₂R, or:
two R′ on the same nitrogen are taken together with their intervening atoms to form an optionally substituted heterocyclic or heteroaryl ring, or
two R′ on the same carbon are taken together with their intervening atoms to form an optionally substituted aryl, carbocyclic, heterocyclic, or heteroaryl ring.

In some embodiments, R′ is —R, —C(O)R, —CO₂R, or —SO₂R, wherein R is as defined above and described herein.

In some embodiments, R′ is —R, wherein R is as defined and described above and herein. In some embodiments, R′ is hydrogen.

In some embodiments, R′ is —C(O)R, wherein R is as defined above and described herein. In some embodiments, R′ is —CO₂R, wherein R is as defined above and described herein. In some embodiments, R′ is —SO₂R, wherein R is as defined above and described herein.

In some embodiments, two R′ on the same nitrogen are taken together with their intervening atoms to form an optionally substituted heterocyclic or heteroaryl ring. In some embodiments, two R′ on the same carbon are taken together with their intervening atoms to form an optionally substituted aryl, carbocyclic, heterocyclic, or heteroaryl ring.

In some embodiments, -Cy- is an optionally substituted bivalent ring selected from phenylene, carbocyclylene, arylene, heteroarylene, and heterocyclylene.

In some embodiments, -Cy- is optionally substituted phenylene. In some embodiments, -Cy- is optionally substituted carbocyclylene. In some embodiments, -Cy- is optionally substituted arylene. In some embodiments, -Cy- is optionally substituted heteroarylene. In some embodiments, -Cy- is optionally substituted heterocyclylene.

In some embodiments, each of X, Y and Z is independently —O—, —S—, —N(-L-R¹)—, or L, wherein each of L and R¹ is independently as defined above and described below.

In some embodiments, X is —O—. In some embodiments, X is —S—. In some embodiments, X is —O— or —S—. In some embodiments, an oligonucleotide comprises at least one internucleotidic linkage of Formula I wherein X is —O—. In some embodiments, an oligonucleotide comprises at least one internucleotidic linkage of Formula I wherein X is —S—. In some embodiments, an oligonucleotide comprises at least one internucleotidic linkage of Formula I wherein X is —O—, and at least one internucleotidic linkage of Formula I wherein X is —S—. In some embodiments, an oligonucleotide comprises at least one internucleotidic linkage of Formula I wherein X is —O—, and at least one internucleotidic linkage of Formula I wherein X is —S—, and at least one internucleotidic linkage of Formula I wherein L is an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R)S(O)₂—, —SC(O)—, —C(O)S—, —OC(O)—, or —C(O)O—.

In some embodiments, X is —N(-L-R¹)—. In some embodiments, X is —N(R¹)—. In some embodiments, X is —N(R¹)—. In some embodiments, X is —N(R)—. In some embodiments, X is —NH—.

In some embodiments, X is L. In some embodiments, X is a covalent bond. In some embodiments, X is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, X is an optionally substituted C₁-C₁₀ alkylene or C₁-C₁₀ alkenylene. In some embodiments, X is methylene.

In some embodiments, Y is —O—. In some embodiments, Y is —S—.

In some embodiments, Y is —N(-L-R¹)—. In some embodiments, Y is —N(R¹)—. In some embodiments, Y is —N(R¹)—. In some embodiments, Y is —N(R)—. In some embodiments, Y is —NH—.

In some embodiments, Y is L. In some embodiments, Y is a covalent bond. In some embodiments, Y is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, Y is an optionally substituted C₁-C₁₀ alkylene or C₁-C₁₀ alkenylene. In some embodiments, Y is methylene. In some embodiments, Y is L, wherein at least one methylene units of L is optionally substituted replaced with —O—, —S—, or —N(R¹)—, wherein Y is connected to P L through —O—, —S—, or —N(R¹)—. In some embodiments, Y is L, wherein L is -L³-G-. In some embodiments, G is bonded to P^{L}. In some embodiments, G is —O—. In some embodiments, G is —S—. In some embodiments, G is —N(R′)—.

In some embodiments, Z is —O—. In some embodiments, Z is —S—.

In some embodiments, Z is —N(-L-R¹)—. In some embodiments, Z is —N(R¹)—. In some embodiments, Z is —N(R′)—. In some embodiments, Z is —N(R)—. In some embodiments, Z is —NH—.

In some embodiments, Z is L. In some embodiments, Z is a covalent bond. In some embodiments, Z is or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—. In some embodiments, Z is an optionally substituted C₁-C₁₀ alkylene or C₁-C₁₀ alkenylene. In some embodiments, Z is methylene. In some embodiments, Z is L, wherein at least one methylene units of L is optionally substituted replaced with —O—, —S—, or —N(R¹)—, wherein Z is connected to P L through —O—, —S—, or —N(R¹)—. In some embodiments, Z is L, wherein L is -L³-G-. In some embodiments, G is bonded to P^{L}. In some embodiments, G is —O—. In some embodiments, G is —S—. In some embodiments, G is —N(R¹)—.

In some embodiments, L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—.

In some embodiments, L is a covalent bond. In some embodiments, L is an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, -S(O)₂—, —S(O)₂N(R′)—, —N(R′)S(O)₂—, —SC(O)—, —C(O)S—, —OC(O)—, or —C(O)O—.

In some embodiments, L has the structure of wherein:
L¹ is an optionally substituted group selected from

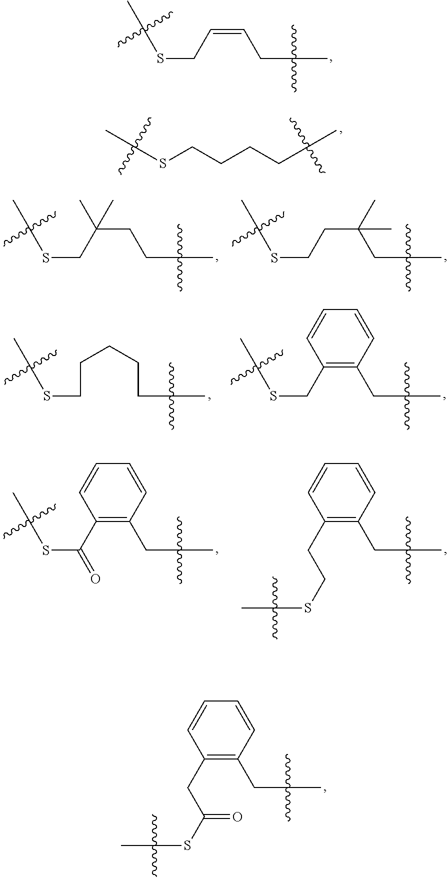

C₁-C₆ alkylene, C₁-C₆ alkenylene, carbocyclylene, arylene, C₁-C₆ heteroalkylene, heterocyclylene, and heteroarylene;
V is selected from —O—, —S—, —NR′—, C(R′)₂, —S—S—, —B—S—S—C—,

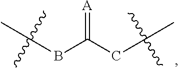

and an optionally substituted group selected from C₁-C₆ alkylene, arylene, C₁-C₆ heteroalkylene, heterocyclylene, and heteroarylene;
A is ═O, ═S, ═NR′, or ═C(R′)₂;
each of B and C is independently —O—, —S—, —NR′—, —C(R′)₂—, or an optionally substituted group selected from C₁-C₆ alkylene, carbocyclylene, arylene, heterocyclylene, and heteroarylene; and each R′ is independently as defined above and described herein.

In some embodiments, L¹ is

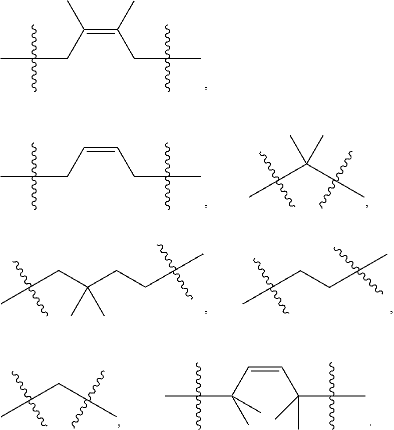

In some embodiments, L¹ is

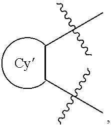 [SMILES: *CC*]

wherein Ring Cy′ is an optionally substituted arylene, carbocyclylene, heteroarylene, or heterocyclylene. In some embodiments, L¹ is optionally substituted

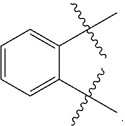 [SMILES: *c1ccccc1*]

In some embodiments, L¹ is

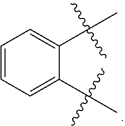 [SMILES: *c1ccccc1*]

In some embodiments, L¹ is connected to X. In some embodiments, L¹ is an optionally substituted group selected from

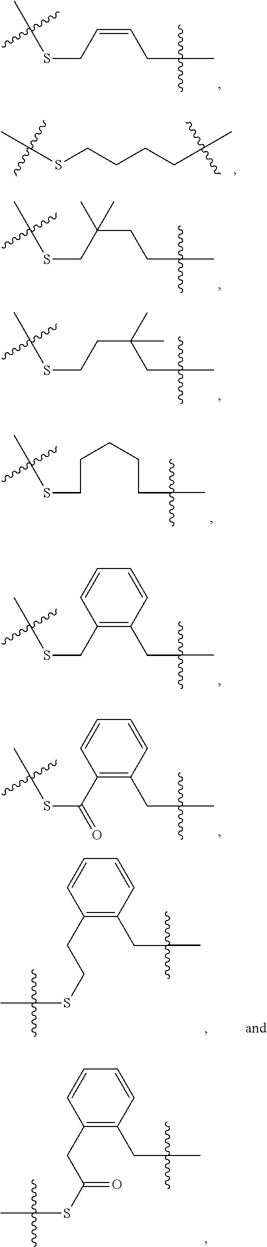

and the sulfur atom being connected to V. In some embodiments, L¹ is an optionally substituted group selected from

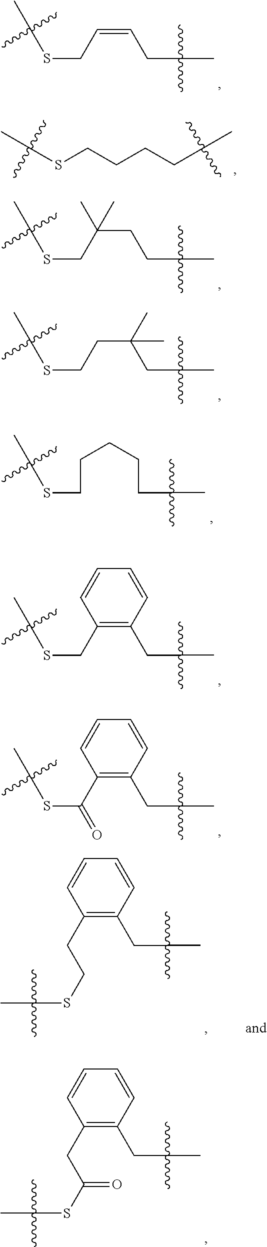

and the carbon atom being connected to X.

In some embodiments, L has the structure of:

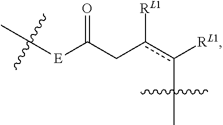

wherein:
E is —O—, —S—, —NR′ or —C(R′)₂—;
[symbol] is a single or double bond;
the two R^{L1} are taken together with the two carbon atoms to which they are bound to form an optionally substituted aryl, carbocyclic, heteroaryl or heterocyclic ring; and each R′ is independently as defined above and described herein.

In some embodiments, L has the structure of:

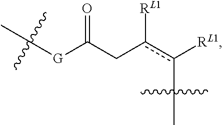

wherein:
G is —O—, —S—, or —NR′;
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, L has the structure of:

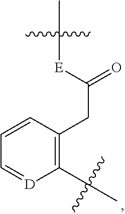

wherein:
E is —O—, —S—, —NR′ or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))—, or ═C(CF₃)—; and
each R′ is independently as defined above and described herein.

In some embodiments, L has the structure of:

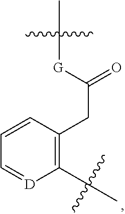

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—.

In some embodiments, L has the structure of:

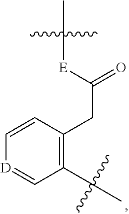

wherein:
E is —O—, —S—, —NR′ or —C(R′)₂—;
D is ═N—, ═C(F)—, ═C(Cl)—, ═C(Br)—, ═C(I)—, ═C(CN)—, ═C(NO₂)—, ═C(CO₂—(C₁-C₆ aliphatic))—, or ═C(CF₃)—; and
each R′ is independently as defined above and described herein.

In some embodiments, L has the structure of:

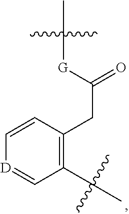

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—.

In some embodiments, L has the structure of:

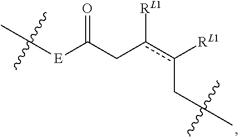

wherein:
E is —O—, —S—, —NR′— or —C(R′)₂—;
[symbol] is a single or double bond;
the two R^{L1} are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

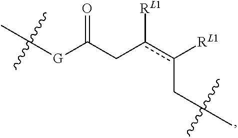

wherein:
G is —O—, —S—, or —NR′;
[symbol] is a single or double bond;
the two R′ are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

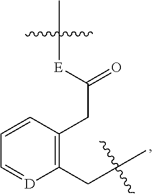

wherein:
E is —O—, —S—, —NR′ or —C(R′)₂—;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

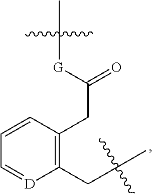

wherein:
G is —O—, —S—, or —NR′;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

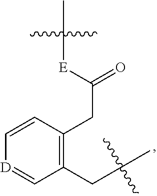

wherein:
E is —O—, —S—, —NR′— or —C(R′)₂—;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

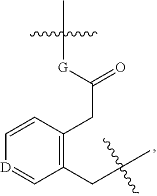

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

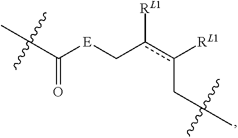

wherein:
E is —O—, —S—, —NR′— or —C(R′)₂—;
[symbol] is a single or double bond;
the two R^{L1} are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

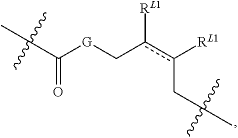

wherein:
G is —O—, —S—, or —NR′;
[symbol] is a single or double bond;
the two R^{L1} are taken together with the two carbon atoms to which they are bound to form an optionally substituted aryl, C₃-C₁₀ carbocyclic, heteroaryl or heterocyclic ring; and each R′ is independently as defined above and described herein.

In some embodiments, L has the structure of:

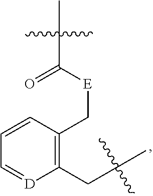

wherein:
E is —O—, —S—, —NR′— or —C(R′)₂—;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

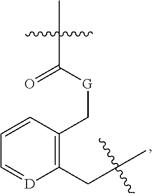

wherein:
G is —O—, —S—, or —NR′;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
R′ is as defined above and described herein.

In some embodiments, L has the structure of:

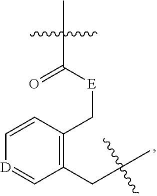

wherein:
E is —O—, —S—, —NR′— or —C(R′)₂—;
D is ═N—, ═C(F)—, ═C(Cl)—, ═C(Br)—, ═C(CN)—, ═C(NO₂)—, ═C(CO₂—(C₁-C₆ aliphatic))-, or ═C(CF₃)—; and
each R′ is independently as defined above and described herein.

In some embodiments, L has the structure of:

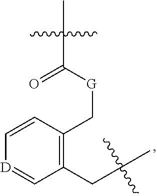

wherein:
G is —O—, —S—, or —NR′;
D is ═N—, ═C(F)—, ═C(Cl)—, ═C(Br)—, ═C(I)—, ═C(CN)—, ═C(NO₂)—, ═C(CO₂—(C₁-C₆ aliphatic))-, or ═C(CF₃)—; and
R′ is as defined above and described herein.

In some embodiments, L has the structure of:

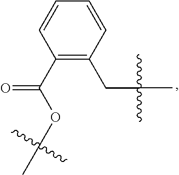 [SMILES: *Cc1ccccc1C(=O)O*]

wherein the phenyl ring is optionally substituted. In some embodiments, the phenyl ring is not substituted. In some embodiments, the phenyl ring is substituted.

In some embodiments, L has the structure of:

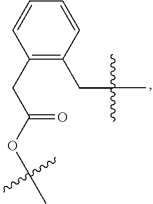 [SMILES: *Cc1ccccc1CC(=O)O*]

wherein the phenyl ring is optionally substituted. In some embodiments, the phenyl ring is not substituted. In some embodiments, the phenyl ring is substituted.

In some embodiments, L has the structure of:

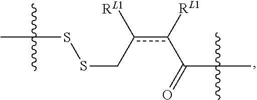

wherein:
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, L has the structure of:

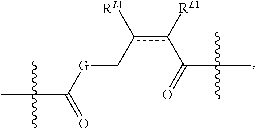

wherein:
G is —O—, —S—, or —NR′;
[symbol] is a single or double bond; and
the two R^{L1} are taken together with the two carbon atoms to which they are bound to form an optionally substituted aryl, C₃-C₁₀ carbocyclic, heteroaryl or heterocyclic ring.

In some embodiments, E is —O—, —S—, —NR′— or —C(R′)₂—, wherein each R′ independently as defined above and described herein. In some embodiments, E is —O—, —S—, or —NR′—. In some embodiments, E is —O—, —S—, or —NH—. In some embodiments, E is —O—. In some embodiments, E is —S—. In some embodiments, E is —NH—.

In some embodiments, G is —O—, —S—, or —NR′—, wherein each R′ independently as defined above and described herein. In some embodiments, G is —O—, —S—, or —NH—. In some embodiments, G is —O—. In some embodiments, G is —S—. In some embodiments, G is —NH—.

In some embodiments, L is -L³-G-, wherein:
L³ is an optionally substituted C₁-C₅ alkylene or alkenylene, wherein one or more methylene units are optionally and independently replaced by —O—, —S—, —N(R¹)—, —C(O)—, —C(S)—, —C(NR′)—, —S(O)—, —S(O)₂—, or

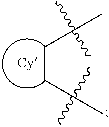 [SMILES: *CC*]

and
wherein each of G, R′ and Ring Cy′ is independently as defined above and described herein.

In some embodiments, L is -L³-S—, wherein L³ is as defined above and described herein. In some embodiments, L is -L³-O—, wherein L³ is as defined above and described herein. In some embodiments, L is -L³-N(R¹)—, wherein each of L³ and R′ is independently as defined above and described herein. In some embodiments, L is -L³-NH—, wherein each of L³ and R′ is independently as defined above and described herein.

In some embodiments, L 3 is an optionally substituted Cs alkylene or alkenylene, wherein one or more methylene units are optionally and independently replaced by —O—, —S—, —N(R¹)—, —C(O)—, —C(S)—, —C(NR′)—, —S(O)—, —S(O)₂—, or

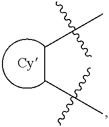 [SMILES: *CC*]

and each of R′ and Ring Cy′ is independently as defined above and described herein. In some embodiments, L³ is an optionally substituted C₅ alkylene. In some embodiments, -L³-G- is

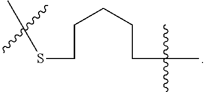 [SMILES: *CCCCCS*]

In some embodiments, L³ is an optionally substituted C₄ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

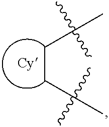 [SMILES: *CC*]

and each of R′ and Cy′ is independently as defined above and described herein.

In some embodiments, -L³-G- is

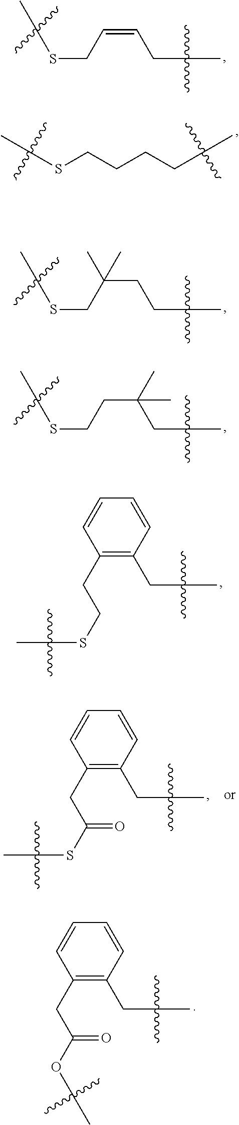

In some embodiments, L³ is an optionally substituted C₃ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

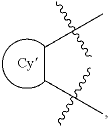 [SMILES: *CC*]

and each of R′ and Cy′ is independently as defined above and described herein.

In some embodiments, -L³-G- is

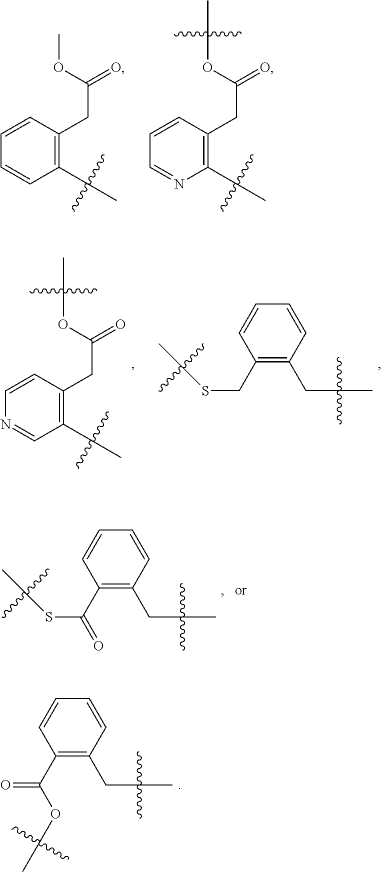 [SMILES: *c1ccccc1CC(=O)OC ; *OC(=O)Cc1cccnc1* ; *OC(=O)Cc1ccncc1* ; *Cc1ccccc1CS* ; *Cc1ccccc1C(=O)S* ; *Cc1ccccc1C(=O)O*]

In some embodiments, L is

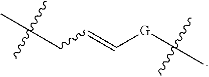

In some embodiments, L is

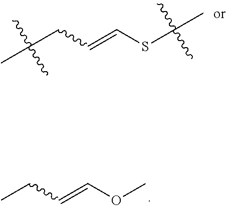

In some embodiments, L is

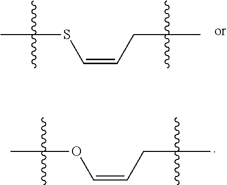 [SMILES: *C/C=C\S* ; *C/C=C\O*]

In some embodiments, L³ is an optionally substituted C₂ alkylene or alkenylene, wherein one or more methylene units are optionally and independently replaced by —O—, —S—, —N(R′)—, —C(O)—, —C(S)—, —C(NR′)—, —S(O)—, —S(O)₂—, or

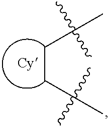 [SMILES: *CC*]

and each of R′ and Cy′ is independently as defined above and described herein.

In some embodiments, -L³-G- is

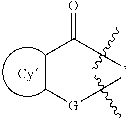

wherein each of G and Cy′ is independently as defined above and described herein. In some embodiments, L is

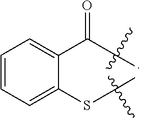 [SMILES: *Sc1ccccc1C(*)=O]

In some embodiments, L is -L⁴-G-, wherein L⁴ is an optionally substituted C₁-C₂ alkylene; and G is as defined above and described herein. In some embodiments, L is -L⁴-G-, wherein L⁴ is an optionally substituted C₁-C₂ alkylene; G is as defined above and described herein; and G is connected to In some embodiments, L is -L⁴-G-, wherein L⁴ is an optionally substituted methylene; G is as defined above and described herein; and G is connected to In some embodiments, L is -L⁴-G-, wherein L⁴ is methylene; G is as defined above and described herein; and G is connected to R¹. In some embodiments, L is -L⁴-G-, wherein L⁴ is an optionally substituted —(CH₂)₂—; G is as defined above and described herein; and G is connected to In some embodiments, L is -L⁴-G-, wherein L⁴ is —(CH₂)₂—; G is as defined above and described herein; and G is connected to R¹.

In some embodiments, L is

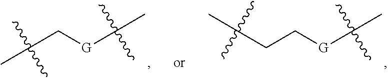

wherein G is as defined above and described herein, and G is connected to R¹. In some embodiments, L is

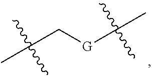

wherein G is as defined above and described herein, and G is connected to R¹. In some embodiments, L is

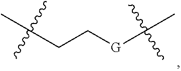

wherein G is as defined above and described herein, and G is connected to R¹. In some embodiments, L is

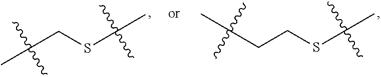

wherein the sulfur atom is connected to R¹. In some embodiments, L is

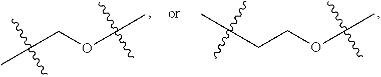

wherein the oxygen atom is connected to R¹.

In some embodiments, L is

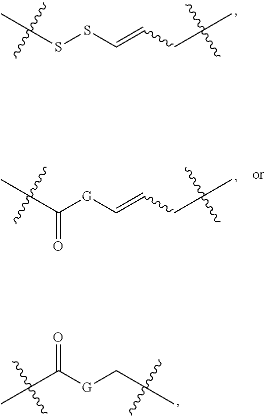

wherein G is as defined above and described herein.

In some embodiments, L is —S—R^{L3}— or —S—C(O)—R^{L3}—, wherein R^{L3} is an optionally substituted, linear or branched, C₁-C₉ alkylene, wherein one or more methylene units are optionally and independently replaced by an optionally substituted C₁-C₆ alkylene, C₁-C₆ alkenylene, —≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each of R′ and -Cy- is independently as defined above and described herein. In some embodiments, L is —S—R^{L3}— or —S—C(O)—R^{L3}—, wherein R^{L3} is an optionally substituted C₁-C₆ alkylene. In some embodiments, L is —S—R^{L3}— or —S—C(O)—R^{L3}—, wherein R^{L3} is an optionally substituted C₁-C₆ alkenylene. In some embodiments, L is —S—R^{L3}— or —S—C(O)—R^{L3}—, wherein R^{L3} is an optionally substituted C₁-C₆ alkylene wherein one or more methylene units are optionally and independently replaced by an optionally substituted C₁-C₆ alkenylene, arylene, or heteroarylene. In some embodiments, In some embodiments, R^{L3} is an optionally substituted —S—(C₁-C₆ alkenylene)-, —S—(C₁-C₆ alkylene)-, —S—(C₁-C₆ alkylene)-arylene-(C₁-C₆ alkylene)-, —S—CO-arylene-(C₁-C₆ alkylene)-, or —S—CO—(C₁-C₆ alkylene)-arylene-(C₁-C₆ alkylene)-.

In some embodiments, L is

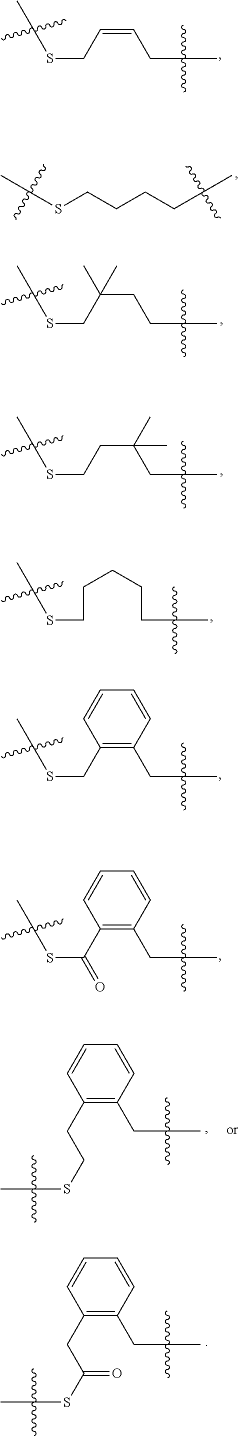

In some embodiments, L is

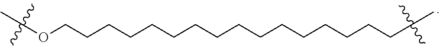 [SMILES: *CCCCCCCCCCCCCCCCO*]

In some embodiments, L is

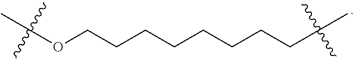 [SMILES: *CCCCCCCCO*]

In some embodiments,

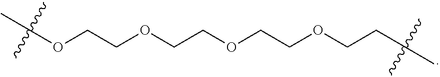 [SMILES: *CCOCCOCCOCCO*]

In some embodiments, the sulfur atom in the L embodiments described above and herein is connected to X. In some embodiments, the sulfur atom in the L embodiments described above and herein is connected to R¹.

In some embodiments, R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is halogen, R, or an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein.

In some embodiments, R¹ is hydrogen. In some embodiments, R¹ is halogen. In some embodiments, R¹ is —F. In some embodiments, R¹ is —Cl. In some embodiments, R¹ is —Br. In some embodiments, R¹ is —I.

In some embodiments, R¹ is R wherein R is as defined above and described herein.

In some embodiments, R¹ is hydrogen. In some embodiments, R¹ is an optionally substituted group selected from C₁-C₅₀ aliphatic, phenyl, carbocyclyl, aryl, heteroaryl, and heterocyclyl.

In some embodiments, R¹ is an optionally substituted C₁-C₅₀ aliphatic. In some embodiments, R¹ is an optionally substituted C₁-C₁₀ aliphatic. In some embodiments, R¹ is an optionally substituted C₁-C₆ aliphatic. In some embodiments, R¹ is an optionally substituted C₁-C₆ alkyl. In some embodiments, R¹ is optionally substituted, linear or branched hexyl. In some embodiments, R¹ is optionally substituted, linear or branched pentyl. In some embodiments, R¹ is optionally substituted, linear or branched butyl. In some embodiments, R¹ is optionally substituted, linear or branched propyl. In some embodiments, R¹ is optionally substituted ethyl. In some embodiments, R¹ is optionally substituted methyl.

In some embodiments, R¹ is optionally substituted phenyl. In some embodiments, R¹ is substituted phenyl. In some embodiments, R¹ is phenyl.

In some embodiments, R¹ is optionally substituted carbocyclyl. In some embodiments, R¹ is optionally substituted C₃-C₁₀ carbocyclyl. In some embodiments, R¹ is optionally substituted monocyclic carbocyclyl. In some embodiments, R¹ is optionally substituted cycloheptyl. In some embodiments, R¹ is optionally substituted cyclohexyl. In some embodiments, R¹ is optionally substituted cyclopentyl. In some embodiments, R¹ is optionally substituted cyclobutyl. In some embodiments, R¹ is an optionally substituted cyclopropyl. In some embodiments, R¹ is optionally substituted bicyclic carbocyclyl.

In some embodiments, R¹ is an optionally substituted C₁-C₅₀ polycyclic hydrocarbon. In some embodiments, R¹ is an optionally substituted C₁-C₅₀ polycyclic hydrocarbon wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is optionally substituted

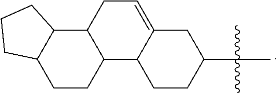 [SMILES: *C1CCC2C(=CCC3C4CCCC4CCC23)C1]

In some embodiments, R¹ is

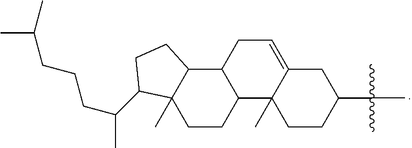 [SMILES: *C1CCC2(C)C(=CCC3C2CCC2(C)C(C(C)CCCC(C)C)CCC32)C1]

In some embodiments, R¹ is optionally substituted

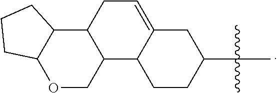 [SMILES: *C1CCC2C(=CCC3C4CCCC4OCC23)C1]

In some embodiments, R¹ is an optionally substituted C₁-C₅₀ aliphatic comprising one or more optionally substituted polycyclic hydrocarbon moieties. In some embodiments, R¹ is an optionally substituted C₁-C₅₀ aliphatic comprising one or more optionally substituted polycyclic hydrocarbon moieties, wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is an optionally substituted C₁-C₅₀ aliphatic comprising one or more optionally substituted

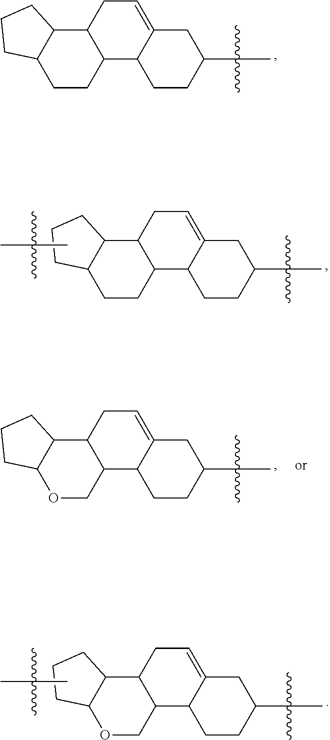

In some embodiments, R¹ is

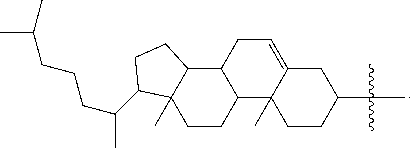 [SMILES: *C1CCC2(C)C(=CCC3C2CCC2(C)C(C(C)CCCC(C)C)CCC32)C1]

In some embodiments, R¹ is

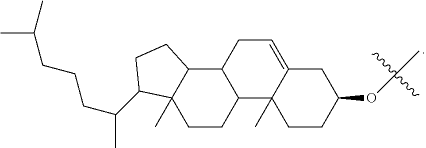 [SMILES: *O[C@H]1CCC2(C)C(=CCC3C2CCC2(C)C(C(C)CCCC(C)C)CCC32)C1]

In some embodiments, R¹ is

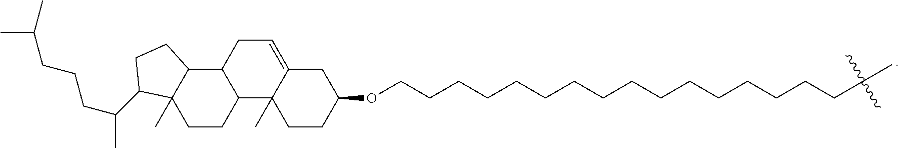 [SMILES: *CCCCCCCCCCCCCCCCO[C@H]1CCC2(C)C(=CCC3C2CCC2(C)C(C(C)CCCC(C)C)CCC32)C1]

In some embodiments, R¹ is

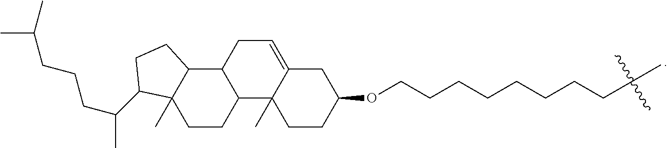 [SMILES: *CCCCCCCCO[C@H]1CCC2(C)C(=CCC3C2CCC2(C)C(C(C)CCCC(C)C)CCC32)C1]

In some embodiments, R¹ is

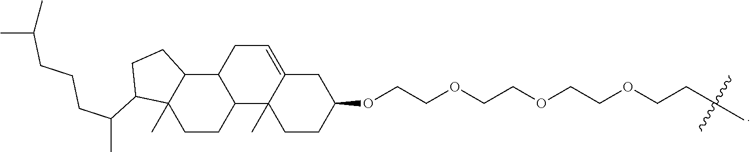 [SMILES: *CCOCCOCCOCCO[C@H]1CCC2(C)C(=CCC3C2CCC2(C)C(C(C)CCCC(C)C)CCC32)C1]

In some embodiments, R¹ is an optionally substituted aryl. In some embodiments, R¹ is an optionally substituted bicyclic aryl ring.

In some embodiments, R¹ is an optionally substituted heteroaryl. In some embodiments, R¹ is an optionally substituted 5-6 membered monocyclic heteroaryl ring having 1-3 heteroatoms independently selected from nitrogen, sulfur, and oxygen. In some embodiments, R¹ is a substituted 5-6 membered monocyclic heteroaryl ring having 1-3 heteroatoms independently selected from nitrogen, oxygen, and sulfur. In some embodiments, R¹ is an unsubstituted 5-6 membered monocyclic heteroaryl ring having 1-3 heteroatoms independently selected from nitrogen, sulfur, and oxygen.

In some embodiments, R¹ is an optionally substituted 5-membered monocyclic heteroaryl ring having 1-3 heteroatoms independently selected from nitrogen, sulfur, and oxygen. In some embodiments, R¹ is an optionally substituted 6 membered monocyclic heteroaryl ring having 1-3 heteroatoms independently selected from nitrogen, oxygen, and sulfur.

In some embodiments, R¹ is an optionally substituted 5-membered monocyclic heteroaryl ring having 1 heteroatom selected from nitrogen, oxygen, and sulfur. In some embodiments, R¹ is selected from pyrrolyl, furanyl, and thienyl.

In some embodiments, R¹ is an optionally substituted 5-membered heteroaryl ring having 2 heteroatoms independently selected from nitrogen, oxygen, and sulfur. In certain embodiments, R¹ is an optionally substituted 5-membered heteroaryl ring having 1 nitrogen atom, and an additional heteroatom selected from sulfur and oxygen. Example R¹ groups include optionally substituted pyrazolyl, imidazolyl, thiazolyl, isothiazolyl, oxazolyl or isoxazolyl.

In some embodiments, R¹ is a 6-membered heteroaryl ring having 1-3 nitrogen atoms. In other embodiments, R¹ is an optionally substituted 6-membered heteroaryl ring having 1-2 nitrogen atoms. In some embodiments, R¹ is an optionally substituted 6-membered heteroaryl ring having 2 nitrogen atoms. In certain embodiments, R¹ is an optionally substituted 6-membered heteroaryl ring having 1 nitrogen. Example R¹ groups include optionally substituted pyridinyl, pyrimidinyl, pyrazinyl, pyridazinyl, triazinyl, or tetrazinyl.

In certain embodiments, R¹ is an optionally substituted 8-10 membered bicyclic heteroaryl ring having 1-4 heteroatoms independently selected from nitrogen, oxygen, and sulfur. In some embodiments, R¹ is an optionally substituted 5,6-fused heteroaryl ring having 1-4 heteroatoms independently selected from nitrogen, oxygen, and sulfur. In other embodiments, le is an optionally substituted 5,6-fused heteroaryl ring having 1-2 heteroatoms independently selected from nitrogen, oxygen, and sulfur. In certain embodiments, R¹ is an optionally substituted 5,6-fused heteroaryl ring having 1 heteroatom independently selected from nitrogen, oxygen, and sulfur. In some embodiments, R¹ is an optionally substituted indolyl. In some embodiments, R¹ is an optionally substituted azabicyclo[3.2.1]octanyl. In certain embodiments, R¹ is an optionally substituted 5,6-fused heteroaryl ring having 2 heteroatoms independently selected from nitrogen, oxygen, and sulfur. In some embodiments, R¹ is an optionally substituted azaindolyl. In some embodiments, R¹ is an optionally substituted benzimidazolyl. In some embodiments, R¹ is an optionally substituted benzothiazolyl. In some embodiments, R¹ is an optionally substituted benzoxazolyl. In some embodiments, R¹ is an optionally substituted indazolyl. In certain embodiments, R¹ is an optionally substituted 5,6-fused heteroaryl ring having 3 heteroatoms independently selected from nitrogen, oxygen, and sulfur.

In certain embodiments, R¹ is an optionally substituted 6,6-fused heteroaryl ring having 1-4 heteroatoms independently selected from nitrogen, oxygen, and sulfur. In some embodiments, R¹ is an optionally substituted 6,6-fused heteroaryl ring having 1-2 heteroatoms independently selected from nitrogen, oxygen, and sulfur. In other embodiments, R¹ is an optionally substituted 6,6-fused heteroaryl ring having 1 heteroatom independently selected from nitrogen, oxygen, and sulfur. In some embodiments, R¹ is an optionally substituted quinolinyl. In some embodiments, R¹ is an optionally substituted isoquinolinyl. According to one aspect, R¹ is an optionally substituted 6,6-fused heteroaryl ring having 2 heteroatoms independently selected from nitrogen, oxygen, and sulfur. In some embodiments, R¹ is a quinazoline or a quinoxaline.

In some embodiments, R¹ is an optionally substituted heterocyclyl. In some embodiments, R¹ is an optionally substituted 3-7 membered saturated or partially unsaturated heterocyclic ring having 1-2 heteroatoms independently selected from nitrogen, oxygen, and sulfur. In some embodiments, R¹ is a substituted 3-7 membered saturated or partially unsaturated heterocyclic ring having 1-2 heteroatoms independently selected from nitrogen, oxygen, and sulfur. In some embodiments, R¹ is an unsubstituted 3-7 membered saturated or partially unsaturated heterocyclic ring having 1-2 heteroatoms independently selected from nitrogen, oxygen, and sulfur.

In some embodiments, R¹ is an optionally substituted heterocyclyl. In some embodiments, R¹ is an optionally substituted 6 membered saturated or partially unsaturated heterocyclic ring having 1-2 heteroatoms independently selected from nitrogen, oxygen, and sulfur. In some embodiments, R¹ is an optionally substituted 6 membered partially unsaturated heterocyclic ring having 2 heteroatoms independently selected from nitrogen, oxygen, and sulfur. In some embodiments, R¹ is an optionally substituted 6 membered partially unsaturated heterocyclic ring having 2 oxygen atoms.

In certain embodiments, R¹ is a 3-7 membered saturated or partially unsaturated heterocyclic ring having 1-2 heteroatoms independently selected from nitrogen, oxygen, and sulfur. In certain embodiments, R¹ is oxiranyl, oxetanyl, tetrahydrofuranyl, tetrahydropyranyl, oxepaneyl, aziridineyl, azetidineyl, pyrrolidinyl, piperidinyl, azepanyl, thiiranyl, thietanyl, tetrahydrothiophenyl, tetrahydrothiopyranyl, thiepanyl, dioxolanyl, oxathiolanyl, oxazolidinyl, imidazolidinyl, thiazolidinyl, dithiolanyl, dioxanyl, morpholinyl, oxathianyl, piperazinyl, thiomorpholinyl, dithianyl, dioxepanyl, oxazepanyl, oxathiepanyl, dithiepanyl, diazepanyl, dihydrofuranonyl, tetrahydropyranonyl, oxepanonyl, pyrolidinonyl, piperidinonyl, azepanonyl, dihydrothiophenonyl, tetrahydrothiopyranonyl, thiepanonyl, oxazolidinonyl, oxazinanonyl, oxazepanonyl, dioxolanonyl, dioxanonyl, dioxepanonyl, oxathiolinonyl, oxathianonyl, oxathiepanonyl, thiazolidinonyl, thiazinanonyl, thiazepanonyl, imidazolidinonyl, tetrahydropyrimidinonyl, diazepanonyl, imidazolidinedionyl, oxazolidinedionyl, thiazolidinedionyl, dioxolanedionyl, oxathiolanedionyl, piperazinedionyl, morpholinedionyl, thiomorpholinedionyl, tetrahydropyranyl, tetrahydrofuranyl, morpholinyl, thiomorpholinyl, piperidinyl, piperazinyl, pyrrolidinyl, tetrahydrothiophenyl, or tetrahydrothiopyranyl. In some embodiments, R¹ is an optionally substituted 5-membered saturated or partially unsaturated heterocyclic ring having 1-2 heteroatoms independently selected from nitrogen, oxygen, and sulfur.

In certain embodiments, R¹ is an optionally substituted 5-6 membered partially unsaturated monocyclic ring having 1-2 heteroatoms independently selected from nitrogen, oxygen, and sulfur. In certain embodiments, R¹ is an optionally substituted tetrahydropyridinyl, dihydrothiazolyl, dihydrooxazolyl, or oxazolinyl group.

In some embodiments, R¹ is an optionally substituted 8-10 membered bicyclic saturated or partially unsaturated heterocyclic ring having 1-4 heteroatoms independently selected from nitrogen, oxygen, and sulfur. In some embodiments, R¹ is an optionally substituted indolinyl. In some embodiments, R¹ is an optionally substituted isoindolinyl. In some embodiments, R¹ is an optionally substituted 1, 2, 3, 4-tetrahydroquinoline. In some embodiments, R¹ is an optionally substituted 1, 2, 3, 4-tetrahydroisoquinoline.

In some embodiments, R¹ is an optionally substituted C₁-C₁₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, wherein each variable is independently as defined above and described herein. In some embodiments, R¹ is an optionally substituted C₁-C₁₀ aliphatic wherein one or more methylene units are optionally and independently replaced by an optionally -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —OC(O)—, or —C(O)O—, wherein each R′ is independently as defined above and described herein. In some embodiments, R¹ is an optionally substituted C₁-C₁₀ aliphatic wherein one or more methylene units are optionally and independently replaced by an optionally -Cy-, —O—, —S—, —S—S—, —N(R′)—, —C(O)—, —OC(O)—, or —C(O)O—, wherein each R′ is independently as defined above and described herein.

In some embodiments, R¹ is

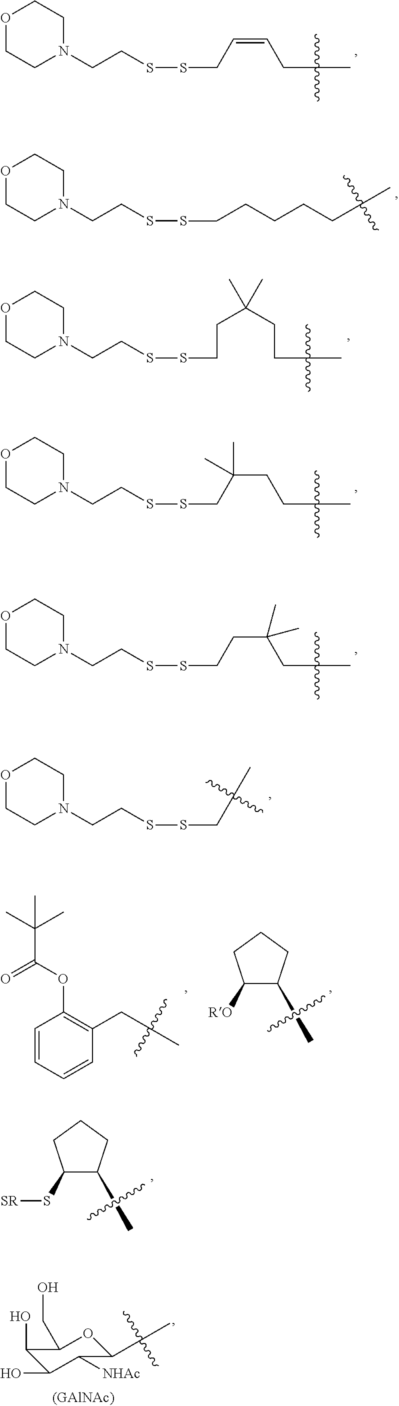

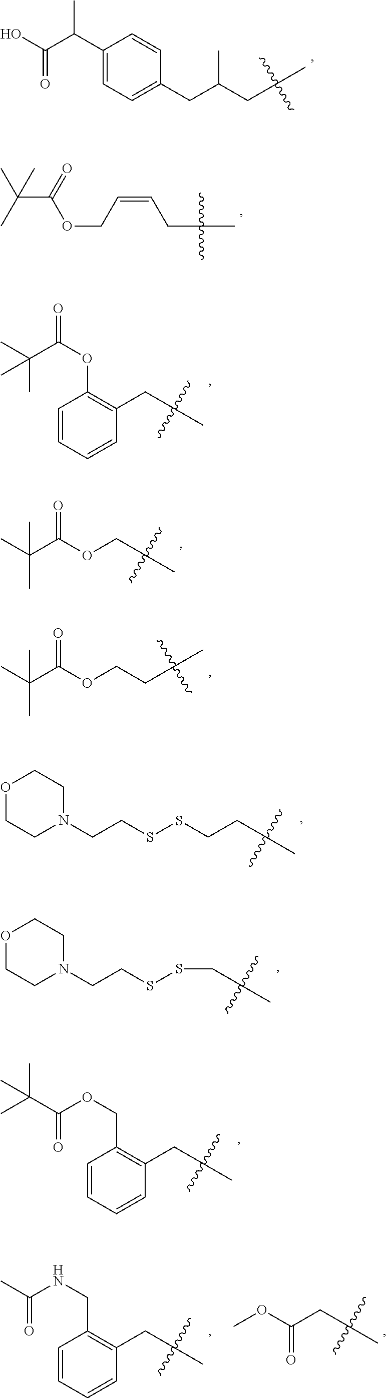

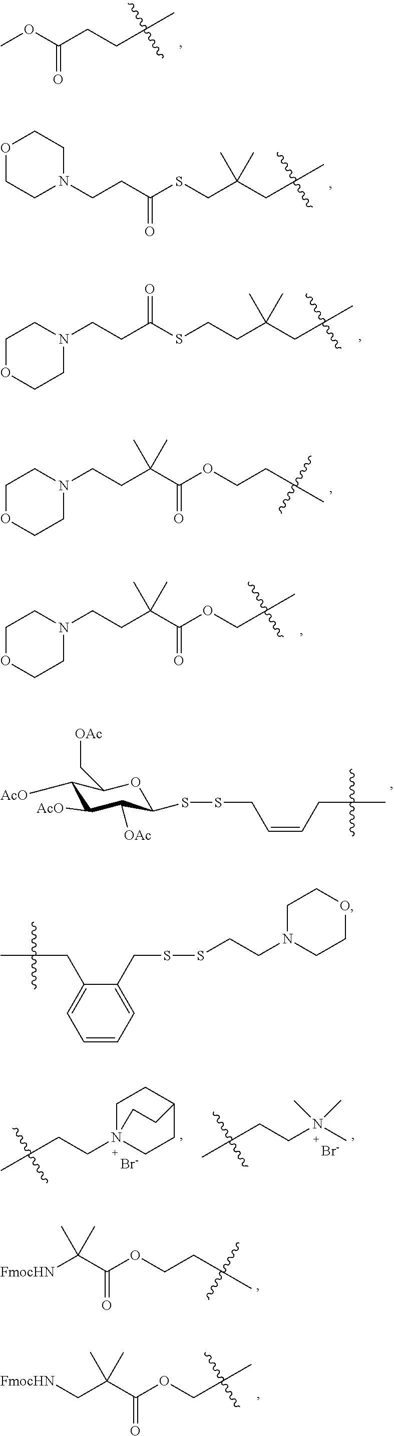

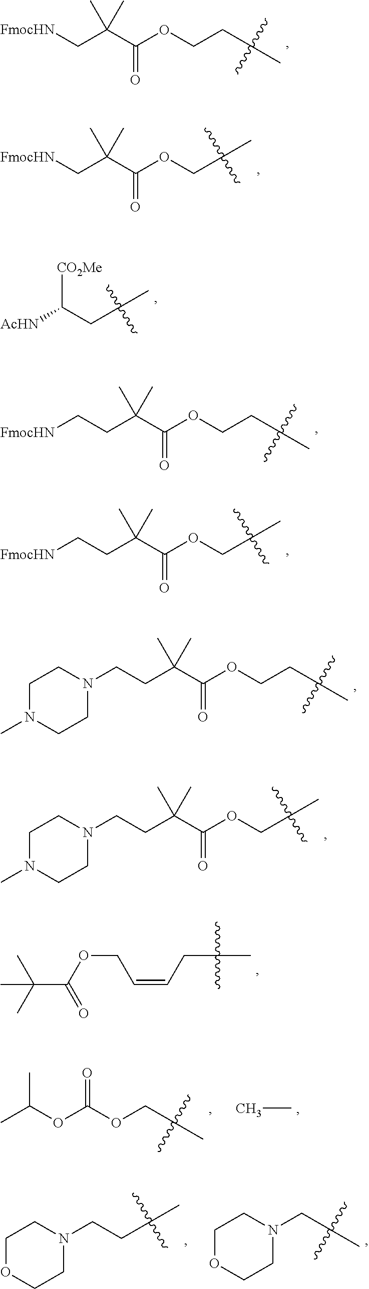

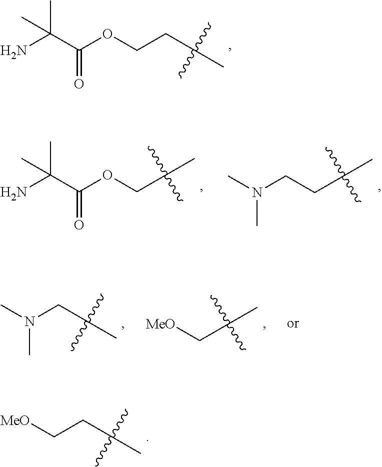

In some embodiments, R¹ is CH₃—,

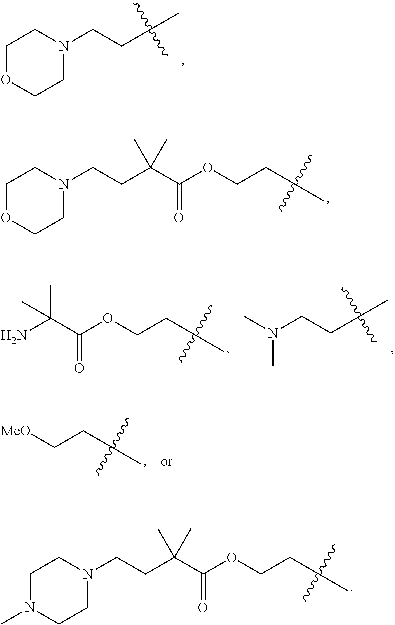

In some embodiments, R¹ comprises a terminal optionally substituted —(CH₂)₂— moiety which is connected to L. Examples of such R¹ groups are depicted below:

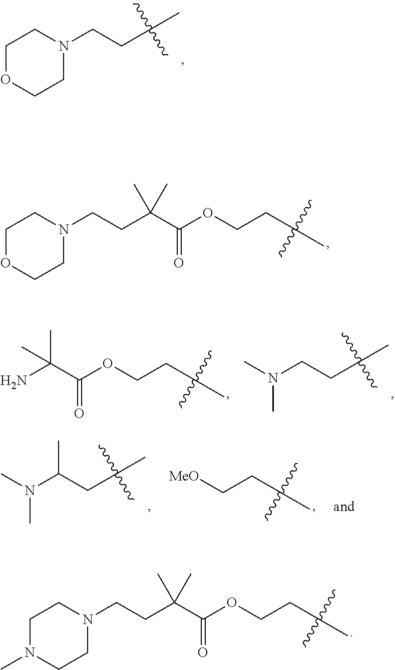

In some embodiments, R¹ comprises a terminal optionally substituted —(CH₂)— moiety which is connected to L. Example such R¹ groups are depicted below:

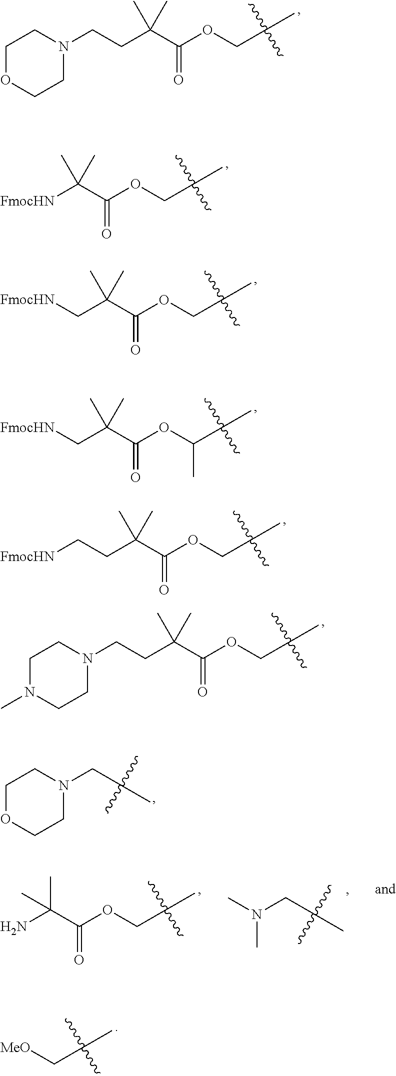

In some embodiments, R¹ is —S—R^{L2}, wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each of R′ and -Cy- is independently as defined above and described herein. In some embodiments, R¹ is —S—R^{L2}, wherein the sulfur atom is connected with the sulfur atom in L group.

In some embodiments, R¹ is —C(O)—R^{L2}, wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each of R′ and -Cy- is independently as defined above and described herein. In some embodiments, R¹ is —C(O)—R^{L2}, wherein the carbonyl group is connected with Gin L group. In some embodiments, R¹ is —C(O)—R^{L2}, wherein the carbonyl group is connected with the sulfur atom in L group.

In some embodiments, R^{L2} is optionally substituted C₁-C₉ aliphatic. In some embodiments, R^{L2} is optionally substituted C₁-C₉ alkyl. In some embodiments, R^{L2} is optionally substituted C₁-C₉ alkenyl. In some embodiments, R^{L2} is optionally substituted C₁-C₉ alkynyl. In some embodiments, R^{L2} is an optionally substituted C₁-C₉ aliphatic wherein one or more methylene units are optionally and independently replaced by -Cy- or —C(O)—. In some embodiments, R^{L2} is an optionally substituted C₁-C₉ aliphatic wherein one or more methylene units are optionally and independently replaced by -Cy-. In some embodiments, R^{L2} is an optionally substituted C₁-C₉ aliphatic wherein one or more methylene units are optionally and independently replaced by an optionally substituted heterocycylene. In some embodiments, R^{L2} is an optionally substituted C₁-C₉ aliphatic wherein one or more methylene units are optionally and independently replaced by an optionally substituted arylene. In some embodiments, R^{L2} is an optionally substituted C₁-C₉ aliphatic wherein one or more methylene units are optionally and independently replaced by an optionally substituted heteroarylene. In some embodiments, R^{L2} is an optionally substituted C₁-C₉ aliphatic wherein one or more methylene units are optionally and independently replaced by an optionally substituted C₃-C₁₀ carbocyclylene. In some embodiments, R^{L2} is an optionally substituted C₁-C₉ aliphatic wherein two methylene units are optionally and independently replaced by -Cy- or —C(O)—. In some embodiments, R^{L2} is an optionally substituted C₁-C₉ aliphatic wherein two methylene units are optionally and independently replaced by -Cy- or —C(O)—. Example R^{L2} groups are depicted below:

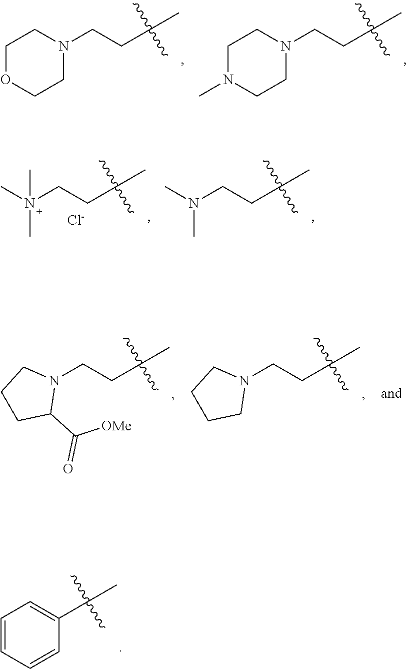

In some embodiments, R¹ is hydrogen, or an optionally substituted group selected from

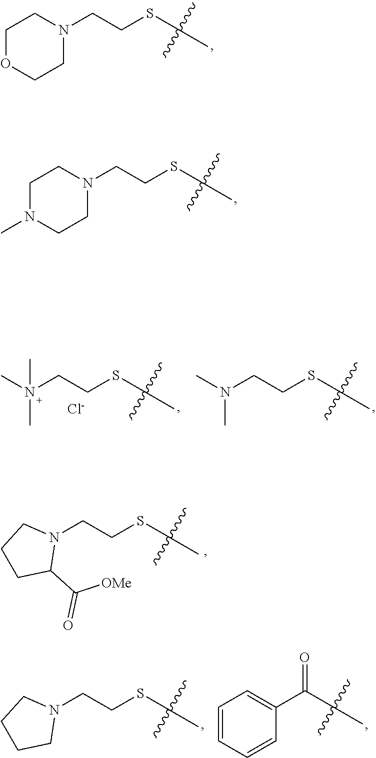

—S—(C₁-C₁₀ aliphatic), C₁-C₁₀ aliphatic, aryl, C₁-C₆ heteroalkyl, heteroaryl and heterocyclyl. In some embodiments, R¹ is

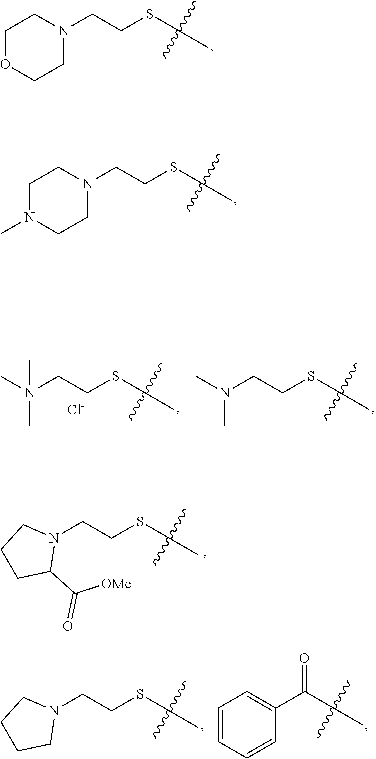

or —S—(C₁-C₁₀ aliphatic). In some embodiments, R¹ is

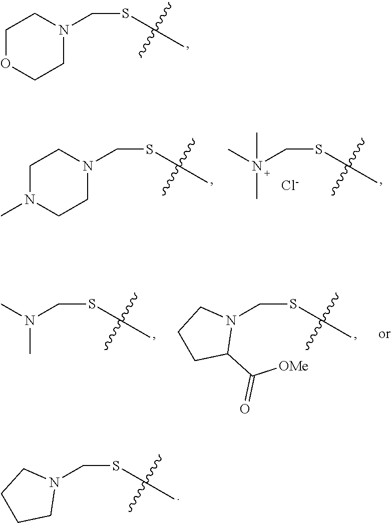

In some embodiments, R¹ is an optionally substituted group selected from —S—(C₁-C₆ aliphatic), C₁-C₁₀ aliphatic, C₁-C₆ heteroaliphatic, aryl, heterocyclyl and heteroaryl.

In some embodiments. R¹ is

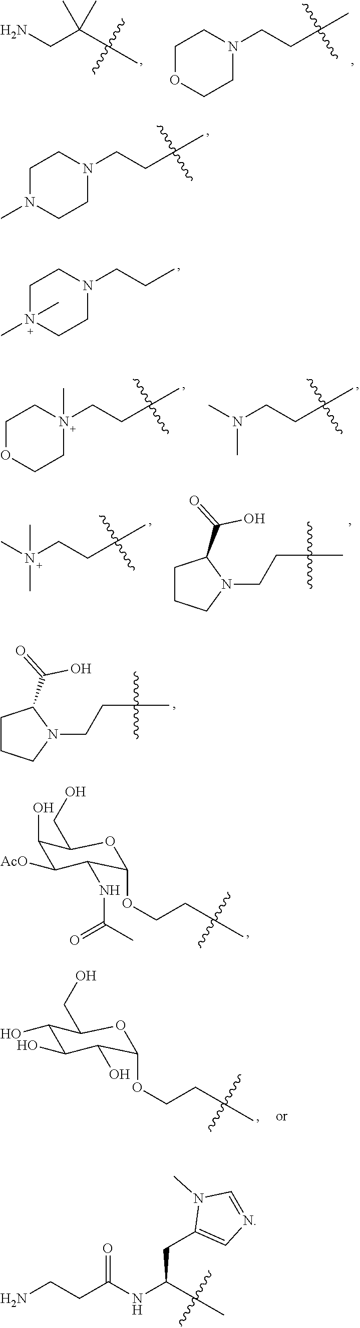

In some embodiments, the sulfur atom in the R¹ embodiments described above and herein is connected with the sulfur atom, G, E, or —C(O)— moiety in the L embodiments described above and herein. In some embodiments, the —C(O)— moiety in the R¹ embodiments described above and herein is connected with the sulfur atom, G, E, or —C(O)— moiety in the L embodiments described above and herein.

In some embodiments, -L-R¹ is any combination of the L embodiments and R¹ embodiments described above and herein.

In some embodiments, -L-R¹ is -L³-G-R¹ wherein each variable is independently as defined above and described herein.

In some embodiments, -L-R¹ is -L⁴-G-R¹ wherein each variable is independently as defined above and described herein.

In some embodiments, -L-R¹ is -L³-G-S—R^{L2}, wherein each variable is independently as defined above and described herein.

In some embodiments, -L-R¹ is -L³-G-C(O)-R^{L2}, wherein each variable is independently as defined above and described herein.

In some embodiments, -L-R¹ is

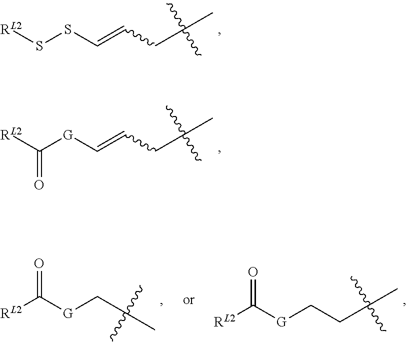

wherein R^{L2} is an optionally substituted C₁-C₉ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—, and each G is independently as defined above and described herein.

In some embodiments, -L-R¹ is —R^{L3}—S—S—R^{L2}, wherein each variable is independently as defined above and described herein. In some embodiments, -L-R¹ is —R^{L3}—C(O)—S—S—R^{L2}, wherein each variable is independently as defined above and described herein.

In some embodiments, -L-R¹ has the structure of:

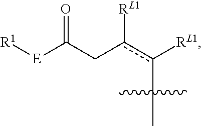

wherein each variable is independently as defined above and described herein.

In some embodiments, -L-R¹ has the structure of:

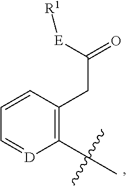

wherein each variable is independently as defined above and described herein.

In some embodiments, -L-R¹ has the structure of:

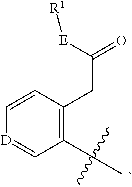

wherein each variable is independently as defined above and described herein.

In some embodiments, -L-R¹ has the structure of:

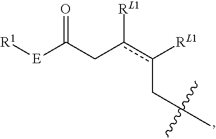

wherein each variable is independently as defined above and described herein.

In some embodiments, -L-R¹ has the structure of:

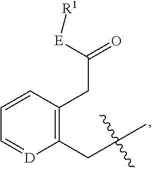

wherein each variable is independently as defined above and described herein.

In some embodiments, -L-R¹ has the structure of:

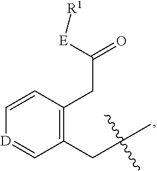

wherein each variable is independently as defined above and described herein.

In some embodiments, -L-R¹ has the structure of:

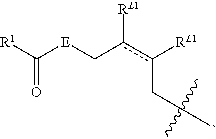

wherein each variable is independently as defined above and described herein.

In some embodiments, -L-R¹ has the structure of:

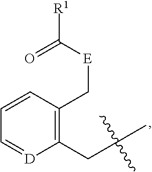

wherein each variable is independently as defined above and described herein.

In some embodiments, -L-R¹ has the structure of:

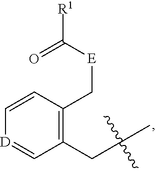

wherein each variable is independently as defined above and described herein.

In some embodiments, -L-R¹ has the structure of:

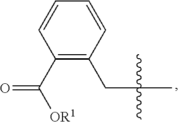

wherein each variable is independently as defined above and described herein.

In some embodiments, -L-R¹ has the structure of:

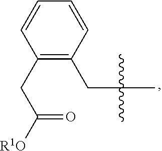

wherein each variable is independently as defined above and described herein.

In some embodiments, -L-R¹ has the structure of:

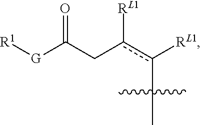

wherein each variable is independently as defined above and described herein.

In some embodiments, -L-R¹ has the structure of:

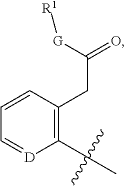

wherein each variable is independently as defined above and described herein.

In some embodiments, -L-R¹ has the structure of:

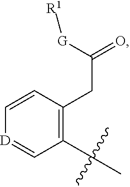

wherein each variable is independently as defined above and described herein.

In some embodiments, -L-R¹ has the structure of:

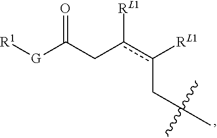

wherein each variable is independently as defined above and described herein.

In some embodiments, -L-R¹ has the structure of:

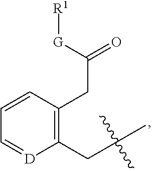

wherein each variable is independently as defined above and described herein.

In some embodiments, -L-R¹ has the structure of:

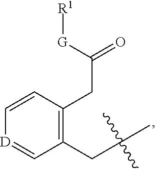

wherein each variable is independently as defined above and described herein.

In some embodiments, -L-R¹ has the structure of:

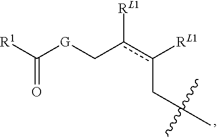

wherein each variable is independently as defined above and described herein.

In some embodiments, -L-R¹ has the structure of:

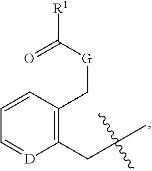

wherein each variable is independently as defined above and described herein.

In some embodiments, -L-R¹ has the structure of:

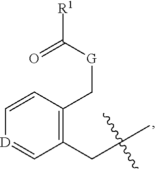

wherein each variable is independently as defined above and described herein.

In some embodiments, -L-R¹ has the structure of:

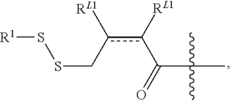

wherein each variable is independently as defined above and described herein.

In some embodiments, L has the structure of:

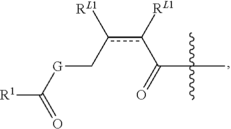

wherein each variable is independently as defined above and described herein.

In some embodiments, —X-L-R¹ has the structure of:

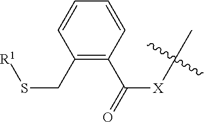

wherein:
the phenyl ring is optionally substituted, and
each of R¹ and X is independently as defined above and described herein.

In some embodiments, -L-R¹ is

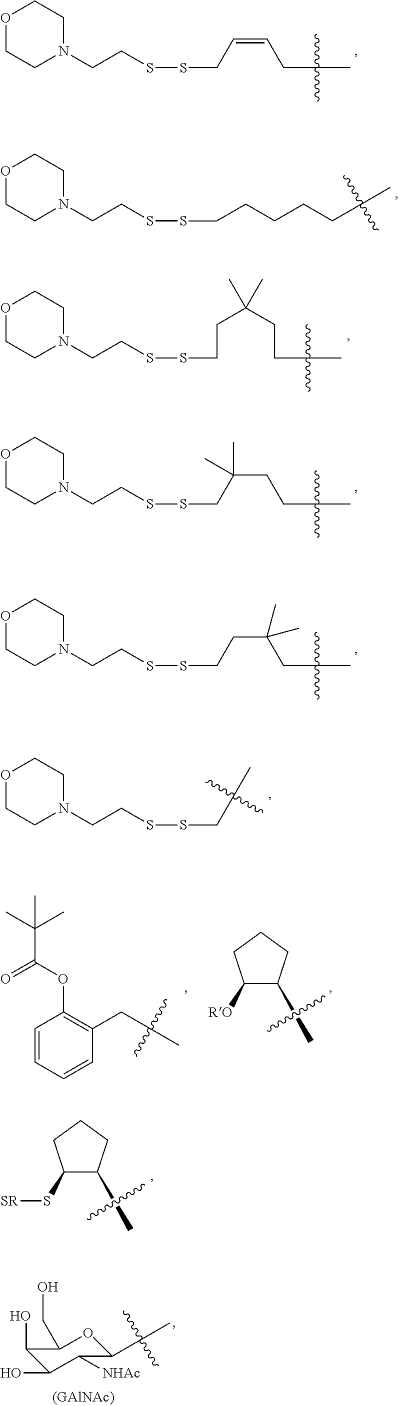

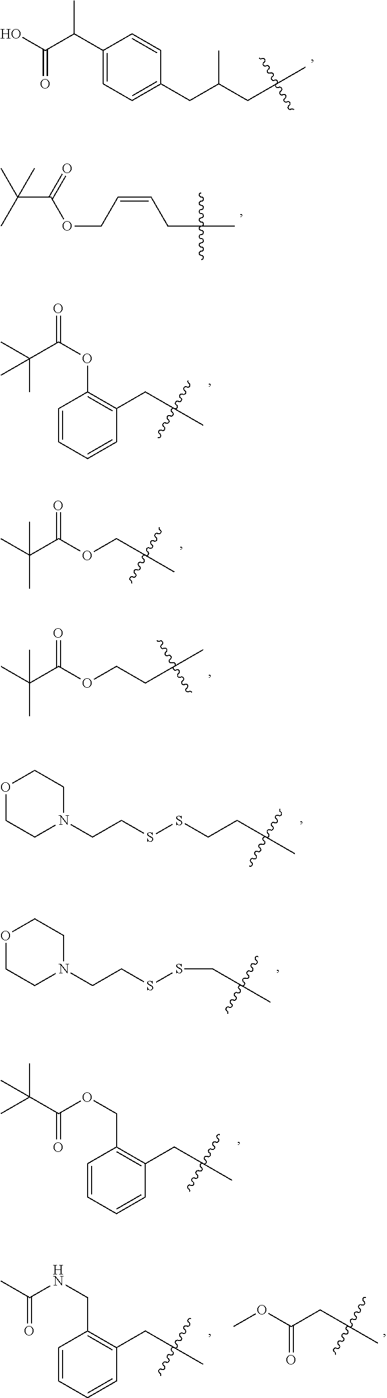

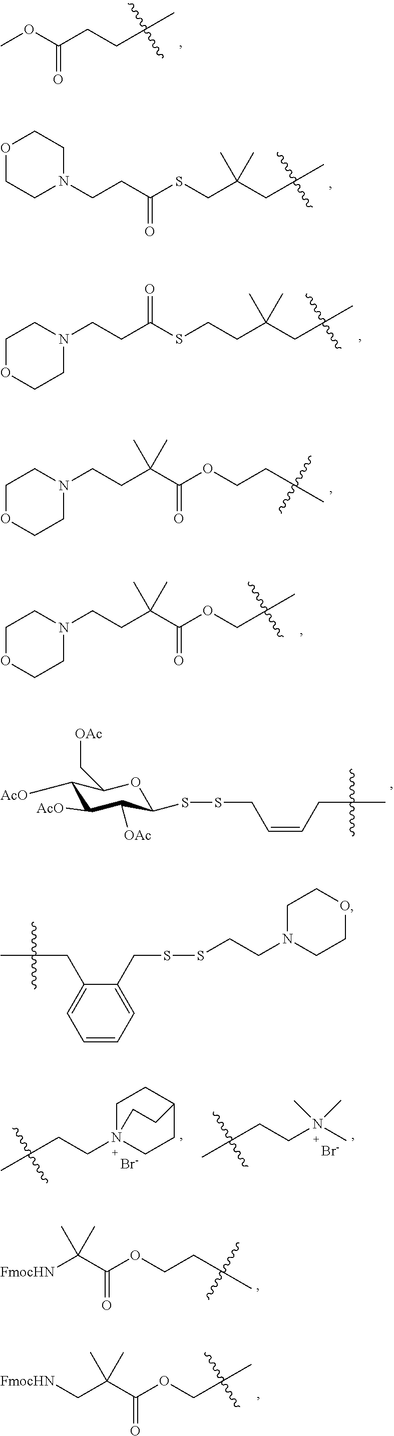

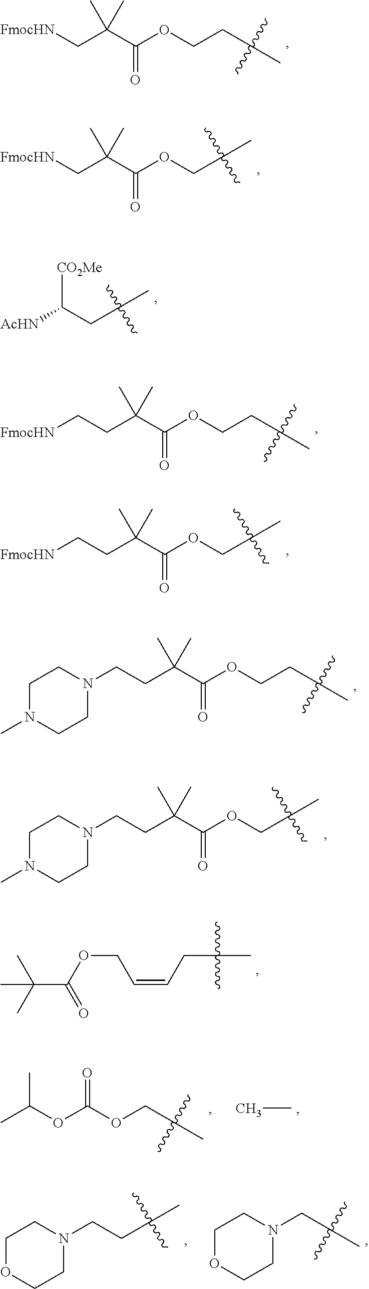

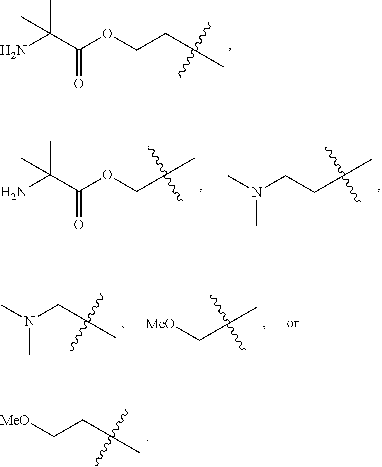

In some embodiments, -L-R¹ is:

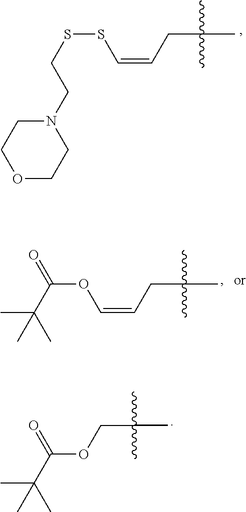

In some embodiments, -L-R¹ is CH₃—,

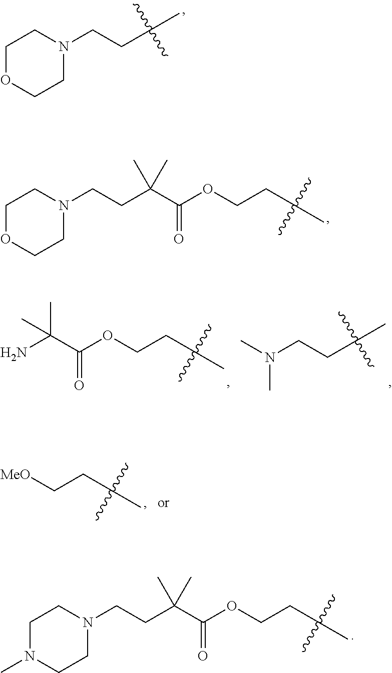

In some embodiments, -L-R¹ is

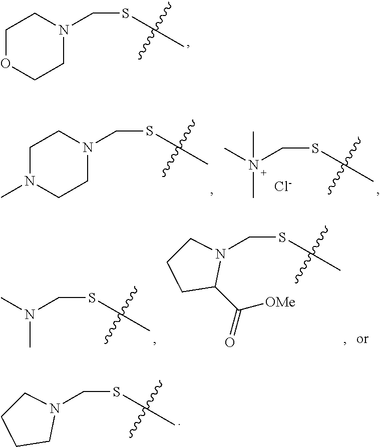

In some embodiments, -L-R¹ comprises a terminal optionally substituted —(CH₂)₂— moiety which is connected to X. In some embodiments, -L-R¹ comprises a terminal —(CH₂)₂— moiety which is connected to X. Examples of such -L-R¹ moieties are depicted below:

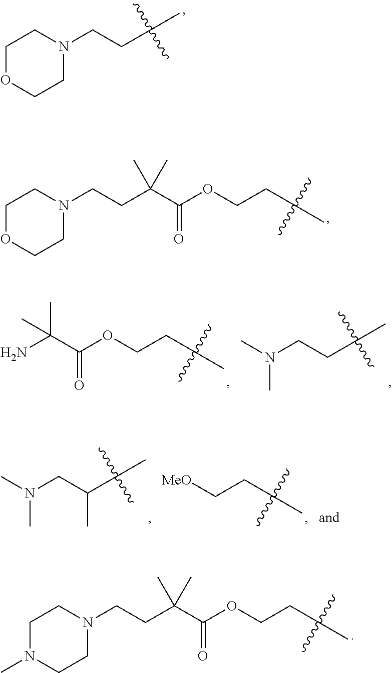

In some embodiments, -L-R¹ comprises a terminal optionally substituted —(CH₂)— moiety which is connected to X. In some embodiments, -L-R¹ comprises a terminal —(CH₂)— moiety which is connected to X. Examples of such -L-R¹ moieties are depicted below:

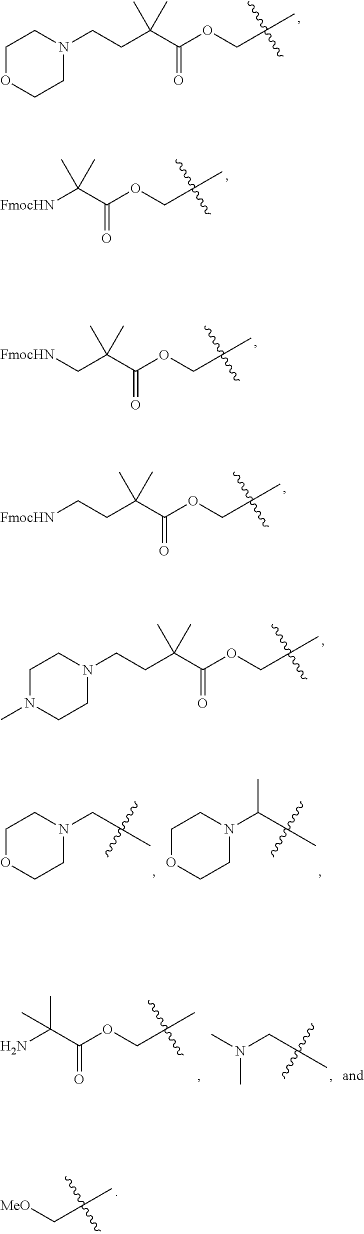

In some embodiments, -L-R¹ is

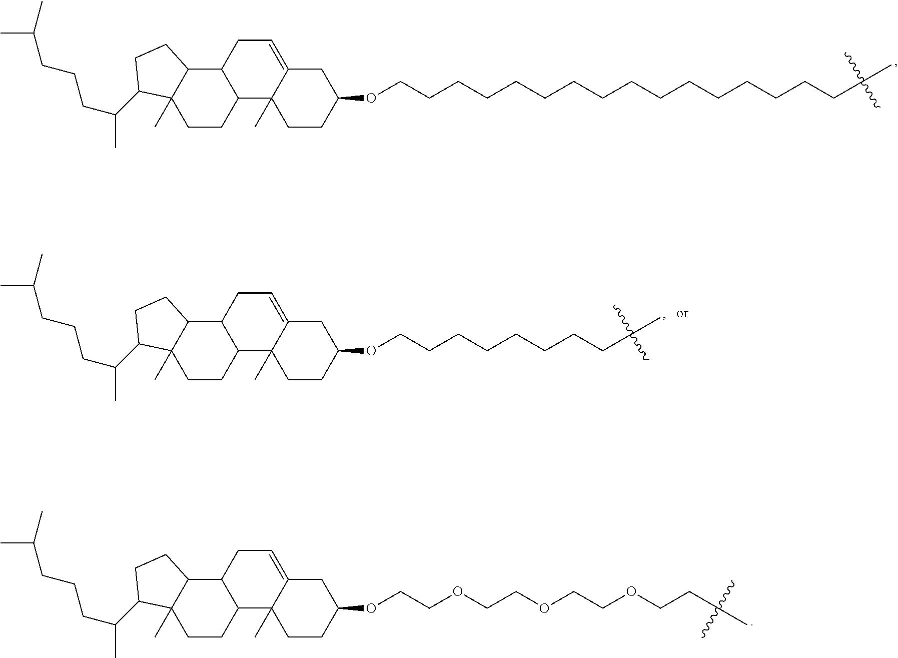 [SMILES: *CCCCCCCCCCCCCCCCO[C@H]1CCC2(C)C(=CCC3C2CCC2(C)C(C(C)CCCC(C)C)CCC32)C1 ; *CCCCCCCCO[C@H]1CCC2(C)C(=CCC3C2CCC2(C)C(C(C)CCCC(C)C)CCC32)C1 ; *CCOCCOCCOCCO[C@H]1CCC2(C)C(=CCC3C2CCC2(C)C(C(C)CCCC(C)C)CCC32)C1]

In some embodiments, -L-R¹ is CH₃—,

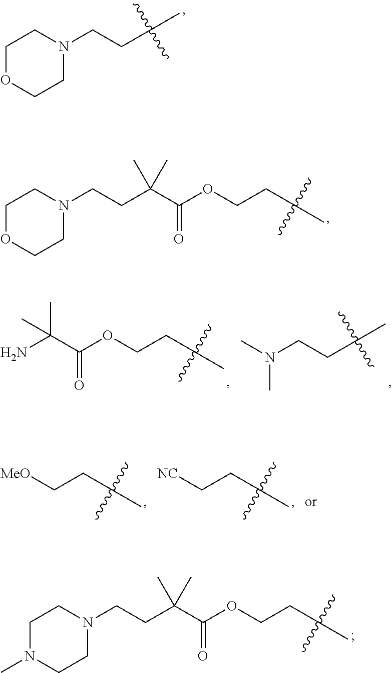

and X is —S—.

In some embodiments, -L-R¹ is CH₃—,

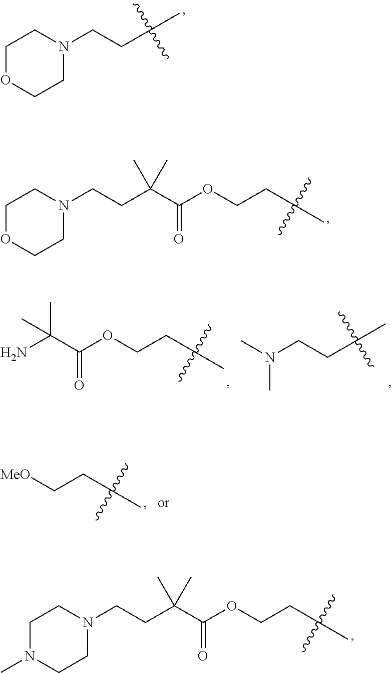

X is —S—, W is O, Y is —O—, and Z is —O—.

In some embodiments, R¹ is

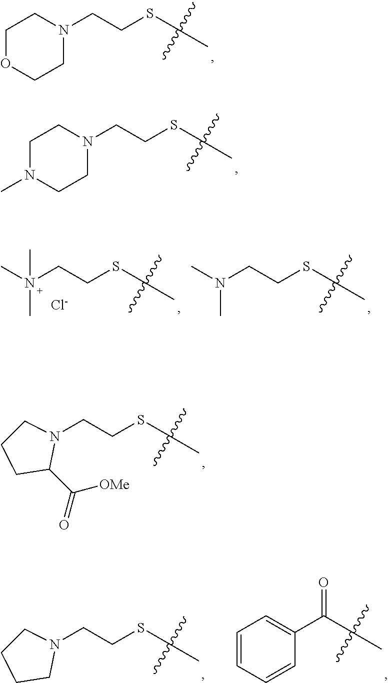

or —S—(C₁-C₁₀ aliphatic).

In some embodiments, R¹ is

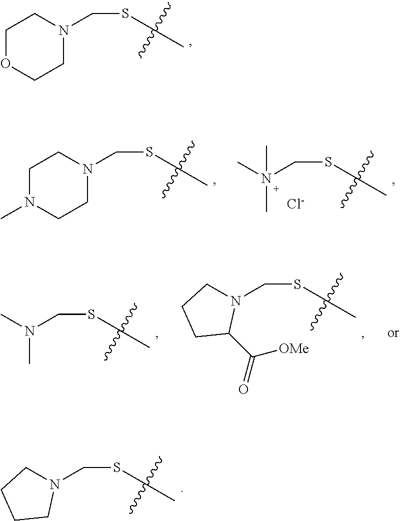

In some embodiments, X is —O— or —S—, and R¹ is

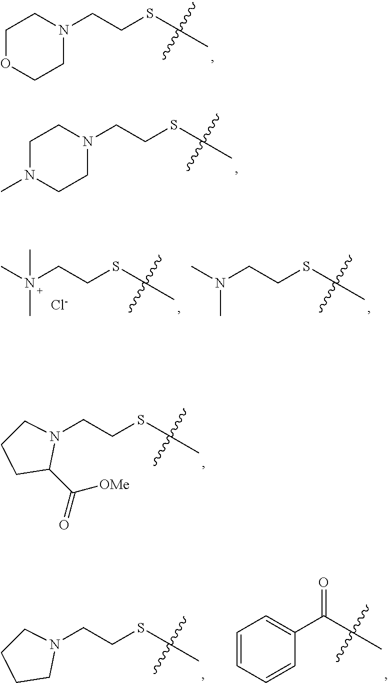

or —S—(C₁-C₁₀ aliphatic).

In some embodiments, X is —O— or —S—, and R¹ is

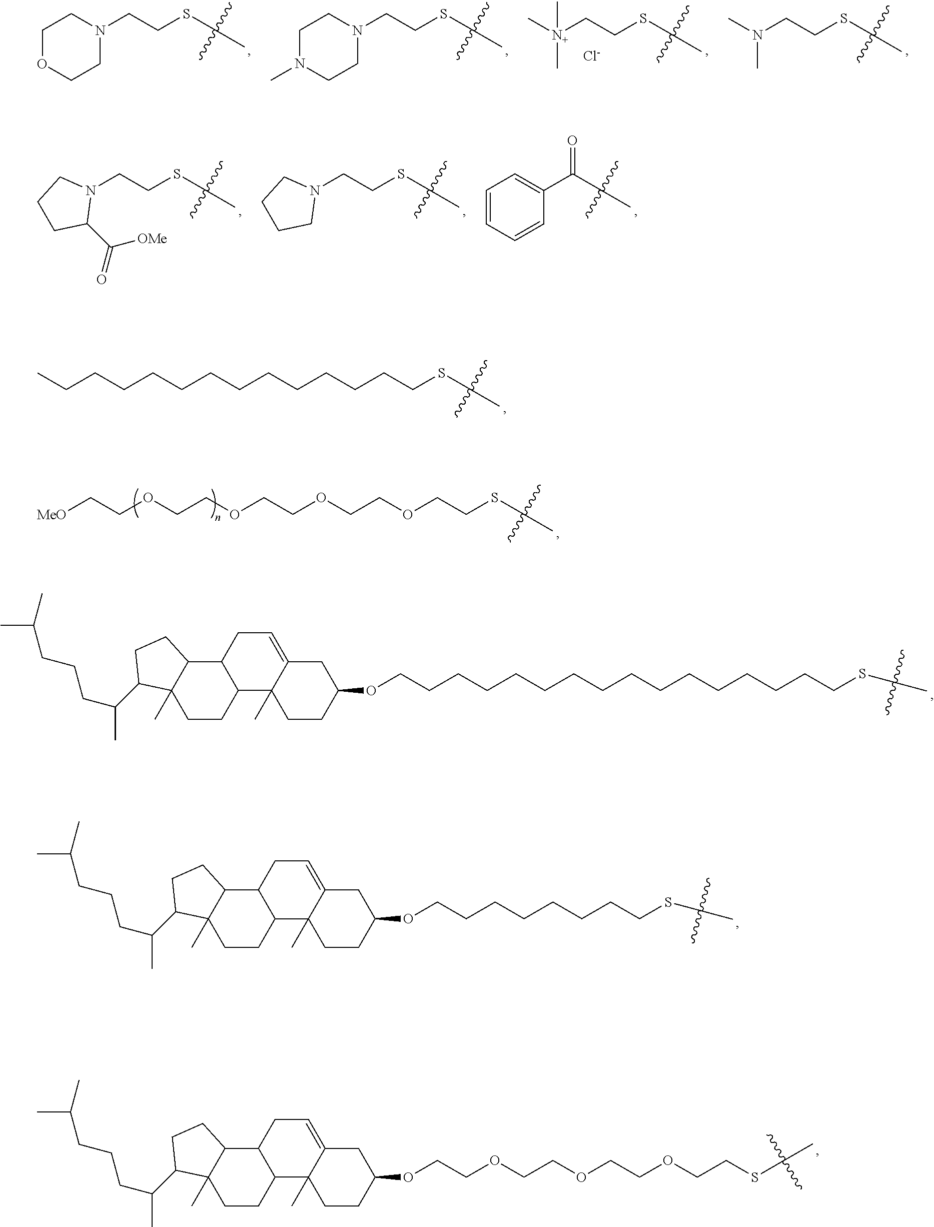

—S—(C₁-C₁₀ aliphatic) or —S—(C₁-C₅₀ aliphatic).

In some embodiments, L is a covalent bond and -L-R¹ is R¹.

In some embodiments, -L-R¹ is not hydrogen.

In some embodiments, —X-L-R¹ is R¹ is

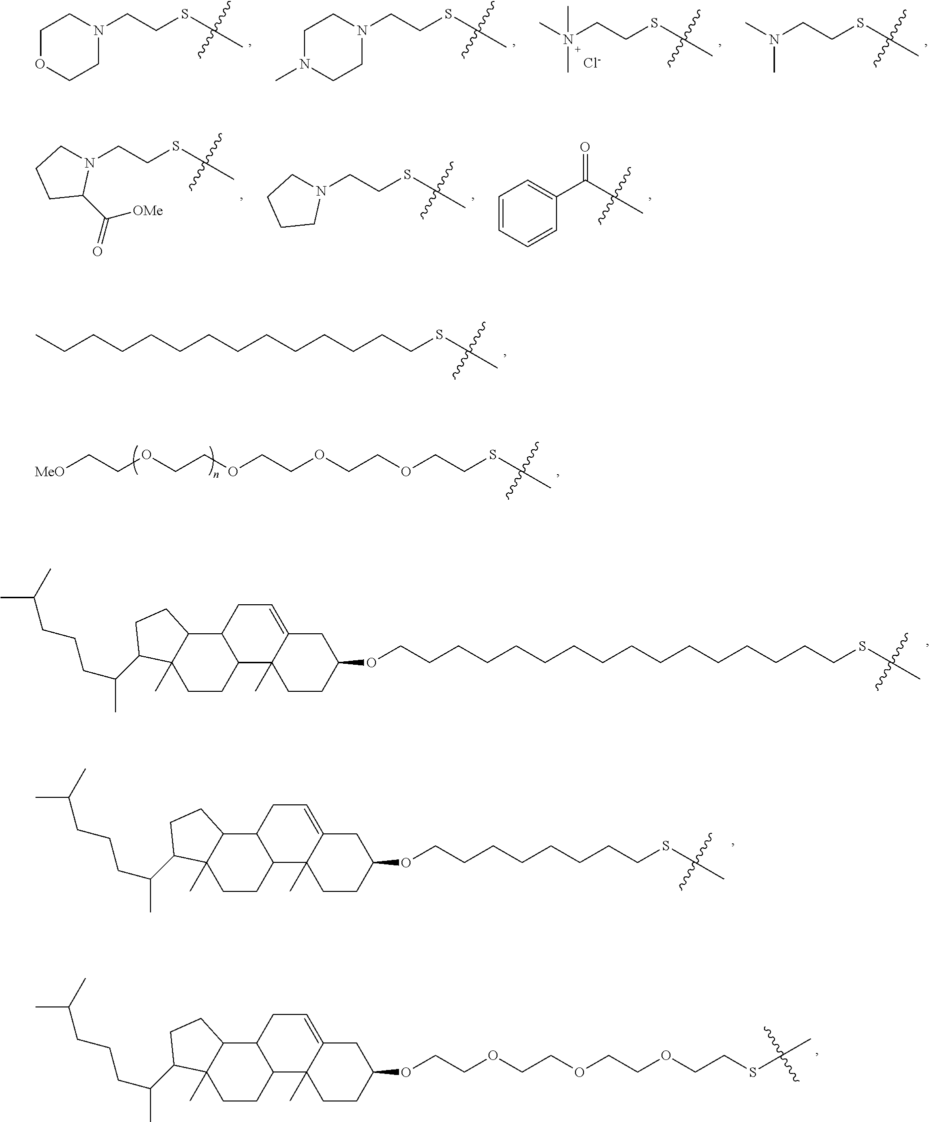

—S—(C₁-C₁₀ aliphatic) or —S—(C₁-C₅₀ aliphatic).

In some embodiments, —X-L-R¹ is has the structure of

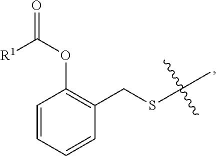

wherein the

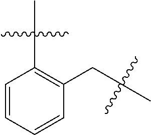 [SMILES: *Cc1ccccc1*]

moiety is optionally substituted. In some embodiments, —X-L-R¹ is

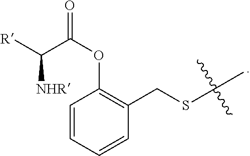

In some embodiments, —X-L-R¹ is

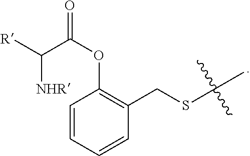

In some embodiments, —X-L-R¹ is

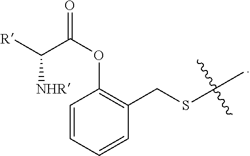

In some embodiments, —X-L-R¹ has the structure of

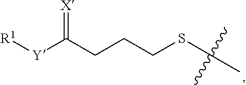

wherein X′ is O or S, Y′ is —O—, —S— or —NR′—, and the

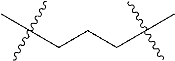 [SMILES: *CCC*]

moiety is optionally substituted. In some embodiments, Y′ is —O—, —S— or —NH—. In some embodiments,

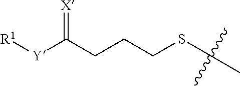

is

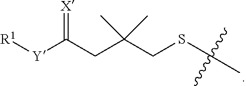

In some embodiments,

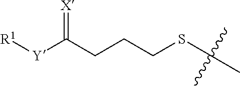

is

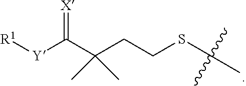

In some embodiments,

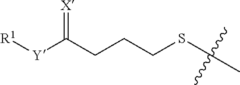

is

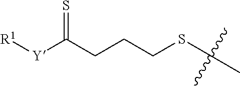

In some embodiments, —X-L-R¹ has the structure of

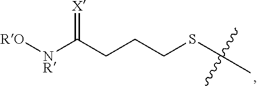

wherein X′ is O or S, and the

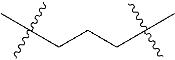 [SMILES: *CCC*]

moiety is optionally substituted. In some embodiments,

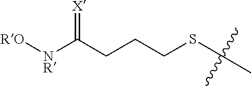

is

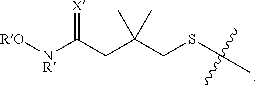

In some embodiments, —X-L-R¹ is

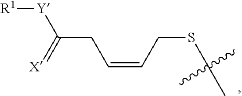

wherein the

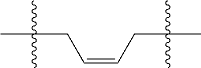 [SMILES: *C/C=C\C*]

is optionally substituted. In some embodiments, —X-L-R¹ is

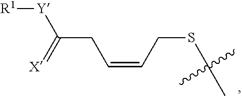

wherein the

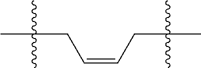 [SMILES: *C/C=C\C*]

is substituted. In some embodiments, —X-L-R¹ is

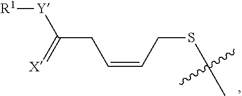

wherein the

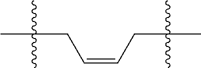 [SMILES: *C/C=C\C*]

is unsubstituted.

In some embodiments, —X-L-R¹ is R¹—C(O)—S-L^{x}-S—, wherein L^{x} is an optionally substituted group selected from

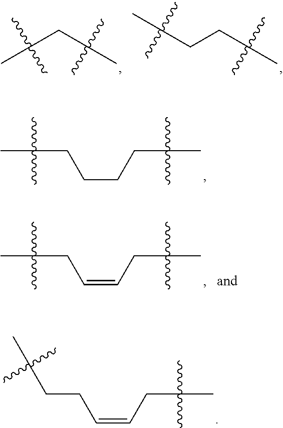

In some embodiments, L^{x} is

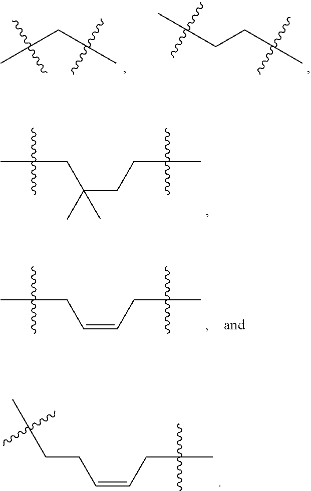

In some embodiments, —X-L-R¹ is (CH₃)₃C—S—S-L^{x}-S—. In some embodiments, —X-L-R¹ is R¹—C(═X′)—Y′—C(R)₂—S-L^{x}-S—. In some embodiments, —X-L-R¹ is R—C(═X′)—Y′—CH₂—S-L^{x}-S—. In some embodiments, —X-L-R¹ is

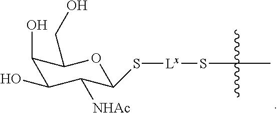

As will be appreciated by a person skilled in the art, many of the —X-L-R¹ groups described herein are cleavable and can be converted to —X⁻ after administration to a subject. In some embodiments, —X-L-R¹ is cleavable. In some embodiments, —X-L-R¹ is —S-L-R¹, and is converted to —S⁻ after administration to a subject. In some embodiments, the conversion is promoted by an enzyme of a subject. As appreciated by a person skilled in the art, methods of determining whether the —S-L-R¹ group is converted to —S⁻ after administration is widely known and practiced in the art, including those used for studying drug metabolism and pharmacokinetics.

In some embodiments, the internucleotidic linkage having the structure of Formula I is

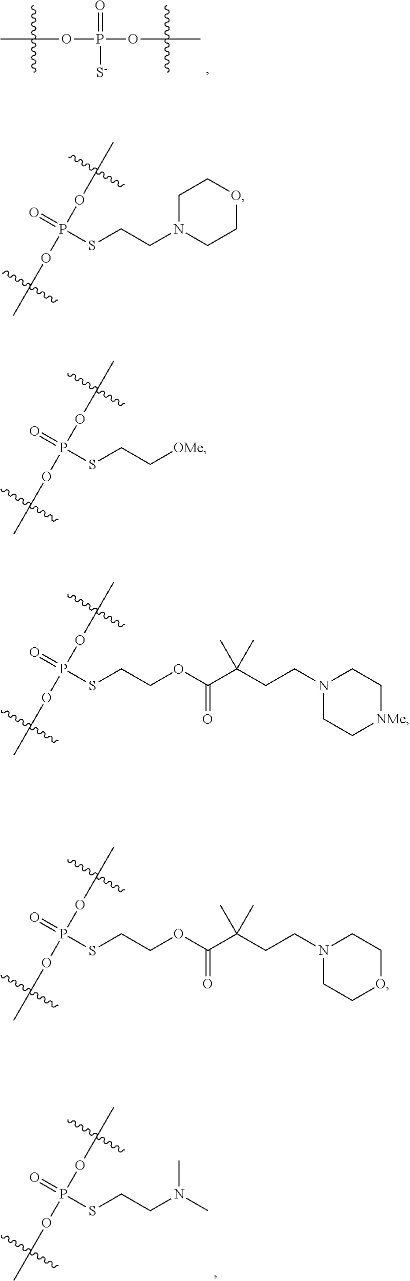 [SMILES: *OP(=O)([S-])O* ; *OP(=O)(O*)SCCN1CCOCC1 ; *OP(=O)(O*)SCCOC ; *OP(=O)(O*)SCCOC(=O)C(C)(C)CCN1CCN(C)CC1 ; *OP(=O)(O*)SCCOC(=O)C(C)(C)CCN1CCOCC1 ; *OP(=O)(O*)SCCN(C)C]

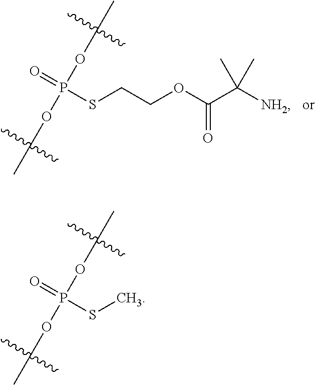 [SMILES: *OP(=O)(O*)SCCOC(=O)C(C)(C)N ; *OP(=O)(O*)SC]

In some embodiments, the internucleotidic linkage of Formula I has the structure of Formula I-a:

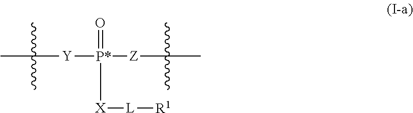

wherein each variable is independently as defined above and described herein, as in Formula I.

In some embodiments, the internucleotidic linkage of Formula I has the structure of Formula I-b:

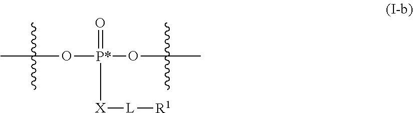

wherein each variable is independently as defined above and described herein, as in Formula I.

In some embodiments, the internucleotidic linkage of Formula I is an phosphorothioate triester linkage having the structure of Formula I-c:

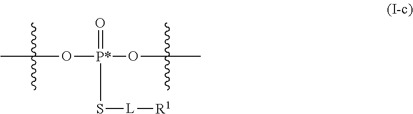

wherein:
P* is an asymmetric phosphorus atom and is either Rp or Sp;
L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, and heterocyclylene;
each R is independently hydrogen, or an optionally substituted group selected from C₁-C₆ aliphatic, phenyl, carbocyclyl, aryl, heteroaryl, and heterocyclyl;
each

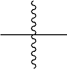

independently represents a connection to a nucleoside; and
R¹ is not —H when L is a covalent bond.

In some embodiments, the internucleotidic linkage having the structure of Formula I is

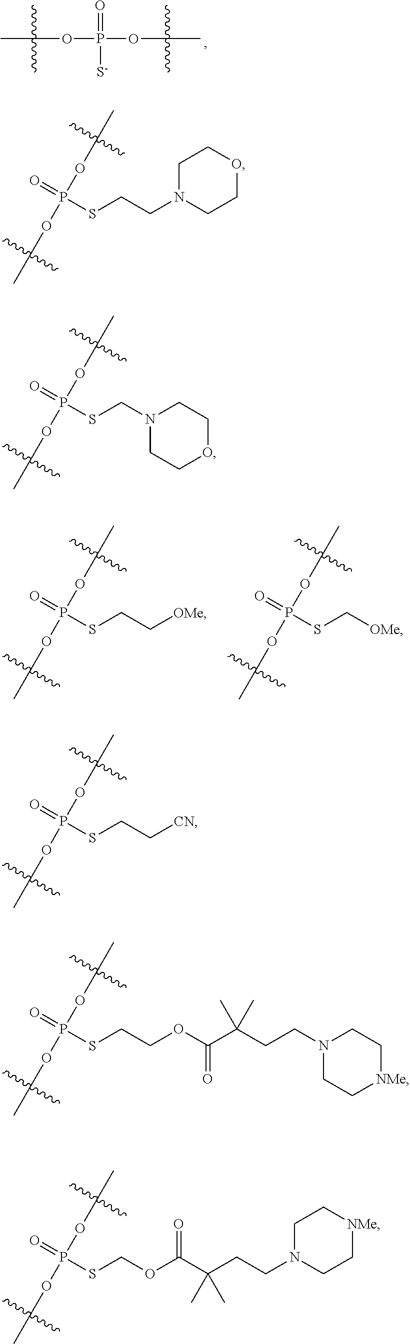

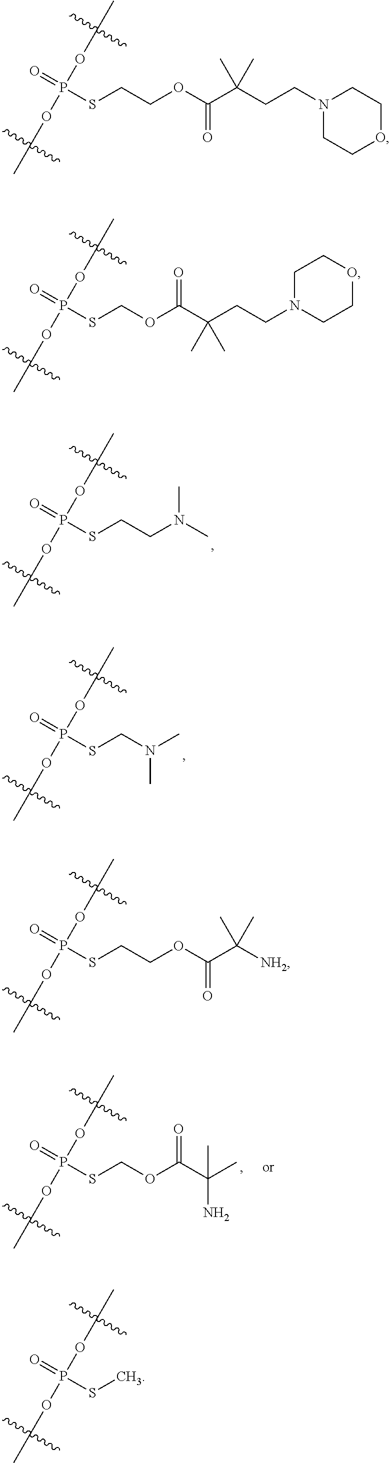

In some embodiments, the internucleotidic linkage having the structure of Formula I-c is

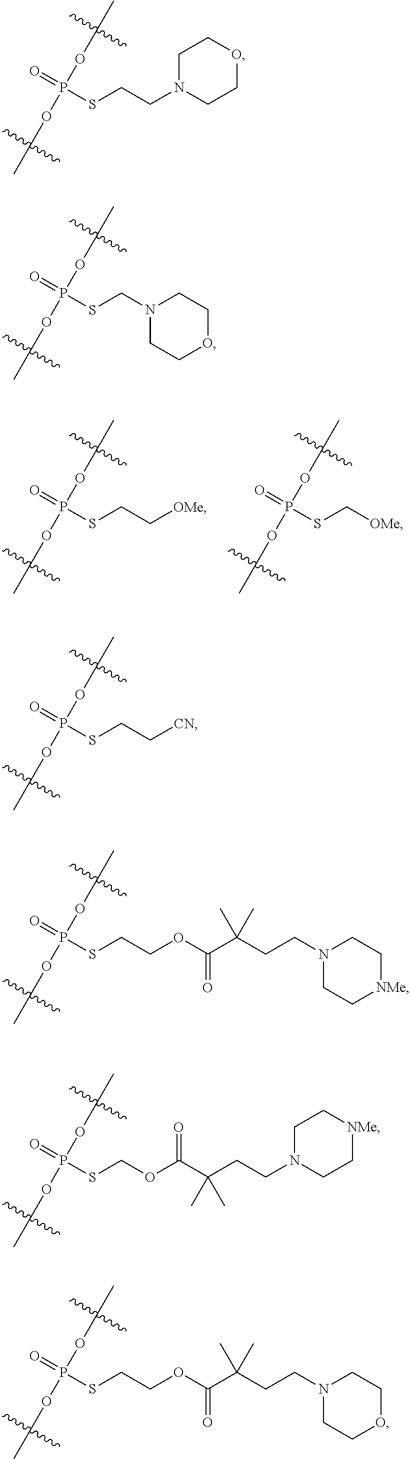

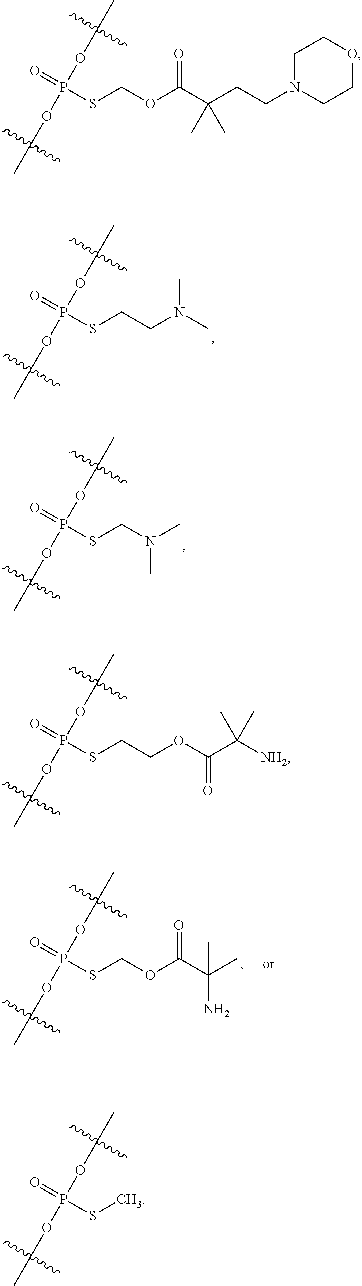 [SMILES: *OP(=O)(O*)SCOC(=O)C(C)(C)CCN1CCOCC1 ; *OP(=O)(O*)SCCN(C)C ; *OP(=O)(O*)SCN(C)C ; *OP(=O)(O*)SCCOC(=O)C(C)(C)N ; *OP(=O)(O*)SCOC(=O)C(C)(C)N ; *OP(=O)(O*)SC]

In some embodiments, the present disclosure provides a chirally controlled oligonucleotide comprising one or more phosphate diester linkages, and one or more modified internucleotide linkages having the formula of I-a, I-b, or I-c.

In some embodiments, a modified internucleotidic linkage has the structure of I. In some embodiments, a modified internucleotidic linkage has the structure of I-a. In some embodiments, a modified internucleotidic linkage has the structure of I-b. In some embodiments, a modified internucleotidic linkage has the structure of I-c.

In some embodiments, a modified internucleotidic linkage is phosphorothioate. Examples of internucleotidic linkages having the structure of Formula I are widely known in the art. In some embodiments, a modified internucleotidic linkage is selected from those described in, for example: US 20110294124, US 20120316224, US 20140194610, US 20150211006, US 20150197540, WO 2015107425, PCT/US2016/043542, and PCT/US2016/043598, each of which is incorporated herein by reference. In some embodiments, a modified internucleotidic linkage is a vinylphosphonate. Whittaker et al. 2008 Tetrahedron Letters 49: 6984-6987.

Non-limiting examples of internucleotidic linkages also include those described in the art, including, but not limited to, those described in any of: Gryaznov, S.; Chen, J.-K. J. Am. Chem. Soc. 1994, 116, 3143, Jones et al. J. Org. Chem. 1993, 58, 2983, Koshkin et al. 1998 Tetrahedron 54: 3607-3630, Lauritsen et al. 2002 Chem. Comm. 5: 530-531, Lauritsen et al. 2003 Bioo. Med. Chem. Lett. 13: 253-256, Mesmaeker et al. Angew. Chem., Int. Ed. Engl. 1994, 33, 226, Petersen et al. 2003 TRENDS Biotech. 21: 74-81, Schultz et al. 1996 Nucleic Acids Res. 24: 2966, Ts′o et al. Ann. N. Y. Acad. Sci. 1988, 507, 220, and Vasseur et al. J. Am. Chem. Soc. 1992, 114, 4006.

Oligonucleotides of the provided technologies can be of various lengths. In some embodiments, provided oligonucleotides comprise 5, 6, 7, 8, 9, 10, 11, 12, 13, 14, 15, 16, 17, 18, 19, 20, 21, 22, 23, 24, 25, 26, 27, 28, 29, 30, 40, 50 or more bases. In some embodiments, provided oligonucleotides comprise 10, 11, 12, 13, 14, 15, 16, 17, 18, 19, 20, 21, 22, 23, 24, 25, 26, 27, 28, 29, 30, 40, 50 or more bases. In some embodiments, provided oligonucleotides comprise 15, 16, 17, 18, 19, 20, 21, 22, 23, 24, 25, 26, 27, 28, 29, 30, 40, 50 or more bases. In some embodiments, provided oligonucleotides comprise 15 or more bases. In some embodiments, provided oligonucleotides comprise 16 or more bases. In some embodiments, provided oligonucleotides comprise 17 or more bases. In some embodiments, provided oligonucleotides comprise 18 or more bases. In some embodiments, provided oligonucleotides comprise 19 or more bases. In some embodiments, provided oligonucleotides comprise 20 or more bases. In some embodiments, provided oligonucleotides comprise 21 or more bases. In some embodiments, provided oligonucleotides comprise 22 or more bases. In some embodiments, provided oligonucleotides comprise 23 or more bases. In some embodiments, provided oligonucleotides comprise 24 or more bases. In some embodiments, provided oligonucleotides comprise 25 or more bases. In some embodiments, provided oligonucleotides comprise 26 or more bases. In some embodiments, provided oligonucleotides comprise 27 or more bases. In some embodiments, provided oligonucleotides comprise 28 or more bases. In some embodiments, provided oligonucleotides comprise 29 or more bases. In some embodiments, provided oligonucleotides comprise 30 or more bases. In some embodiments, provided oligonucleotides comprise 40 or more bases. In some embodiments, provided oligonucleotides comprise 50 or more bases. In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are 15 mers. In some embodiments, provided oligonucleotides are 16 mers. In some embodiments, provided oligonucleotides are 17 mers. In some embodiments, provided oligonucleotides are 18 mers. In some embodiments, provided oligonucleotides are 19 mers. In some embodiments, provided oligonucleotides are 20 mers. In some embodiments, provided oligonucleotides are 21 mers. In some embodiments, provided oligonucleotides are 22 mers. In some embodiments, provided oligonucleotides are 23 mers. In some embodiments, provided oligonucleotides are 24 mers. In some embodiments, provided oligonucleotides are 25 mers. In some embodiments, provided oligonucleotides are 26 mers. In some embodiments, provided oligonucleotides are 27 mers. In some embodiments, provided oligonucleotides are 28 mers. In some embodiments, provided oligonucleotides are 29 mers. In some embodiments, provided oligonucleotides are 30 mers.

In some embodiments, the present disclosure provides a chirally controlled oligonucleotide comprising at least one phosphate diester internucleotidic linkage and at least one phosphorothioate triester linkage having the structure of Formula I-c. In some embodiments, the present disclosure provides a chirally controlled oligonucleotide comprising at least one phosphate diester internucleotidic linkage and at least two phosphorothioate triester linkages having the structure of Formula I-c. In some embodiments, the present disclosure provides a chirally controlled oligonucleotide comprising at least one phosphate diester internucleotidic linkage and at least three phosphorothioate triester linkages having the structure of Formula I-c. In some embodiments, the present disclosure provides a chirally controlled oligonucleotide comprising at least one phosphate diester internucleotidic linkage and at least four phosphorothioate triester linkages having the structure of Formula I-c. In some embodiments, the present disclosure provides a chirally controlled oligonucleotide comprising at least one phosphate diester internucleotidic linkage and at least five phosphorothioate triester linkages having the structure of Formula I-c.

In some embodiments, the present disclosure provides a chirally controlled oligonucleotide comprising a sequence found in any oligonucleotide disclosed herein. In some embodiments, the present disclosure provides a chirally controlled oligonucleotide comprising a sequence found in any oligonucleotide disclosed herein, wherein one or more U is replaced with T. In some embodiments, the present disclosure provides a chirally controlled oligonucleotide comprising a sequence found in any oligonucleotide disclosed herein, wherein the said sequence has over 50% identity with the sequence of any oligonucleotide disclosed herein.

In some embodiments, the present disclosure provides a chirally controlled oligonucleotide comprising a sequence found in any oligonucleotide disclosed herein, wherein the said sequence has over 60% identity with the sequence of any oligonucleotide disclosed herein. In some embodiments, the present disclosure provides a chirally controlled oligonucleotide comprising a sequence found in any oligonucleotide disclosed herein, wherein the said sequence has over 70% identity with the sequence of any oligonucleotide disclosed herein. In some embodiments, the present disclosure provides a chirally controlled oligonucleotide comprising a sequence found in any oligonucleotide disclosed herein, wherein the said sequence has over 80% identity with the sequence of any oligonucleotide disclosed herein. In some embodiments, the present disclosure provides a chirally controlled oligonucleotide comprising a sequence found in any oligonucleotide disclosed herein, wherein the said sequence has over 90% identity with the sequence of any oligonucleotide disclosed herein. In some embodiments, the present disclosure provides a chirally controlled oligonucleotide comprising a sequence found in any oligonucleotide disclosed herein, wherein the said sequence has over 95% identity with the sequence of any oligonucleotide disclosed herein.

In some embodiments, the present disclosure provides a chirally controlled oligonucleotide comprising the sequence of any oligonucleotide disclosed herein. In some embodiments, the present disclosure provides a chirally controlled oligonucleotide having the sequence of any oligonucleotide disclosed herein. In some embodiments, the present disclosure provides a chirally controlled oligonucleotide comprising a sequence found in any oligonucleotide disclosed herein, wherein the oligonucleotides have a pattern of backbone linkages, pattern of backbone chiral centers, and/or pattern of backbone phosphorus modifications described herein.

In some embodiments, the present disclosure provides a chirally controlled oligonucleotide comprising a sequence found in any oligonucleotide disclosed herein. In some embodiments, the present disclosure provides a chirally controlled oligonucleotide comprising a sequence found in any oligonucleotide disclosed herein, wherein one or more T is substituted with U. In some embodiments, the present disclosure provides a chirally controlled oligonucleotide comprising a sequence found in any oligonucleotide disclosed herein, wherein the said sequence has over 50% identity with the sequence of any oligonucleotide disclosed herein. In some embodiments, the present disclosure provides a chirally controlled oligonucleotide comprising a sequence found in any oligonucleotide disclosed herein, wherein the said sequence has over 60% identity with the sequence of any oligonucleotide disclosed herein. In some embodiments, the present disclosure provides a chirally controlled oligonucleotide comprising a sequence found in any oligonucleotide disclosed herein, wherein the said sequence has over 70% identity with the sequence of any oligonucleotide disclosed herein. In some embodiments, the present disclosure provides a chirally controlled oligonucleotide comprising a sequence found in any oligonucleotide disclosed herein, wherein the said sequence has over 80% identity with the sequence of any oligonucleotide disclosed herein. In some embodiments, the present disclosure provides a chirally controlled oligonucleotide comprising a sequence found in any oligonucleotide disclosed herein, wherein the said sequence has over 90% identity with the sequence of any oligonucleotide disclosed herein. In some embodiments, the present disclosure provides a chirally controlled oligonucleotide comprising a sequence found in any oligonucleotide disclosed herein, wherein the said sequence has over 95% identity with the sequence of any oligonucleotide disclosed herein. In some embodiments, the present disclosure provides a chirally controlled oligonucleotide comprising the sequence of any oligonucleotide disclosed herein. In some embodiments, the present disclosure provides a chirally controlled oligonucleotide having the sequence of any oligonucleotide disclosed herein.

In some embodiments, the present disclosure provides a chirally controlled oligonucleotide comprising a sequence found in any oligonucleotide disclosed herein, wherein at least one internucleotidic linkage has a chiral linkage phosphorus. In some embodiments, the present disclosure provides a chirally controlled oligonucleotide comprising a sequence found in any oligonucleotide disclosed herein, wherein at least one internucleotidic linkage has the structure of Formula I. In some embodiments, the present disclosure provides a chirally controlled oligonucleotide comprising a sequence found in any oligonucleotide disclosed herein, wherein each internucleotidic linkage has the structure of Formula I. In some embodiments, the present disclosure provides a chirally controlled oligonucleotide comprising a sequence found in any oligonucleotide disclosed herein, wherein at least one internucleotidic linkage has the structure of Formula I-c. In some embodiments, the present disclosure provides a chirally controlled oligonucleotide comprising a sequence found in any oligonucleotide disclosed herein, wherein each internucleotidic linkage has the structure of Formula I-c. In some embodiments, the present disclosure provides a chirally controlled oligonucleotide comprising a sequence found in any oligonucleotide disclosed herein, wherein at least one internucleotidic linkage is

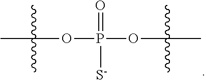 [SMILES: *OP(=O)([S-])O*]

In some embodiments, the present disclosure provides a chirally controlled oligonucleotide comprising a sequence found in any oligonucleotide disclosed herein, wherein each internucleotidic linkage is

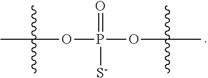 [SMILES: *OP(=O)([S-])O*]

In some embodiments, the present disclosure provides a chirally controlled oligonucleotide comprising a sequence found in any oligonucleotide disclosed herein, wherein at least one internucleotidic linkage is

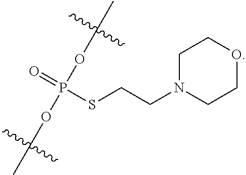 [SMILES: *OP(=O)(O*)SCCN1CCOCC1]

In some embodiments, the present disclosure provides a chirally controlled oligonucleotide comprising a sequence found in any oligonucleotide disclosed herein, wherein each internucleotidic linkage is

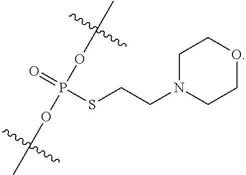 [SMILES: *OP(=O)(O*)SCCN1CCOCC1]

In some embodiments, the present disclosure provides a chirally controlled oligonucleotide comprising the sequence of any oligonucleotide disclosed herein, wherein at least one internucleotidic linkage has a chiral linkage phosphorus. In some embodiments, the present disclosure provides a chirally controlled oligonucleotide comprising the sequence of any oligonucleotide disclosed herein, wherein at least one internucleotidic linkage has the structure of Formula I. In some embodiments, the present disclosure provides a chirally controlled oligonucleotide comprising the sequence of any oligonucleotide disclosed herein, wherein each internucleotidic linkage has the structure of Formula I. In some embodiments, the present disclosure provides a chirally controlled oligonucleotide comprising the sequence of any oligonucleotide disclosed herein, wherein at least one internucleotidic linkage has the structure of Formula I-c. In some embodiments, the present disclosure provides a chirally controlled oligonucleotide comprising the sequence of any oligonucleotide disclosed herein, wherein each internucleotidic linkage has the structure of Formula I-c.

In some embodiments, the present disclosure provides a chirally controlled oligonucleotide comprising the sequence of any oligonucleotide disclosed herein, wherein at least one internucleotidic linkage is

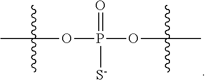 [SMILES: *OP(=O)([S-])O*]

In some embodiments, the present disclosure provides a chirally controlled oligonucleotide comprising the sequence of any oligonucleotide disclosed herein, wherein each internucleotidic linkage is

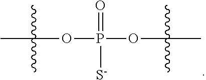 [SMILES: *OP(=O)([S-])O*]

In some embodiments, the present disclosure provides a chirally controlled oligonucleotide comprising the sequence of any oligonucleotide disclosed herein, wherein at least one internucleotidic linkage is

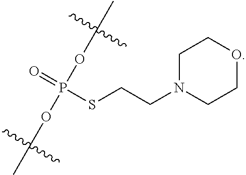 [SMILES: *OP(=O)(O*)SCCN1CCOCC1]

In some embodiments, the present disclosure provides a chirally controlled oligonucleotide comprising the sequence of any oligonucleotide disclosed herein, wherein each internucleotidic linkage is

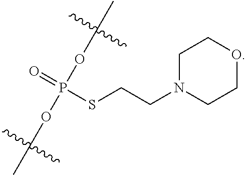 [SMILES: *OP(=O)(O*)SCCN1CCOCC1]

In some embodiments, the present disclosure provides a chirally controlled oligonucleotide having the sequence of any oligonucleotide disclosed herein, wherein at least one internucleotidic linkage has a chiral linkage phosphorus. In some embodiments, the present disclosure provides a chirally controlled oligonucleotide having the sequence of any oligonucleotide disclosed herein, wherein at least one internucleotidic linkage has the structure of Formula I. In some embodiments, the present disclosure provides a chirally controlled oligonucleotide having the sequence of any oligonucleotide disclosed herein, wherein each internucleotidic linkage has the structure of Formula I. In some embodiments, the present disclosure provides a chirally controlled oligonucleotide having the sequence of any oligonucleotide disclosed herein, wherein at least one internucleotidic linkage has the structure of Formula I-c. In some embodiments, the present disclosure provides a chirally controlled oligonucleotide having the sequence of any oligonucleotide disclosed herein, wherein each internucleotidic linkage has the structure of Formula I-c.

In some embodiments, the present disclosure provides a chirally controlled oligonucleotide having the sequence of any oligonucleotide disclosed herein, wherein at least one internucleotidic linkage is

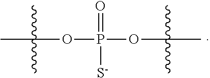 [SMILES: *OP(=O)([S-])O*]

In some embodiments, the present disclosure provides a chirally controlled oligonucleotide having the sequence of any oligonucleotide disclosed herein, wherein each internucleotidic linkage is

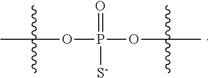 [SMILES: *OP(=O)([S-])O*]

In some embodiments, the present disclosure provides a chirally controlled oligonucleotide having the sequence of any oligonucleotide disclosed herein, wherein at least one internucleotidic linkage is

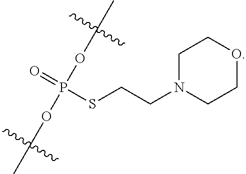 [SMILES: *OP(=O)(O*)SCCN1CCOCC1]

In some embodiments, the present disclosure provides a chirally controlled oligonucleotide having the sequence of any oligonucleotide disclosed herein, wherein each internucleotidic linkage is

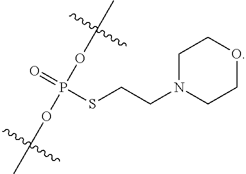 [SMILES: *OP(=O)(O*)SCCN1CCOCC1]

In some embodiments, the present disclosure provides a chirally controlled oligonucleotide having the sequence of any oligonucleotide disclosed herein, wherein at least one linkage phosphorus is Rp. It is understood by a person of ordinary skill in the art that in certain embodiments wherein the chirally controlled oligonucleotide comprises an RNA sequence, each T is independently and optionally replaced with U. In some embodiments, the present disclosure provides a chirally controlled oligonucleotide having the sequence of any oligonucleotide disclosed herein, wherein each linkage phosphorus is Rp. In some embodiments, the present disclosure provides a chirally controlled oligonucleotide having the sequence of any oligonucleotide disclosed herein, wherein at least one linkage phosphorus is Sp. In some embodiments, the present disclosure provides a chirally controlled oligonucleotide having the sequence of any oligonucleotide disclosed herein, wherein each linkage phosphorus is Sp. In some embodiments, the present disclosure provides a chirally controlled oligonucleotide having the sequence of any oligonucleotide disclosed herein, wherein the oligonucleotide is a blockmer. In some embodiments, the present disclosure provides a chirally controlled oligonucleotide having the sequence of any oligonucleotide disclosed herein, wherein the oligonucleotide is a stereoblockmer. In some embodiments, the present disclosure provides a chirally controlled oligonucleotide having the sequence of any oligonucleotide disclosed herein, wherein the oligonucleotide is a P-modification blockmer. In some embodiments, the present disclosure provides a chirally controlled oligonucleotide having the sequence of any oligonucleotide disclosed herein, wherein the oligonucleotide is a linkage blockmer. In some embodiments, the present disclosure provides a chirally controlled oligonucleotide having the sequence of any oligonucleotide disclosed herein, wherein the oligonucleotide is an altmer. In some embodiments, the present disclosure provides a chirally controlled oligonucleotide having the sequence of any oligonucleotide disclosed herein, wherein the oligonucleotide is a stereoaltmer. In some embodiments, the present disclosure provides a chirally controlled oligonucleotide having the sequence of any oligonucleotide disclosed herein, wherein the oligonucleotide is a P-modification altmer. In some embodiments, the present disclosure provides a chirally controlled oligonucleotide having the sequence of any oligonucleotide disclosed herein, wherein the oligonucleotide is a linkage altmer. In some embodiments, the present disclosure provides a chirally controlled oligonucleotide having the sequence of any oligonucleotide disclosed herein, wherein the oligonucleotide is a unimer. In some embodiments, the present disclosure provides a chirally controlled oligonucleotide having the sequence of any oligonucleotide disclosed herein, wherein the oligonucleotide is a stereounimer. In some embodiments, the present disclosure provides a chirally controlled oligonucleotide having the sequence of any oligonucleotide disclosed herein, wherein the oligonucleotide is a P-modification unimer. In some embodiments, the present disclosure provides a chirally controlled oligonucleotide having the sequence of any oligonucleotide disclosed herein, wherein the oligonucleotide is a linkage unimer. In some embodiments, the present disclosure provides a chirally controlled oligonucleotide having the sequence of any oligonucleotide disclosed herein, wherein the oligonucleotide is a gapmer. In some embodiments, the present disclosure provides a chirally controlled oligonucleotide having the sequence of any oligonucleotide disclosed herein, wherein the oligonucleotide is a skipmer.

In some embodiments, the present disclosure provides a chirally controlled oligonucleotide having the sequence of any oligonucleotide disclosed herein, wherein each cytosine is optionally and independently replaced by 5-methylcytosine. In some embodiments, the present disclosure provides a chirally controlled oligonucleotide having the sequence of any oligonucleotide disclosed herein, wherein at least one cytosine is optionally and independently replaced by 5-methylcytosine. In some embodiments, the present disclosure provides a chirally controlled oligonucleotide having the sequence of any oligonucleotide disclosed herein, wherein each cytosine is optionally and independently replaced by 5-methylcytosine.

In some embodiments, a chirally controlled oligonucleotide is designed such that one or more nucleotides comprise a phosphorus modification prone to “autorelease” under certain conditions. That is, under certain conditions, a particular phosphorus modification is designed such that it self-cleaves from the oligonucleotide to provide, e.g., a phosphate diester such as those found in naturally occurring DNA and RNA. In some embodiments, such a phosphorus modification has a structure of wherein each of L and R¹ is independently as defined above and described herein. In some embodiments, an autorelease group comprises a morpholino group. In some embodiments, an autorelease group is characterized by the ability to deliver an agent to the internucleotidic phosphorus linker, which agent facilitates further modification of the phosphorus atom such as, e.g., desulfurization. In some embodiments, the agent is water and the further modification is hydrolysis to form a phosphate diester as is found in naturally occurring DNA and RNA.

In some embodiments, a chirally controlled oligonucleotide is designed such that the resulting pharmaceutical properties are improved through one or more particular modifications at phosphorus. It is well documented in the art that certain oligonucleotides are rapidly degraded by nucleases and exhibit poor cellular uptake through the cytoplasmic cell membrane (Poijarvi-Virta et al., Curr. Med. Chem. (2006), 13(28); 3441-65; Wagner et al., Med. Res. Rev. (2000), 20(6):417-51; Peyrottes et al., Mini Rev. Med. Chem. (2004), 4(4):395-408; Gosselin et al., (1996), 43(1):196-208; Bologna et al., (2002), Antisense & Nucleic Acid Drug Development 12:33-41). For instance, Vives et al., (Nucleic Acids Research (1999), 27(20):4071-76) found that tert-butyl SATE pro-oligonucleotides displayed markedly increased cellular penetration compared to the parent oligonucleotide.

In some embodiments, a modification at a linkage phosphorus is characterized by its ability to be transformed to a phosphate diester, such as those present in naturally occurring DNA and RNA, by one or more esterases, nucleases, and/or cytochrome P450 enzymes, including but not limited to: CYP1A1, CYP1A2, CYP1B1 (Family: CYP1); CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1 (CYP2); CYP3A4, CYP3A5, CYP3A7, CYP3A43 (CYP3); CYP4A11, CYP4A22, CYP4B1, CYP4F2, CYP4F3, CYP4F8, CYP4F11, CYP4F12, CYP4F22, CYP4V2, CYP4X1, CYP4Z1 (CYP4); CYP5A1 (CYP5); CYP7A1, CYP7B1 (CYP7); CYP8A1 (prostacyclin synthase), CYP8B1 (bile acid biosynthesis) (CYP8); CYP11A1, CYP11B1, CYP11B2 (CYP11); CYP17A1 (CYP17); CYP19A1 (CYP19); CYP20A1 (CYP20); CYP21A2 (CYP21); CYP24A1 (CYP24); CYP26A1, CYP2XXX1, CYP26C1 (CYP26); CYP27A1 (bile acid biosynthesis), CYP27B1 (vitamin D3 1-alpha hydroxylase, activates vitamin D3), CYP27C1 (unknown function) (CYP27); CYP39A1 (CYP39); CYP46A1 (CYP46); or CYP51A1 (lanosterol 14-alpha demethylase) (CYP51).

In some embodiments, a modification at phosphorus results in a P-modification moiety characterized in that it acts as a pro-drug, e.g., the P-modification moiety facilitates delivery of an oligonucleotide to a desired location prior to removal. For instance, in some embodiments, a P-modification moiety results from PEGylation at the linkage phosphorus. One of skill in the relevant arts will appreciate that various PEG chain lengths are useful and that the selection of chain length will be determined in part by the result that is sought to be achieved by PEGylation. For instance, in some embodiments, PEGylation is effected in order to reduce RES uptake and extend in vivo circulation lifetime of an oligonucleotide.

In some embodiments, a PEGylation reagent for use in accordance with the present disclosure is of a molecular weight of about 300 g/mol to about 100,000 g/mol. In some embodiments, a PEGylation reagent is of a molecular weight of about 300 g/mol to about 10,000 g/mol. In some embodiments, a PEGylation reagent is of a molecular weight of about 300 g/mol to about 5,000 g/mol. In some embodiments, a PEGylation reagent is of a molecular weight of about 500 g/mol. In some embodiments, a PEGylation reagent of a molecular weight of about 1000 g/mol. In some embodiments, a PEGylation reagent is of a molecular weight of about 3000 g/mol. In some embodiments, a PEGylation reagent is of a molecular weight of about 5000 g/mol.

In certain embodiments, a PEGylation reagent is PEG500. In certain embodiments, a PEGylation reagent is PEG1000. In certain embodiments, a PEGylation reagent is PEG3000. In certain embodiments, a PEGylation reagent is PEG5000.

In some embodiments, a P-modification moiety is characterized in that it acts as a PK enhancer, e.g., lipids, PEGylated lipids, etc.

In some embodiments, a P-modification moiety is characterized in that it acts as an agent which promotes cell entry and/or endosomal escape, such as a membrane-disruptive lipid or peptide.

In some embodiments, a P-modification moiety is characterized in that it acts as a targeting agent. In some embodiments, a P-modification moiety is or comprises a targeting agent. The phrase “targeting agent,” as used herein, is an entity that is associates with a payload of interest (e.g., with an oligonucleotide or oligonucleotide composition) and also interacts with a target site of interest so that the payload of interest is targeted to the target site of interest when associated with the targeting agent to a materially greater extent than is observed under otherwise comparable conditions when the payload of interest is not associated with the targeting agent. A targeting agent may be, or comprise, any of a variety of chemical moieties, including, for example, small molecule moieties, nucleic acids, polypeptides, carbohydrates, etc. Targeting agents are described further by Adarsh et al., “Organelle Specific Targeted Drug Delivery—A Review,” International Journal of Research in Pharmaceutical and Biomedical Sciences, 2011, p. 895.

Examples of such targeting agents include, but are not limited to, proteins (e.g. Transferrin), oligopeptides (e.g., cyclic and acylic RGD-containing oligopedptides), antibodies (monoclonal and polyclonal antibodies, e.g. IgG, IgA, IgM, IgD, IgE antibodies), sugars/carbohydrates (e.g., monosaccharides and/or oligosaccharides (mannose, mannose-6-phosphate, galactose, and the like)), vitamins (e.g., folate), or other small biomolecules. In some embodiments, a targeting moiety is a steroid molecule (e.g., bile acids including cholic acid, deoxycholic acid, dehydrocholic acid; cortisone; digoxigenin; testosterone; cholesterol; cationic steroids such as cortisone having a trimethylaminomethyl hydrazide group attached via a double bond at the 3-position of the cortisone ring, etc.). In some embodiments, a targeting moiety is a lipophilic molecule (e.g., alicyclic hydrocarbons, saturated and unsaturated fatty acids, waxes, terpenes, and polyalicyclic hydrocarbons such as adamantine and buckminsterfullerenes). In some embodiments, a lipophilic molecule is a terpenoid such as vitamin A, retinoic acid, retinal, or dehydroretinal. In some embodiments, a targeting moiety is a peptide.

In some embodiments, a P-modification moiety is a targeting agent of formula —X-L-R¹ wherein each of X, L, and R¹ are as defined in Formula I, disclosed herein.

In some embodiments, a P-modification moiety is characterized in that it facilitates cell specific delivery.

In some embodiments, a P-modification moiety is characterized in that it falls into one or more of the above-described categories. For instance, in some embodiments, a P-modification moiety acts as a PK enhancer and a targeting ligand. In some embodiments, a P-modification moiety acts as a pro-drug and an endosomal escape agent. One of skill in the relevant arts would recognize that numerous other such combinations are possible and are contemplated by the present disclosure.

In some embodiments, a carbocyclyl, aryl, heteroaryl, or heterocyclyl group, or a bivalent or polyvalent group thereof, is a C₃-C₃₀ carbocyclyl, aryl, heteroaryl, or heterocyclyl group, or a bivalent and/or polyvalent group thereof.

### Bases (Nucleobases)

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any nucleobase described herein or known in the art.

In some embodiments, a nucleobase present in a provided oligonucleotide is a natural nucleobase or a modified nucleobase derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products). Example modified nucleobases are disclosed in Chiu and Rana, RNA, 2003, 9, 1034-1048, Limbach et al. Nucleic Acids Research, 1994, 22, 2183-2196 and Revankar and Rao, Comprehensive Natural Products Chemistry, vol. 7, 313. In some embodiments, a modified nucleobase is substituted uracil, thymine, adenine, cytosine, or guanine. In some embodiments, a modified nucleobase is a functional replacement, e.g., in terms of hydrogen bonding and/or base pairing, of uracil, thymine, adenine, cytosine, or guanine. In some embodiments, a nucleobase is optionally substituted uracil, thymine, adenine, cytosine, 5-methylcytosine, or guanine. In some embodiments, a nucleobase is uracil, thymine, adenine, cytosine, 5-methylcytosine, or guanine.

In some embodiments, a modified base is optionally substituted adenine, cytosine, guanine, thymine, or uracil. In some embodiments, a modified nucleobase is independently adenine, cytosine, guanine, thymine or uracil, modified by one or more modifications by which:
(1) a nucleobase is modified by one or more optionally substituted groups independently selected from acyl, halogen, amino, azide, alkyl, alkenyl, alkynyl, aryl, heteroalkyl, heteroalkenyl, heteroalkynyl, heterocyclyl, heteroaryl, carboxyl, hydroxyl, biotin, avidin, streptavidin, substituted silyl, and combinations thereof;
(2) one or more atoms of a nucleobase are independently replaced with a different atom selected from carbon, nitrogen and sulfur;
(3) one or more double bonds in a nucleobase are independently hydrogenated; or
(4) one or more aryl or heteroaryl rings are independently inserted into a nucleobase.

Compounds represented by the following general formulae are also contemplated as modified nucleobases:

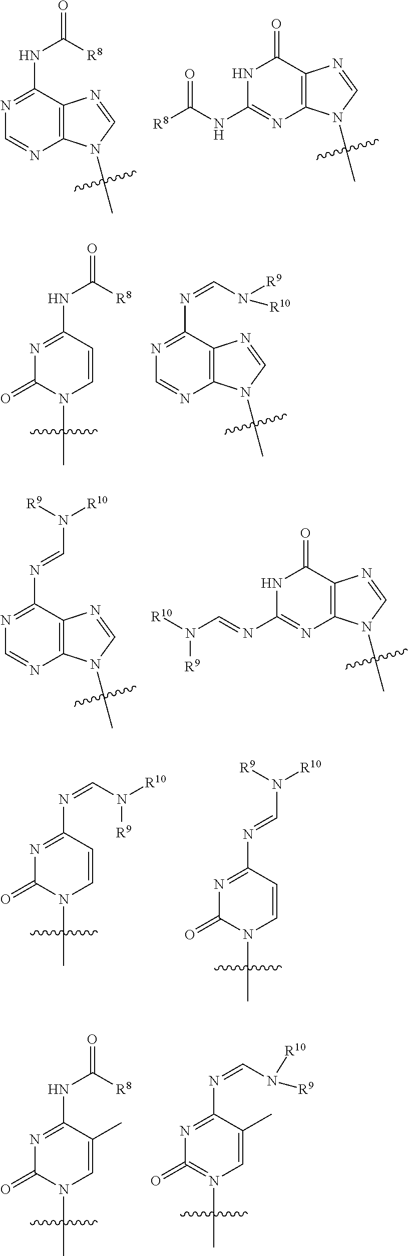

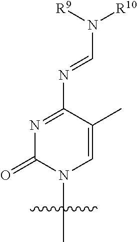

wherein R⁸ is an optionally substituted, linear or branched group selected from aliphatic, aryl, aralkyl, aryloxylalkyl, carbocyclyl, heterocyclyl and heteroaryl group having 1 to 15 carbon atoms, including, by way of example only, a methyl, isopropyl, phenyl, benzyl, or phenoxymethyl group; and each of R⁹ and R¹⁰ is independently an optionally substituted group selected from linear or branched aliphatic, carbocyclyl, aryl, heterocyclyl and heteroaryl.

Modified nucleobases also include expanded-size nucleobases in which one or more aryl rings, such as phenyl rings, have been added. Nucleic base replacements described in the Glen Research catalog (www.glenresearch.com); Krueger A T et al, Acc. Chem. Res., 2007, 40, 141-150; Kool, E T, Acc. Chem. Res., 2002, 35, 936-943; Benner S. A., et al., Nat. Rev. Genet., 2005, 6, 553-543; Romesberg, F. E., et al., Curr. Opin. Chem. Biol., 2003, 7, 723-733; Hirao, I., Curr. Opin. Chem. Biol., 2006, 10, 622-627, are contemplated as useful for the synthesis of the nucleic acids described herein. Some examples of these expanded-size nucleobases are shown below:

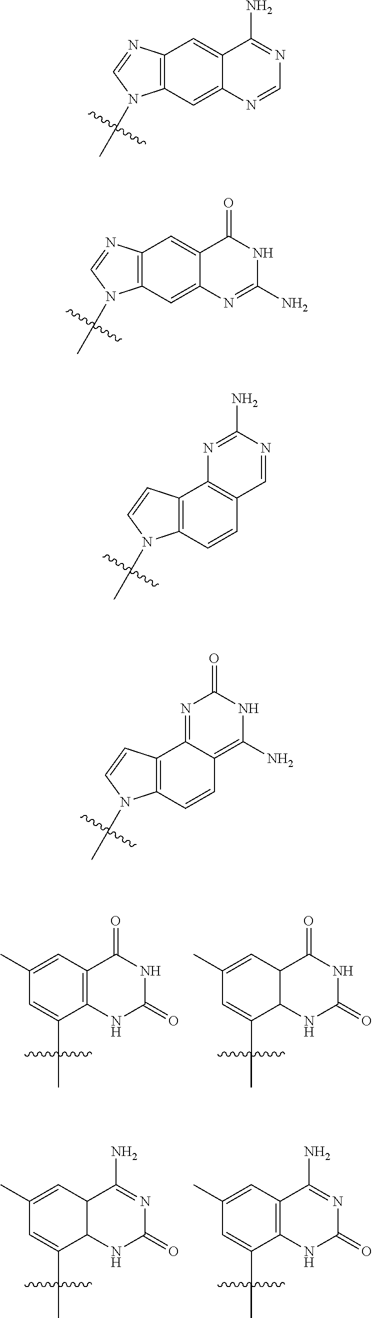

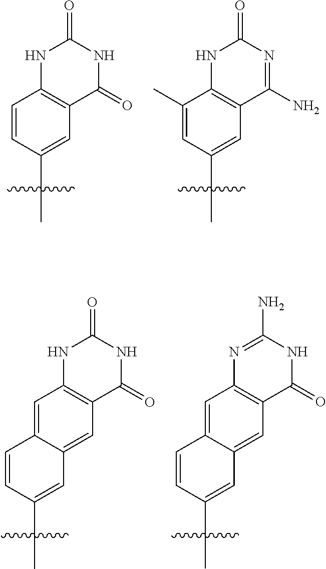

Herein, modified nucleobases also encompass structures that are not considered nucleobases but are other moieties such as, but not limited to, corrin- or porphyrin-derived rings. Porphyrin-derived base replacements have been described in Morales-Rojas, H and Kool, ET, Org. Lett., 2002, 4, 4377-4380. Shown below is an example of a porphyrin-derived ring which can be used as a base replacement:

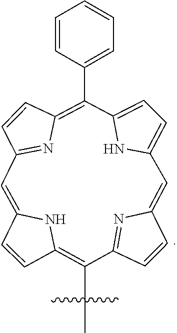 [SMILES: *c1c2nc(cc3ccc([nH]3)c(-c3ccccc3)c3nc(cc4ccc1[nH]4)C=C3)C=C2]

In some embodiments, modified nucleobases are of any one of the following structures, optionally substituted:

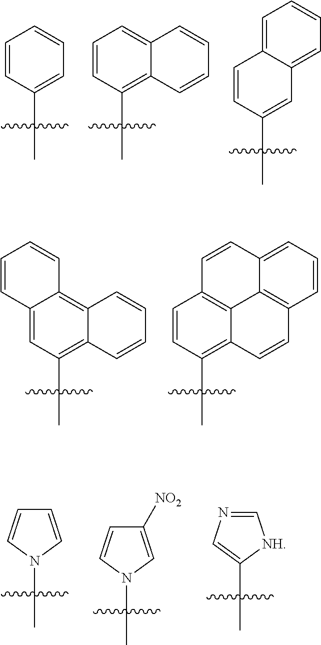

In some embodiments, a modified nucleobase is fluorescent. Examples of such fluorescent modified nucleobases include phenanthrene, pyrene, stillbene, isoxanthine, isozanthopterin, terphenyl, terthiophene, benzoterthiophene, coumarin, lumazine, tethered stillbene, benzo-uracil, and naphtho-uracil, as shown below:

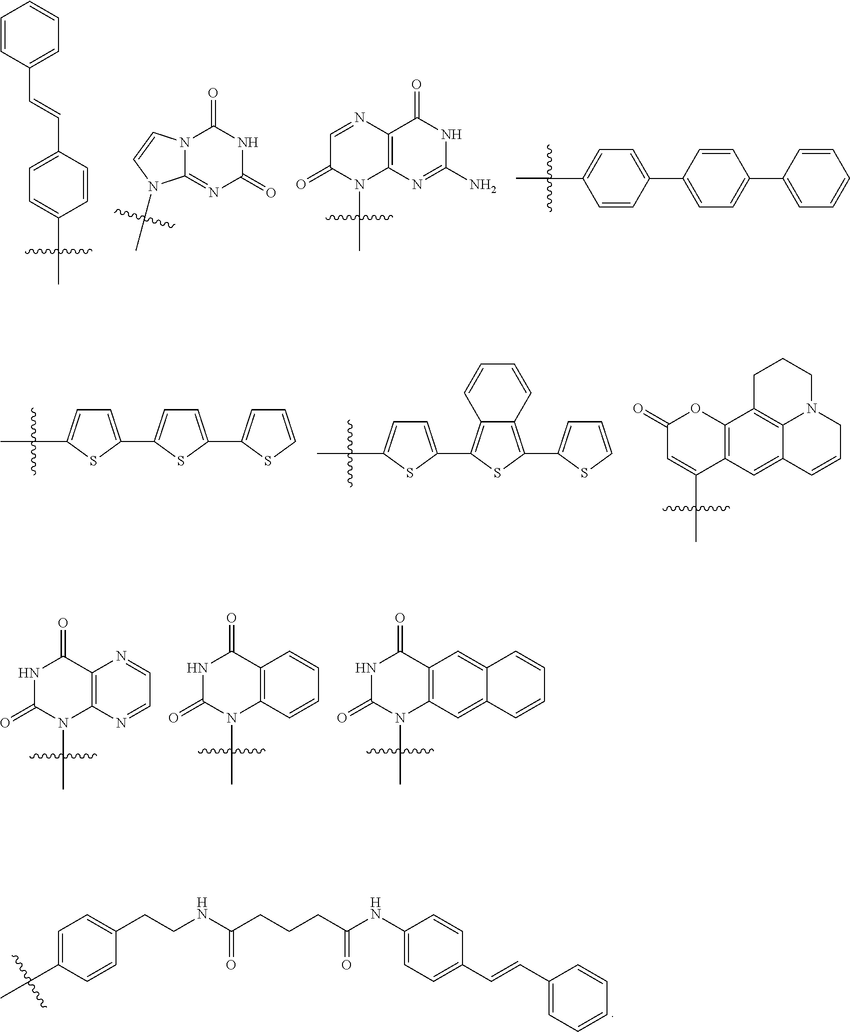

In some embodiments, a nucleobase or modified nucleobase is selected from: C5-propyne T, C5-propyne C, C5-Thiazole, Phenoxazine, 2-Thio-thymine, 5-Triazolylphenyl-thymine, Diaminopurine, and N²-Aminopropylguanine.

In some embodiments, a modified nucleobase is selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4- thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size- expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2- one (G-clamp). Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Example United States patents that teach the preparation of certain of the above noted modified nucleobases as well as other modified nucleobases include without limitation, US2003/0158403, U.S. Pat. Nos. 3,687,808; 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,750,692; 5,763,588; 5,830,653; and 6,005,096.

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, other nucleosides can also be used in the process disclosed herein and include nucleosides that incorporate modified nucleobases, or nucleobases covalently bound to modified sugars. Some examples of nucleosides that incorporate modified nucleobases include 4-acetylcytidine; 5-(carboxyhydroxylmethyl)uridine; 2″-O-methylcytidine; 5-carboxymethylaminomethyl-2-thiouridine; 5-carboxymethylaminomethyluridine; dihydrouridine; 2″-O-methylpseudouridine; beta,D-galactosylqueosine; 2″-O-methylguanosine; N⁶-isopentenyladenosine; 1-methyladenosine; 1-methylpseudouridine; 1-methylguanosine; 1-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2″-O-methyl-5-methyluridine; and 2″-O-methyluridine.

In some embodiments, nucleosides include 6-modified bicyclic nucleosides that have either (R) or (S)-chirality at the 6-position and include the analogs described in U.S. Pat. No. 7,399,845. In other embodiments, nucleosides include 5″-modified bicyclic nucleosides that have either (R) or (S)-chirality at the 5-position and include the analogs described in US Patent Application Publication No. 20070287831.

In some embodiments, a nucleobase or modified nucleobase comprises one or more biomolecule binding moieties such as e.g., antibodies, antibody fragments, biotin, avidin, streptavidin, receptor ligands, or chelating moieties. In other embodiments, a nucleobase or modified nucleobase is 5-bromouracil, 5-iodouracil, or 2,6-diaminopurine. In some embodiments, a nucleobase or modified nucleobase is modified by substitution with a fluorescent or biomolecule binding moiety. In some embodiments, the substituent on a nucleobase or modified nucleobase is a fluorescent moiety. In some embodiments, the substituent on a nucleobase or modified nucleobase is biotin or avidin.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the modified nucleobases, sugars, and internucleotidic linkages of each of which are incorporated by reference.

In some embodiments, a base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms. In some embodiments, a modified base is optionally substituted A, T, C, G or U, wherein one or more —NH₂ are independently and optionally replaced with —C(-L-R¹)₃, one or more —NH— are independently and optionally replaced with —C(-L-R¹)₂—, one or more ═N— are independently and optionally replaced with —C(-L-R¹)—, one or more ═CH— are independently and optionally replaced with ═N—, and one or more ═O are independently and optionally replaced with ═S, ═N(-L-R¹), or ═C(-L-R¹)₂, wherein two or more -L-R¹ are optionally taken together with their intervening atoms to form a 3-30 membered bicyclic or polycyclic ring having 0-10 heteroatom ring atoms, wherein the modified base is different than the natural A, T, C, G and U. In some embodiments, a base is optionally substituted A, T, C, G or U. In some embodiments, a modified base is substituted A, T, C, G or U, wherein the modified base is different than the natural A, T, C, G and U.

In some embodiments, a nucleoside is any described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts'o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 20070900071; WO 20070900071; or WO 2016/079181.

Example nucleobases are also described in US 20110294124, US 20120316224, US 20140194610, US 20150211006, US 20150197540, WO 2015107425, PCT/US2016/043542, and PCT/US2016/043598, each of which is incorporated herein by reference. Various additional nucleobases are described in the art and can be utilized in accordance with the present disclosure.

Sugars

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides comprise any sugar described herein or known in the art.

In some embodiments, provided oligonucleotides capable of directing single-stranded RNA interference comprise one or more modified sugar moieties beside the natural sugar moieties.

The most common naturally occurring nucleotides are comprised of ribose sugars linked to the nucleobases adenosine (A), cytosine (C), guanine (G), and thymine (T) or uracil (U). Also contemplated are modified nucleotides wherein a phosphate group or linkage phosphorus in the nucleotides can be linked to various positions of a sugar or modified sugar. As non-limiting examples, the phosphate group or linkage phosphorus can be linked to the 2″, 3″, 4″ or 5″ hydroxyl moiety of a sugar or modified sugar. Nucleotides that incorporate modified nucleobases as described herein are also contemplated in this context. In some embodiments, nucleotides or modified nucleotides comprising an unprotected —OH moiety are used in accordance with methods of the present disclosure.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base (nucleobase), modified base or base analog described herein or known in the art. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base described herein or known in the art in combination with any other structural element or modification described herein, including but not limited to, base sequence or portion thereof, sugar; internucleotidic linkage; stereochemistry or pattern thereof; additional chemical moiety, including but not limited to, a targeting moiety, lipid moiety, a GalNAc moiety, etc.; 5′-end structure; 5′-end region; 5′ nucleotide moiety; seed region; post-seed region; 3′-end region; 3′-terminal dinucleotide; 3′-end cap; GC content; long GC stretch; pattern of modifications of sugars, bases or internucleotidic linkages; format or any structural element thereof, and/or any other structural element or modification described herein; and in some embodiments, the present disclosure pertains to multimers of any such oligonucleotides.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any sugar.

In some embodiments, a sugar has a structure of:

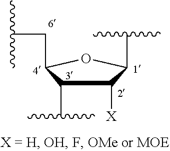

Modified sugars can be incorporated into a provided oligonucleotide. In some embodiments, a modified sugar contains one or more substituents at the 2″ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O-(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE. Also contemplated herein are modified sugars described in WO 2001/088198; and Martin et al., Helv. Chico. Acta, 1995, 78, 486-504. In some embodiments, a modified sugar comprises one or more groups selected from a substituted silyl group, an RNA cleaving group, a reporter group, a fluorescent label, an intercalator, a group for improving the pharmacokinetic properties of a nucleic acid, a group for improving the pharmacodynamic properties of a nucleic acid, or other substituents having similar properties. In some embodiments, modifications are made at one or more of the the 2′, 3′, 4′, 5′, or 6′ positions of the sugar or modified sugar, including the 3′ position of the sugar on the 3′-terminal nucleotide or in the 5′ position of the 5′-terminal nucleotide.

In some embodiments, a 2′-modification is 2′-F.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe.

Modified sugars also include locked nucleic acids (LNAs). In some embodiments, two substituents on sugar carbon atoms are taken together to form a bivalent moiety. In some embodiments, two substituents are on two different sugar carbon atoms. In some embodiments, a formed bivalent moiety has the structure of -L- as defined herein. In some embodiments, -L- is —O—CH₂—, wherein —CH₂— is optionally substituted. In some embodiments, -L- is —O—CH₂—. In some embodiments, -L- is —O—CH(Et)-. In some embodiments, -L- is between C₂ and C₄ of a sugar moiety. In some embodiments, a locked nucleic acid has the structure indicated below. A locked nucleic acid of the structure below is indicated, wherein Ba represents a nucleobase or modified nucleobase as described herein, and wherein R^{2s} is —OCH₂C4′-. In some embodiments, a modified nucleoside has a structure of:

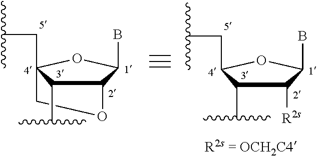

Wherein B is a base.

In some embodiments, a modified sugar is an ENA such as those described in, e.g., Seth et al., J Am Chem Soc. 2010 Oct. 27; 132(42): 14942-14950. In some embodiments, a modified sugar is any of those found in an XNA (xenonucleic acid), for instance, arabinose, anhydrohexitol, threose, 2′ fluoroarabinose, or cyclohexene.

Modified sugars include cyclobutyl or cyclopentyl moieties in place of the pentofuranosyl sugar. Representative United States patents that teach the preparation of such modified sugar structures include, but are not limited to, U.S. Pat. Nos. 4,981,957; 5,118,800; 5,319,080; and 5,359,044. Some modified sugars that are contemplated include sugars in which the oxygen atom within the ribose ring is replaced by nitrogen, sulfur, selenium, or carbon. In some embodiments, a modified sugar is a modified ribose wherein the oxygen atom within the ribose ring is replaced with nitrogen, and wherein the nitrogen is optionally substituted with an alkyl group (e.g., methyl, ethyl, isopropyl, etc).

Non-limiting examples of modified sugars include glycerol, which form glycerol nucleic acid (GNA). One example of a GNA is shown below and is described in Zhang, R et al., J. Am. Chem. Soc., 2008, 130, 5846-5847; Zhang L, et al., J. Am. Chem. Soc., 2005, 127, 4174-4175 and Tsai C H et al., PNAS, 2007, 14598-14603. In some embodiments, a nucleoside has a structure of:

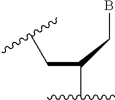

Wherein B is a base.

A flexible nucleic acid (FNA) based on the mixed acetal aminal of formyl glycerol, is described in Joyce G F et al., PNAS, 1987, 84, 4398-4402 and Heuberger B D and Switzer C, J. Am. Chem. Soc., 2008, 130, 412-413. In some embodiments, a nucleoside has a structure of:

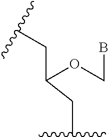

Wherein B is a base.

Additional non-limiting examples of modified sugars and/or modified nucleosides and/or modified nucleotides include hexopyranosyl (6′ to 4′), pentopyranosyl (4′ to 2′), pentopyranosyl (4′ to 3′), 5′-deoxy-5′-C-malonyl, squaryldiamide, and tetrofuranosyl (3′ to 2′) sugars. In some embodiments, a modified nucleoside comprises a hexopyranosyl (6′ to 4′) sugar and has the structure of any one in the following formulae:

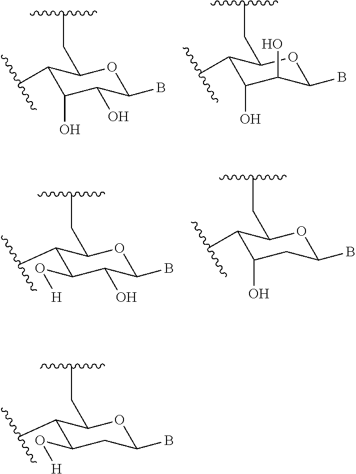

wherein X^{s} corresponds to the P-modification group “—XLR¹” described herein wherein XLR¹ is equivalent to X-L-R¹ and X, L, and R¹ are as defined in Formula I, disclosed herein, and B is a base.

In some embodiments, a modified nucleotide comprises a pentopyranosyl (4′ to 2′) sugar and has a structure of any one in the following formulae:

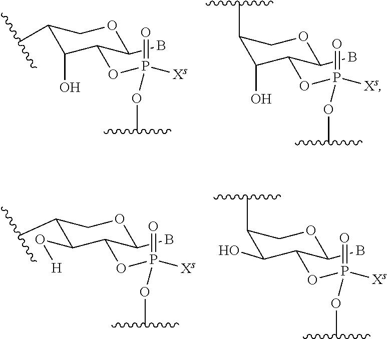

wherein X^{s} corresponds to the P-modification group “—XLR¹” described herein, wherein XLR¹ is equivalent to X-L-R¹ and X, L, and R¹ are as defined in Formula I, disclosed herein, and B is a base.

In some embodiments, a modified nucleotide comprises a pentopyranosyl (4′ to 3′) sugar and is of any one in the following formulae:

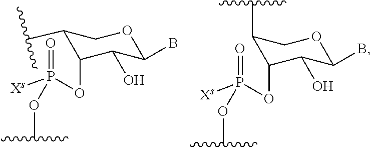

wherein X^{s} corresponds to the P-modification group “—XLR¹” described herein, wherein XLR¹ is equivalent to X-L-R¹ and X, L, and R¹ are as defined in Formula I, disclosed herein, and B is a base.

In some embodiments, a modified nucleotide comprises a tetrofuranosyl (3′ to 2′) sugar and is of either in the following formulae:

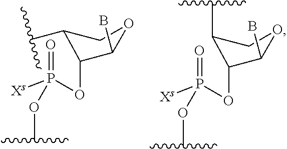

wherein X^{s} corresponds to the P-modification group “—XLR¹” described herein, wherein XLR¹ is equivalent to X-L-R¹ and X, L, and R¹ are as defined in Formula I, disclosed herein, and B is a base.

In some embodiments, a modified nucleotide comprises a modified sugar and is of any one in the following formulae:

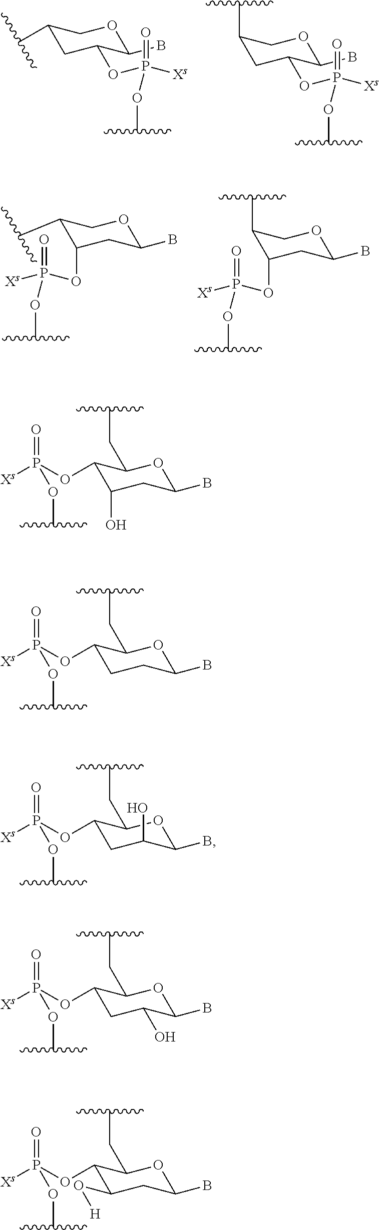

wherein X^{s} corresponds to the P-modification group “—XLR¹” described herein, wherein XLR¹ is equivalent to X-L-R¹ and X, L, and R¹ are as defined in Formula I, disclosed herein, and B is a base.

In some embodiments, one or more hydroxyl group in a sugar moiety is optionally and independently replaced with halogen, R′—N(R′)₂, —OR′, or —SR′, wherein each R′ is independently as defined above and described herein.

In some embodiments, a modified nucleotide is as illustrated below, wherein Xₛ corresponds to the P-modification group “—XLR¹” described herein, wherein XLR¹ is equivalent to X-L-R¹ and X, L, and R¹ are as defined in Formula I, disclosed herein, B is a base, and X¹ is selected from —S—, —Se—, —CH₂—, —NMe-, —NEt- and —NiPr—

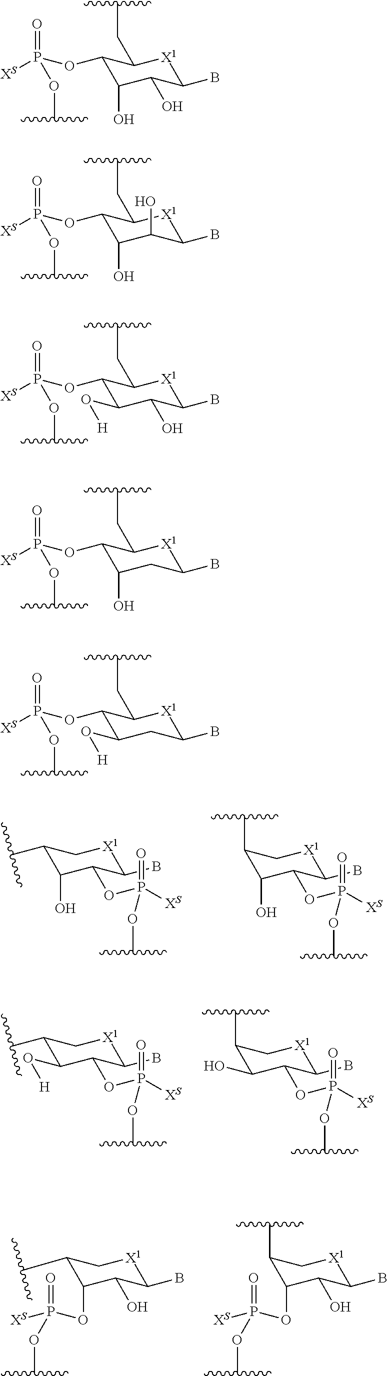

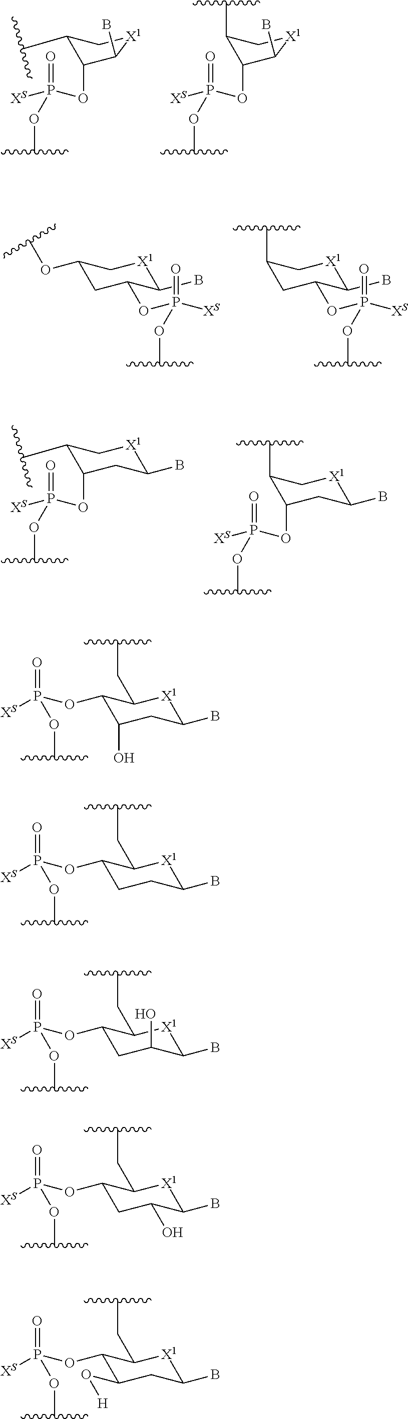

In some embodiments, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more (e.g., 55%, 60%, 65%, 70%, 75%, 80%, 85%, 90%, 95% or more), inclusive, of the sugars in a single-stranded RNAi agent are modified. In some embodiments, only purine residues are modified (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more [e.g., 55%, 60%, 65%, 70%, 75%, 80%, 85%, 90%, 95% or more] of the purine residues are modified). In some embodiments, only pyrimidine residues are modified (e.g., about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more [e.g., 55%, 60%, 65%, 70%, 75%, 80%, 85%, 90%, 95% or more] of the pyridimine residues are modified). In some embodiments, both purine and pyrimidine residues are modified.

Modified sugars can be prepared by methods known in the art, including, but not limited to: A. Eschenmoser, Science (1999), 284:2118; M. Bohringer et al, Helv. Chim. Acta (1992), 75:1416-1477; M. Egli et al, J. Am. Chem. Soc. (2006), 128(33):10847-56; A. Eschenmoser in Chemical Synthesis: Gnosis to Prognosis, C. Chatgilialoglu and V. Sniekus, Ed., (Kluwer Academic, Netherlands, 1996), p. 293; K.-U. Schoning et al, Science (2000), 290:1347-1351; A. Eschenmoser et al, Helv. Chim. Acta (1992), 75:218; J. Hunziker et al, Helv. Chim. Acta (1993), 76:259; G. Otting et al, Helv. Chim. Acta (1993), 76:2701; K. Groebke et al, Helv. Chim. Acta (1998), 81:375; and A. Eschenmoser, Science (1999), 284:2118. Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310). In some embodiments, a modified sugar is any of those described in PCT Publication No. WO2012/030683, incorporated herein by reference, and/or depicted herein. In some embodiments, a modified sugar is any modified sugar described in any of: Gryaznov, S; Chen, J.-K. J. Am. Chem. Soc. 1994, 116, 3143; Hendrix et al. 1997 Chem. Eur. J. 3: 110; Hyrup et al. 1996 Bioorg. Med. Chem. 4: 5; Jepsen et al. 2004 Oligo. 14: 130-146; Jones et al. J. Org. Chem. 1993, 58, 2983; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Mesmaeker et al. Angew. Chem., Int. Ed. Engl. 1994, 33, 226; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Pallan et al. 2012 Chem. Comm. 48: 8195-8197; Petersen et al. 2003 TRENDS Biotech. 21: 74-81; Rajwanshi et al. 1999 Chem. Commun. 1395-1396; Schultz et al. 1996 Nucleic Acids Res. 24: 2966; Seth et al. 2009 J. Med. Chem. 52: 10-13; Seth et al. 2010 J. Med. Chem. 53: 8309-8318; Seth et al. 2010 J. Org. Chem. 75: 1569-1581; Seth et al. 2012 Bioo. Med. Chem. Lett. 22: 296-299; Seth et al. 2012 Mol. Ther-Nuc. Acids. 1, e47; Seth, Punit P; Siwkowski, Andrew; Allerson, Charles R; Vasquez, Guillermo; Lee, Sam; Prakash, Thazha P; Kinberger, Garth; Migawa, Michael T; Gaus, Hans; Bhat, Balkrishen; et al. From Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Ts′o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 20070900071; WO 20070900071; or WO 2016/079181.

In some embodiments, a modified sugar moiety is an optionally substituted pentose or hexose moiety. In some embodiments, a modified sugar moiety is an optionally substituted pentose moiety. In some embodiments, a modified sugar moiety is an optionally substituted hexose moiety. In some embodiments, a modified sugar moiety is an optionally substituted ribose or hexitol moiety. In some embodiments, a modified sugar moiety is an optionally substituted ribose moiety. In some embodiments, a modified sugar moiety is an optionally substituted hexitol moiety.

In some embodiments, an example modified nucleotide is selected from:

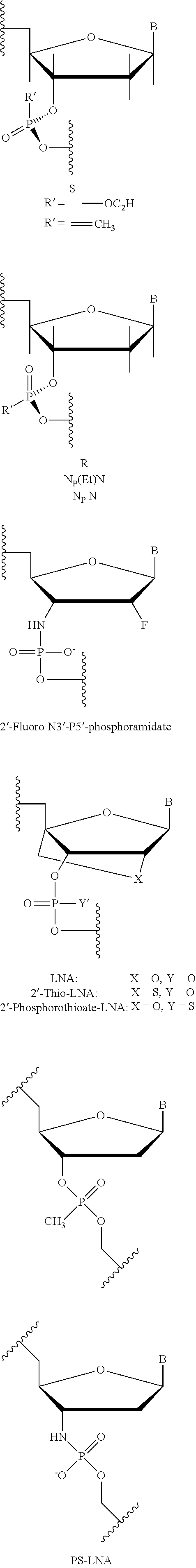

In some embodiments, a nucleotide has a structure selected from any of:

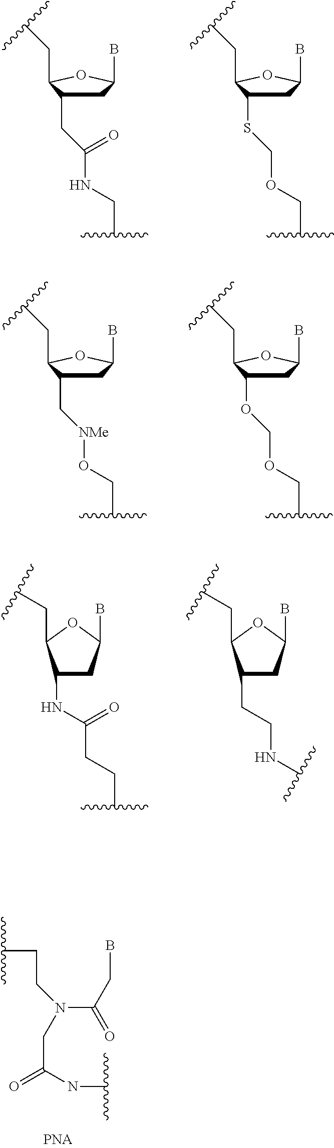

In some embodiments, a modified nucleoside has a structure selected from:

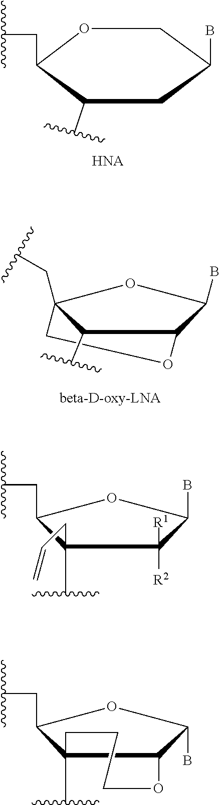

wherein R¹ and R² are independently —H, —F, —OMe, -MOE or substituted or unsubstituted C₁₋₆ alkyl;

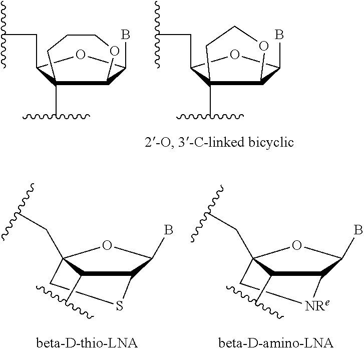

where R^{e} is substituted or unsubstituted C₁₋₆ alkyl or H

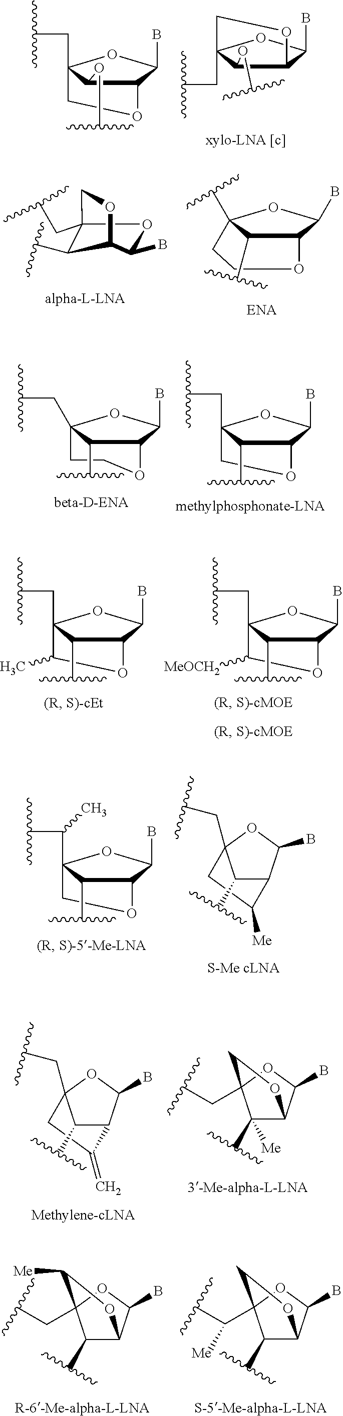

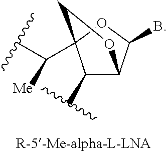

In some embodiments, a nucleotide and adjacent nucleoside have the structure of:

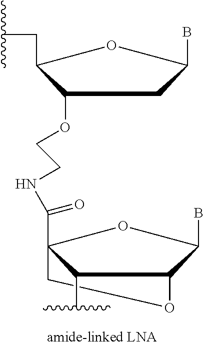

In some embodiments, R¹ is R as defined and described. In some embodiments, R² is R. In some embodiments, R^{e} is R. In some embodiments, R^{e} is H, CH₃, Bn, COCF₃, benzoyl, benzyl, pyren-1-ylcarbonyl, pyren-1-ylmethyl, 2-aminoethyl. In some embodiments, a non-limiting example internucleotidic linkage or sugar is or comprises a component of any of: N-methanocarba, C3-amide, Formacetal, Thioformacetal, MMI, PMO (phosphorodiamidate linked morpholino), PNA (peptide nucleic acid), LNA, cMOE BNA, cEt BNA, α-L-NA or a related analog, HNA, Me-ANA, MOE-ANA, Ara-FHNA, FHNA, R-6′-Me-FHNA, S-6′-Me-FHNA, ENA, or c-ANA. In some embodiments, a non-limiting example internucleotidic linkage or sugar is or comprises a component of any of those described in Allerson et al. 2005 J. Med. Chem. 48: 901-4; BMCL 2011 21: 1122; BMCL 2011 21: 588; BMCL 2012 22: 296; Chattopadhyaya et al. 2007 J. Am. Chem. Soc. 129: 8362; Chem. Bio. Chem. 2013 14: 58; Curr. Prot. Nucl. Acids Chem. 2011 1.24.1; Egli et al. 2011 J. Am. Chem. Soc. 133: 16642; Hendrix et al. 1997 Chem. Eur. J. 3: 110; Hyrup et al. 1996 Bioorg. Med. Chem. 4: 5; Imanishi 1997 Tet. Lett. 38: 8735; J. Am. Chem. Soc. 1994, 116, 3143; J. Med. Chem. 2009 52: 10; J. Org. Chem. 2010 75: 1589; Jepsen et al. 2004 Oligo. 14: 130-146; Jones et al. J. Org. Chem. 1993, 58, 2983; Jung et al. 2014 ACIEE 53: 9893; Kodama et al. 2014 AGDS; Koizumi 2003 BMC 11: 2211; Koizumi et al. 2003 Nuc. Acids Res. 12: 3267-3273; Koshkin et al. 1998 Tetrahedron 54: 3607-3630; Kumar et al. 1998 Bioo. Med. Chem. Let. 8: 2219-2222; Lauritsen et al. 2002 Chem. Comm. 5: 530-531; Lauritsen et al. 2003 Bioo. Med. Chem. Lett. 13: 253-256; Lima et al. 2012 Cell 150: 883-894; Mesmaeker et al. Angew. Chem., Int. Ed. Engl. 1994, 33, 226; Migawa et al. 2013 Org. Lett. 15: 4316; Mol. Ther. Nucl. Acids 2012 1: e47; Morita et al. 2001 Nucl. Acids Res. Supp. 1: 241-242; Morita et al. 2002 Bioo. Med. Chem. Lett. 12: 73-76; Morita et al. 2003 Bioo. Med. Chem. Lett. 2211-2226; Murray et al. 2012 Nucl. Acids Res. 40: 6135; Nielsen et al. 1997 Chem. Soc. Rev. 73; Nielsen et al. 1997 J. Chem. Soc. Perkins Transl. 1: 3423-3433; Obika et al. 1997 Tetrahedron Lett. 38 (50): 8735-8; Obika et al. 1998 Tetrahedron Lett. 39: 5401-5404; Obika et al. 2008 J. Am. Chem. Soc. 130: 4886; Obika et al. 2011 Org. Lett. 13: 6050; Oestergaard et al. 2014 JOC 79: 8877; Pallan et al. 2012 Biochem. 51: 7; Pallan et al. 2012 Chem. Comm. 48: 8195-8197; Petersen et al. 2003 TRENDS Biotech. 21: 74-81; Prakash et al. 2010 J. Med. Chem. 53: 1636; Prakash et al. 2015 Nucl. Acids Res. 43: 2993-3011; Prakash et al. 2016 Bioorg. Med. Chem. Lett. 26: 2817-2820; Rajwanshi et al. 1999 Chem. Commun. 1395-1396; Schultz et al. 1996 Nucleic Acids Res. 24: 2966; Seth et al. 2008 Nucl. Acid Sym. Ser. 52: 553; Seth et al. 2009 J. Med. Chem. 52: 10-13; Seth et al. 2010 J. Am. Chem. Soc. 132: 14942; Seth et al. 2010 J. Med. Chem. 53: 8309-8318; Seth et al. 2010 J. Org. Chem. 75: 1569-1581; Seth et al. 2011 BMCL 21: 4690; Seth et al. 2012 Bioo. Med. Chem. Lett. 22: 296-299; Seth et al. 2012 Mol. Ther-Nuc. Acids. 1, e47; Seth et al., Nucleic Acids Symposium Series (2008), 52(1), 553-554; Singh et al. 1998 Chem. Comm. 1247-1248; Singh et al. 1998 J. Org. Chem. 63: 10035-39; Singh et al. 1998 J. Org. Chem. 63: 6078-6079; Sorensen 2003 Chem. Comm. 2130-2131; Starrup et al. 2010 Nucl. Acids Res. 38: 7100; Swayze et al. 2007 Nucl. Acids Res. 35: 687; Ts′o et al. Ann. N. Y. Acad. Sci. 1988, 507, 220; Van Aerschot et al. 1995 Angew. Chem. Int. Ed. Engl. 34: 1338; Vasseur et al. J. Am. Chem. Soc. 1992, 114, 4006; WO 20070900071; WO 2016/079181; U.S. Pat. Nos. 6,326,199; 6,066,500; and 6,440,739, the base and sugar modifications of each of which is herein incorporated by reference.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any sugar described herein or known in the art. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any sugar described herein or known in the art in combination with any other structural element or modification described herein, including but not limited to, base sequence or portion thereof, base; internucleotidic linkage; stereochemistry or pattern thereof; additional chemical moiety, including but not limited to, a targeting moiety, lipid moiety, a GalNAc moiety, etc.; 5′-end structure; 5′-end region; 5′ nucleotide moiety; seed region; post-seed region; 3′-end region; 3′-terminal dinucleotide; 3′-end cap; GC content; long GC stretch; pattern of modifications of sugars, bases or internucleotidic linkages; format or any structural element thereof, and/or any other structural element or modification described herein; and in some embodiments, the present disclosure pertains to multimers of any such oligonucleotides. Various additional sugars are described in the art and can be utilized in accordance with the present disclosure.

Base Sequence of an Oligonucleotide

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can comprise any base sequence or portion thereof, described herein, wherein a portion is a span of at least 15 contiguous bases, or a span of at least 15 contiguous bases with 1-5 mismatches.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence described herein. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence or portion thereof, described herein. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence or portion thereof, described herein, wherein a portion is a span of 15 contiguous bases, or a span of 15 contiguous bases with 1-5 mismatches.

The sequence of a single-stranded RNAi agent has a sufficient length and identity to a transcript target to mediate target-specific RNA interference. In some embodiments, the RNAi agent is complementary to a portion of a transcript target sequence.

The base sequence of a single-stranded RNAi agent is complementary to that of a target transcript. As used herein, “target transcript sequence,” “target sequence”, “target gene”, and the like, refer to a contiguous portion of the nucleotide sequence of an mRNA molecule formed during the transcription of a gene, e.g., a target gene, including mRNA that is a product of RNA processing of a primary transcription product.

The terms “complementary,” “fully complementary” and “substantially complementary” herein may be used with respect to the base matching between the strand of a single-stranded RNAi agent and a target sequence or between an antisense oligonucleotide and a target sequence, as will be understood from the context of their use. A strand of a single-stranded RNAi agent or antisense oligonucleotide or other oligonucleotide is complementary to that of a target sequence when each base of the single-stranded RNAi agent, antisense oligonucleotide or other oligonucleotide is capable of base-pairing with a sequential base on the target strand, when maximally aligned. As a non-limiting example, if a target sequence has, for example, a base sequence of 5′-GCAUAGCGAGCGAGGGAAAAC-3′ (SEQ ID NO: 2), an oligonucleotide with a base sequence of 5′GUUUUCCCUCGCUCGCUAUGC-3′ (SEQ ID NO: 3) is complementary or fully complementary to such a target sequence. It is noted, of course, that substitution of T for U, or vice versa, does not alter the amount of complementarity.

As used herein, a polynucleotide that is “substantially complementary” to a target sequence is largely or mostly complementary but not 100% complementary. In some embodiments, a sequence (e.g., a strand of a single-stranded RNAi agent or an antisense oligonucleotide) which is substantially complementary has 1, 2, 3, 4 or 5 mismatches from a sequence which is 100% complementary to the target sequence. In the case of a single-stranded RNAi agent, this disclosure notes that the 5′ terminal nucleotide (N1) in many cases has a mismatch from the complement of a target sequence. Similarly, in a single-stranded RNAi agent, the 3′-terminal dinucleotide, if present, can be a mismatch from the complement of the target sequence. As a non-limiting example, if a target sequence has, for example, a base sequence of 5′-GCAUAGCGAGCGAGGGAAAAC-3′ (SEQ ID NO: 4), a single-stranded RNAi agent with a base sequence of 5′ TUUUUCCCUCGCUCGCUAUTU-3′ (SEQ ID NO: 5) is substantially complementary to such a target sequence.

The present disclosure presents, in Table 1A and elsewhere, various single-stranded RNAi agents and antisense oligonucleotides and other oligonucleotides, each of which has a defined base sequence. In some embodiments, the disclosure encompasses any oligonucleotide having a base sequence which is, comprises, or comprises a portion of the base sequence of any various single-stranded RNAi agent, antisense oligonucleotide and other oligonucleotide disclosed herein. In some embodiments, the disclosure encompasses any oligonucleotide having a base sequence which is, comprises, or comprises a portion of the base sequence of any various single-stranded RNAi agent, antisense oligonucleotide and other oligonucleotide disclosed herein, which has any chemical modification, stereochemistry, format, structural feature (e.g., if the oligonucleotide is a single-stranded RNAi agent, the 5′-end structure, 5′-end region, 5′ nucleotide moiety, seed region, post-seed region, 3′-end region, 3′-terminal dinucleotide, 3′-end cap, or any structure, pattern or portion thereof), and/or any other modification described herein (e.g., conjugation with another moiety, such as a targeting moiety, carbohydrate moiety, a GalNAc moiety, lipid moiety, etc.; and/or multimerization).

In some embodiments, an oligonucleotide has a base sequence which is, comprises or comprises a portion of the base sequence of any oligonucleotide disclosed herein.

In some embodiments, an oligonucleotide targets ACVR2B and has a base sequence which is, comprises or comprises a portion of the base sequence of any oligonucleotide disclosed herein.

In some embodiments, an oligonucleotide targets APOB and has a base sequence which is, comprises or comprises a portion of: the base sequence of any oligonucleotide disclosed herein.

In some embodiments, an oligonucleotide targets FXI and has a base sequence which is, comprises or comprises a portion of the base sequence of any oligonucleotide disclosed herein.

In some embodiments, an oligonucleotide targets KRT and has a base sequence which is, comprises or comprises a portion of the base sequence of any oligonucleotide disclosed herein.

In some embodiments, an oligonucleotide targets KRT14 and has a base sequence which is, comprises or comprises a portion of the base sequence of any oligonucleotide disclosed herein.

In some embodiments, an oligonucleotide targets MSTN and has a base sequence which is, comprises or comprises a portion of the base sequence of any oligonucleotide disclosed herein.

In some embodiments, an oligonucleotide targets MSTN-R and has a base sequence which is, comprises or comprises a portion of the base sequence of any oligonucleotide disclosed herein.

In some embodiments, an oligonucleotide targets PCSK9 and has a base sequence which is, comprises or comprises a portion of the base sequence of any oligonucleotide disclosed herein.

In some embodiments, an oligonucleotide targets APOC3 and has a base sequence which is, comprises or comprises a portion of: AAAGCTGGACAAGAAGCTA (SEQ ID NO: 6).

In some embodiments, an oligonucleotide targets APOC3 and has a base sequence which is, comprises or comprises a portion of: AACCTTAGCTGGGTCTGCCA (SEQ ID NO: 7).

In some embodiments, an oligonucleotide targets APOC3 and has a base sequence which is, comprises or comprises a portion of: AAGCAGCTTCTTGTCCAGC (SEQ ID NO: 8).

In some embodiments, an oligonucleotide targets APOC3 and has a base sequence which is, comprises or comprises a portion of: AAGGGAGGCATCCTCGGCCT (SEQ ID NO: 9).

In some embodiments, an oligonucleotide targets APOC3 and has a base sequence which is, comprises or comprises a portion of: AAGTTGGTCTGACCTCAGGG (SEQ ID NO: 10).

In some embodiments, an oligonucleotide targets APOC3 and has a base sequence which is, comprises or comprises a portion of: AATAAAGCTGGACAAGAAGCTGCTAT (SEQ ID NO: 11).

In some embodiments, an oligonucleotide targets APOC3 and has a base sequence which is, comprises or comprises a portion of: AATACTGTCCCTTTTAAGC (SEQ ID NO: 12).

In some embodiments, an oligonucleotide targets APOC3 and has a base sequence which is, comprises or comprises a portion of: ACACCACCCTCTCAACTTCA (SEQ ID NO: 13).

In some embodiments, an oligonucleotide targets APOC3 and has a base sequence which is, comprises or comprises a portion of: ACACCCATGTCCCCACTGGA (SEQ ID NO: 14).

In some embodiments, an oligonucleotide targets APOC3 and has a base sequence which is, comprises or comprises a portion of: ACCGCCAAGGATGCACTGAGCAGC (SEQ ID NO: 15).

In some embodiments, an oligonucleotide targets APOC3 and has a base sequence which is, comprises or comprises a portion of: ACTTGTCCAGCTTTATTGG (SEQ ID NO: 16).

In some embodiments, an oligonucleotide targets APOC3 and has a base sequence which is, comprises or comprises a portion of: AGCAGCGTGCAGGAGTCCCAGGTG (SEQ ID NO: 17).

In some embodiments, an oligonucleotide targets APOC3 and has a base sequence which is, comprises or comprises a portion of: AGCAGCTTCTTGTCCAGC (SEQ ID NO: 18).

In some embodiments, an oligonucleotide targets APOC3 and has a base sequence which is, comprises or comprises a portion of: AGCAGCTTCTTGTCCAGCT (SEQ ID NO: 19).

In some embodiments, an oligonucleotide targets APOC3 and has a base sequence which is, comprises or comprises a portion of: AGCAGCTTCTTGTCCAG (SEQ ID NO: 20).

In some embodiments, an oligonucleotide targets APOC3 and has a base sequence which is, comprises or comprises a portion of: AGCCACGGCTGAAGTTGGTC (SEQ ID NO: 21).

In some embodiments, an oligonucleotide targets APOC3 and has a base sequence which is, comprises or comprises a portion of: AGCCATCGGTCACCCAGCCC (SEQ ID NO: 22).

In some embodiments, an oligonucleotide targets APOC3 and has a base sequence which is, comprises or comprises a portion of: AGCTTCTTGTCCAGCTTTAT (SEQ ID NO: 23).

In some embodiments, an oligonucleotide targets APOC3 and has a base sequence which is, comprises or comprises a portion of: AGCTTCTTGTCCAG (SEQ ID NO: 24).

In some embodiments, an oligonucleotide targets APOC3 and has a base sequence which is, comprises or comprises a portion of: AGCTTCTTGTCCAGCTTT (SEQ ID NO: 25).

In some embodiments, an oligonucleotide targets APOC3 and has a base sequence which is, comprises or comprises a portion of: AGCTTCTTGTCCAGCTTTATTT (SEQ ID NO: 26).

In some embodiments, an oligonucleotide targets APOC3 and has a base sequence which is, comprises or comprises a portion of: AGCTTCTTGTCCAGCT (SEQ ID NO: 27).

In some embodiments, an oligonucleotide targets APOC3 and has a base sequence which is, comprises or comprises a portion of: AGCTTCTTGTCCAGCTTT (SEQ ID NO: 28).

In some embodiments, an oligonucleotide targets APOC3 and has a base sequence which is, comprises or comprises a portion of: AGCTTCTTGTCCAGCTTTA (SEQ ID NO: 29).

In some embodiments, an oligonucleotide targets APOC3 and has a base sequence which is, comprises or comprises a portion of: AGGCATCCTCGGCCTCTGAA (SEQ ID NO: 30).

In some embodiments, an oligonucleotide targets APOC3 and has a base sequence which is, comprises or comprises a portion of: AGGCCAGCATGCCTGGAGGG (SEQ ID NO: 31).

In some embodiments, an oligonucleotide targets APOC3 and has a base sequence which is, comprises or comprises a portion of: AGGCCAGCATGCCTGGAGG (SEQ ID NO: 32).

In some embodiments, an oligonucleotide targets APOC3 and has a base sequence which is, comprises or comprises a portion of: AGGGAGGCATCCTCGGCCTC (SEQ ID NO: 33).

In some embodiments, an oligonucleotide targets APOC3 and has a base sequence which is, comprises or comprises a portion of: AGGGTTACATGAAGCACGC (SEQ ID NO: 34).

In some embodiments, an oligonucleotide targets APOC3 and has a base sequence which is, comprises or comprises a portion of: AGGTCTCAGGCAGCCACGGC (SEQ ID NO: 35).

In some embodiments, an oligonucleotide targets APOC3 and has a base sequence which is, comprises or comprises a portion of: AGGTCTCAGGCAGCCACG(SEQ ID NO: 36).

In some embodiments, an oligonucleotide targets APOC3 and has a base sequence which is, comprises or comprises a portion of: AGGTCTCAGGCAGCCACGG (SEQ ID NO: 37).

In some embodiments, an oligonucleotide targets APOC3 and has a base sequence which is, comprises or comprises a portion of: AGTCCAGCTTTATTGGGAG (SEQ ID NO: 38).

In some embodiments, an oligonucleotide targets APOC3 and has a base sequence which is, comprises or comprises a portion of: ATAGCAGCTTCTTGTCGAGC (SEQ ID NO: 39).

In some embodiments, an oligonucleotide targets APOC3 and has a base sequence which is, comprises or comprises a portion of: ATAGCAGCTTCTTGTCCAGC (SEQ ID NO: 40).

In some embodiments, an oligonucleotide targets APOC3 and has a base sequence which is, comprises or comprises a portion of: ATAGCAGCTTCTTGTCCA (SEQ ID NO: 41).

In some embodiments, an oligonucleotide targets APOC3 and has a base sequence which is, comprises or comprises a portion of: ATAGCAGCTTCTTGTCCAG (SEQ ID NO: 42).

In some embodiments, an oligonucleotide targets APOC3 and has a base sequence which is, comprises or comprises a portion of: ATCCTTGGCGGTCTTGGTGG (SEQ ID NO: 43).

In some embodiments, an oligonucleotide targets APOC3 and has a base sequence which is, comprises or comprises a portion of: ATCGGTCACCCAGCCCCTGG (SEQ ID NO: 44).

In some embodiments, an oligonucleotide targets APOC3 and has a base sequence which is, comprises or comprises a portion of: ATCTTGTCCAGCTTTATTG (SEQ ID NO: 45).

In some embodiments, an oligonucleotide targets APOC3 and has a base sequence which is, comprises or comprises a portion of: ATGAAGCACGCCACCAAGA (SEQ ID NO: 46).

In some embodiments, an oligonucleotide targets APOC3 and has a base sequence which is, comprises or comprises a portion of: ATGCACTGAGCAGCGTGCAGGAGTCCCAGGTG (SEQ ID NO: 47).

In some embodiments, an oligonucleotide targets APOC3 and has a base sequence which is, comprises or comprises a portion of: ATGTCCAGCTTTATTGGGA (SEQ ID NO: 48).

In some embodiments, an oligonucleotide targets APOC3 and has a base sequence which is, comprises or comprises a portion of: CAATAAAGCTGGACAAGAAGCTA (SEQ ID NO: 49).

In some embodiments, an oligonucleotide targets APOC3 and has a base sequence which is, comprises or comprises a portion of: CACCAAGACCGCCAAGGATGCACTGAGCAG (SEQ ID NO: 50).

In some embodiments, an oligonucleotide targets APOC3 and has a base sequence which is, comprises or comprises a portion of: CACCCATTGGGACTGGGATC (SEQ ID NO: 51).

In some embodiments, an oligonucleotide targets APOC3 and has a base sequence which is, comprises or comprises a portion of: CACGCTGCTCAGTGCATCCT (SEQ ID NO: 52).

In some embodiments, an oligonucleotide targets APOC3 and has a base sequence which is, comprises or comprises a portion of: CACGGCTGAAGTTGGTCTGA (SEQ ID NO: 53).

In some embodiments, an oligonucleotide targets APOC3 and has a base sequence which is, comprises or comprises a portion of: CACTGAGAATACTGTCCCAA (SEQ ID NO: 54).

In some embodiments, an oligonucleotide targets APOC3 and has a base sequence which is, comprises or comprises a portion of: CACTGAGAATACTGTCCC (SEQ ID NO: 55).

In some embodiments, an oligonucleotide targets APOC3 and has a base sequence which is, comprises or comprises a portion of: CAGCCGTGGCTGCCTGAGACCTCA (SEQ ID NO: 56).

In some embodiments, an oligonucleotide targets APOC3 and has a base sequence which is, comprises or comprises a portion of: CAGCCGTGGCTGCCTGAGACCTCAA (SEQ ID NO: 57).

In some embodiments, an oligonucleotide targets APOC3 and has a base sequence which is, comprises or comprises a portion of: CAGCTTCTTGTCCAGCTTTA (SEQ ID NO: 58).

In some embodiments, an oligonucleotide targets APOC3 and has a base sequence which is, comprises or comprises a portion of: CAGCTTCTTGTCCAGCTT (SEQ ID NO: 59).

In some embodiments, an oligonucleotide targets APOC3 and has a base sequence which is, comprises or comprises a portion of: CAGCTTCTTGTCCAGCTTT (SEQ ID NO: 60).

In some embodiments, an oligonucleotide targets APOC3 and has a base sequence which is, comprises or comprises a portion of: CAGGGGCTGGGTGACCGATGGC (SEQ ID NO: 61).

In some embodiments, an oligonucleotide targets APOC3 and has a base sequence which is, comprises or comprises a portion of: CAGTGCATCCTTGGCGGTCT (SEQ ID NO: 62).

In some embodiments, an oligonucleotide targets APOC3 and has a base sequence which is, comprises or comprises a portion of: CATAGCAGCTTCTTGTCCAG (SEQ ID NO: 63).

In some embodiments, an oligonucleotide targets APOC3 and has a base sequence which is, comprises or comprises a portion of: CATCCTCGGCCTCTGAAGCT (SEQ ID NO: 64).

In some embodiments, an oligonucleotide targets APOC3 and has a base sequence which is, comprises or comprises a portion of: CATCCTTGGCGGTCTTGGTG (SEQ ID NO: 65).

In some embodiments, an oligonucleotide targets APOC3 and has a base sequence which is, comprises or comprises a portion of: CATCCTCGGCCTCTGAAGC (SEQ ID NO: 66).

In some embodiments, an oligonucleotide targets APOC3 and has a base sequence which is, comprises or comprises a portion of: CATCCTTGGCGGTCTTGG (SEQ ID NO: 67).

In some embodiments, an oligonucleotide targets APOC3 and has a base sequence which is, comprises or comprises a portion of: CATCGGTCACCCAGCCCCTG (SEQ ID NO: 68).

In some embodiments, an oligonucleotide targets APOC3 and has a base sequence which is, comprises or comprises a portion of: CCACCAAGACCGCCAAGGATGCAC (SEQ ID NO: 69).

In some embodiments, an oligonucleotide targets APOC3 and has a base sequence which is, comprises or comprises a portion of: CCACCTGGGACTCCTGCACG (SEQ ID NO: 70).

In some embodiments, an oligonucleotide targets APOC3 and has a base sequence which is, comprises or comprises a portion of: CCACCTGGGACTCCTGCAC (SEQ ID NO: 71).

In some embodiments, an oligonucleotide targets APOC3 and has a base sequence which is, comprises or comprises a portion of: CCACGGCTGAAGTTGGTCTG (SEQ ID NO: 72).

In some embodiments, an oligonucleotide targets APOC3 and has a base sequence which is, comprises or comprises a portion of: CCAGCTTTATTAGGGACAGC (SEQ ID NO: 73).

In some embodiments, an oligonucleotide targets APOC3 and has a base sequence which is, comprises or comprises a portion of: CCAGCTTTATTGGGAGGCC (SEQ ID NO: 74).

In some embodiments, an oligonucleotide targets APOC3 and has a base sequence which is, comprises or comprises a portion of: CCAGCTTTATTAGGGAC (SEQ ID NO: 75).

In some embodiments, an oligonucleotide targets APOC3 and has a base sequence which is, comprises or comprises a portion of: CCAGCTTTATTGGGAGGC (SEQ ID NO: 76).

In some embodiments, an oligonucleotide targets APOC3 and has a base sequence which is, comprises or comprises a portion of: CCAGGAGCGCCAGGAGGGCA (SEQ ID NO: 77).

In some embodiments, an oligonucleotide targets APOC3 and has a base sequence which is, comprises or comprises a portion of: CCATCGGTCACCCAGCCCCT (SEQ ID NO: 78).

In some embodiments, an oligonucleotide targets APOC3 and has a base sequence which is, comprises or comprises a portion of: CCATCGGTCACCCAGCCC (SEQ ID NO: 79).

In some embodiments, an oligonucleotide targets APOC3 and has a base sequence which is, comprises or comprises a portion of: CCCTGGAGATTGCAGGAC (SEQ ID NO: 80).

In some embodiments, an oligonucleotide targets APOC3 and has a base sequence which is, comprises or comprises a portion of: CCGTGGCTGCCTGAGACCT (SEQ ID NO: 81).

In some embodiments, an oligonucleotide targets APOC3 and has a base sequence which is, comprises or comprises a portion of: CCTCAGTCTGCTTCGCAC (SEQ ID NO: 82).

In some embodiments, an oligonucleotide targets APOC3 and has a base sequence which is, comprises or comprises a portion of: CCTCAGTCTGCTTCGCACCTTC (SEQ ID NO: 83).

In some embodiments, an oligonucleotide targets APOC3 and has a base sequence which is, comprises or comprises a portion of: CCTCAGTCTGCTTCGCACCT (SEQ ID NO: 84).

In some embodiments, an oligonucleotide targets APOC3 and has a base sequence which is, comprises or comprises a portion of: CCTCCAGGCATGCTGGCCT (SEQ ID NO: 85).

In some embodiments, an oligonucleotide targets APOC3 and has a base sequence which is, comprises or comprises a portion of: CCTCCCAATAAAGCTGGACA (SEQ ID NO: 86).

In some embodiments, an oligonucleotide targets APOC3 and has a base sequence which is, comprises or comprises a portion of: CCTCGGCCTCTGAAGCTCG (SEQ ID NO: 87).

In some embodiments, an oligonucleotide targets APOC3 and has a base sequence which is, comprises or comprises a portion of: CCTCGGCCTCTGAAGCTCGG (SEQ ID NO: 88).

In some embodiments, an oligonucleotide targets APOC3 and has a base sequence which is, comprises or comprises a portion of: CCTCTGAAAGTGGATTACCA (SEQ ID NO: 89).

In some embodiments, an oligonucleotide targets APOC3 and has a base sequence which is, comprises or comprises a portion of: CCTCTGAAGCTCGGGCAGAG (SEQ ID NO: 90).

In some embodiments, an oligonucleotide targets APOC3 and has a base sequence which is, comprises or comprises a portion of: CCTGGAGATTGCAGGACCCA (SEQ ID NO: 91).

In some embodiments, an oligonucleotide targets APOC3 and has a base sequence which is, comprises or comprises a portion of: CCTGGGACTCCTGCACGCTG (SEQ ID NO: 92).

In some embodiments, an oligonucleotide targets APOC3 and has a base sequence which is, comprises or comprises a portion of: CCTTGCAGGAACCCCAGCA (SEQ ID NO: 93).

In some embodiments, an oligonucleotide targets APOC3 and has a base sequence which is, comprises or comprises a portion of: CCTTGGCGGTCTTGGTGGCG (SEQ ID NO: 94).

In some embodiments, an oligonucleotide targets APOC3 and has a base sequence which is, comprises or comprises a portion of: CCTTGGCGGTCTTGGTGGC (SEQ ID NO: 95).

In some embodiments, an oligonucleotide targets APOC3 and has a base sequence which is, comprises or comprises a portion of: CGGCCTCTGAAGCTCGGGC (SEQ ID NO: 96).

In some embodiments, an oligonucleotide targets APOC3 and has a base sequence which is, comprises or comprises a portion of: CGGCCTCTGAAGCTCGGGCA (SEQ ID NO: 97).

In some embodiments, an oligonucleotide targets APOC3 and has a base sequence which is, comprises or comprises a portion of: CGGTCACCCAGCCCCTGGC (SEQ ID NO: 98).

In some embodiments, an oligonucleotide targets APOC3 and has a base sequence which is, comprises or comprises a portion of: CGTGCTTCATGTAACCCTGC (SEQ ID NO: 99).

In some embodiments, an oligonucleotide targets APOC3 and has a base sequence which is, comprises or comprises a portion of: CTCAGGCAGCCACGGCT (SEQ ID NO: 100).

In some embodiments, an oligonucleotide targets APOC3 and has a base sequence which is, comprises or comprises a portion of: CTCCTGCACGCTGCTCAGTG (SEQ ID NO: 101).

In some embodiments, an oligonucleotide targets APOC3 and has a base sequence which is, comprises or comprises a portion of: CTCCTGCTTGACCACCCATT (SEQ ID NO: 102).

In some embodiments, an oligonucleotide targets APOC3 and has a base sequence which is, comprises or comprises a portion of: CTCCTTGGGTCCTGCAACTCCAGGGCTGC (SEQ ID NO: 103).

In some embodiments, an oligonucleotide targets APOC3 and has a base sequence which is, comprises or comprises a portion of: CTCGGCCTCTGAAGCTCGGG (SEQ ID NO: 104).

In some embodiments, an oligonucleotide targets APOC3 and has a base sequence which is, comprises or comprises a portion of: CTCTGAAGCTCGGGCAGAGG (SEQ ID NO: 105).

In some embodiments, an oligonucleotide targets APOC3 and has a base sequence which is, comprises or comprises a portion of: CTGAAGCCATCGGTCACCCA (SEQ ID NO: 106).

In some embodiments, an oligonucleotide targets APOC3 and has a base sequence which is, comprises or comprises a portion of: CTGCATGGCACCTCTGTTCC (SEQ ID NO: 107).

In some embodiments, an oligonucleotide targets APOC3 and has a base sequence which is, comprises or comprises a portion of: CTGCCTCTAGGGATGAACTG (SEQ ID NO: 108).

In some embodiments, an oligonucleotide targets APOC3 and has a base sequence which is, comprises or comprises a portion of: CTGCTGGGCCACCTGGGACT (SEQ ID NO: 109).

In some embodiments, an oligonucleotide targets APOC3 and has a base sequence which is, comprises or comprises a portion of: CTGCTGGGCCACCTGGGAC (SEQ ID NO: 110).

In some embodiments, an oligonucleotide targets APOC3 and has a base sequence which is, comprises or comprises a portion of: CTGGAGATTGCAGGACCC (SEQ ID NO: 111).

In some embodiments, an oligonucleotide targets APOC3 and has a base sequence which is, comprises or comprises a portion of: CTGGAGCACCGTTAAGGACAAG (SEQ ID NO: 112).

In some embodiments, an oligonucleotide targets APOC3 and has a base sequence which is, comprises or comprises a portion of: CTGGCCTGCTGGGCCACCTG (SEQ ID NO: 113).

In some embodiments, an oligonucleotide targets APOC3 and has a base sequence which is, comprises or comprises a portion of: CTGGCCTCCCAATAAAGCTGGACA (SEQ ID NO: 114).

In some embodiments, an oligonucleotide targets APOC3 and has a base sequence which is, comprises or comprises a portion of: CTGGGACTCCTGCACGCT (SEQ ID NO: 115).

In some embodiments, an oligonucleotide targets APOC3 and has a base sequence which is, comprises or comprises a portion of: CTTCAGCCGTGGCTGCCTGAGACCTCAATA (SEQ ID NO: 116).

In some embodiments, an oligonucleotide targets APOC3 and has a base sequence which is, comprises or comprises a portion of: CTTCTTGTCCAGCTTTATTG (SEQ ID NO: 117).

In some embodiments, an oligonucleotide targets APOC3 and has a base sequence which is, comprises or comprises a portion of: CTTCTTGTCCAGCTTTAT (SEQ ID NO: 118).

In some embodiments, an oligonucleotide targets APOC3 and has a base sequence which is, comprises or comprises a portion of: CTTCTTGTCCAGCTTTATT (SEQ ID NO: 119).

In some embodiments, an oligonucleotide targets APOC3 and has a base sequence which is, comprises or comprises a portion of: CTTGGCGGTCTTGGTGGCGT (SEQ ID NO: 120).

In some embodiments, an oligonucleotide targets APOC3 and has a base sequence which is, comprises or comprises a portion of: CTTGGGTCCTGCAATCTCCAGGGCT (SEQ ID NO: 121).

In some embodiments, an oligonucleotide targets APOC3 and has a base sequence which is, comprises or comprises a portion of: CTTGGTGGCGTGCTTCATG (SEQ ID NO: 122).

In some embodiments, an oligonucleotide targets APOC3 and has a base sequence which is, comprises or comprises a portion of: CTTGGTGGCGTGCTTCAT (SEQ ID NO: 123).

In some embodiments, an oligonucleotide targets APOC3 and has a base sequence which is, comprises or comprises a portion of: CTTGTCCAGCTTTATTG (SEQ ID NO: 124).

In some embodiments, an oligonucleotide targets APOC3 and has a base sequence which is, comprises or comprises a portion of: CTTGTCCAGCTTTATTGG (SEQ ID NO: 125).

In some embodiments, an oligonucleotide targets APOC3 and has a base sequence which is, comprises or comprises a portion of: CTTGTCCAGCTTTATTGGG (SEQ ID NO: 126).

In some embodiments, an oligonucleotide targets APOC3 and has a base sequence which is, comprises or comprises a portion of: CTTGTCCAGCTTTATTGGGA (SEQ ID NO: 127).

In some embodiments, an oligonucleotide targets APOC3 and has a base sequence which is, comprises or comprises a portion of: CTTGTCCTTAACGGTGCTCC (SEQ ID NO: 128).

In some embodiments, an oligonucleotide targets APOC3 and has a base sequence which is, comprises or comprises a portion of: GAAGCCATCGGTCACCCAG (SEQ ID NO: 129).

In some embodiments, an oligonucleotide targets APOC3 and has a base sequence which is, comprises or comprises a portion of: GAAGCCATCGGTCACCCA (SEQ ID NO: 130).

In some embodiments, an oligonucleotide targets APOC3 and has a base sequence which is, comprises or comprises a portion of: GAAGGGAGGCATCCTCGGCC (SEQ ID NO: 131).

In some embodiments, an oligonucleotide targets APOC3 and has a base sequence which is, comprises or comprises a portion of: GAAGTTGGTCTGACCTCAG (SEQ ID NO: 132).

In some embodiments, an oligonucleotide targets APOC3 and has a base sequence which is, comprises or comprises a portion of: GAAGTTGGTCTGACCTCA (SEQ ID NO: 133).

In some embodiments, an oligonucleotide targets APOC3 and has a base sequence which is, comprises or comprises a portion of: GACCCTGAGGTCAGACCAA (SEQ ID NO: 134).

In some embodiments, an oligonucleotide targets APOC3 and has a base sequence which is, comprises or comprises a portion of: GAGAACTCCTCTGTAGGCA (SEQ ID NO: 135).

In some embodiments, an oligonucleotide targets APOC3 and has a base sequence which is, comprises or comprises a portion of: GAGAAGGGAGGCATCCTCG (SEQ ID NO: 136).

In some embodiments, an oligonucleotide targets APOC3 and has a base sequence which is, comprises or comprises a portion of: GAGGCATCCTCGGCCTCTGA (SEQ ID NO: 137).

In some embodiments, an oligonucleotide targets APOC3 and has a base sequence which is, comprises or comprises a portion of: GAGGTCTCAGGCAGCCACG (SEQ ID NO: 138).

In some embodiments, an oligonucleotide targets APOC3 and has a base sequence which is, comprises or comprises a portion of: GAGGTCAGACCAACTTCA (SEQ ID NO: 139).

In some embodiments, an oligonucleotide targets APOC3 and has a base sequence which is, comprises or comprises a portion of: GAGGTCTCAGGCAGCCACGG (SEQ ID NO: 140).

In some embodiments, an oligonucleotide targets APOC3 and has a base sequence which is, comprises or comprises a portion of: GAGGTCTCAGGCAGCCAC (SEQ ID NO: 141).

In some embodiments, an oligonucleotide targets APOC3 and has a base sequence which is, comprises or comprises a portion of: GCACTGAGCAGCGTGCAGGAGTCCCAGGT (SEQ ID NO: 142).

In some embodiments, an oligonucleotide targets APOC3 and has a base sequence which is, comprises or comprises a portion of: GCAGCTTCTTGTCCAGCT (SEQ ID NO: 143).

In some embodiments, an oligonucleotide targets APOC3 and has a base sequence which is, comprises or comprises a portion of: GCAGCTTCTTGTCCAGCTT (SEQ ID NO: 144).

In some embodiments, an oligonucleotide targets APOC3 and has a base sequence which is, comprises or comprises a portion of: GCAGGAGTCCCAGGTGGCCCAGCAGG (SEQ ID NO: 145).

In some embodiments, an oligonucleotide targets APOC3 and has a base sequence which is, comprises or comprises a portion of: GCATCCTTGGCGGTCTTGG (SEQ ID NO: 146).

In some embodiments, an oligonucleotide targets APOC3 and has a base sequence which is, comprises or comprises a portion of: GCATCCTTGGCGGTCTTGGT (SEQ ID NO: 147).

In some embodiments, an oligonucleotide targets APOC3 and has a base sequence which is, comprises or comprises a portion of: GCATCCTTGGCGGTCTTG (SEQ ID NO: 148).

In some embodiments, an oligonucleotide targets APOC3 and has a base sequence which is, comprises or comprises a portion of: GCCACGGCTGAAGTTGGTCT (SEQ ID NO: 149).

In some embodiments, an oligonucleotide targets APOC3 and has a base sequence which is, comprises or comprises a portion of: GCCATCGGTCACCCAGCCCC (SEQ ID NO: 150).

In some embodiments, an oligonucleotide targets APOC3 and has a base sequence which is, comprises or comprises a portion of: GCCATCGGTCACCCAGCCC (SEQ ID NO: 151).

In some embodiments, an oligonucleotide targets APOC3 and has a base sequence which is, comprises or comprises a portion of: GCCCCTGGCCTGCTGGGCCA (SEQ ID NO: 152).

In some embodiments, an oligonucleotide targets APOC3 and has a base sequence which is, comprises or comprises a portion of: GCCCTGGAGATTGCAGGACC (SEQ ID NO: 153).

In some embodiments, an oligonucleotide targets APOC3 and has a base sequence which is, comprises or comprises a portion of: GCCGTGGCTGCCTGAGACCTCAAT (SEQ ID NO: 154).

In some embodiments, an oligonucleotide targets APOC3 and has a base sequence which is, comprises or comprises a portion of: GCCTGACAAAGGCCCTGTGA (SEQ ID NO: 155).

In some embodiments, an oligonucleotide targets APOC3 and has a base sequence which is, comprises or comprises a portion of: GCCTCCCAATAAAGCTGGA (SEQ ID NO: 156).

In some embodiments, an oligonucleotide targets APOC3 and has a base sequence which is, comprises or comprises a portion of: GCCTCTAGGGATGAACTGA (SEQ ID NO: 157).

In some embodiments, an oligonucleotide targets APOC3 and has a base sequence which is, comprises or comprises a portion of: GCCTCTGAAGCTCGGGCAGA (SEQ ID NO: 158).

In some embodiments, an oligonucleotide targets APOC3 and has a base sequence which is, comprises or comprises a portion of: GCGGTCTTGGTGGCGTGC (SEQ ID NO: 159).

In some embodiments, an oligonucleotide targets APOC3 and has a base sequence which is, comprises or comprises a portion of: GCGTGCTTCATGTAACCCTG (SEQ ID NO: 160).

In some embodiments, an oligonucleotide targets APOC3 and has a base sequence which is, comprises or comprises a portion of: GCTCAGTGCATCCTTGGCG (SEQ ID NO: 161).

In some embodiments, an oligonucleotide targets APOC3 and has a base sequence which is, comprises or comprises a portion of: GCTCAGTGCATCCTTGGC (SEQ ID NO: 162).

In some embodiments, an oligonucleotide targets APOC3 and has a base sequence which is, comprises or comprises a portion of: GCTCCTGCTTGACCACCCAT (SEQ ID NO: 163).

In some embodiments, an oligonucleotide targets APOC3 and has a base sequence which is, comprises or comprises a portion of: GCTGAAGTTGGTCTGACCTC (SEQ ID NO: 164).

In some embodiments, an oligonucleotide targets APOC3 and has a base sequence which is, comprises or comprises a portion of: GCTGGCCTCCCAATAAAGCTGGACAAGAAG (SEQ ID NO: 165).

In some embodiments, an oligonucleotide targets APOC3 and has a base sequence which is, comprises or comprises a portion of: GCTGGGCCACCTGGGAC (SEQ ID NO: 166).

In some embodiments, an oligonucleotide targets APOC3 and has a base sequence which is, comprises or comprises a portion of: GCTTAAAAGGGACAGTA (SEQ ID NO: 167).

In some embodiments, an oligonucleotide targets APOC3 and has a base sequence which is, comprises or comprises a portion of: GCTTAAAAGGGACAGTATT (SEQ ID NO: 168).

In some embodiments, an oligonucleotide targets APOC3 and has a base sequence which is, comprises or comprises a portion of: GCTTCAGAGGCCGAGGATG (SEQ ID NO: 169).

In some embodiments, an oligonucleotide targets APOC3 and has a base sequence which is, comprises or comprises a portion of: GCTTCTTGTCCAGCTTTA (SEQ ID NO: 170).

In some embodiments, an oligonucleotide targets APOC3 and has a base sequence which is, comprises or comprises a portion of: GCTTCTTGTCCAGCTTT (SEQ ID NO: 171).

In some embodiments, an oligonucleotide targets APOC3 and has a base sequence which is, comprises or comprises a portion of: GCTTCTTGTCCAGCTTTAT (SEQ ID NO: 172).

In some embodiments, an oligonucleotide targets APOC3 and has a base sequence which is, comprises or comprises a portion of: GGACTCCTGCACGCTGCTCA (SEQ ID NO: 173).

In some embodiments, an oligonucleotide targets APOC3 and has a base sequence which is, comprises or comprises a portion of: GGAGCACCGTTAAGGACAAGT (SEQ ID NO: 174).

In some embodiments, an oligonucleotide targets APOC3 and has a base sequence which is, comprises or comprises a portion of: GGAGCAGCTGCCTCTAGGG (SEQ ID NO: 175).

In some embodiments, an oligonucleotide targets APOC3 and has a base sequence which is, comprises or comprises a portion of: GGAGCAGCTGCCTCTAGGGA (SEQ ID NO: 176).

In some embodiments, an oligonucleotide targets APOC3 and has a base sequence which is, comprises or comprises a portion of: GGAGGCATCCTCGGCCTCTG (SEQ ID NO: 177).

In some embodiments, an oligonucleotide targets APOC3 and has a base sequence which is, comprises or comprises a portion of: GGAGTCCCAGGTGGCCCAGCAGGC (SEQ ID NO: 178).

In some embodiments, an oligonucleotide targets APOC3 and has a base sequence which is, comprises or comprises a portion of: GGCAGAGGCCAGGAGCGCCA (SEQ ID NO: 179).

In some embodiments, an oligonucleotide targets APOC3 and has a base sequence which is, comprises or comprises a portion of: GGCATCCTCGGCCTCTGAAG (SEQ ID NO: 180).

In some embodiments, an oligonucleotide targets APOC3 and has a base sequence which is, comprises or comprises a portion of: GGCCAGGGGCTGGGTGACCGATGGCTTCAG (SEQ ID NO: 181).

In some embodiments, an oligonucleotide targets APOC3 and has a base sequence which is, comprises or comprises a portion of: GGCCAGGGGCTGGGTGACCGATGGCTTCAGT (SEQ ID NO: 182).

In some embodiments, an oligonucleotide targets APOC3 and has a base sequence which is, comprises or comprises a portion of: GGCCTCCCAATAAAGCTGGACA (SEQ ID NO: 183).

In some embodiments, an oligonucleotide targets APOC3 and has a base sequence which is, comprises or comprises a portion of: GGCCTCCCAATAAAGCTGGACAAG (SEQ ID NO: 184).

In some embodiments, an oligonucleotide targets APOC3 and has a base sequence which is, comprises or comprises a portion of: GGCCTCCCAATAAAGCTGGACAAGAA (SEQ ID NO: 185).

In some embodiments, an oligonucleotide targets APOC3 and has a base sequence which is, comprises or comprises a portion of: GGCGGTCTTGGTGGCGTGC (SEQ ID NO: 186).

In some embodiments, an oligonucleotide targets APOC3 and has a base sequence which is, comprises or comprises a portion of: GGCGGTCTTGGTGGCGTGCT (SEQ ID NO: 187).

In some embodiments, an oligonucleotide targets APOC3 and has a base sequence which is, comprises or comprises a portion of: GGCTGGGTGACCGATGGCTTCAGT (SEQ ID NO: 188).

In some embodiments, an oligonucleotide targets APOC3 and has a base sequence which is, comprises or comprises a portion of: GGGACAGTATTCTCAGTGA (SEQ ID NO: 189).

In some embodiments, an oligonucleotide targets APOC3 and has a base sequence which is, comprises or comprises a portion of: GGGACTCCTGCACGCTGCT (SEQ ID NO: 190).

In some embodiments, an oligonucleotide targets APOC3 and has a base sequence which is, comprises or comprises a portion of: GGGACTCCTGCACGCTGCTC (SEQ ID NO: 191).

In some embodiments, an oligonucleotide targets APOC3 and has a base sequence which is, comprises or comprises a portion of: GGGAGGCATCCTCGGCCTCT (SEQ ID NO: 192).

In some embodiments, an oligonucleotide targets APOC3 and has a base sequence which is, comprises or comprises a portion of: GGGCCTCCCAAGGCAAACCC (SEQ ID NO: 193).

In some embodiments, an oligonucleotide targets APOC3 and has a base sequence which is, comprises or comprises a portion of: GGGCTCCTGCTTGACCACC (SEQ ID NO: 194).

In some embodiments, an oligonucleotide targets APOC3 and has a base sequence which is, comprises or comprises a portion of: GGGCTGCATGGCACCTCTGT (SEQ ID NO: 195).

In some embodiments, an oligonucleotide targets APOC3 and has a base sequence which is, comprises or comprises a portion of: GGGCTGGGTGACCGATGGC (SEQ ID NO: 196).

In some embodiments, an oligonucleotide targets APOC3 and has a base sequence which is, comprises or comprises a portion of: GGTCTGACCTCAGGGTCCA (SEQ ID NO: 197).

In some embodiments, an oligonucleotide targets APOC3 and has a base sequence which is, comprises or comprises a portion of: GGTCTCAGGCAGCCACGG (SEQ ID NO: 198).

In some embodiments, an oligonucleotide targets APOC3 and has a base sequence which is, comprises or comprises a portion of: GGTCTTGGTGGCGTGCTTCA (SEQ ID NO: 199).

In some embodiments, an oligonucleotide targets APOC3 and has a base sequence which is, comprises or comprises a portion of: GTCCAGCTTTATTGGGAGG (SEQ ID NO: 200).

In some embodiments, an oligonucleotide targets APOC3 and has a base sequence which is, comprises or comprises a portion of: GTCCAGCTTATTGGGAGGC (SEQ ID NO: 201).

In some embodiments, an oligonucleotide targets APOC3 and has a base sequence which is, comprises or comprises a portion of: GTCCAGCTTTATTG (SEQ ID NO: 202).

In some embodiments, an oligonucleotide targets APOC3 and has a base sequence which is, comprises or comprises a portion of: GTCCAGCTTTATTGGG (SEQ ID NO: 203).

In some embodiments, an oligonucleotide targets APOC3 and has a base sequence which is, comprises or comprises a portion of: GTCCAGCTTTATTGGGA (SEQ ID NO: 204).

In some embodiments, an oligonucleotide targets APOC3 and has a base sequence which is, comprises or comprises a portion of: GTCCAGCTTTATTGGGAG (SEQ ID NO: 205).

In some embodiments, an oligonucleotide targets APOC3 and has a base sequence which is, comprises or comprises a portion of: GTCCAGCTTTATTGGGAGGC (SEQ ID NO: 206).

In some embodiments, an oligonucleotide targets APOC3 and has a base sequence which is, comprises or comprises a portion of: GTCCAGCTTTATTGGGAGGCCA (SEQ ID NO: 207).

In some embodiments, an oligonucleotide targets APOC3 and has a base sequence which is, comprises or comprises a portion of: GTCCAGCTTTATTGGGAGGCCT (SEQ ID NO: 208).

In some embodiments, an oligonucleotide targets APOC3 and has a base sequence which is, comprises or comprises a portion of: GTCCAGCTTTATTGGGAT (SEQ ID NO: 209).

In some embodiments, an oligonucleotide targets APOC3 and has a base sequence which is, comprises or comprises a portion of: GTCCCAGGTGGCCCAGCAG (SEQ ID NO: 210).

In some embodiments, an oligonucleotide targets APOC3 and has a base sequence which is, comprises or comprises a portion of: GTCTCAGGCAGCCACGGCTG (SEQ ID NO: 211).

In some embodiments, an oligonucleotide targets APOC3 and has a base sequence which is, comprises or comprises a portion of: GTCTTGGTGGCGTGCTTCAT (SEQ ID NO: 212).

In some embodiments, an oligonucleotide targets APOC3 and has a base sequence which is, comprises or comprises a portion of: GTGCAGGAGTCCCAGGTGG (SEQ ID NO: 213).

In some embodiments, an oligonucleotide targets APOC3 and has a base sequence which is, comprises or comprises a portion of: GTGCATCCTTGGCGGTCTTG (SEQ ID NO: 214).

In some embodiments, an oligonucleotide targets APOC3 and has a base sequence which is, comprises or comprises a portion of: GTGCATCCTTGGCGGTCTT (SEQ ID NO: 215).

In some embodiments, an oligonucleotide targets APOC3 and has a base sequence which is, comprises or comprises a portion of: GTTCCTGGAGCAGCTGCCT (SEQ ID NO: 216).

In some embodiments, an oligonucleotide targets APOC3 and has a base sequence which is, comprises or comprises a portion of: GTTGCTTAAAAGGGACAGTATTCTC (SEQ ID NO: 217).

In some embodiments, an oligonucleotide targets APOC3 and has a base sequence which is, comprises or comprises a portion of: GTTTATGCCCCTGGGCCTGA (SEQ ID NO: 218).

In some embodiments, an oligonucleotide targets APOC3 and has a base sequence which is, comprises or comprises a portion of: TCTTGTCCAGCTTTATTGG (SEQ ID NO: 219).

In some embodiments, an oligonucleotide targets APOC3 and has a base sequence which is, comprises or comprises a portion of: TCTTGTCCAGCTTTATT (SEQ ID NO: 220).

In some embodiments, an oligonucleotide targets APOC3 and has a base sequence which is, comprises or comprises a portion of: TCTTGTCCAGCTTTATTG (SEQ ID NO: 221).

In some embodiments, an oligonucleotide targets APOC3 and has a base sequence which is, comprises or comprises a portion of: TGGCGGTCTTGGTGGCGTG (SEQ ID NO: 222).

In some embodiments, an oligonucleotide targets APOC3 and has a base sequence which is, comprises or comprises a portion of: TGGGTCCTGCAATCTCCAGGGCT (SEQ ID NO: 223).

In some embodiments, an oligonucleotide targets APOC3 and has a base sequence which is, comprises or comprises a portion of: TGGTCTGACCTCAGGGTCC (SEQ ID NO: 224).

In some embodiments, an oligonucleotide targets APOC3 and has a base sequence which is, comprises or comprises a portion of: TGGTCTGACCTCAGGGTC (SEQ ID NO: 225).

In some embodiments, an oligonucleotide targets APOC3 and has a base sequence which is, comprises or comprises a portion of: TGGTGGCGTGCTTCATGTA (SEQ ID NO: 226).

In some embodiments, an oligonucleotide targets APOC3 and has a base sequence which is, comprises or comprises a portion of: TGTCCAGCTTTATTGGG (SEQ ID NO: 227).

In some embodiments, an oligonucleotide targets APOC3 and has a base sequence which is, comprises or comprises a portion of: TGTCCAGCTTTATTGGGA (SEQ ID NO: 228).

In some embodiments, an oligonucleotide targets APOC3 and has a base sequence which is, comprises or comprises a portion of: TGTCCAGCTTTATTGGGAG (SEQ ID NO: 229).

In some embodiments, an oligonucleotide targets APOC3 and has a base sequence which is, comprises or comprises a portion of: TGTCCTTAACGGTGCTC (SEQ ID NO: 230).

In some embodiments, an oligonucleotide targets PNPLA3 and has a base sequence which is, comprises or comprises a portion of: AAAGGCATGAAGCAGGAA (SEQ ID NO: 231).

In some embodiments, an oligonucleotide targets PNPLA3 and has a base sequence which is, comprises or comprises a portion of: AAAGGCATGAAGCAGGAACA (SEQ ID NO: 232).

In some embodiments, an oligonucleotide targets PNPLA3 and has a base sequence which is, comprises or comprises a portion of: AACGTCTCCATGGCGGGGGTAACAAGA (SEQ ID NO: 233).

In some embodiments, an oligonucleotide targets PNPLA3 and has a base sequence which is, comprises or comprises a portion of: AAGGCATGAAGCAGGAAC (SEQ ID NO: 234).

In some embodiments, an oligonucleotide targets PNPLA3 and has a base sequence which is, comprises or comprises a portion of: AAGGCATGAAGCAGGAACAT (SEQ ID NO: 235).

In some embodiments, an oligonucleotide targets PNPLA3 and has a base sequence which is, comprises or comprises a portion of: AAGGCCACTGTAGAAAGGCATGAA (SEQ ID NO: 236).

In some embodiments, an oligonucleotide targets PNPLA3 and has a base sequence which is, comprises or comprises a portion of: AAGGCCACTGTAGAAAGGCATGAAG (SEQ ID NO: 237).

In some embodiments, an oligonucleotide targets PNPLA3 and has a base sequence which is, comprises or comprises a portion of: AAGGCCACTGTAGAAAG (SEQ ID NO: 238).

In some embodiments, an oligonucleotide targets PNPLA3 and has a base sequence which is, comprises or comprises a portion of: AAGGCCACTGTAGAAAGG (SEQ ID NO: 239).

In some embodiments, an oligonucleotide targets PNPLA3 and has a base sequence which is, comprises or comprises a portion of: AAGGCCACTGTAGAAAGGC (SEQ ID NO: 240).

In some embodiments, an oligonucleotide targets PNPLA3 and has a base sequence which is, comprises or comprises a portion of: AAGGCCACTGTAGAAAGGCA (SEQ ID NO: 241).

In some embodiments, an oligonucleotide targets PNPLA3 and has a base sequence which is, comprises or comprises a portion of: AAGGGACCCTCTGCACTGGG (SEQ ID NO: 242).

In some embodiments, an oligonucleotide targets PNPLA3 and has a base sequence which is, comprises or comprises a portion of: AAGGGCATGAAGCAGGAACA (SEQ ID NO: 243).

In some embodiments, an oligonucleotide targets PNPLA3 and has a base sequence which is, comprises or comprises a portion of: ACCCCCGCCATGGAGACGT (SEQ ID NO: 244).

In some embodiments, an oligonucleotide targets PNPLA3 and has a base sequence which is, comprises or comprises a portion of: ACCCCCGCCATGGAGACG (SEQ ID NO: 245).

In some embodiments, an oligonucleotide targets PNPLA3 and has a base sequence which is, comprises or comprises a portion of: ACCCCCGCCATGGAGACGTT (SEQ ID NO: 246).

In some embodiments, an oligonucleotide targets PNPLA3 and has a base sequence which is, comprises or comprises a portion of: ACCCCGCGGTCCATCCTCAGGTCCAGC (SEQ ID NO: 247).

In some embodiments, an oligonucleotide targets PNPLA3 and has a base sequence which is, comprises or comprises a portion of: ACCCTGCCTCAGTGTCTCG (SEQ ID NO: 248).

In some embodiments, an oligonucleotide targets PNPLA3 and has a base sequence which is, comprises or comprises a portion of: ACCTGAGGATGGACCGCG (SEQ ID NO: 249).

In some embodiments, an oligonucleotide targets PNPLA3 and has a base sequence which is, comprises or comprises a portion of: ACCTGAGGATGGACCGCGGG (SEQ ID NO: 250).

In some embodiments, an oligonucleotide targets PNPLA3 and has a base sequence which is, comprises or comprises a portion of: ACGTTGTCACTCACTCCTCC (SEQ ID NO: 251).

In some embodiments, an oligonucleotide targets PNPLA3 and has a base sequence which is, comprises or comprises a portion of: ACTGTAGAAAGGCATGAAGCAGGAA (SEQ ID NO: 252).

In some embodiments, an oligonucleotide targets PNPLA3 and has a base sequence which is, comprises or comprises a portion of: ACTGTAGAAAGGCATGAA (SEQ ID NO: 253).

In some embodiments, an oligonucleotide targets PNPLA3 and has a base sequence which is, comprises or comprises a portion of: ACTGTAGAAAGGCATGAAGC (SEQ ID NO: 254).

In some embodiments, an oligonucleotide targets PNPLA3 and has a base sequence which is, comprises or comprises a portion of: AGAAAGGCATGAAGCAGGA (SEQ ID NO: 255).

In some embodiments, an oligonucleotide targets PNPLA3 and has a base sequence which is, comprises or comprises a portion of: AGAAAGGCATGAAGCAGGAA (SEQ ID NO: 256).

In some embodiments, an oligonucleotide targets PNPLA3 and has a base sequence which is, comprises or comprises a portion of: AGAAAGGCATGAAGCAGGAACATA (SEQ ID NO: 257).

In some embodiments, an oligonucleotide targets PNPLA3 and has a base sequence which is, comprises or comprises a portion of: AGAAAGGCATGAAGCAG (SEQ ID NO: 258).

In some embodiments, an oligonucleotide targets PNPLA3 and has a base sequence which is, comprises or comprises a portion of: AGAAAGGCATGAAGCAGG (SEQ ID NO: 259).

In some embodiments, an oligonucleotide targets PNPLA3 and has a base sequence which is, comprises or comprises a portion of: AAATGCCTTTCTACAGTGGCA (SEQ ID NO: 260).

In some embodiments, an oligonucleotide targets PNPLA3 and has a base sequence which is, comprises or comprises a portion of: AATCATGCCTTTCTACAGTGGCA (SEQ ID NO: 261).

In some embodiments, an oligonucleotide targets PNPLA3 and has a base sequence which is, comprises or comprises a portion of: ACATGGGCCAGCCTACCCCC (SEQ ID NO: 262).

In some embodiments, an oligonucleotide targets PNPLA3 and has a base sequence which is, comprises or comprises a portion of: ACCTGTGAGGTCACCCACTG (SEQ ID NO: 263).

In some embodiments, an oligonucleotide targets PNPLA3 and has a base sequence which is, comprises or comprises a portion of: ATCTTGTTACCCCCGCCATG (SEQ ID NO: 264).

In some embodiments, an oligonucleotide targets PNPLA3 and has a base sequence which is, comprises or comprises a portion of: ATGCCTTTCTACAGTGGCA (SEQ ID NO: 265).

In some embodiments, an oligonucleotide targets PNPLA3 and has a base sequence which is, comprises or comprises a portion of: GCCACTGTAGAAAGGCATGATT (SEQ ID NO: 266).

In some embodiments, an oligonucleotide targets PNPLA3 and has a base sequence which is, comprises or comprises a portion of: GCCACTGTAGAAAGGCATTT (SEQ ID NO: 267).

In some embodiments, an oligonucleotide targets PNPLA3 and has a base sequence which is, comprises or comprises a portion of: TGCCACTGTAGAAAGGCATGATT (SEQ ID NO: 268).

In some embodiments, an oligonucleotide targets PNPLA3 and has a base sequence which is, comprises or comprises a portion of: TCATGCCTTTCTACAGTGGCA (SEQ ID NO: 269).

In some embodiments, an oligonucleotide targets PNPLA3 and has a base sequence which is, comprises or comprises a portion of: TGCCACTGTAGAAAGGCATTT (SEQ ID NO: 270).

In some embodiments, an oligonucleotide targets PNPLA3 and has a base sequence which is, comprises or comprises a portion of: AGAGGCTGGGATCCTCCACG (SEQ ID NO: 271).

In some embodiments, an oligonucleotide targets PNPLA3 and has a base sequence which is, comprises or comprises a portion of: AGCCACTGTAGAAAGGCATGA (SEQ ID NO: 272).

In some embodiments, an oligonucleotide targets PNPLA3 and has a base sequence which is, comprises or comprises a portion of: AGCGAGCCTGGGCGAGAGGG (SEQ ID NO: 273).

In some embodiments, an oligonucleotide targets PNPLA3 and has a base sequence which is, comprises or comprises a portion of: AGCGCTCTCTACCCTGCCTC (SEQ ID NO: 274).

In some embodiments, an oligonucleotide targets PNPLA3 and has a base sequence which is, comprises or comprises a portion of: AGCTGGTGGACATTGGCCGG (SEQ ID NO: 275).

In some embodiments, an oligonucleotide targets PNPLA3 and has a base sequence which is, comprises or comprises a portion of: AGGCATGAAGCAGGAACA (SEQ ID NO: 276).

In some embodiments, an oligonucleotide targets PNPLA3 and has a base sequence which is, comprises or comprises a portion of: AGGCATGAAGCAGGAACATA (SEQ ID NO: 277).

In some embodiments, an oligonucleotide targets PNPLA3 and has a base sequence which is, comprises or comprises a portion of: AGGCCACTGTAGAAAGGCATGAAGC (SEQ ID NO: 278).

In some embodiments, an oligonucleotide targets PNPLA3 and has a base sequence which is, comprises or comprises a portion of: AGGCCACTGTAGAAAGGC (SEQ ID NO: 279).

In some embodiments, an oligonucleotide targets PNPLA3 and has a base sequence which is, comprises or comprises a portion of: AGGCCACTGTAGAAAGGCAT (SEQ ID NO: 280).

In some embodiments, an oligonucleotide targets PNPLA3 and has a base sequence which is, comprises or comprises a portion of: AGGCTGGGATCCTCCACGTC (SEQ ID NO: 281).

In some embodiments, an oligonucleotide targets PNPLA3 and has a base sequence which is, comprises or comprises a portion of: AGGGACCCTCTGCACTGGGC (SEQ ID NO: 282).

In some embodiments, an oligonucleotide targets PNPLA3 and has a base sequence which is, comprises or comprises a portion of: ATAAGGCCACTGTAGAAAGG (SEQ ID NO: 283).

In some embodiments, an oligonucleotide targets PNPLA3 and has a base sequence which is, comprises or comprises a portion of: ATAAGGCCACTGTAGAAA (SEQ ID NO: 284).

In some embodiments, an oligonucleotide targets PNPLA3 and has a base sequence which is, comprises or comprises a portion of: ATCTTGTTACCCCCGCCATG (SEQ ID NO: 285).

In some embodiments, an oligonucleotide targets PNPLA3 and has a base sequence which is, comprises or comprises a portion of: ATGACACCAGGAAGCCCAGTGCAGAGG (SEQ ID NO: 286).

In some embodiments, an oligonucleotide targets PNPLA3 and has a base sequence which is, comprises or comprises a portion of: ATGTTCCGACTCCTGGCC (SEQ ID NO: 287).

In some embodiments, an oligonucleotide targets PNPLA3 and has a base sequence which is, comprises or comprises a portion of: ATTTGGGACCTGGAGGCGGG (SEQ ID NO: 288).

In some embodiments, an oligonucleotide targets PNPLA3 and has a base sequence which is, comprises or comprises a portion of: CACATGGGCCAGCCTACCCC (SEQ ID NO: 289).

In some embodiments, an oligonucleotide targets PNPLA3 and has a base sequence which is, comprises or comprises a portion of: CACCCCTTCCCACAGCATGG (SEQ ID NO: 290).

In some embodiments, an oligonucleotide targets PNPLA3 and has a base sequence which is, comprises or comprises a portion of: CACTGTAGAAAGGCATGAAGCAGGA (SEQ ID NO: 291).

In some embodiments, an oligonucleotide targets PNPLA3 and has a base sequence which is, comprises or comprises a portion of: CACTGTAGAAAGGCATGA (SEQ ID NO: 292).

In some embodiments, an oligonucleotide targets PNPLA3 and has a base sequence which is, comprises or comprises a portion of: CACTGTAGAAAGGCATGAAG (SEQ ID NO: 293).

In some embodiments, an oligonucleotide targets PNPLA3 and has a base sequence which is, comprises or comprises a portion of: CATGAAGCAGGAACATAC (SEQ ID NO: 294).

In some embodiments, an oligonucleotide targets PNPLA3 and has a base sequence which is, comprises or comprises a portion of: CATGAAGCAGGAACATACCA (SEQ ID NO: 295).

In some embodiments, an oligonucleotide targets PNPLA3 and has a base sequence which is, comprises or comprises a portion of: CCACTGTAGAAAGGCATGAAGCAGG (SEQ ID NO: 296).

In some embodiments, an oligonucleotide targets PNPLA3 and has a base sequence which is, comprises or comprises a portion of: CCACTGTAGAAAGGCATG (SEQ ID NO: 297).

In some embodiments, an oligonucleotide targets PNPLA3 and has a base sequence which is, comprises or comprises a portion of: CCACTGTAGAAAGGCATGAA (SEQ ID NO: 298).

In some embodiments, an oligonucleotide targets PNPLA3 and has a base sequence which is, comprises or comprises a portion of: CCAGCACCTTGAGATCCGGG (SEQ ID NO: 299).

In some embodiments, an oligonucleotide targets PNPLA3 and has a base sequence which is, comprises or comprises a portion of: CCCAGCACCTTGAGATCCGG (SEQ ID NO: 300).

In some embodiments, an oligonucleotide targets PNPLA3 and has a base sequence which is, comprises or comprises a portion of: CCCCCAGGCAGGAGCCAAGCACAGCAG (SEQ ID NO: 301).

In some embodiments, an oligonucleotide targets PNPLA3 and has a base sequence which is, comprises or comprises a portion of: CCCCCGCCATGGAGACGT (SEQ ID NO: 302).

In some embodiments, an oligonucleotide targets PNPLA3 and has a base sequence which is, comprises or comprises a portion of: CCCCCGCCATGGAGACGTTT (SEQ ID NO: 303).

In some embodiments, an oligonucleotide targets PNPLA3 and has a base sequence which is, comprises or comprises a portion of: CCCCGCCATGGAGACGTT (SEQ ID NO: 304).

In some embodiments, an oligonucleotide targets PNPLA3 and has a base sequence which is, comprises or comprises a portion of: CCCTGCCTCAGTGTCTCGGC (SEQ ID NO: 305).

In some embodiments, an oligonucleotide targets PNPLA3 and has a base sequence which is, comprises or comprises a portion of: CCGACTCCTGGCCTTCCGCA (SEQ ID NO: 306).

In some embodiments, an oligonucleotide targets PNPLA3 and has a base sequence which is, comprises or comprises a portion of: CCGACTCCTGGCCTTCCGC (SEQ ID NO: 307).

In some embodiments, an oligonucleotide targets PNPLA3 and has a base sequence which is, comprises or comprises a portion of: CCTCAGTGTCTCGGCCAGGG (SEQ ID NO: 308).

In some embodiments, an oligonucleotide targets PNPLA3 and has a base sequence which is, comprises or comprises a portion of: CCTGAGGATGGACCGCGGGG (SEQ ID NO: 309).

In some embodiments, an oligonucleotide targets PNPLA3 and has a base sequence which is, comprises or comprises a portion of: CCTGCCTCAGTGTCTCGGCC (SEQ ID NO: 310).

In some embodiments, an oligonucleotide targets PNPLA3 and has a base sequence which is, comprises or comprises a portion of: CCTGCTGTGCTTGGCTCCT (SEQ ID NO: 311).

In some embodiments, an oligonucleotide targets PNPLA3 and has a base sequence which is, comprises or comprises a portion of: CCTGCTGTGCTTGGCTCCTG (SEQ ID NO: 312).

In some embodiments, an oligonucleotide targets PNPLA3 and has a base sequence which is, comprises or comprises a portion of: CCTGGGCGAGAGGGTGTCCA (SEQ ID NO: 313).

In some embodiments, an oligonucleotide targets PNPLA3 and has a base sequence which is, comprises or comprises a portion of: CCTGTGAGGTCACCCACTGC (SEQ ID NO: 314).

In some embodiments, an oligonucleotide targets PNPLA3 and has a base sequence which is, comprises or comprises a portion of: CCTGTTGGCTGCTCACTGGC (SEQ ID NO: 315).

In some embodiments, an oligonucleotide targets PNPLA3 and has a base sequence which is, comprises or comprises a portion of: CGAACTGCACCCCTTCCCAC (SEQ ID NO: 316).

In some embodiments, an oligonucleotide targets PNPLA3 and has a base sequence which is, comprises or comprises a portion of: CGACCTCAGGATCCATCCCT (SEQ ID NO: 317).

In some embodiments, an oligonucleotide targets PNPLA3 and has a base sequence which is, comprises or comprises a portion of: CGACTCCTGGCCTTCCGCAC (SEQ ID NO: 318).

In some embodiments, an oligonucleotide targets PNPLA3 and has a base sequence which is, comprises or comprises a portion of: CGCCACTGTAGAAAGGCATGA (SEQ ID NO: 319).

In some embodiments, an oligonucleotide targets PNPLA3 and has a base sequence which is, comprises or comprises a portion of: CGGCCAGGGCATTCCCAGCG (SEQ ID NO: 320).

In some embodiments, an oligonucleotide targets PNPLA3 and has a base sequence which is, comprises or comprises a portion of: CTCAGAGGCTGGGATCCTCC (SEQ ID NO: 321).

In some embodiments, an oligonucleotide targets PNPLA3 and has a base sequence which is, comprises or comprises a portion of: CTCAGGCAGCGGGTCGCCCC (SEQ ID NO: 322).

In some embodiments, an oligonucleotide targets PNPLA3 and has a base sequence which is, comprises or comprises a portion of: CTCCTGCTGTGCTTGGCTCC (SEQ ID NO: 323).

In some embodiments, an oligonucleotide targets PNPLA3 and has a base sequence which is, comprises or comprises a portion of: CTCGGCCAGGGCATTCCCA (SEQ ID NO: 324).

In some embodiments, an oligonucleotide targets PNPLA3 and has a base sequence which is, comprises or comprises a portion of: CTCTGCTGGACAGCCCTTGG (SEQ ID NO: 325).

In some embodiments, an oligonucleotide targets PNPLA3 and has a base sequence which is, comprises or comprises a portion of: CTGAGGATGGACCGCGGG (SEQ ID NO: 326).

In some embodiments, an oligonucleotide targets PNPLA3 and has a base sequence which is, comprises or comprises a portion of: CTGCACTGGGCTTCCTGGT (SEQ ID NO: 327).

In some embodiments, an oligonucleotide targets PNPLA3 and has a base sequence which is, comprises or comprises a portion of: CTGCACTGGGCTTCCTGGTG (SEQ ID NO: 328).

In some embodiments, an oligonucleotide targets PNPLA3 and has a base sequence which is, comprises or comprises a portion of: CTGCCTCAGTGTCTCGGCCA (SEQ ID NO: 329).

In some embodiments, an oligonucleotide targets PNPLA3 and has a base sequence which is, comprises or comprises a portion of: CTGCTAGACTCGCCTCCTC (SEQ ID NO: 330).

In some embodiments, an oligonucleotide targets PNPLA3 and has a base sequence which is, comprises or comprises a portion of: CTGCTCCAGCGGGATACCG (SEQ ID NO: 331).

In some embodiments, an oligonucleotide targets PNPLA3 and has a base sequence which is, comprises or comprises a portion of: CTGCTCCAGCGGGATACCGG (SEQ ID NO: 332).

In some embodiments, an oligonucleotide targets PNPLA3 and has a base sequence which is, comprises or comprises a portion of: CTGCTGGACAGCCCTTGGG (SEQ ID NO: 333).

In some embodiments, an oligonucleotide targets PNPLA3 and has a base sequence which is, comprises or comprises a portion of: CTGCTGGACAGCCCTTGGGG (SEQ ID NO: 334).

In some embodiments, an oligonucleotide targets PNPLA3 and has a base sequence which is, comprises or comprises a portion of: CTGCTGTGCTTGGCTCCTGC (SEQ ID NO: 335).

In some embodiments, an oligonucleotide targets PNPLA3 and has a base sequence which is, comprises or comprises a portion of: CTGGACCTGAGGATGGACCG (SEQ ID NO: 336).

In some embodiments, an oligonucleotide targets PNPLA3 and has a base sequence which is, comprises or comprises a portion of: CTGGGCATGGCGACCTCAGG (SEQ ID NO: 337).

In some embodiments, an oligonucleotide targets PNPLA3 and has a base sequence which is, comprises or comprises a portion of: CTGGTGGACATTGGCCGGGA (SEQ ID NO: 338).

In some embodiments, an oligonucleotide targets PNPLA3 and has a base sequence which is, comprises or comprises a portion of: CTGTAGAAAGGCATGAAGCA (SEQ ID NO: 339).

In some embodiments, an oligonucleotide targets PNPLA3 and has a base sequence which is, comprises or comprises a portion of: CTGTAGAAAGGCATGAAGCAGGAAC (SEQ ID NO: 340).

In some embodiments, an oligonucleotide targets PNPLA3 and has a base sequence which is, comprises or comprises a portion of: CTGTAGAAAGGCATGAAG (SEQ ID NO: 341).

In some embodiments, an oligonucleotide targets PNPLA3 and has a base sequence which is, comprises or comprises a portion of: CTGTAGAAAGGCATGAAGCA (SEQ ID NO: 342).

In some embodiments, an oligonucleotide targets PNPLA3 and has a base sequence which is, comprises or comprises a portion of: CTGTTGGCTGCTCACTGGCA (SEQ ID NO: 343).

In some embodiments, an oligonucleotide targets PNPLA3 and has a base sequence which is, comprises or comprises a portion of: CTTGTTACCCCCGCCATGG (SEQ ID NO: 344).

In some embodiments, an oligonucleotide targets PNPLA3 and has a base sequence which is, comprises or comprises a portion of: CTTGTTACCCCCGCCATGGA (SEQ ID NO: 345).

In some embodiments, an oligonucleotide targets PNPLA3 and has a base sequence which is, comprises or comprises a portion of: GAAAGGCATGAAGCAGGA (SEQ ID NO: 346).

In some embodiments, an oligonucleotide targets PNPLA3 and has a base sequence which is, comprises or comprises a portion of: GAAAGGCATGAAGCAGGAAC (SEQ ID NO: 347).

In some embodiments, an oligonucleotide targets PNPLA3 and has a base sequence which is, comprises or comprises a portion of: GAAGCCCAGTGCAGAGGGTCCCTTACT (SEQ ID NO: 348).

In some embodiments, an oligonucleotide targets PNPLA3 and has a base sequence which is, comprises or comprises a portion of: GAAGGGCATGAAGCAGGAAC (SEQ ID NO: 349).

In some embodiments, an oligonucleotide targets PNPLA3 and has a base sequence which is, comprises or comprises a portion of: GACCCTCTGCACTGGGCTTC (SEQ ID NO: 350).

In some embodiments, an oligonucleotide targets PNPLA3 and has a base sequence which is, comprises or comprises a portion of: GACCCTCTGCACTGGGCT (SEQ ID NO: 351).

In some embodiments, an oligonucleotide targets PNPLA3 and has a base sequence which is, comprises or comprises a portion of: GACCTGAGGATGGACCGC (SEQ ID NO: 352).

In some embodiments, an oligonucleotide targets PNPLA3 and has a base sequence which is, comprises or comprises a portion of: GACCTGAGGATGGACCGCGG (SEQ ID NO: 353).

In some embodiments, an oligonucleotide targets PNPLA3 and has a base sequence which is, comprises or comprises a portion of: GACGAACTGCACCCCTTCCC (SEQ ID NO: 354).

In some embodiments, an oligonucleotide targets PNPLA3 and has a base sequence which is, comprises or comprises a portion of: GACTCCTGGCCTTCCGCACA (SEQ ID NO: 355).

In some embodiments, an oligonucleotide targets PNPLA3 and has a base sequence which is, comprises or comprises a portion of: GATAAGGCCACTGTAGAAAG (SEQ ID NO: 356).

In some embodiments, an oligonucleotide targets PNPLA3 and has a base sequence which is, comprises or comprises a portion of: GCACTGGGCTTCCTGGTGT (SEQ ID NO: 357).

In some embodiments, an oligonucleotide targets PNPLA3 and has a base sequence which is, comprises or comprises a portion of: GCAGAGACCCTGTCGGAGG (SEQ ID NO: 358).

In some embodiments, an oligonucleotide targets PNPLA3 and has a base sequence which is, comprises or comprises a portion of: GCATGAAGCAGGAACATACC (SEQ ID NO: 359).

In some embodiments, an oligonucleotide targets PNPLA3 and has a base sequence which is, comprises or comprises a portion of: GCATGAAGCAGGAACATA (SEQ ID NO: 360).

In some embodiments, an oligonucleotide targets PNPLA3 and has a base sequence which is, comprises or comprises a portion of: GCCACTGTAGAAAGGCATGA (SEQ ID NO: 361).

In some embodiments, an oligonucleotide targets PNPLA3 and has a base sequence which is, comprises or comprises a portion of: GCCACTGTAGAAAGGCATGAAGCAG (SEQ ID NO: 362).

In some embodiments, an oligonucleotide targets PNPLA3 and has a base sequence which is, comprises or comprises a portion of: GCCACTGTAGAAAGGCATGA (SEQ ID NO: 363).

In some embodiments, an oligonucleotide targets PNPLA3 and has a base sequence which is, comprises or comprises a portion of: GCCACTGTAGAAACGCAT (SEQ ID NO: 364).

In some embodiments, an oligonucleotide targets PNPLA3 and has a base sequence which is, comprises or comprises a portion of: GCCACTGTAGAAAGCCAT (SEQ ID NO: 365).

In some embodiments, an oligonucleotide targets PNPLA3 and has a base sequence which is, comprises or comprises a portion of: GCCACTGTAGAAAGCCATGA (SEQ ID NO: 366).

In some embodiments, an oligonucleotide targets PNPLA3 and has a base sequence which is, comprises or comprises a portion of: GCCACTGTAGAAAGGAATGA (SEQ ID NO: 367).

In some embodiments, an oligonucleotide targets PNPLA3 and has a base sequence which is, comprises or comprises a portion of: GCCACTGTAGAAAGGC (SEQ ID NO: 368).

In some embodiments, an oligonucleotide targets PNPLA3 and has a base sequence which is, comprises or comprises a portion of: GCCACTGTAGAAAGGCA (SEQ ID NO: 369).

In some embodiments, an oligonucleotide targets PNPLA3 and has a base sequence which is, comprises or comprises a portion of: GCCACTGTAGAAAGGCAA (SEQ ID NO: 370).

In some embodiments, an oligonucleotide targets PNPLA3 and has a base sequence which is, comprises or comprises a portion of: GCCACTGTAGAAAGGCAGG (SEQ ID NO: 371).

In some embodiments, an oligonucleotide targets PNPLA3 and has a base sequence which is, comprises or comprises a portion of: GCCACTGTAGAAAGGCAT (SEQ ID NO: 372).

In some embodiments, an oligonucleotide targets PNPLA3 and has a base sequence which is, comprises or comprises a portion of: GCCACTGTAGAAAGGCATGA (SEQ ID NO: 373).

In some embodiments, an oligonucleotide targets PNPLA3 and has a base sequence which is, comprises or comprises a portion of: GCCACTGTAGAAAGGCATCA (SEQ ID NO: 374).

In some embodiments, an oligonucleotide targets PNPLA3 and has a base sequence which is, comprises or comprises a portion of: GCCACTGTAGAAAGGCATG (SEQ ID NO: 375).

In some embodiments, an oligonucleotide targets PNPLA3 and has a base sequence which is, comprises or comprises a portion of: GCCACTGTAGAAAGGCATGA (SEQ ID NO: 376).

In some embodiments, an oligonucleotide targets PNPLA3 and has a base sequence which is, comprises or comprises a portion of: GCCACTGTAGAAAGGCATGAA (SEQ ID NO: 377).

In some embodiments, an oligonucleotide targets PNPLA3 and has a base sequence which is, comprises or comprises a portion of: GCCACTGTAGAAAGGCATGAAG (SEQ ID NO: 378).

In some embodiments, an oligonucleotide targets PNPLA3 and has a base sequence which is, comprises or comprises a portion of: GCCACTGTAGAAAGGCATGT (SEQ ID NO: 379).

In some embodiments, an oligonucleotide targets PNPLA3 and has a base sequence which is, comprises or comprises a portion of: GCCACTGTAGAAAGGCGG (SEQ ID NO: 380).

In some embodiments, an oligonucleotide targets PNPLA3 and has a base sequence which is, comprises or comprises a portion of: GCCACTGTAGAAAGGCGTGA (SEQ ID NO: 381).

In some embodiments, an oligonucleotide targets PNPLA3 and has a base sequence which is, comprises or comprises a portion of: GCCACTGTAGAAAGGCTT (SEQ ID NO: 382).

In some embodiments, an oligonucleotide targets PNPLA3 and has a base sequence which is, comprises or comprises a portion of: GCCACTGTAGAAAGGCTTGA (SEQ ID NO: 383).

In some embodiments, an oligonucleotide targets PNPLA3 and has a base sequence which is, comprises or comprises a portion of: GCCACTGTAGAAAGGGAT (SEQ ID NO: 384).

In some embodiments, an oligonucleotide targets PNPLA3 and has a base sequence which is, comprises or comprises a portion of: GCCACTGTAGAAAGGTATGA (SEQ ID NO: 385).

In some embodiments, an oligonucleotide targets PNPLA3 and has a base sequence which is, comprises or comprises a portion of: GCCACTGTAGAATGGCAT (SEQ ID NO: 386).

In some embodiments, an oligonucleotide targets PNPLA3 and has a base sequence which is, comprises or comprises a portion of: GCCACTGTAGAGAGGCATGA (SEQ ID NO: 387).

In some embodiments, an oligonucleotide targets PNPLA3 and has a base sequence which is, comprises or comprises a portion of: GCCTCAGTGTCTCGGCCAGG (SEQ ID NO: 388).

In some embodiments, an oligonucleotide targets PNPLA3 and has a base sequence which is, comprises or comprises a portion of: GCCTGGGCGAGAGGGTGTCC (SEQ ID NO: 389).

In some embodiments, an oligonucleotide targets PNPLA3 and has a base sequence which is, comprises or comprises a portion of: GCCTGTTGGCTGCTCACTGG (SEQ ID NO: 390).

In some embodiments, an oligonucleotide targets PNPLA3 and has a base sequence which is, comprises or comprises a portion of: GCGCTCTCTACCCTGCCTCA (SEQ ID NO: 391).

In some embodiments, an oligonucleotide targets PNPLA3 and has a base sequence which is, comprises or comprises a portion of: GCTCCAGCGGGATACCGGA (SEQ ID NO: 392).

In some embodiments, an oligonucleotide targets PNPLA3 and has a base sequence which is, comprises or comprises a portion of: GCTCGGCCTCCAGTTCCA (SEQ ID NO: 393).

In some embodiments, an oligonucleotide targets PNPLA3 and has a base sequence which is, comprises or comprises a portion of: GCTGGTGGACATTGGCCGGG (SEQ ID NO: 394).

In some embodiments, an oligonucleotide targets PNPLA3 and has a base sequence which is, comprises or comprises a portion of: GCTGTAGCGAGCCTGGGCG (SEQ ID NO: 395).

In some embodiments, an oligonucleotide targets PNPLA3 and has a base sequence which is, comprises or comprises a portion of: GCTGTGCTTGGCTCCTGCC (SEQ ID NO: 396).

In some embodiments, an oligonucleotide targets PNPLA3 and has a base sequence which is, comprises or comprises a portion of: GGACCCTCTGCACTGGGC (SEQ ID NO: 397).

In some embodiments, an oligonucleotide targets PNPLA3 and has a base sequence which is, comprises or comprises a portion of: GGACCCTCTGCACTGGGCTT (SEQ ID NO: 398).

In some embodiments, an oligonucleotide targets PNPLA3 and has a base sequence which is, comprises or comprises a portion of: GGACCTGAGGATGGACCGC (SEQ ID NO: 399).

In some embodiments, an oligonucleotide targets PNPLA3 and has a base sequence which is, comprises or comprises a portion of: GGACCTGAGGATGGACCGCG (SEQ ID NO: 400).

In some embodiments, an oligonucleotide targets PNPLA3 and has a base sequence which is, comprises or comprises a portion of: GGATAAGGCCACTGTAGAAA (SEQ ID NO: 401).

In some embodiments, an oligonucleotide targets PNPLA3 and has a base sequence which is, comprises or comprises a portion of: GGCATGAAGCAGGAACAT (SEQ ID NO: 402).

In some embodiments, an oligonucleotide targets PNPLA3 and has a base sequence which is, comprises or comprises a portion of: GGCATGAAGCAGGAACATAC (SEQ ID NO: 403).

In some embodiments, an oligonucleotide targets PNPLA3 and has a base sequence which is, comprises or comprises a portion of: GGCCACTGTAGAAAGGCATGAAGCA (SEQ ID NO: 404).

In some embodiments, an oligonucleotide targets PNPLA3 and has a base sequence which is, comprises or comprises a portion of: GGCCACTGTAGAAAGGCATG (SEQ ID NO: 405).

In some embodiments, an oligonucleotide targets PNPLA3 and has a base sequence which is, comprises or comprises a portion of: GGCCACTGTAGAAAGGCA (SEQ ID NO: 406).

In some embodiments, an oligonucleotide targets PNPLA3 and has a base sequence which is, comprises or comprises a portion of: GGCCACTGTAGAAAGGCATGA (SEQ ID NO: 407).

In some embodiments, an oligonucleotide targets PNPLA3 and has a base sequence which is, comprises or comprises a portion of: GGCTGGGATCCTCCACGTCA (SEQ ID NO: 408).

In some embodiments, an oligonucleotide targets PNPLA3 and has a base sequence which is, comprises or comprises a portion of: GGGACCCTCTGCACTGGGCT (SEQ ID NO: 409).

In some embodiments, an oligonucleotide targets PNPLA3 and has a base sequence which is, comprises or comprises a portion of: GGGAGGCCTGTTGGCTGCTC (SEQ ID NO: 410).

In some embodiments, an oligonucleotide targets PNPLA3 and has a base sequence which is, comprises or comprises a portion of: GGGATCCTCCACGTCACAG (SEQ ID NO: 411).

In some embodiments, an oligonucleotide targets PNPLA3 and has a base sequence which is, comprises or comprises a portion of: GGGCGAGAGGGTGTCCAGG (SEQ ID NO: 412).

In some embodiments, an oligonucleotide targets PNPLA3 and has a base sequence which is, comprises or comprises a portion of: GGGTGGCCTCTGCTTTGGTC (SEQ ID NO: 413).

In some embodiments, an oligonucleotide targets PNPLA3 and has a base sequence which is, comprises or comprises a portion of: GGTCTCTGCTGGACAGCCCT (SEQ ID NO: 414).

In some embodiments, an oligonucleotide targets PNPLA3 and has a base sequence which is, comprises or comprises a portion of: GGTGGACATTGGCCGGGAG (SEQ ID NO: 415).

In some embodiments, an oligonucleotide targets PNPLA3 and has a base sequence which is, comprises or comprises a portion of: GGTGGCCTCTGCTTTGGTCT (SEQ ID NO: 416).

In some embodiments, an oligonucleotide targets PNPLA3 and has a base sequence which is, comprises or comprises a portion of: GTAGAAAGGCATGAAGCAG (SEQ ID NO: 417).

In some embodiments, an oligonucleotide targets PNPLA3 and has a base sequence which is, comprises or comprises a portion of: GTAGAAAGGCATGAAGCAGGAACA (SEQ ID NO: 418).

In some embodiments, an oligonucleotide targets PNPLA3 and has a base sequence which is, comprises or comprises a portion of: GTAGAAAGGCATGAAGCAGGAACAT (SEQ ID NO: 419).

In some embodiments, an oligonucleotide targets PNPLA3 and has a base sequence which is, comprises or comprises a portion of: GTAGAAAGGCATGAAGC (SEQ ID NO: 420).

In some embodiments, an oligonucleotide targets PNPLA3 and has a base sequence which is, comprises or comprises a portion of: GTAGAAAGGCATGAAGCA (SEQ ID NO: 421).

In some embodiments, an oligonucleotide targets PNPLA3 and has a base sequence which is, comprises or comprises a portion of: GTAGAAAGGCATGAAGCAG (SEQ ID NO: 422).

In some embodiments, an oligonucleotide targets PNPLA3 and has a base sequence which is, comprises or comprises a portion of: GTAGAAAGGCATGAAGCAGG (SEQ ID NO: 423).

In some embodiments, an oligonucleotide targets PNPLA3 and has a base sequence which is, comprises or comprises a portion of: GTCACTGTAGAAAGGCATGA (SEQ ID NO: 424).

In some embodiments, an oligonucleotide targets PNPLA3 and has a base sequence which is, comprises or comprises a portion of: GTGCTTGGCTCCTGCCTGG (SEQ ID NO: 425).

In some embodiments, an oligonucleotide targets PNPLA3 and has a base sequence which is, comprises or comprises a portion of: GTGCTTGGCTCCTGCCTGGG (SEQ ID NO: 426).

In some embodiments, an oligonucleotide targets PNPLA3 and has a base sequence which is, comprises or comprises a portion of: GTTACCCCCGCCATGGAGA (SEQ ID NO: 427).

In some embodiments, an oligonucleotide targets PNPLA3 and has a base sequence which is, comprises or comprises a portion of: GTTCCGACTCCTGGCCTTC (SEQ ID NO: 428).

In some embodiments, an oligonucleotide targets PNPLA3 and has a base sequence which is, comprises or comprises a portion of: GTTCCGACTCCTGGCCTTCC (SEQ ID NO: 429).

In some embodiments, an oligonucleotide targets PNPLA3 and has a base sequence which is, comprises or comprises a portion of: TACCCCCGCCATGGAGACG (SEQ ID NO: 430).

In some embodiments, an oligonucleotide targets PNPLA3 and has a base sequence which is, comprises or comprises a portion of: TCCGACTCCTGGCCTTCCG (SEQ ID NO: 431).

In some embodiments, an oligonucleotide targets PNPLA3 and has a base sequence which is, comprises or comprises a portion of: TGTTACCCCCGCCATGG (SEQ ID NO: 432).

In some embodiments, an oligonucleotide targets PNPLA3 and has a base sequence which is, comprises or comprises a portion of: TGTTACCCCCGCCATGGAG (SEQ ID NO: 433).

In some embodiments, the present disclosure discloses an oligonucleotide of a sequence recited herein. In some embodiments, the present disclosure discloses an oligonucleotide of a sequence recited herein, wherein the oligonucleotide is capable of directing a decrease in the expression and/or level of a target gene or its gene product. In some embodiments, an oligonucleotide of a recited sequence is a single-stranded RNAi agent. In some embodiments, an oligonucleotide of a recited sequence is an antisense oligonucleotide which directs RNase H-mediated knockdown. In some embodiments, an oligonucleotide of a recited sequence directs both RNA interference and RNase H-mediated knockdown. In some embodiments, an oligonucleotide of a recited sequence comprises any structure described herein (e.g., any 5′-end structure, 5′-end region, 5′ nucleotide moiety, seed region, post-seed region, 3′-terminal dinucleotide, 3′-end cap, or any portion of any of these structures, or any chemistry, stereochemistry, additional chemical moiety, etc., described herein). If the oligonucleotide is a ssRNAi agent, the sequence can be preceded by a T (as a non-limiting example, a 2′-deoxy T, 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, or 5′-(S)-PH T) or the first nucleobase is replaced by a T (as a non-limiting example, a 2′-deoxy T, 5′-(R)-Me OH T, 5′-(R)-Me PO T, 5′-(R)-Me PS T, 5′-(R)-Me PH T, 5′-(S)-Me OH T, 5′-(S)-Me PO T, 5′-(S)-Me PS T, or 5′-(S)-PH T) and/or followed by a 3′-terminal dinucleotide (e.g., as non-limiting examples: TT, UU, TU, etc.). In various sequences, U can be replaced by T or vice versa, or a sequence can comprise a mixture of U and T. In some embodiments, an oligonucleotide has a length of no more than about 49, 45, 40, 30, 35, 25, 23 total nucleotides. In some embodiments, a portion is a span of at least 15, 16, 17, 18, 19, 20, 21, 22, 23, 24, or 25 total nucleotides with 0-3 mismatches. In some embodiments, a portion is a span of at least 15, 16, 17, 18, 19, 20, 21, 22, 23, 24, or 25 total nucleotides with 0-3 mismatches, wherein a span with 0 mismatches is complementary and a span with 1 or more mismatches is a non-limiting example of substantial complementarity. In some embodiments, wherein the sequence recited above starts with a U at the 5′-end, the U can be deleted and/or replaced by another base. In some embodiments, the disclosure encompasses any oligonucleotide having a base sequence which is or comprises or comprises a portion of the base sequence of any oligonucleotide disclosed herein, which has a format or a portion of a format disclosed herein.

In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence described herein. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence or portion thereof, described herein. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence or portion thereof, described herein, wherein a portion is a span of 15 contiguous bases, or a span of 15 contiguous bases with 1-5 mismatches. In some embodiments, an oligonucleotide, an oligonucleotide that directs RNA interference, an oligonucleotide that directs RNase H-mediated knockdown, or an oligonucleotide that directs both RNA interference and RNase H-mediated knockdown can comprise any base sequence or portion thereof described herein in combination with any other structural element or modification described herein, including but not limited to, sugar, base; internucleotidic linkage; stereochemistry or pattern thereof; additional chemical moiety, including but not limited to, a targeting moiety, lipid moiety, a GalNAc moiety, etc.; 5′-end structure; 5′-end region; 5′ nucleotide moiety; seed region; post-seed region; 3′-end region; 3′-terminal dinucleotide; 3′-end cap; GC content; long GC stretch; pattern of modifications of sugars, bases or internucleotidic linkages; format or any structural element thereof, and/or any other structural element or modification described herein; and in some embodiments, the present disclosure pertains to multimers of any such oligonucleotides. Non-limiting examples of oligonucleotides having various base sequences are disclosed in Table 1A, below.

Lengthy table referenced here
US20240132894A1-20240425-T00001
Please refer to the end of the specification for access instructions.

The disclosure notes that some sequences, due to their length, are divided into multiple lines; however, these sequences, as are all oligonucleotides in Table 1A, are single-stranded (unless otherwise noted).

Moieties and Modifications Listed in the Tables (or Compounds Used to Construct Oligonucleotides Comprising these Moieties or Modifications:

| | |
|---|---|
| IT | 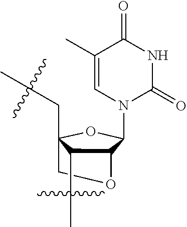 [SMILES: *C[C@]12CO[C@@H](C1*)[C@H](n1cc(C)c(=O)[nH]c1=O)O2] |
| | if between 5′-end groups and/or internucleotidic linkages (e.g., in WV-3819); |
| | 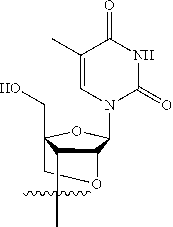 [SMILES: *C1[C@@H]2OC[C@]1(CO)O[C@H]2n1cc(C)c(=O)[nH]c1=O] |
| | if at 5′-end and without 5′-end groups (e.g., in WV-3818); |
| | 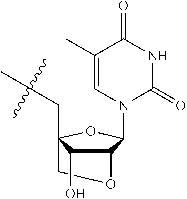 [SMILES: *C[C@]12CO[C@@H](C1O)[C@H](n1cc(C)c(=O)[nH]c1=O)O2] |
| | if at 3′-end (e.g., in WV-7821). |
| IG | 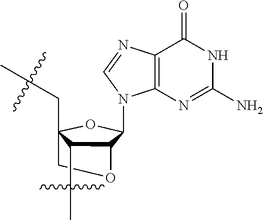 [SMILES: *C[C@]12CO[C@@H](C1*)[C@H](n1cnc3c(=O)[nH]c(N)nc31)O2] |
| | if between 5′-end groups and/or internucleotidic linkages (e.g., in WV-6689); |
| | 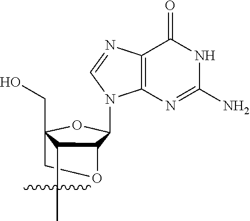 [SMILES: *C1[C@@H]2OC[C@]1(CO)O[C@H]2n1cnc2c(=O)[nH]c(N)nc21] |
| | if at 5′-end and without 5′-end groups (e.g., in WV-6711); |
| | 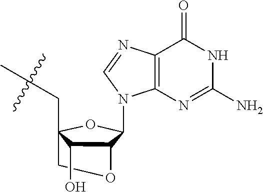 [SMILES: *C[C@]12CO[C@@H](C1O)[C@H](n1cnc3c(=O)[nH]c(N)nc31)O2] |
| | if at 3′-end (e.g., in WV-7827). |
| IA | 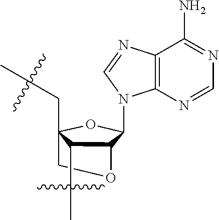 [SMILES: *C[C@]12CO[C@@H](C1*)[C@H](n1cnc3c(N)ncnc31)O2] |
| | if between 5′-end groups and/or internucleotidic linkages (e.g., in WV-6692); |
| | 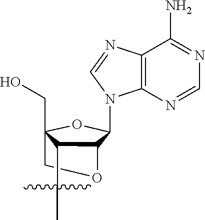 [SMILES: *C1[C@@H]2OC[C@]1(CO)O[C@H]2n1cnc2c(N)ncnc21] |
| | if at 5′-end and without 5′-end groups (e.g., in WV-6710); |
| | 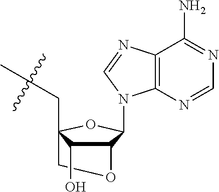 [SMILES: *C[C@]12CO[C@@H](C1O)[C@H](n1cnc3c(N)ncnc31)O2] |
| | if at 3′-end (e.g., in WV-7817). |
| Im5C | 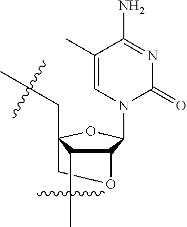 [SMILES: *C[C@]12CO[C@@H](C1*)[C@H](n1cc(C)c(N)nc1=O)O2] |
| | if between 5′-end groups and/or internucleotidic linkages (e.g., in WV-6690); |
| | 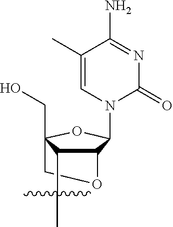 [SMILES: *C1[C@@H]2OC[C@]1(CO)O[C@H]2n1cc(C)c(N)nc1=O] |
| | if at 5′-end and without 5′-end groups (e.g., in WV-7817); |
| | 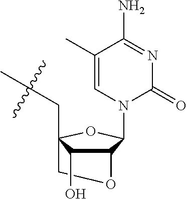 [SMILES: *C[C@]12CO[C@@H](C1O)[C@H](n1cc(C)c(N)nc1=O)O2] |
| | if at 3′-end (e.g., in WV-7818). |
| MeOT | 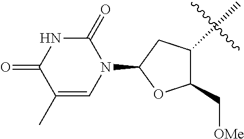 [SMILES: COC[C@H]1O[C@@H](n2cc(C)c(=O)[nH]c2=O)C[C@@H]1C] |
| Mod001 | 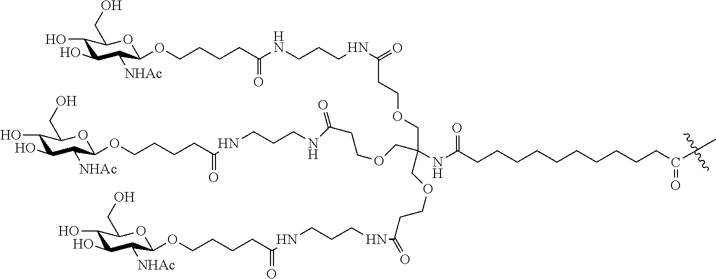 |
| Mod022 | CH₃CH₂CH₂—; connected to 5′-end of oligonucleotide chain through either a phosphate |
| | linkage (O or PO) or phosphorothioate linkage (* if the phosphorothioate not chirally |
| | controlled; can also be Sp if chirally controlled and has an Sp configuration, and Rp if |
| | chirally controlled and has an Rp configuration) as illustrated. |
| PH | 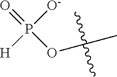 [SMILES: *O[PH](=O)[O-]] |
| Mod023 | 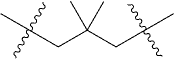 [SMILES: *CC(C)(C)C*] |
| | connected to 5′-end of oligonucleotide chain through either a phosphate |
| | linkage (O or PO) or phosphorothioate linkage (* if the phosphorothioate not chirally |
| | controlled; can also be Sp if chirally controlled and has an Sp configuration, and Rp if |
| | chirally controlled and has an Rp configuration) as illustrated. |
| VPT | 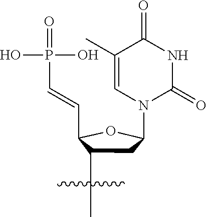 |
| Mod034 | 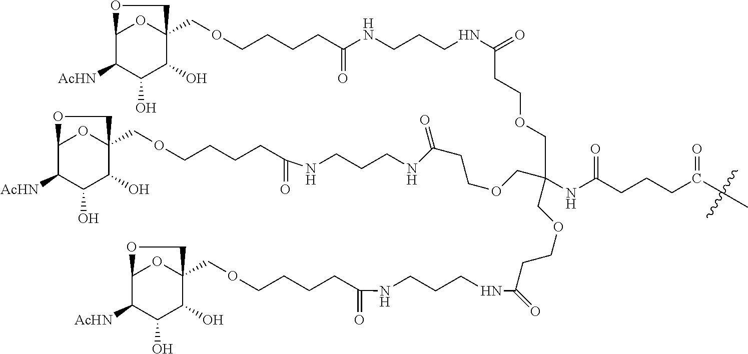 [SMILES: *C(=O)CCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| Mod035 | 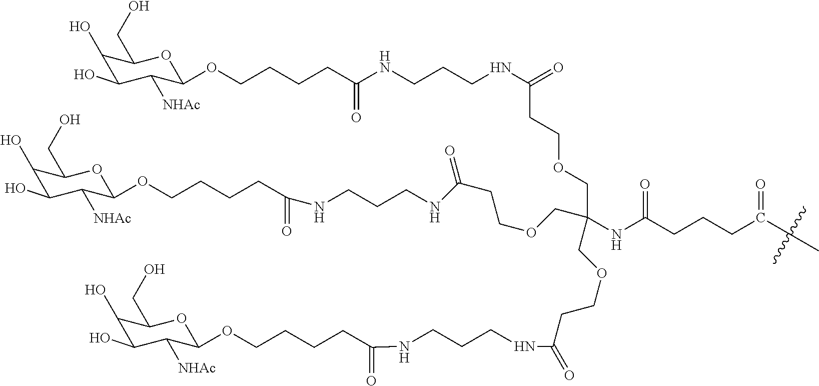 [SMILES: *C(=O)CCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O] |
| Mod036 | 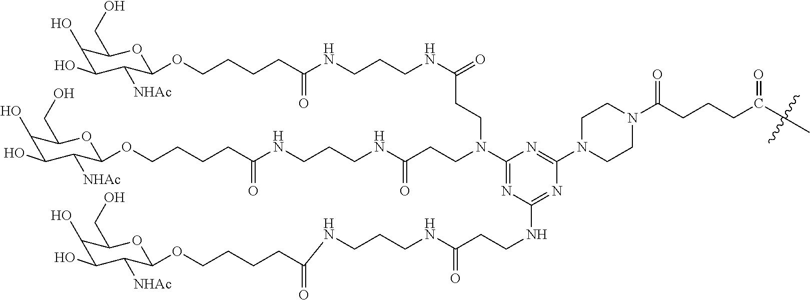 [SMILES: *C(=O)CCCC(=O)N1CCN(c2nc(NCCC(=O)NCCCNC(=O)CCCCOC3OC(CO)C(O)C(O)C3NC(C)=O)nc(N(CCC(=O)NCCCNC(=O)CCCCOC3OC(CO)C(O)C(O)C3NC(C)=O)CCC(=O)NCCCNC(=O)CCCCOC3OC(CO)C(O)C(O)C3NC(C)=O)n2)CC1] |
| Mod038 | 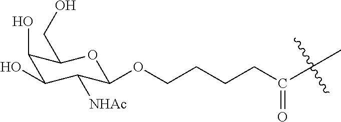 [SMILES: *C(=O)CCCCOC1OC(CO)C(O)C(O)C1NC(C)=O] |
| Mod039 | 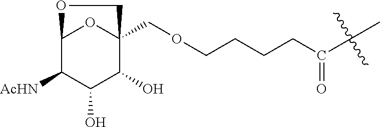 [SMILES: *C(=O)CCCCOC[C@@]12CO[C@@H](O1)[C@H](NC(C)=O)[C@@H](O)[C@H]2O] |
| Mod040 | 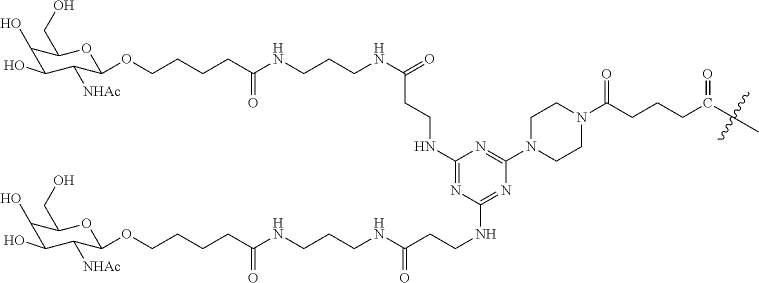 [SMILES: *C(=O)CCCC(=O)N1CCN(c2nc(NCCC(=O)NCCCNC(=O)CCCCOC3OC(CO)C(O)C(O)C3NC(C)=O)nc(NCCC(=O)NCCCNC(=O)CCCCOC3OC(CO)C(O)C(O)C3NC(C)=O)n2)CC1] |
| Mod041 | 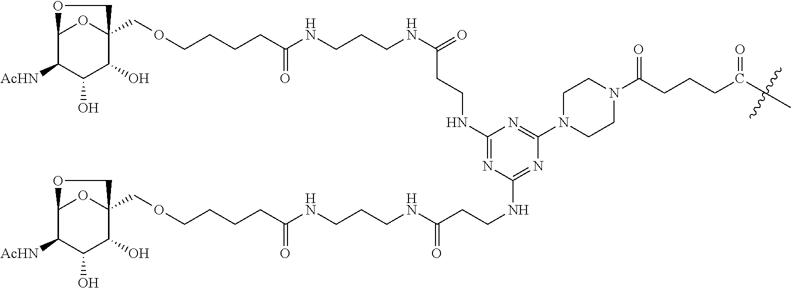 [SMILES: *C(=O)CCCC(=O)N1CCN(c2nc(NCCC(=O)NCCCNC(=O)CCCCOC[C@@]34CO[C@@H](O3)[C@H](NC(C)=O)[C@@H](O)[C@H]4O)nc(NCCC(=O)NCCCNC(=O)CCCCOC[C@@]34CO[C@@H](O3)[C@H](NC(C)=O)[C@@H](O)[C@H]4O)n2)CC1] |
| Mod079 | 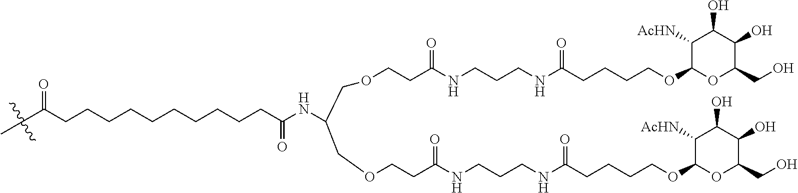 [SMILES: *C(=O)CCCCCCCCCCC(=O)NC(COCCC(=O)NCCCNC(=O)CCCCO[C@@H]1O[C@H](CO)[C@H](O)[C@H](O)[C@H]1NC(C)=O)COCCC(=O)NCCCNC(=O)CCCCO[C@@H]1O[C@H](CO)[C@H](O)[C@H](O)[C@H]1NC(C)=O] |
| Mod080 | 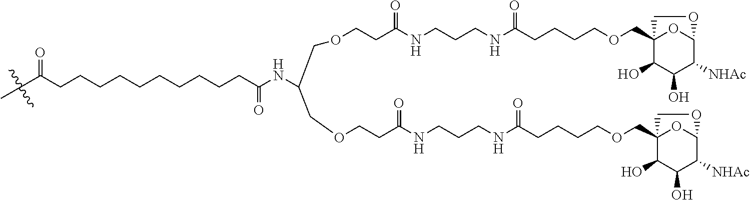 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| Mod081 | 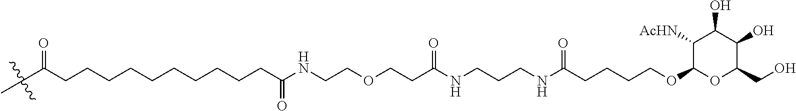 [SMILES: *C(=O)CCCCCCCCCCC(=O)NCCOCCC(=O)NCCCNC(=O)CCCCO[C@@H]1O[C@H](CO)[C@H](O)[C@H](O)[C@H]1NC(C)=O] |
| Mod082 | 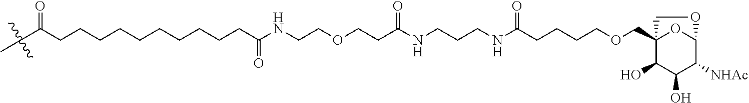 [SMILES: *C(=O)CCCCCCCCCCC(=O)NCCOCCC(=O)NCCCNC(=O)CCCCOC[C@@]12CO[C@@H](O1)[C@H](NC(C)=O)[C@@H](O)[C@H]2O] |
| Mod083 | 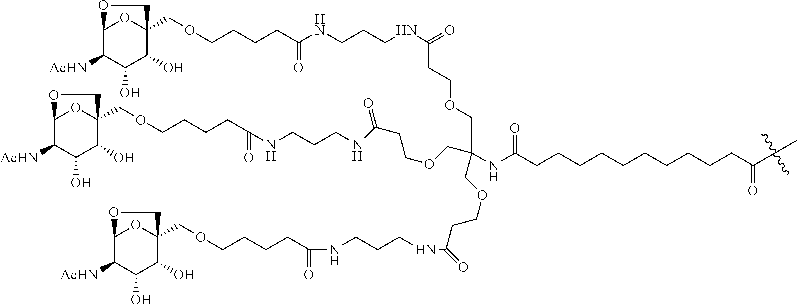 [SMILES: *C(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| 5mp | 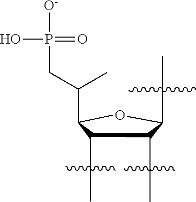 |
| 5MR | 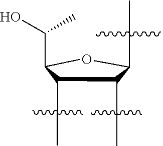 |
| 5mrp | 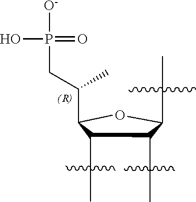 |
| 5MS | 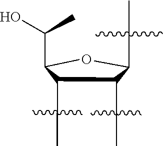 |
| 5msp | 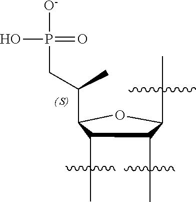 |
| 5mvp | 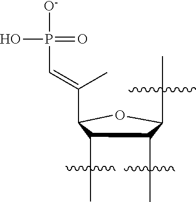 |
| 5pacet | 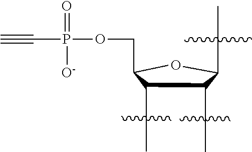 |
| 5ptz | 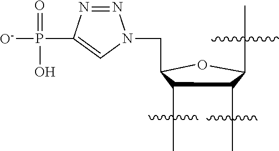 |
| 5tz | 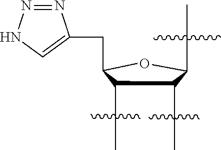 |
| 5tzpo | 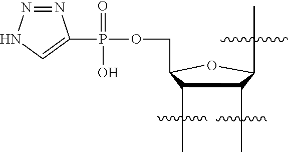 |
| 5mpdT | 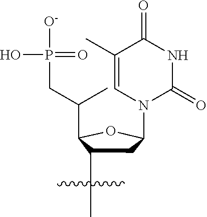 |
| 5MRdT | 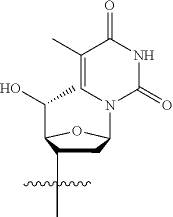 |
| 5mrpdT | 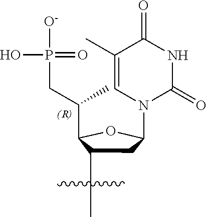 |
| 5MSdT | 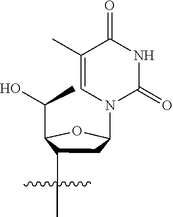 |
| 5mspdT | 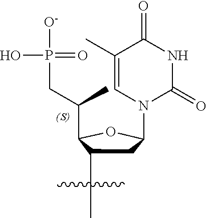 |
| 5mvpdT | 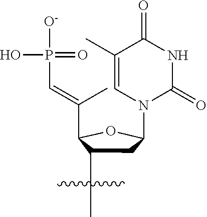 |
| 5pacetdT | 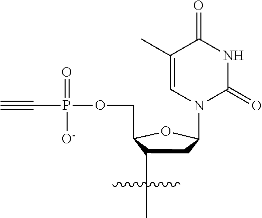 |
| 5ptzdT | 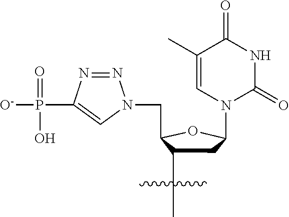 |
| 5tzdT | 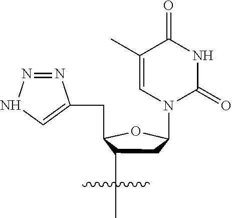 |
| 5tzpodT | 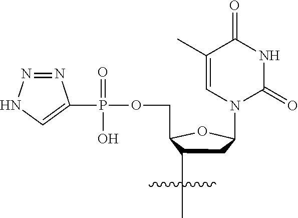 |
| tbclc6T | 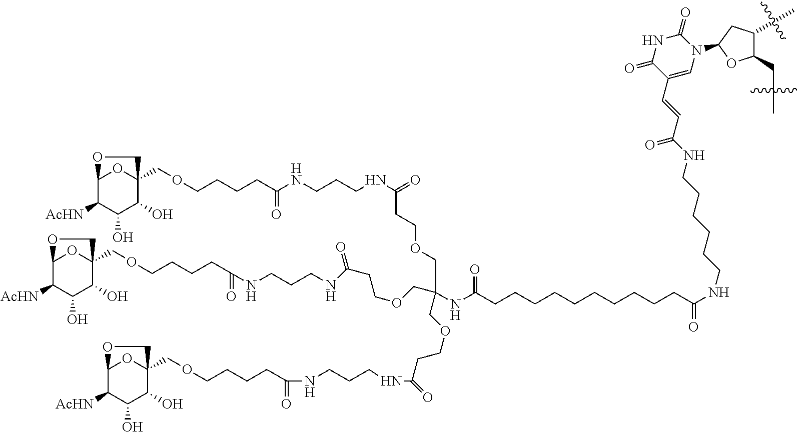 [SMILES: *C[C@H]1O[C@@H](n2cc(/C=C/C(=O)NCCCCCCNC(=O)CCCCCCCCCCC(=O)NC(COCCC(=O)NCCCNC(=O)CCCCOC[C@@]34CO[C@@H](O3)[C@H](NC(C)=O)[C@@H](O)[C@H]4O)(COCCC(=O)NCCCNC(=O)CCCCOC[C@@]34CO[C@@H](O3)[C@H](NC(C)=O)[C@@H](O)[C@H]4O)COCCC(=O)NCCCNC(=O)CCCCOC[C@@]34CO[C@@H](O3)[C@H](NC(C)=O)[C@@H](O)[C@H]4O)c(=O)[nH]c2=O)C[C@@H]1*] |
| bbclc6T | 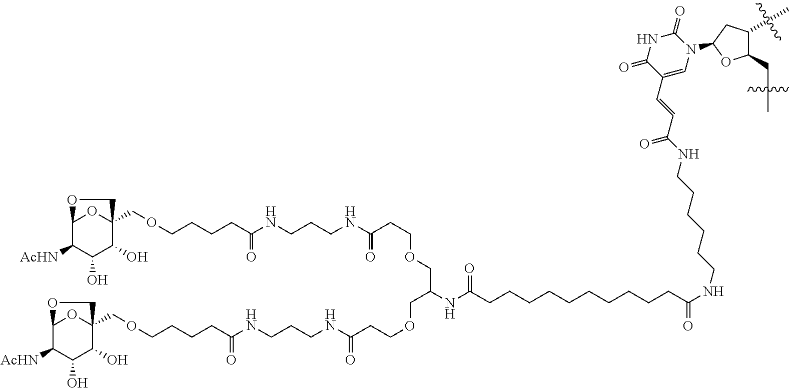 [SMILES: *C[C@H]1O[C@@H](n2cc(/C=C/C(=O)NCCCCCCNC(=O)CCCCCCCCCCC(=O)NC(COCCC(=O)NCCCNC(=O)CCCCOC[C@@]34CO[C@@H](O3)[C@H](NC(C)=O)[C@@H](O)[C@H]4O)COCCC(=O)NCCCNC(=O)CCCCOC[C@@]34CO[C@@H](O3)[C@H](NC(C)=O)[C@@H](O)[C@H]4O)c(=O)[nH]c2=O)C[C@@H]1*] |
| L009 | —CH₂CH₂CH₂—. When L009 is present at the 5′-end of an oligonucleotide |
| | without a Mod, one end of L009 is connected to —OH and the other end connected |
| | to a 5′-carbon of the oligonucleotide chain via a linkage as indicated (e.g., in WV- |
| | 9261, via a stereorandom phosphorothioate linkage (“*”)). |

In some embodiments, a linker, e.g., L009, L010, etc., can replace a sugar, and is bonded on either end to an internucleotidic linkage. For example:

WV-9266 comprises . . . *mAL009*mUfG* . . . , which represents, from 5′ to 3′, a phosphorothioate (*), a sugar which is 2′-OMe (m) attached to a base (A), a phosphodiester linkage (not indicated), a L009 linker (L009), a phosphorothioate (*), a sugar which is 2′-OMe attached to a base which is U (mU), a phosphodiester linkage (not indicated), a sugar which is 2′-F (f) attached to a base (G) and a phosphorothioate.

WV-9267 comprises . . . *mAfC*L009fG* . . . , which represents, from 5′ to 3′, a phosphorothioate (*), a sugar which is 2′-OMe (m) attached to a base (A), a phosphodiester (not indicated), a sugar which is 2′-F (f) attached to a base (C), a phosphorothioate, a L009 linker (L009), a phosphodiester linkage (not indicated), a sugar which is 2′-F (f) attached to a base (G), and a phosphorothioate.

| | |
|---|---|
| L010 | 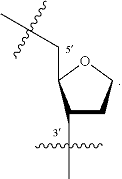 |
| | L010 is connected in the same |
| | fashion as typically in DNA (the 5′- |
| | carbon of a first sugar is connected to |
| | a 3′-carbon of a second sugar via an |
| | internucleotidic linkage, and the 3′-carbon |
| | of the first sugar is connected to the |
| | 5′-carbon of a third sugar via an internucleotidic |
| | linkage). When L010 is present |
| | at the 5′-end of an oligonucleotide without |
| | a Mod, the 5′-carbon of L010 is |
| | connected to —OH and the 3′-carbon |
| | connected to a 5′-carbon of the |
| | oligonucleotide chain via a linkage as |
| | indicated (e.g., in WV-9250, via a |
| | stereorandom phosphorothioate linkage (“*”)). |

In some embodiments, L010 can replace a sugar, and L010 is bonded on either end to an internucleotidic linkage.

| | |
|---|---|
| n001, nX | 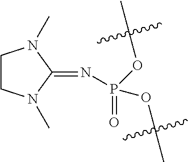 [SMILES: *OP(=O)(N=C1N(C)CCN1C)O*] |
| VPT | 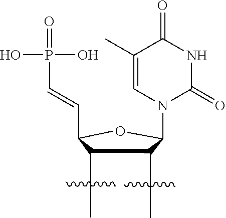 |
| VP | 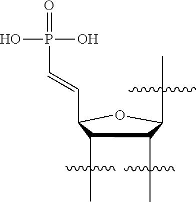 |
| VQ | 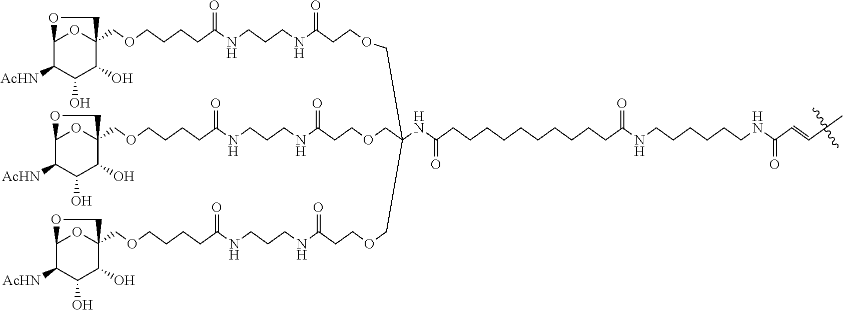 [SMILES: */C=C/C(=O)NCCCCCCNC(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| VR | 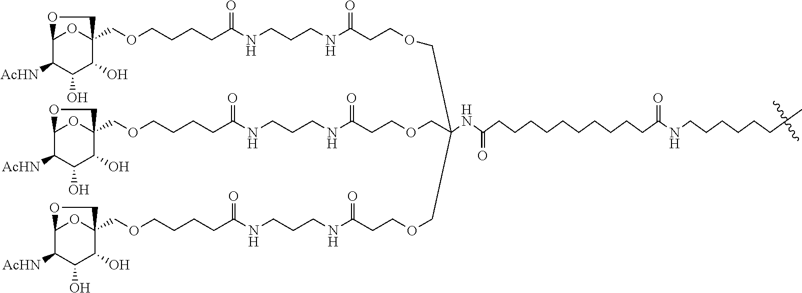 [SMILES: *CCCCCCNC(=O)CCCCCCCCCCC(=O)NC(COCCC(=O)NCCCNC(=O)CCCCOC[C@@]12CO[C@@H](O1)[C@H](NC(C)=O)[C@@H](O)[C@H]2O)(COCCC(=O)NCCCNC(=O)CCCCOC[C@@]12CO[C@@H](O1)[C@H](NC(C)=O)[C@@H](O)[C@H]2O)COCCC(=O)NCCCNC(=O)CCCCOC[C@@]12CO[C@@H](O1)[C@H](NC(C)=O)[C@@H](O)[C@H]2O] |
| VS | 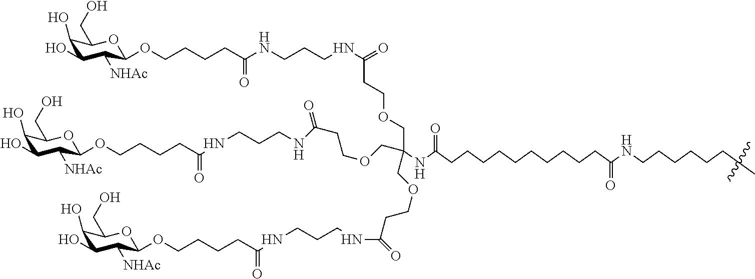 |
| VT | 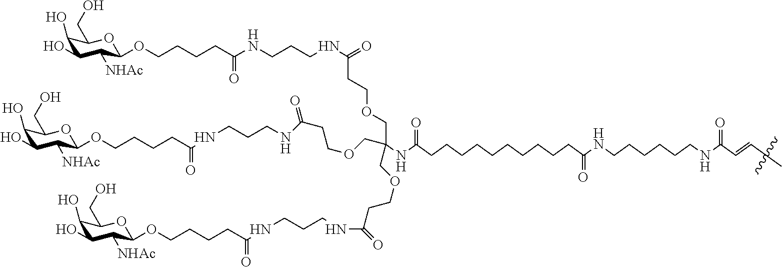 |

### Additional Abbreviations:

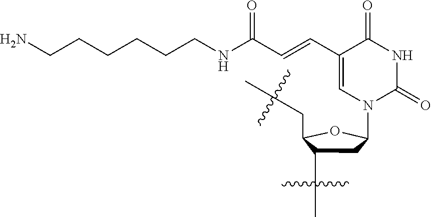

AMC6T:

eo: 2′-MOE

F, f: 2′-F

GaNC6T:

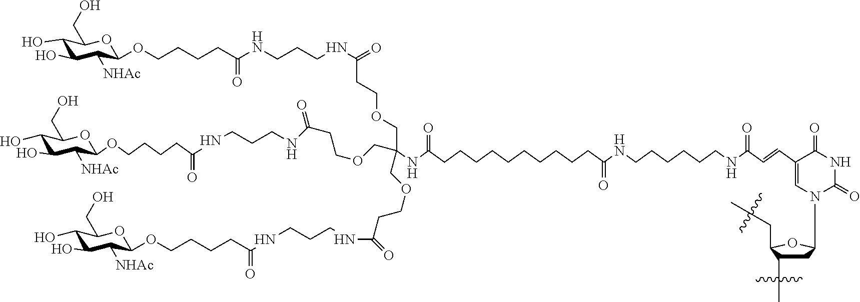

1: 2′-O—CH₂-4′

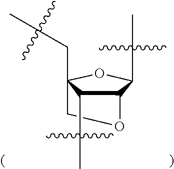 [SMILES: *C[C@]12CO[C@@H](C1*)[C@H](*)O2]

lmU:

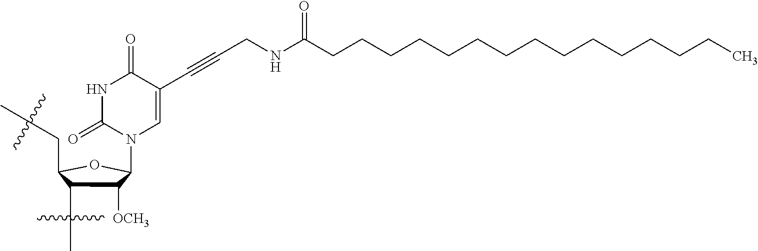

L001: —NH—(CH₂)₆-linker (C6 linker, C6 amine linker or C6 amino linker), connected to Mod, if any (if no Mod, —H, e.g., in WV-8240), through —NH—, and the 5′-end (e.g., in WV-2406) or 3′-end of oligonucleotide chain through either a phosphate linkage (O or PO) or phosphorothioate linkage (* if the phosphorothioate not chirally controlled; can also be Sp if chirally controlled and has an Sp configuration, and Rp if chirally controlled and has an Rp configuration) as illustrated. For example, in WV-2406, L001 is connected to Mod001 through —NH— (forming an amide group —C(O)—NH—), and is connected to the oligonucleotide chain through a phosphate linkage (OXXXXXXXXXXXXXXXXXXX); in WV-2422, L001 is not connected to any Mod, but —H, through —NH—, and is connected to the oligonucleotide chain through a phosphate linkage (OXXXXXXXXXXXXXXXXXXX)

L003:

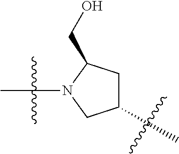 [SMILES: *N1C[C@@H](C)C[C@@H]1CO]

linker, connected to Mod, if any (if no Mod, —H, e.g., in WV-2426), through its amino group, and the 5′-end (e.g., in WV-2407) or 3′-end (e.g., in WV-8070) of oligonucleotide chain through either a phosphate linkage (O or PO) or phosphorothioate linkage (* if the phosphorothioate not chirally controlled; can also be Sp if chirally controlled and has an Sp configuration, and Rp if chirally controlled and has an Rp configuration) as illustrated. For example, in WV-2407, L003 is connected to Mod001 through its amino group (forming an amide group

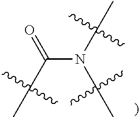 [SMILES: *C(=O)N(*)*]

and is connected to the 5′-end of oligonucleotide chain through a phosphate linkage (OXXXXXXXXXXXXXXXXXXX); in WV-2426, L001 is not connected to any Mod, but —H, through —NH—, and is connected to the oligonucleotide chain through a phosphate linkage (OXXXXXXXXXXXXXXXXXXX); in WV-8070, L003 is connected to Mod001 through its amino group (forming an amide group

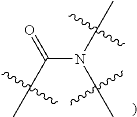 [SMILES: *C(=O)N(*)*]

and is connected to the 3′-end of oligonucleotide chain through a phosphate linkage ( . . . XXXXXXXXXXXXXXXXXXXO)
m: 2′-OMe
m5: methyl at 5-position of C (nucleobase is 5-methylcytosine)
m5Ceo: 5-methyl 2′-methoxyethyl C
OMe: 2′-OMe
O, PO: phoshodiester (phosphate); can be an end group (typically “PO”; for example in WV-4260: POT*fC* . . . ), or a linkage, e.g., a linkage
between linker and oligonucleotide chain, an internucleotidic linkage, etc.
*, PS: Phosphorothioate; can be an end group (typically “PS”, for example, in WV-2653: PST*fA* . . . ), or a linkage, e.g., a linkage between
linker and oligonucleotide chain, an internucleotidic linkage, etc.
R, Rp: Phosphorothioate in Rp conformation
S, Sp: Phosphorothioate in Sp conformation
X: Stereorandom phosphorothioate

In some embodiments, a provided oligonucleotide comprises one or more moieties and/or modifications listed in the Tables, e.g., those described above (base modifications, sugar modifications, modified internucleotidic linkages, linkers, 5′-end groups, additional moieties (e.g., targeting moieties, carbohydrate-containing moieties, etc.), etc), and optionally and independently a linkage/stereochemistry pattern listed in the Tables (e.g., a part of Table 1A). In some embodiments, a provided oligonucleotide comprises a linkage/stereochemistry pattern in the Tables, and optionally and independently one or more moieties and/or modifications listed in the Tables. In some embodiments, a provided oligonucleotide composition is a composition of an oligonucleotide listed in Table 1A. In some embodiments, a provided oligonucleotide composition is a single-stranded RNAi agent listed in Table 1A or otherwise described herein. In some embodiments, example properties of provided oligonucleotides were demonstrated.

In some embodiments, a provided oligonucleotide has a structure of any of formats illustrated in FIG. 1.

In some embodiment, the present disclosure provides a composition comprising a single-stranded oligonucleotide capable of knocking down a target gene, wherein the oligonucleotide has or comprises the structure of:

5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{mz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′
or a salt thereof,
wherein:
each of N1 to N27 is independently a nucleoside;
each of PX0 to PX13 independently has the structure of an internucleotidic linkage;
mz to wz are independently 0 to 10;
wherein -(N26-PX26-N27-PX27)_{yz}- is a 3′-terminal dinucleotide, and (CAP)_{zz} is a 3′-end cap, and yz and zz are 0 or 1.

In some embodiment, the present disclosure provides a composition comprising an oligonucleotide, e.g., a single-stranded oligonucleotide capable of knocking down a target gene, wherein the oligonucleotide has or comprises the structure of:

5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{mz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′
or a salt thereof,
wherein:
each of N1 to N27 is independently a nucleoside residue;
each of PX1 to PX26 is independently an internucleotidic linkage;
PX0 is —H, —OH, -L^{PX}-H, or -L-H;
L^{PX} has the structure of an internucleotidic linkage;
L is as described in the present disclosure;
PX27 is —H, —OH, -L^{PX}-H, or -L-H when zz is —O—, and is —O—, -L^{PX}-, or -L- when zz is not 0;
mz, nz, pz, rz, sz, tz, vz, and wz are independently 0 to 10;
wherein N26-PX26-N27-PX27 is 3′-terminal dinucleotide;
CAP is a 3′-end cap;
yz and zz are independently 0 or 1.

In some embodiments, either yz=1 and _{zz}=0; or _{yz}=0 and zz=1.

In some embodiments, each of _{mz, nz} and _{pz} is an integer from 0 to 10, and the total of _{mz}+_{nz}+_{pz} is an integer from 8 to 20.

In some embodiments, the oligonucleotide comprises at least 15 total bases.

In some embodiments, (a) at least one of _{mz, nz} or _{pz} is 1 or more; and at least one of PX9, PX10 or PX11 is a phosphodiester; (b) at least one of _{mz, nz} or _{pz} is 1 or more; and at least one of PX9, PX10 or PX11 is a phosphorothioate; (c) N13 is a nucleoside which is an abasic nucleoside and/or is 2′-modified; and/or (d) N12 is a 2′-deoxy nucleoside.

In some embodiments, knocking down a target gene comprises the step of decreasing the expression, stability and/or activity of the target gene or its gene product.

In some embodiments, knocking down a target gene comprises the step of decreasing the expression, stability and/or activity of the target gene mRNA.

In some embodiments, knocking down a target gene comprises the step of decreasing the expression, stability and/or activity of the target gene mRNA via RNA interference.

In some embodiments, knocking down a target gene comprises the step of decreasing the expression, stability and/or activity of the target gene mRNA via RNase H-mediated knockdown.

In some embodiments, knocking down a target gene comprises the step of decreasing the expression, stability and/or activity of the target gene mRNA via RNA interference and/or RNase H-mediated knockdown.

In some embodiments, a provided single-stranded oligonucleotide capable of directing RNA interference, wherein the oligonucleotide has or comprises the structure of:

5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′
wherein: the oligonucleotide comprises multiple nucleosides (each independently represented by any of N1 to N27) and multiple internucleotidic linkages (each independently represented by any of PX0 to PX12). In some embodiments, an internucleotidic linkage is a modified internucleotidic linkage. In some embodiments, a provided single-stranded RNAi agent comprises a 3′-terminal dinucleotide (N26-PX26-N27-PX27)_{yz}, and does not comprise a 3′-end cap, wherein yz=1 and zz=0. In some embodiments, a provided single-stranded RNAi agent comprises a 3′-end cap (CAP)_{zz}, and does not comprise a 3′-terminal dinucleotide (N26-PX26-N27-PX27)_{yz}, and yz=0 and zz=1.

In some embodiments, any of PX0 to PX13 can be an internucleotidic linkage independently selected from: phosphodiester, phosphorothioate and phosphorodithioate, or any modified or variant of an internucleotidic linkage described herein or known in the art. In some embodiments, a phosphorothioate is random (wherein an oligonucleotide composition comprises both oligonucleotides with Rp and Sp chirality at the phosphorothioate this position). In some embodiments, a phosphorothioate is chirally controlled (wherein an oligonucleotide composition comprises oligonucleotides wherein a plurality or majority of the oligonucleotides have a Sp chirality at the phosphorothioate at this position, or a plurality or majority of the oligonucleotides have a Rp chirality at the phosphorothioate at this position.

In some embodiments, 1 or more of PX0 to PX13 are a Sp phosphorothioate. In some embodiments, 2 or more of PX0 to PX13 are a Sp phosphorothioate. In some embodiments, 3 or more of PX0 to PX13 are a Sp phosphorothioate. In some embodiments, 4 or more of PX0 to PX13 are a Sp phosphorothioate. In some embodiments, 5 or more of PX0 to PX13 are a Sp phosphorothioate. In some embodiments, 6 or more of PX0 to PX13 are a Sp phosphorothioate. In some embodiments, 7 or more of PX0 to PX13 are a Sp phosphorothioate. In some embodiments, 8 or more of PX0 to PX13 are a Sp phosphorothioate. In some embodiments, 9 or more of PX0 to PX13 are a Sp phosphorothioate. In some embodiments, 10 or more of PX0 to PX13 are a Sp phosphorothioate. In some embodiments, 11 or more of PX0 to PX13 are a Sp phosphorothioate. In some embodiments, 12 or more of PX0 to PX13 are a Sp phosphorothioate. In some embodiments, 13 or more of PX0 to PX13 are a Sp phosphorothioate. In some embodiments, 14 or more of PX0 to PX13 are a Sp phosphorothioate. In some embodiments, 15 or more of PX0 to PX13 are a Sp phosphorothioate. In some embodiments, 16 or more of PX0 to PX13 are a Sp phosphorothioate. In some embodiments, 17 or more of PX0 to PX13 are a Sp phosphorothioate. In some embodiments, 18 or more of PX0 to PX13 are a Sp phosphorothioate. In some embodiments, 19 or more of PX0 to PX13 are a Sp phosphorothioate. In some embodiments, 20 or more of PX0 to PX13 are a Sp phosphorothioate. In some embodiments, 21 or more of PX0 to PX13 are a Sp phosphorothioate. In some embodiments, 2 or more of PX0 to PX13 are a Rp phosphorothioate. In some embodiments, 3 or more of PX0 to PX13 are a Rp phosphorothioate. In some embodiments, 4 or more of PX0 to PX13 are a Rp phosphorothioate. In some embodiments, 5 or more of PX0 to PX13 are a Rp phosphorothioate. In some embodiments, 6 or more of PX0 to PX13 are a Rp phosphorothioate. In some embodiments, 7 or more of PX0 to PX13 are a Rp phosphorothioate. In some embodiments, 8 or more of PX0 to PX13 are a Rp phosphorothioate. In some embodiments, 9 or more of PX0 to PX13 are a Rp phosphorothioate. In some embodiments, 10 or more of PX0 to PX13 are a Rp phosphorothioate. In some embodiments, 11 or more of PX0 to PX13 are a Rp phosphorothioate. In some embodiments, 12 or more of PX0 to PX13 are a Rp phosphorothioate. In some embodiments, 13 or more of PX0 to PX13 are a Rp phosphorothioate. In some embodiments, 14 or more of PX0 to PX13 are a Rp phosphorothioate. In some embodiments, 15 or more of PX0 to PX13 are a Rp phosphorothioate. In some embodiments, 16 or more of PX0 to PX13 are a Rp phosphorothioate. In some embodiments, 17 or more of PX0 to PX13 are a Rp phosphorothioate. In some embodiments, 18 or more of PX0 to PX13 are a Rp phosphorothioate. In some embodiments, 19 or more of PX0 to PX13 are a Rp phosphorothioate. In some embodiments, 20 or more of PX0 to PX13 are a Rp phosphorothioate. In some embodiments, 21 or more of PX0 to PX13 are a Rp phosphorothioate.

In some embodiments, each of N1 to N27 are independently a nucleoside. In some embodiments, a nucleoside is a 2′-modified nucleoside or an abasic nucleoside (which comprises a sugar but lacks a nucleobase).

In some embodiments, 1 or more of N1 to N27 is an abasic nucleoside. In some embodiments, 1 or more of N1 to N27 is an 2′-deoxy nucleoside (DNA). 2 or more of N1 to N27 is an 2′-deoxy nucleoside (DNA). In some embodiments, 1 or more of N1 to N27 is a 2′-modified nucleoside. In some embodiments, 5 or more of N1 to N27 is a 2′-modified nucleoside. In some embodiments, 10 or more of N1 to N27 is a 2′-modified nucleoside. In some embodiments, 15 or more of N1 to N27 is a 2′-modified nucleoside. In some embodiments, 20 or more of N1 to N27 is a 2′-modified nucleoside. In some embodiments, 1 or more of N1 to N27 is a 2′-F nucleoside. In some embodiments, 5 or more of N1 to N27 is a 2′-F nucleoside. In some embodiments, 10 or more of N1 to N27 is a 2′-F nucleoside. In some embodiments, 15 or more of N1 to N27 is a 2′-F nucleoside. In some embodiments, 20 or more of N1 to N27 is a 2′-F nucleoside. In some embodiments, 21 or more of N1 to N27 is a 2′-F nucleoside. In some embodiments, 1 or more of N1 to N27 is a 2′-OMe nucleoside. In some embodiments, 10 or more of N1 to N27 is a 2′-OMe nucleoside.

Various examples of single-stranded RNAi agents of the structure of 5′-PX0-N1-PX1-N2-PX2-N3-PX3-N4-PX4-N5-PX5-N6-PX6-N7-PX7-N8-PX8-N9-PX9-N10-PX10-N11-PX11-N12-PX12-N13-PX13-N14-PX14-N15-PX15-N16-PX16-N17-PX17-(N18-PX18)_{mz}-(N19-PX19)_{nz}-(N20-PX20)_{pz}-(N21-PX21)_{rz}-(N22-PX22)_{sz}-(N23-PX23)_{tz}-(N24-PX24)_{vz}-(N25-PX25)_{wz}-(N26-PX26-N27-PX27)_{yz}-(CAP)_{zz}-3′ are disclosed herein, for example, in Table 1A and in the Figures and Tables. Various non-limiting examples of these single-stranded RNAi agents, and various structural elements, are described below and herein. These structural elements include, but are not limited to, the 5′-end structure, 5′-end region, the seed region, the target sequence, and length, the 3′-end region, and optional moieties conjugated to the single-stranded RNAi agent.

The present disclosure presents many non-limiting examples of oligonucleotides capable of mediating single-stranded RNA interference (e.g., single-stranded RNAi agents). Experimental data (not shown) demonstrated that various putative single-stranded RNAi agents were, in fact, capable of mediating RNA interference. In some experiments, an in vitro Ago-2 assay was used, including the use of a RNA test substrate WV-2372 (APOC3). The band representing the RNA test substrate is absent in the presence of oligonucleotides WV-1308 and WV-2420, indicating that these oligonucleotides are single-stranded RNAi agents capable of mediating RNA interference. The remaining lanes are controls: Substrate in the absence of negative control ASO WV-2134; substrate in the presence of negative control ASO WV-2134, which does not mediate RNA interference; substrate in the absence of test oligonucleotide WV-1308; substrate in the absence of test oligonucleotide WV-2420; substrate alone; no substrate, with added WV-2134; and no substrate, with added WV-1308. Also performed (data not shown) was an in vitro Ago-2 assay, using a APOC3 mRNA as a test substrate in a 3′ RACE assay in Hep3B cells. A cleavage product of the APOC3 mRNA in the presence of test oligonucleotide WV-3021 was detected, the product corresponding to cleavage of the mRNA at a site corresponding to a cut between positions 10 and 11 of WV-3021. An artifactual cleavage product was also detected. Experimental data (not shown) demonstrated that dual mechanism oligonucleotide WV-2111 is capable of mediating knockdown by both RNase H and RNA interference. In an experiment, several oligonucleotides were capable of mediating RNA interference. The RNA test substrate was WV-2372. The experiment showed disappearance of the RNA test substrate in the presence of test oligonucleotides WV-1308; WV-2114; WV-2386; and WV-2387, indicating that all these oligonucleotides are capable of acting as single-stranded RNAi agents mediating RNA interference. The remaining lanes are controls. Thus, the experiment showed that oligonucleotides WV-1308, WV-2114, WV-2386, and WV-2387 were all able to mediate RNA interference. Thus, the experiments showed that several single-stranded RNAi agents (e.g., WV-1308, WV-2420, WV-3021, WV-2111, WV-2114, WV-2386, and WV-2387) are capable of mediating RNA interference. The present disclosure presents many non-limiting examples of oligonucleotides, having any of various sequences, formats, modifications, 5′-end regions, seed regions, post-seed regions, and 3′-end regions, and which are capable of mediating single-stranded RNA interference (e.g., single-stranded RNAi agents).

Formats of Oligonucleotides

In some embodiments, provided oligonucleotides are capable of directing a decrease in the expression and/or level of a target gene or its gene product. In some embodiments, provided oligonucleotides are capable of directing a decrease in the expression and/or level of a target gene or its gene product via RNA interference. In some embodiments, provided oligonucleotides are capable of directing a decrease in the expression and/or level of a target gene or its gene product via a biochemical mechanism which does not involve RNA interference or RISC (including, but not limited to, RNaseH-mediated knockdown or steric hindrance of gene expression). In some embodiments, provided oligonucleotides are capable of directing a decrease in the expression and/or level of a target gene or its gene product via RNA interference and/or RNase H-mediated knockdown. In some embodiments, provided oligonucleotides are capable of directing a decrease in the expression and/or level of a target gene or its gene product by sterically blocking translation after annealing to a target gene mRNA, and/or by altering or interfering with mRNA splicing and/or exon inclusion or exclusion. In some embodiments, provided oligonucleotides can have any format or portion thereof or structural element thereof described herein or known in the art.

In some embodiments, an oligonucleotide can have any format or structural element thereof described herein or known in the art.

In some embodiments, an oligonucleotide capable of directing a decrease in the expression and/or level of a target gene or its gene product can have any format or structural element thereof described herein or known in the art.

LENGTHY TABLES
The patent application contains a lengthy table section. A copy of the table is available in electronic form from the USPTO web site (https://seqdata.uspto.gov/?pageRequest=docDetail&DocID=US20240132894A1). An electronic copy of the table will also be available from the USPTO upon request and payment of the fee set forth in 37 CFR 1.19(b)(3).

1-61. (canceled)

62. A method for reducing level and/or activity of a transcript or a protein encoded thereby, comprising administering to a system expressing the transcript an oligonucleotide composition comprising a plurality of oligonucleotides that are structurally identical, wherein:
the oligonucleotides of the plurality comprise a targeting-binding sequence that is complementary to a target sequence in the transcript;
the oligonucleotides of the plurality are about 10 to 50 nucleotides in length;
at least 5 internucleotidic linkages in each oligonucleotide of the plurality are chirally controlled; and
the oligonucleotides of the plurality each independently comprise a 5′-end group R^{E}, wherein R^{E} is -L-P(O)(XR)₂ or a salt form thereof, wherein each X is independently —O—, —S—, or a covalent bond; wherein L in -L-P(O)(XR)₂ is:
(i) an optionally substituted, linear or branched C₃-C₁₀ aliphatic group;
(ii) an optionally substituted, linear or branched C₁-C₁₀ aliphatic group, wherein one or more methylene unit of L are independently replaced with -Cy-, wherein each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
(iii) —CH(R)—O—, wherein R in —CH(R)—O— is —H or optionally substituted C₁₋₄ aliphatic, wherein O is connected to P; or
(iv) optionally substituted (E)-CH═CH—;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms.

63. The method of claim 62, wherein the level of the oligonucleotides of the plurality is (90%)ⁿ-100% of all oligonucleotides in the composition, wherein n is the number of chirally controlled internucleotidic linkages, and n is 5-25.

64. The method of claim 62, wherein all internucleotidic linkages of each oligonucleotide of the plurality are independently chiral internucleotidic linkages.

65. The method of claim 62, wherein at least 20% of the nucleotidic units of each oligonucleotide of the plurality independently comprise a 2′-substitution.

66. The method of claim 65, wherein each oligonucleotide of the plurality independently comprises a 2′-F modified sugar.

67. The method of claim 65, wherein each oligonucleotide of the plurality independently comprises a 2′-OR¹ modified sugar, wherein R¹ is optionally substituted C₁₋₆ alkyl.

68. The method of claim 65, wherein each oligonucleotide of the plurality independently comprises a modified sugar comprising 2′-L-, wherein L connects C2 and C4 of the modified sugar.

69. The method of claim 62, wherein the target-binding sequence has a length of at least 15 bases, wherein each base is optionally substituted adenine, cytosine, guanosine, thymine, or uracil, and wherein the target sequence comprises one or more allelic sites, wherein an allelic site is a SNP or a mutation.

70. The method of claim 62, wherein the 5′-end sugar of each oligonucleotide of the plurality is 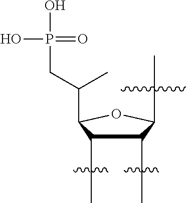
(5mp) or a salt form thereof.

71. The method of claim 62, wherein the 5′-end sugar of each oligonucleotide of the plurality is 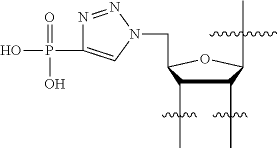
(5ptz) or a salt form thereof wherein at least 50% of the nucleotidic units of each oligonucleotide of the plurality independently comprise a 2′-substitution.

72. The method of claim 62, wherein each oligonucleotide of the plurality independently comprises a phosphorothioate internucleotidic linkage.

73. The method of claim 72, wherein the phosphorothioate internucleotidic linkage is a chirally controlled internucleotidic linkage in the Sp configuration.

74. The method of claim 62, wherein each oligonucleotide of the plurality independently comprises at least 5 internucleotidic linkages in the Sp configuration.

75. The method of claim 62, wherein at least 60% of chirally controlled internucleotidic linkages independently comprise a Sp linkage phosphorus.

76. The method of claim 62, wherein the 5′-end nucleoside of each oligonucleotide of the plurality is 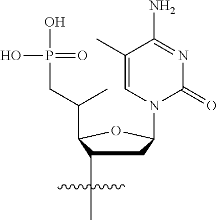
(5mpdT) or a salt form thereof.

77. The method of claim 62, wherein the 5′-end end sugar is 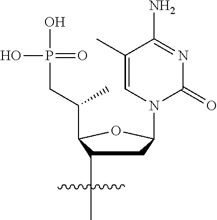
(5mrp) or a salt form thereof.

78. The method of claim 62, wherein the 5′-end end sugar is 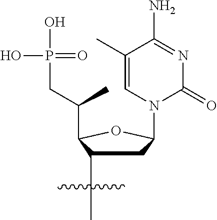
(5msp) or a salt form thereof.

79. A method for allele-specific suppression of a transcript from a nucleic acid sequence for which a plurality of alleles exist within a population, each of which contains a specific nucleotide characteristic sequence element that defines the allele relative to other alleles of the same target nucleic acid sequence, the method comprising steps of:
administering to a subject comprising transcripts of the target nucleic acid sequence an oligonucleotide composition comprising a plurality of oligonucleotides that are structurally identical, wherein:
the oligonucleotides of the plurality are about 10 to 50nucleotides in length;
at least 5 internucleotidic linkages in each oligonucleotide of the plurality are chirally controlled; and
the oligonucleotides of the plurality each independently comprise a 5′-end group R^{E}, wherein R^{E} is -L-P(O)(XR)₂ or a salt form thereof, wherein each X is independently —O—, —S—, or a covalent bond; wherein L in -L-P(O)(XR)₂ is:
(i) an optionally substituted, linear or branched C₃-C₁₀ aliphatic group;
(ii) an optionally substituted, linear or branched C₁-C₁₀ aliphatic group, wherein one or more methylene unit of L are independently replaced with -Cy-, wherein each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
(iii) —CH(R)—O—, wherein R in —CH(R)—O— is —H or optionally substituted C₁₋₄ aliphatic, wherein O is connected to P; or
(iv) optionally substituted (E)-CH═CH—;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms; and
wherein the oligonucleotides of plurality comprise a targeting-binding sequence that is complementary to a target sequence in the nucleic acid sequence, which target sequence comprises a characteristic sequence element that defines a particular allele,
wherein when the composition is administered to a system comprising transcripts of both the target allele and another allele of the same nucleic acid sequence, transcripts of the particular allele are suppressed at a greater level than a level of suppression observed for another allele of the same nucleic acid sequence.

80. A compound having the structure of a formula selected from: 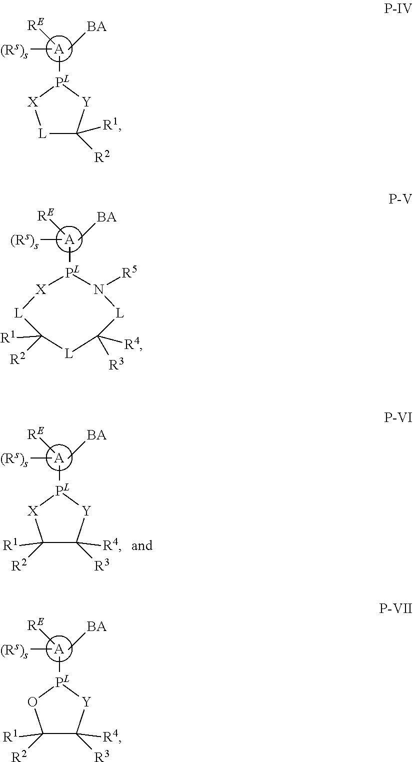
or a salt thereof, wherein:
R^{E} is a 5′-end group;
each BA is independently an optionally substituted group selected from C₁₋₃₀ cycloaliphatic, C₆₋₃₀ aryl, C₅₋₃₀ heteroaryl having 1-10 heteroatoms, C₃₋₃₀ heterocyclyl having 1-10 heteroatoms, a natural nucleobase moiety, and a modified nucleobase moiety;
each R^{s} is independently —F, —Cl, —Br, —I, —CN, —N₃, —NO, —NO₂, -L-R′, -L-OR′, -L-SR′, -L-N(R′)₂, —O-L-OR′, —O-L-SR′, or —O-L-N(R′)₂;
each of R¹, R², R³, R⁴ and R⁵ is independently R^{s};
s is 0-20;
each L is independently a covalent bond, or a bivalent, optionally substituted, linear or branched group selected from a C₁₋₃₀ aliphatic group and a C₁₋₃₀ heteroaliphatic group having 1-10 heteroatoms, wherein one or more methylene units are optionally and independently replaced with C₁₋₆ alkylene, C₁₋₆ alkenylene, —C≡C—, —C(R′)₂—, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, —C(O)O—, —P(O)(OR′)—, —P(O)(SR′)—, —P(O)(R′)—, —P(O)(NR′)—, —P(S)(OR′)—, —P(S)(SR′)—, —P(S)(R′)—, —P(S)(NR′)—, —P(R′)—, —P(OR′)—, —P(SR′)—, —P(NR′)—, —P(OR′)[B(R′)₃]—, —OP(O)(OR′)O—, —OP(O)(SR′)O—, —OP(O)(R′)O—, —OP(O)(NR′)O—, —OP(OR′)O—, —OP(SR′)O—, —OP(NR′)O—, —OP(R′)O—, or —OP(OR′)[B(R′)₃]O—; and one or more carbon atoms are optionally and independently replaced with Cy^{L};
each Cy^{L} is independently an optionally substituted tetravalent group selected from a C₃₋₂₀ cycloaliphatic ring, a C₆₋₂₀ aryl ring, a 5-20 membered heteroaryl ring having 1-10 heteroatoms, and a 3-20 membered heterocyclyl ring having 1-10 heteroatoms;
each Ring A is independently an optionally substituted 3-20 membered monocyclic, bicyclic or polycyclic ring having 0-10 heteroatoms;
P^{L} is P(═W), P, or P→B(R′)₃;
W is —O—, S or Se;
each of X and Y is independently —O—, —S—, —N(-L-R¹)—, or L;
each R′ is independently —R, —C(O)R, —C(O)OR, or —S(O)₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered monocyclic, bicyclic or polycyclic ring having, in addition to the intervening atoms, 0-10 heteroatoms.
